(19) United States
(12) Patent Application Publication
(10) Pub. No.: US 2011/0003711 A1
(43) Pub. Date: Jan. 6, 2011

(54) CELL LINES EXPRESSING GABA RECEPTOR AND METHODS USING THEM

(75) Inventors: Kambiz Shekdar, New York, NY (US); Jessica Langer, Highland Park, NJ (US)

Correspondence Address: Ropes & Gray LLP, Patent Docketing 39/361, 1211 Avenue of the Americas, New York, NY, 10036-8704, US

(73) Assignee: CHROMOCELL CORPORATION, New Brunswick, NJ (US)

(21) Appl. No.: 12/865,497

(22) PCT Filed: Feb. 2, 2009
(86) PCT No.: PCT/US2009/032903

§ 371 Date: Jul. 30, 2010

Related U.S. Application Data
(60) Provisional application No. 61/063,219, filed on Feb. 1, 2008.

(51) Int. Cl. C40B 40/02; C12N 5/10; C12N 15/63; C12Q 1/02; G01N 33/566; C07K 16/28; C07K 2/00

(52) U.S. Cl. 506/14; 435/325; 435/455; 435/29; 435/7.21; 530/387.9; 530/300

(57) ABSTRACT

The invention relates to Gamma-aminobutyric acid receptors (GABA receptors) as well as cells and cell lines stably expressing a GABA receptor. The invention includes cell lines that express various subunit combinations of GABA receptors. The GABA receptor- and GABA receptor subunit-expressing cell lines are highly sensitive, physiologically relevant and produce consistent results. The invention further provides methods of making such cells and cell lines. The GABA receptor- and GABA receptor subunit-expressing cells and cell lines provided herein are useful in identifying modulators of GABA receptors.

### FIELD OF THE INVENTION

The invention relates to Gamma-aminobutyric acid type A receptors (GABA_{A} receptors) as well as cells and cell lines stably expressing a GABA_{A} receptor. The invention includes cell lines that express various subunit combinations of GABA_{A}. The GABA_{A}-expressing cell lines are highly sensitive, physiologically relevant and produce consistent results. The invention further provides methods of making such cells and cell lines. The GABA_{A}-expressing cells and cell lines provided herein are useful in identifying modulators of GABA_{A} receptor.

### BACKGROUND

Gamma-aminobutyric acid (GABA) is the major inhibitory neurotransmitter in the central nervous system (CNS). Indeed, approximately 30% of all synapses use GABA as a transmitter. There are three classes of GABA receptors: GABA_{A} (ligand-gated ion channel), GABA_{B} (G protein-coupled receptor), and GABA_{C} (ligand-gated ion channel). Of these, GABA_{A} receptors that are composed of five subunits are responsible for most of the physiological function of GABA. Chloride flux into the cell resulting from the activation of GABA_{A} receptors hyper-polarizes resting membrane potential and decreases the chances of the post-synaptic neuron propagating an action potential. To date, approximately 19 GABA receptor subunits have been cloned from mammals (6 alpha, 3 beta, 3 gamma, 1 delta, 1 epsilon, 1 theta, 1 pi, and 3 rho subunits). This heterogeneity is further increased by alternate splicing—for example, the two major splice variants of the gamma 2 subunit are termed gamma 2 short and gamma 2 long). In general, GABA_{A} receptors are thought to require 2 alpha subunits, 2 beta subunits and a third “regulatory” subunit (usually gamma or delta).

The GABA_{A} receptors are the targets of a wide range of therapeutic and clinically relevant compounds including benzodiazepines, barbiturates, neuro-steroids, ethanol, certain intravenous anesthetics and more recently developed subtype specific modulators such as zolpidem. These compounds serve as anxiolytics, sedative/hypnotics, anti-epileptics and memory enhancers. Many of these therapeutics cause side effects due to non-specific interactions with other biological pathways. For example, benzodiazepines such as diazepam (Valium) are excellent anxiolytics but cause unwanted sedative effects when used clinically. The binding sites for GABA ligand, its competitive antagonist and benzodiazepines are well understood. However, binding sites for barbiturates are not known, and binding sites for ethanol and neurosteroids are modestly understood.

Specific GABA_{A} subunits are expressed throughout the brain in distinct spatial and developmental patterns and display different responses to known pharmacological modulators. The most abundant subunit combination found in the CNS is alpha 1-beta 2-gamma 2. This subtype represents approximately 40% of GABA_{A} receptors in the brain and it is expressed throughout the CNS. Alpha 1 containing receptors are believed to be responsible for the sedative effects of benzodiazepines. While alpha 2 and alpha 3, expressed in the hippocampus, thalamus, and other CNS locations, are thought to mediate the anti-anxiety effects of the benzodiazepines, specific modulators of these receptors are still being developed. Alpha 5 containing receptors are expressed in the hippocampus and are thought to play a role in learning and memory. Alpha 4 and alpha 6 containing receptors are insensitive to benzodiazepines and often form channels with the delta subunit. Alpha 4 and delta containing receptors are found in the thalamus and the dentate gyms of the hippocampus, whereas co-expression of alpha 6 and delta subunits is limited to cerebellar locations. The minor “regulatory” subunits epsilon and theta are expressed in particular CNS locations such as the cortex, the substantia nigra, amygdala and hypothalamus whereas another minor subunit, pi, is expressed outside the CNS in the uterus and breast tissue (overexpression of pi has been observed in breast cancer). All of the family members are important clinical targets for managing a variety of conditions. For example, mutations in the GABA_{A} receptors have been linked to a variety of diseases using genetic linkage and gene sequencing approaches. Recent evidence implicates specific GABA subunits such as alpha1, gamma2 and delta in the pathologies of certain monogenetic forms of epilepsy. The GABA_{A} alpha2 and delta subunits have also been implicated in alcohol consumption and addiction. Alpha 3 variant sequences show a possible association with multiple sclerosis while alpha 4 has been linked to autism.

Recent evidence has supported a role for many GABA_{A} receptors outside the CNS. For example, GABA_{A} receptors have been found to be expressed in various glandular tissues such as the pancreas and the adrenal cortex. Thus, it may be that GABA mediates function of the autonomic peripheral nervous system. GABA is released from secretory vesicles in pancreatic beta cells and binds GABA_{A} receptors on the alpha cells. GABA_{A} receptors have also been reported to be expressed in airway epithelial cells. The role of GABA signaling in these peripheral systems is still being elucidated.

The rho subunits have been reported as exclusively expressed in the retina. GABA receptors containing these subunits, which are unable to form functional complexes with the canonical alpha and beta subunits, have been termed “GABA_{C}” receptors. Like GABA_{A} receptors, GABA_{C} receptors are composed of five subunits and conduct chloride ions. Reported GABA_{C} receptors comprised solely of rho subunits are thought to be arranged in either homopentamers or heteropentamers. GABA_{C} receptors are more sensitive to the GABA ligand, are slower to initiate a response, and have a more sustained response when compared to GABA_{A}. Additionally, GABA_{C} receptors do not respond to GABA_{A} receptor modulators such as barbiturates, benzodiazepines, and neuroactive steroids.

GABA_{B} receptors are distinct from GABA_{A} and GABA_{C} receptors in that they are G-protein coupled receptors that regulate potassium channels. GABA_{B} receptors also reduce the activity of adenylyl cyclase to induce intracellular calcium release. Like the other GABA receptors, GABA_{B} receptor activity inhibits the progression of action potentials along neurons. GABA_{B} receptors are formed as heterodimers of GABA_{B1} and GABA_{B2} subunits that dimerize in their C-terminal domains. GABA_{B} receptors are activated by GABA ligand and selective agonists such as gamma-Hydroxybutyrate, Phenibut, and Baclofen.

At a cellular level, GABA receptors are expressed both at synapses where they respond to large changes in GABA concentration caused by release of the neurotransmitter into the synaptic space, and extra-synaptically where the receptors respond to lower concentrations of GABA that “leak” from synaptic junctions. The synaptic receptors respond to acute changes in neuronal firing whereas the extra-synaptic receptors are responsible for maintaining overall tone of neuronal networks.

The discovery of new and improved therapeutics that specifically target GABA_{A} receptor family members has been hampered by the lack of robust, physiologically relevant, cell-based systems that are amenable to high through-put formats for identifying and testing GABA_{A} receptor modulators. Cell-based systems are preferred for drug discovery and validation because they provide a functional assay for a compound in a cellular context as opposed to cell-free systems, which provide only a simple binding assay. Moreover, cell-based systems have the advantage of simultaneously testing cytotoxicity. Ideally, cell-based systems should also stably and constitutively express the target protein. It is also desirable for a cell-based system to be reproducible. Further, a complete characterization of GABA_{A} receptor expression, localization, activity and function, subunit combinations, and total subunits per active channel remains unexplored. Given the complexity and diversity of the GABA_{A} subunits, GABA_{A} expression patterns, and pleiotropic effects of GABA_{A} drugs to date, it is clear that additional research is needed to elucidate a more complete picture of the GABA_{A} receptor. The present invention addresses this need.

### SUMMARY OF THE INVENTION

We have discovered new and useful cells and cell lines that express functional GABA receptors. The cells in the cell line may be for example, eukaryotic or mammalian cells. In some embodiments, the cells in the cell line do not express GABA receptors endogenously. In other embodiments, the cells are CHO or 293T cells. While the GABA receptors are preferably mammalian, and more preferably human, any GABA receptor from any species can be expressed in the cells and cell lines of the present invention. In some embodiments, the GABA receptor comprises subunits that are from the same species. Alternatively, one or more GABA receptors may be chimeric, i.e., comprising subunits from two or more sources which can be different species. In some embodiments, the GABA receptor lacks a polypeptide tag at the amino terminus and the carboxy terminus. In some embodiments, the cells and cell lines may be used in a membrane potential dye assay such that the assay has a Z′value of at least 0.4, 0.45, 0.5, 0.55, 0.6, 0.65, 0.7, 0.75, or 0.8. The cells or cell lines may be stable in culture media without selective pressure. In some embodiments, one or more GABA subunits are expressed from an introduced nucleic acid encoding it, while in other embodiments, each GABA subunit is expressed from a separate nucleic acid introduced into the host cell. Further, in some embodiments, more than one GABA subunit is expressed from the same nucleic acid introduced into the host cell, while in other embodiments, one or more GABA subunits are expressed from an endogenous nucleic acid by gene activation.

The GABA receptor expressing cell lines may comprise one or more, two or more, three or more, four or more or five or more subunits from the group consisting of alpha 1(α1), alpha 2(α2), alpha 3(α3), alpha 4(α4), alpha 5(α5), alpha 6(α6), beta 1(β1), beta 2 (short) (β2S), beta 2 (long) (β2L), beta 3 (isoform 1) (β3.1), beta 3 (isoform 2) (β3.2), gamma 1(γ1), gamma 2 (short) (γ2S), gamma 2 (long) (γ2L), gamma 3(γ3), delta(δ), epsilon(ε), pi(π), theta (θ), rho 1 (ρ1), rho 2(ρ2), rho 3(ρ3), GABA_{B} receptor 1A, GABA_{B} receptor 1B, GABA_{B} receptor 1C, GABA_{B} receptor 1D, GABA_{B} receptor 1E, and GABA_{B} receptor 2. In some embodiments the cell lines are GABA_{A} receptor expressing cell lines comprising one or more, two or more, three or more, four or more or five or more subunits from the group consisting of alpha 1, alpha 2, alpha 3, alpha 4, alpha 5, alpha 6, beta 1, beta 2 (short), beta 2 (long), beta 3 (isoform 1), beta 3 (isoform 2), gamma 1, gamma 2 (short), gamma 2 (long), gamma 3, delta, epsilon, pi, and theta. In other embodiments the cell lines are GABA_{B} receptor expressing cell lines comprising one or more, two or more, three or more, four or more or five or more subunits from the group consisting of GABA_{B} receptor 1A, GABA_{B} receptor 1B, GABA_{B} receptor 1C, GABA_{B} receptor 1D, GABA_{B} receptor 1E, and GABA_{B} receptor 2. Finally, in some embodiments the cell lines are GABA_{C} receptor expressing cell lines comprising one or more, two or more, three or more, four or more or five or more subunits from the group consisting of rho1, rho2, and rho3.

The cells and cell lines of the present invention may comprise at least one amino acid encoded by a nucleic acid of any one of SEQ ID NOs: 1-28. In some embodiments, the cells and cell lines of the present invention may comprise at least one amino acid encoded by a nucleic acid that is at least 95% identical to any one of SEQ ID NOs: 1-28; a nucleic acid that hybridizes to the reverse-complement of any one of SEQ ID NOs: 1-28 under stringent conditions; or a nucleic acid that is an allelic variant of any one of SEQ ID NOS: 1-28. Further, the cells and cell lines may comprise at least one amino acid selected from any one of SEQ ID NOs: 29-56. In some embodiments, the cells and cell lines may comprise at least one amino acid that is at least 95% identical to any one of SEQ ID NOS: 29-56; an amino acid sequence encoded by a nucleic acid that hybridizes to the reverse-complement of any one of SEQ ID NOs: 1-28 under stringent conditions; or an amino acid encoded by a nucleic acid that is an allelic variant of any one of SEQ ID NOs: 1-28.

In some embodiments, the GABA receptor of the cells and cell lines of the present invention is a GABA_{A} receptor and comprises at least one alpha subunit, at least one beta subunit and at least one gamma or delta subunit. In other embodiments, the GABA_{A} receptor comprises two alpha subunits, two beta subunits and either a gamma or a delta subunit. In some embodiments the GABA receptor is a functional GABA receptor, and in preferred embodiments, the functional GABA receptor is a functional GABA_{A} receptor. Such functional GABA expressing cell lines exhibit a change in intracellular chloride ion concentration (GABA_{A} and GABA_{C}) or intracellular potassium ion concentration (GABA_{B}) when contacted with the GABA ligand. In some embodiments the EC₅₀ value of GABA ligand for chloride ion concentration change is below 3.5 μM or 400 nM.

The present invention also includes a collection of two or more cell lines, wherein: each cell line stably expresses a heterologous GABA receptor subunit or combination of GABA receptor subunits, each cell line stably expresses a different heterologous GABA receptor subunit or combination of GABA receptor subunits, or each cell line stably expresses the same heterologous GABA receptor subunit or combination of GABA receptor subunits. In some embodiments, the collection of GABA receptor expressing cell lines is a collection of GABA_{A} receptor expressing cell lines. In some embodiments, each cell line of the collection of cell lines has a change in intracellular chloride ions in response to GABA ligand, wherein the EC₅₀ value for such a change is between 100 nM and 3500 nM.

The invention also encompasses a method of producing a stable, GABA receptor expressing cell line. In one some embodiments, such a method comprises the steps of: a) introducing into a plurality of cells a nucleic acid encoding one or more GABA receptor subunits; b) introducing into the plurality of cells provided in step a) molecular beacons that detects expression of the GABA receptor subunits; c) isolating a cell that expresses the one or more GABA receptor subunits and, optionally, d) generating a cell line from the cell isolated in step c). The isolation step may include a fluorescence activated cell sorter. Some embodiments of the invention also comprise the cells that express one or more endogenous or heterologous GABA receptor accessory proteins. Heterologous GABA receptor accessory proteins may be introduced into the host cells before, after or simultaneously as the introduction of the GABA receptor subunit or subunits. The GABA receptor subunits and accessory proteins may be expressed from the same or different nucleic acids. In particular embodiments, the GABA receptor used in this method is a GABA_{A} receptor.

The inventions further encompasses a method of identifying a modulator of a GABA receptor. In some embodiments, such a method comprises the steps: a) exposing a cell or cell line that stably expresses one or more GABA receptor subunits to a test compound; and b) detecting a change in a function of the GABA receptor. Alternatively the method may comprise exposing a collection of cell lines to a test compound or exposing a collection of cell lines to a library of different test compounds. In some embodiments, the cells and cell lines of the modulator-identifying method comprise the cells and cell lines of the invention. In some embodiments, the test compound is a GABA receptor agonist or antagonist. In some embodiments, cells, cell lines or collections of cell lines are exposed to a GABA receptor agonist or antagonist prior to or simultaneously as the test compound or library of test compounds. In particular embodiments, the GABA receptor used in this method is a GABA_{A} receptor.

### BRIEF DESCRIPTION OF THE FIGURES

FIG. 1 contains dose response curves from a membrane potential assay of GABA_{A} receptors α1β3γ2s (A1), α2β3γ2s (A2), α3β3γ2s (A3) and α5β3γ2s (A5). The assays measured the response of stable cell lines expressing GABA_{A} receptors to GABA, the GABA_{A} receptor endogenous ligand. The GABA EC₅₀ values for each cell line are also listed.

FIG. 2 contains dose response curves from a membrane potential assay of GABA_{A} receptors α1β3γ2s (A1), α2β3γ2s (A2), α3β3γ2s (A3) and α5β3γ2s (A5). The assay measured the response of stable cell lines expressing GABA_{A} receptors to bicuculline, an antagonist, in the presence of EC₅₀ levels of GABA. Bicuculline IC₅₀ values for each cell line are also listed.

FIG. 3 is a schematic representation of data from high throughput membrane potential assays of known pharmaceutical agents from LOPAC1280 (a collection of 1280 pharmacologically active compounds, including many GABA modulators) against stable cell lines expressing GABA_{A} receptors α1β3γ2s (α1), α2β3γ2s (α2), α3β3γ2s (α3) and α5β3γ2s (a5). The relative activities of the GABA_{A} receptors in response to the eighteen most active compounds are depicted.

FIGS. 4a and 4b contain dose response curves from high throughput membrane potential assays of stable cell lines expressing GABA_{A} receptors α1β3γ2s (A1), α2β3γ2s (A2), α3β3γ2s (A3) and α5β3γ2s (A5). The assays measured the response of stable cell lines expressing GABA_{A} receptors to the following known GABA_{A} modulators in the LOPAC1280 library: propofol (anesthetic), muscimol hydrobromide (agonist), 5-alpha-pegnan-3alpha-ol-20-one (neurosteroid), 5-alpha-pregnan-3alpha-ol-11,20-dione (neurosteroid), isoguvacine hydrochloride, tracazolate, 3-alpha,21-dihydroxy-5-alpha-pregnan-20-one (neurosteroid), and piperidine-4-sulphonic acid (partial agonist). EC₅₀ values for each compound and cell line are also listed.

FIG. 5 contains dose response curves from high throughput membrane potential assays of stable cell lines expressing GABA_{A} receptors α1β3γ2s (A1), α2β3γ2s (A2), α3β3γ2s (A3) and α5β3γ2s (A5). The assay identified four compounds in the LOPAC1280 library not previously described as GABA activators but known to have activities associated with GABA_{A} as shown: etazolate (phosphodiesterase inhibitor), androsterone (steroid hormone), chlormezanone (muscle relaxant), and ivermectin (anti-parasitic known to effect chlorine channels). EC₅₀ values for each compound and cell line is also listed.

FIG. 6 contains dose response curves from high throughput membrane potential assays of stable cell lines expressing GABA_{A} receptors α1β3γ2s (A1), α2β3γ2s (A2), α3β3γ2s (A3) and α5β3γ2s (A5). The assay identified four compounds in the LOPAC1280 library which were previously not known to interact with GABA_{A}. These novel compounds include: dipyrimidole (adenosine deaminase inhibitor), niclosamide (anti-parasitic), tyrphosin A9 (PDGFR inhibitor), and I-Ome-Tyrphosin AG 538 (IGF RTK inhibitor). EC₅₀ values for each cell line are also listed.

FIG. 7 contains electrophysiology assays of stable cell lines expressing GABA_{A} receptors. The top three curves are receptor current traces of whole-cell GABA_{A} α2β3γ2s, α3β3γ2s and α5β3γ2s receptor expressing cell lines in response to 100 μM GABA. The bottom curve is a dose response curve indicating whole-cell GABA_{A} α1β3γ2s receptor currents in response to increasing concentrations of GABA (0.10-100 μM). The shaded region indicates the interval in which peak currents are identified.

FIGS. 8a and 8b. FIG. 8a contains representative dose response curves from a meYFP assay of stable cell lines expressing GABA_{A} receptors α3β3γ2s. The assay measured the response of a stable cell line expressing the GABA_{A} receptor to GABA. An increase in the amount of quenching of the meYFP signal indicates a response to GABA-induced anion uptake. FIG. 8b contains dose response curves from a meYFP assay of stable cell lines expressing GABA_{A} receptors α1β3γ2s (A1), α2β3γ2s (A2), α3β3γ2s (A3) and α5β3γ2s (A5). The assays measured the response of stable cell lines expressing GABA_{A} receptors to GABA. The GABA EC₅₀ values for each cell line are also listed.

### DETAILED DISCLOSURE

Unless otherwise defined, all technical and scientific terms used herein have the same meaning as commonly understood by one of ordinary skill in the art to which this invention belongs. Exemplary methods and materials are described herein, although methods and materials similar or equivalent to those described herein can also be used in the practice or testing of the present invention. All publications and other references mentioned herein are incorporated by reference in their entirety. In case of conflict, the present specification, including definitions, will control. Although a number of documents are cited herein, this citation does not constitute an admission that any of these documents forms part of the common general knowledge in the art. Throughout this specification and claims, the word “comprise,” or variations such as “comprises” or “comprising” will be understood to imply the inclusion of a stated integer or group of integers but not the exclusion of any other integer or group of integers. Unless otherwise required by context, singular terms shall include pluralities and plural terms shall include the singular. The materials, methods, and examples are illustrative only and not intended to be limiting.

In order that the present invention may be more readily understood, certain terms are first defined. Additional definitions are set forth throughout the detailed description.

The term “stable” or “stably expressing” is meant to distinguish the cells and cell lines of the invention from cells with transient expression as the terms “stable expression” and “transient expression” would be understood by a person of skill in the art.

The term “cell line” or “clonal cell line” refers to a population of cells that are all progeny of a single original cell. As used herein, cell lines are maintained in vitro in cell culture and may be frozen in aliquots to establish banks of clonal cells.

The term “stringent conditions” or “stringent hybridization conditions” describe temperature and salt conditions for hybridizing one or more nucleic acid probes to a nucleic acid sample and washing off probes that have not bound specifically to target nucleic acids in the sample. Stringent conditions are known to those skilled in the art and can be found in Current Protocols in Molecular Biology, John Wiley & Sons, N.Y. (1989), 6.3.1-6.3.6. Aqueous and nonaqueous methods are described in that reference and either can be used. An example of stringent hybridization conditions is hybridization in 6×SSC at about 45° C., followed by at least one wash in 0.2×SSC, 0.1% SDS at 60° C. A further example of stringent hybridization conditions is hybridization in 6×SSC at about 45° C., followed by at least one wash in 0.2×SSC, 0.1% SDS at 65° C. Stringent conditions include hybridization in 0.5M sodium phosphate, 7% SDS at 65° C., followed by at least one wash at 0.2×SSC, 1% SDS at 65° C.

The phrase “percent identical” or “percent identity” in connection with amino acid and/or nucleic acid sequences refers to the similarity between at least two different sequences. This percent identity can be determined by standard alignment algorithms, for example, the Basic Local Alignment Tool (BLAST) described by Altshul et al. ((1990) J. Mol. Biol., 215: 403-410); the algorithm of Needleman et al. ((1970) J. Mol. Biol., 48: 444-453); or the algorithm of Meyers et al. ((1988) Comput. Appl. Biosci., 4: 11-17). A set of parameters may be the Blosum 62 scoring matrix with a gap penalty of 12, a gap extend penalty of 4, and a frameshift gap penalty of 5. The percent identity between two amino acid or nucleotide sequences can also be determined using the algorithm of E. Meyers and W. Miller ((1989) CABIOS, 4:11-17) that has been incorporated into the ALIGN program (version 2.0), using a PAM120 weight residue table, a gap length penalty of 12 and a gap penalty of 4. The percent identity is usually calculated by comparing sequences of similar length. Protein analysis software matches similar sequences using measures of similarity assigned to various substitutions, deletions and other modifications, including conservative amino acid substitutions. For instance, the GCG Wisconsin Package (Accelrys, Inc.) contains programs such as “Gap” and “Bestfit” that can be used with default parameters to determine sequence identity between closely related polypeptides, such as homologous polypeptides from different species of organisms or between a wild type protein and a mutation thereof. See, e.g., GCG Version 6.1. Polypeptide sequences also can be compared using FASTA using default or recommended parameters. A program in GCG Version 6.1. FASTA (e.g., FASTA2 and FASTA3) provides alignments and percent sequence identity of the regions of the best overlap between the query and search sequences (Pearson, Methods Enzymol. 183:63-98 (1990); Pearson, Methods Mol. Biol. 132:185-219 (2000)). The length of polypeptide sequences compared for identity will generally be at least about 16 amino acid residues, usually at least about 20 residues, more usually at least about 24 residues, typically at least about 28 residues, and preferably more than about 35 residues. The length of a DNA sequence compared for identity will generally be at least about 48 nucleic acid residues, usually at least about 60 nucleic acid residues, more usually at least about 72 nucleic acid residues, typically at least about 84 nucleic acid residues, and preferably more than about 105 nucleic acid residues.

The phrase “substantially as set out,” “substantially identical” or “substantially homologous” in connection with an amino acid nucleotide sequence means that the relevant amino acid or nucleotide sequence will be identical to or have insubstantial differences (through conserved amino acid substitutions) in comparison to the sequences that are set out. Insubstantial differences include minor amino acid changes, such as 1 or 2 substitutions in a 50 amino acid sequence of a specified region.

The terms “potentiator”, “agonist” or “activator” refer to a compound or substance that activates a biological function of GABA_{A} receptor, e.g. ion conductance via a GABA_{A} receptor. As used herein, a potentiator or activator may act upon all or upon a specific subset of GABA_{A} subunits.

The terms “inhibitor”, “antagonist” or “blocker” refers to a compound or substance that that decreases a biological function of GABA_{A} receptor, e.g. ion conductance via a GABA_{A} receptor. As used herein, an inhibitor or blocker may act upon all or upon a specific subset of GABA_{A} subunits.

The term “modulator” refers to a compound or substance that alters the structure, conformation, biochemical or biophysical properties or functionality of a GABA_{A} receptor either positively or negatively. The modulator can be a GABA_{A} receptor agonist (potentiator or activator) or antagonist (inhibitor or blocker), including partial agonists or antagonists, selective agonists or antagonists and inverse agonists, and can be an allosteric modulator. A substance or compound is a modulator even if its modulating activity changes under different conditions or concentrations or with respect to different forms of GABA_{A} receptor. As used herein, a modulator may affect the ion conductance of a GABA_{A} receptor, the response of a GABA_{A} receptor to another regulatory compound or the selectivity of a GABA_{A} receptor. A modulator may also change the ability of another modulator to affect the function of a GABA_{A} receptor. A modulator may act upon all or upon a specific subset of GABA_{A} subunits. Modulators include, but are not limited to, potentiators, activators, inhibitors, agonists, antagonists, and blockers.

As used herein, “EC₅₀” refers to the concentration of a compound or substance required to induce a half-maximal activating response in the cell or cell line. As used herein, “IC₅₀” refers to the concentration of a compound or substance required to induce a half-maximal inhibitory response in the cell or cell line. EC₅₀ and IC₅₀ values may be determined using techniques that are well-known in the art, for example, a dose-response curve that correlates the concentration of a compound or substance to the response of the GABA_{A}-expressing cell line.

The phrase “functional GABA receptor” refers to a GABA receptor that responds to GABA (its endogenous ligand), an activator, or an inhibitor, in substantially the same way as a GABA receptor in a cell that normally expresses a GABA receptor without engineering. Similarly, the phrases “functional GABA_{A} receptor,” “functional GABA_{B} receptor,” or “functional GABA_{C} receptor” refer to GABA_{A}, GABA_{B}, or GABA_{C} receptors that respond to GABA (their endogenous ligand), an activator, or an inhibitor, in substantially the same way as a GABA_{A}, GABA_{B}, and GABA_{C} receptors in cells that normally expresses GABA_{A}, GABA_{B}, and GABA_{C} receptors without engineering. GABA_{A} receptor behavior can be determined by, for example, physiological activities, and pharmacological responses. Physiological activities include, but are not limited to chloride conductance. Pharmacological responses include, but are not limited to, activation by agonists such as GABA, Isoguvacine HCl, Muscimol, 3-amino-1-propanesulfonic acid sodium salt, Gaboxadol, piperidine-4-sulfonic acid, 6,2′-Dihydroxyflavone, and DEABL; inhibition by antagonists such as Bicuculline, Furosemide, Picrotoxin, SR 95531, U93631, (1S,9R)-(+)-β-Hydrastine, Ethyl β-carboline, 3-Methyl-643-(trifluoromethyl)phenyl, (−)-α-Thujone, Cloflubicyne, Etbicyphat, Etbicythionat, Flucybene, Imidazole-4-acetic acid, Phaclofen, Picrotoxinin, Propybicyphat, Tert-Butyl bicycle[2,2,2], Cicutoxin, and Tetramethylenedisulfotetramine (TETS, DSTA) Flumazenil; potentiation by benzodiazepine positive modulators such as CGS 2025, Chlomezanone, CL 218872, Diazepam, Flunitrazepam, Temazepam, Nimetazepam, Nitrazepam, Bromazepam, Camazepam, Clonazepam, Estazolam, Oxazepa, Lormetazepam, Prazepam, GBLD 345, Hispidulin, L-665,708, Lorazepam, Midazolam, ZK 93423HCl, ZK93426HCl, Zolpidem, Zopiclone, Zaleplon, Eszopiclone, Indiplon, Ocinaplon, Pagoclone, Suriclone, Pazinaclone, Alpidem, Saripidem, Necopidem, Panadiplon (U78875), SX-3228, L-838417, RWJ-51204, and Y-23684; activation and potentiation by neurosteroids such as Alphaxalone, Ganaxolone, 3α, 21-Dihydroxy-5α-pregnan-20-one, 5α-Pregnan-3α-ol-11,20-dione, 5α-Pregnan-3α-ol-20-one, Dehydroisoandrosterone 3-sulfate, and trans-Dehydroandrosterone; activation and potentiation by barbiturates such as pentobarbital, phenobarbital, mephobarbital, secobarbital, amobarbital, butalbital, cyclobarbital, allobarbital, methylphenobarbital, phenobarbital, and vinylbital; activation by anesthetics such as propofol, etomidate and fospropofol; and modulation by other compounds such as Loreclezole Hydrochloride Chlormethiazole, Dihydroergotoxine mesylate, Org 20599, 17-PA, Primidone, SB 205384, SCS, Tracazolate Hydrochloride, U 89843A, U 90042, Valerenic Acid, Guvacine hydrochloride, NO-711 hydrochloride, Vigabatrin, Popofol, Zonisamide, Valproic Acid, Gabapentin, 3-Methyl GABA, N-Arachidonyl GABA, Etifoxine, ethanol, and kavalactones.

A “heterologous” or “introduced” GABA_{A} subunit means that the GABA_{A} subunit is encoded by a polynucleotide introduced into a host cell.

GABA_{A} receptor is a protein that is present in many mammalian tissues, including CNS/brain, airway epithelial cells, pancreas, and adrenal cortex. Without being bound by any theory, we believe that GABA_{A} receptor dysregulation or dysfunction may be linked to many disease states including epilepsy, autism, sclerosis, alcohol consumption and addiction. As novel combinations of GABA subunits are characterized various additional disease states may be attributed to their dysregulation or dysfunction.

GABA_{A} receptor is a membrane spanning multimeric ion channel, typically comprising multiple subunits. While reported GABA_{A} receptors typically comprise two alpha subunits, two beta subunits, and one gamma or delta subunit, any combination of these subunits is envisioned. In some embodiments, the GABA_{A} receptors of the present invention contain one, two, three, four, five or more subunits.

The current invention relates to novel cells and cell lines that have been engineered to express GABA receptor subunits. In preferred embodiments, the GABA receptor subunits are GABA_{A} subunits (SEQ ID NO: 29-47). In some embodiments, the novel cells or cell lines of the invention express a functional GABA_{A} receptor. In some embodiments, the novel cells or cell lines of the invention express a native GABA_{A} receptor. In some embodiments, the GABA receptor subunits are GABA_{B} subunits (SEQ ID NO: 51-56). In some embodiments, the novel cells or cell lines of the invention express a functional GABA_{B} receptor. In some embodiments, the novel cells or cell lines of the invention express a native GABA_{B} receptor. In other embodiments, the GABA receptor subunits are GABA_{C} subunits (SEQ ID NO: 48-50). In yet other embodiments, the novel cells or cell lines of the invention express a functional GABA_{C} receptor. In some embodiments, the novel cells or cell lines of the invention express a native GABA_{C} receptor. In some embodiments the novel cells or cell lines of the invention express a, to date, unreported combination of GABA subunits, including combinations of GABA_{A}, GABA_{B}, and GABA_{C} subunits. In other aspects, the invention provides methods of making and using the novel cells and cell lines.

According to some embodiments of the invention, the novel cells and cell lines are simultaneously transfected with nucleic acids individually encoding GABA subunits. In some embodiments of the invention, the novel cells and cell lines are simultaneously transfected with nucleic acids individually encoding GABA_{A} subunits (SEQ ID NO: 1-19), GABA_{B} subunits (SEQ ID NO: 23-28), or GABA_{C} subunits (SEQ ID NO: 20-22). In some embodiments, the cells and cell lines are triply transfected with nucleic acids individually encoding a GABA_{A} alpha subunit, a GABA_{A} beta subunit, and a GABA_{A} gamma subunit on the same or separate vectors. The novel cell lines of the invention stably express the introduced GABA_{A} subunits.

In a particular embodiment, the novel cells and cell lines express an endogenous GABA receptor subunit as a result of engineered gene activation, i.e., activation of the expression of an endogenous gene, wherein the activation does not naturally occur in a cell without proper treatment. Alternatively, engineered gene activation can be used to increase the expression of a gene that is expressed a cell. Engineered gene activation can be achieved by a number of means known to those skilled in the art. For example, one or more transcription factors or transactivators of transcription of a gene can be over-expressed or induced to express by, e.g., introducing nucleic acids expressing the transcription factors or transactivators into a cell under the control of a constitutive or inducible promoter. If the endogenous gene is known to be under the control of an inducible promoter, expression can be induced by exposing the cell to a known inducer of the gene. In addition, a nucleic acid encoding the endogenous gene itself can be introduced into a cell to obtain an increased level of expression of the gene due to increased copy number in the genome. Furthermore, certain known inhibitors of the expression of an endogenous gene that are expressed by the cell can be knocked down or even knocked out in the cell using techniques well known in the art, e.g., RNAi, thereby increasing the expression of the endogenous gene. In some embodiments, the novels cells and cells lines have at least one, at least two, at least three, at least four, or at least five subunits activated for expression by gene activation.

According to some embodiments of the invention, the novel cells and cell lines are transfected with different combinations of nucleic acids encoding various GABA subunits. In some embodiments, the novel cells and cell lines are transfected with different combinations of nucleic acids encoding various GABA_{A} subunits. For example, the cells or cell lines may be transfected with two different alpha subunits, a beta subunit and a gamma subunit; an alpha subunit, two different beta subunits and a gamma subunit; two different alpha subunits, two different beta subunits, and a gamma subunit; or any combination of GABA_{A} subunits disclosed herein. In some embodiments, the cells and cell lines express combinations of GABA_{A} subunits with GABA_{B} subunits, GABA_{C} subunits, or both GABA_{B} and GABA_{C} subunits.

The present invention encompasses cells expressing one of the following combinations of genes or gene products:

i) A

ii) A and B;

iii) A, B, and C;

iv) A, B, C, and D;

v) A, B, C, D, and E;

vi) A, B, C, D, E, and F;

vii) A, B, C, D, E, F, and G;

viii) A, B, C, D, E, F, G, and H;

ix) A, B, C, D, E, F, G, H, and I;

x) A, B, C, D, E, F, G, H, I, and J;

xi) A, B, C, D, E, F, G, H, I, J, and K

xii) A, B, C, D, E, F, G, H, I, J, K, and L

xiii) A, B, C, D, E, F, G, H, I, J, K, L and M

xiv) A, B, C, D, E, F, G, H, I, J, K, L, M, and N

xv) A, B, C, D, E, F, G, H, I, J, K, L, M, N, and O;

xvi) A, B, C, D, E, F, G, H, I, J, K, L, M, N, O, and P;

xvii) A, B, C, D, E, F, G, H, I, J, K, L, M, N, O, P, and Q

xviii) A, B, C, D, E, F, G, H, I, J, K, L, M, N, O, P, Q, and R;

xix) A, B, C, D, E, F, G, H, I, J, K, L, M, N, O, P, Q, R, and S;

xx) A, B, C, D, E, F, G, H, I, J, K, L, M, N, O, P, Q, R, S, and T;

xxi) A, B, C, D, E, F, G, H, I, J, K, L, M, N, O, P, Q, R, S, T, and U;

xxii) A, B, C, D, E, F, G, H, I, J, K, L, M, N, O, P, Q, R, S, T, U, and V;

xxiii) A, B, C, D, E, F, G, H, I, J, K, L, M, N, O, P, Q, R, S, T, U, V, and W;

xxiv) A, B, C, D, E, F, G, H, I, J, K, L, M, N, O, P, Q, R, S, T, U, V, W, and X;

xxv) A, B, C, D, E, F, G, H, I, J, K, L, M, N, O, P, Q, R, S, T, U, V, W, X, and Y;

xxvi) A, B, C, D, E, F, G, H, I, J, K, L, M, N, O, P, Q, R, S, T, U, V, W, X, Y, and Z;

xxvii) A, B, C, D, E, F, G, H, I, J, K, L, M, N, O, P, Q, R, S, T, U, W, X, Y, Z, and ε; and

xxviii) A, B, C, D, E, F, G, H, I, J, K, L, M, N, O, P, Q, R, S, T, U, W, X, Y, Z, ε, and £,

wherein A is selected from the group consisting of α1, α2, α3, α4, α5, α6, β1, β2S, β2L, β3.1, β3.2, γ1, γ2S, γ2L, γ3, δ, ε, π, θ, ρ1, ρ2, ρ3, GABAB receptor 1A, GABAB receptor 1B, GABAB receptor 1C, GABAB receptor 1D, GABAB receptor 1E, and GABAB receptor 2;

B is selected from the group consisting of α1, α2, α3, α4, α5, α6, β1, β2S, β2L, β3.1, β3.2, γ1, γ2S, γ2L, γ3, δ, ε, π, θ, ρ1, ρ2, ρ3, GABAB receptor 1A, GABAB receptor 1B, GABAB receptor 1C, GABAB receptor 1D, GABAB receptor 1E, and GABAB receptor 2;

C is selected from the group consisting of α1, α2, α3, α4, α5, α6, β1, β2S, β2L, β3.1, β3.2, γ1, γ2S, γ2L, γ3, δ, ε, π, θ, ρ1, ρ2, ρ3, GABAB receptor 1A, GABAB receptor 1B, GABAB receptor 1C, GABAB receptor 1D, GABAB receptor 1E, and GABAB receptor 2;

D is selected from the group consisting of α1, α2, α3, α4, α5, α6, β1, β2S, β2L, β3.1, β3.2, γ1, γ2S, γ2L, γ3, δ, ε, π, θ, ρ1, ρ2, ρ3, GABAB receptor 1A, GABAB receptor 1B, GABAB receptor 1C, GABAB receptor 1D, GABAB receptor 1E, and GABAB receptor 2;

E is selected from the group consisting of α1, α2, α3, α4, α5, α6, β1, β2S, β2L, β3.1, β3.2, γ1, γ2S, γ2L, γ3, δ, ε, π, θ, ρ1, ρ2, ρ3, GABAB receptor 1A, GABAB receptor 1B, GABAB receptor 1C, GABAB receptor 1D, GABAB receptor 1E, and GABAB receptor 2;

F is selected from the group consisting of α1, α2, α3, α4, α5, α6, β1, β2S, β2L, β3.1, β3.2, γ1, γ2S, γ2L, γ3, δ, ε, π, θ, ρ1, ρ2, ρ3, GABAB receptor 1A, GABAB receptor 1B, GABAB receptor 1C, GABAB receptor 1D, GABAB receptor 1E, and GABAB receptor 2;

G is selected from the group consisting of α1, α2, α3, α4, α5, α6, β1, β2S, β2L, β3.1, β3.2, γ1, γ2S, γ2L, γ3, δ, ε, π, θ, ρ1, ρ2, ρ3, GABAB receptor 1A, GABAB receptor 1B, GABAB receptor 1C, GABAB receptor 1D, GABAB receptor 1E, and GABAB receptor 2;

H is selected from the group consisting of α1, α2, α3, α4, α5, α6, β1, β2S, β2L, β3.1, β3.2, γ1, γ2S, γ2L, γ3, δ, ε, π, θ, ρ1, ρ2, ρ3, GABAB receptor 1A, GABAB receptor 1B, GABAB receptor 1C, GABAB receptor 1D, GABAB receptor 1E, and GABAB receptor 2;

I is selected from the group consisting of α1, α2, α3, α4, α5, α6, β1, β2S, β2L, β3.1, β3.2, γ1, γ2S, γ2L, γ3, δ, ε, π, θ, ρ1, ρ2, ρ3, GABAB receptor 1A, GABAB receptor 1B, GABAB receptor 1C, GABAB receptor 1D, GABAB receptor 1E, and GABAB receptor 2;

J is selected from the group consisting of α1, α2, α3, α4, α5, α6, β1, β2S, β2L, β3.1, β3.2, γ1, γ2S, γ2L, γ3, δ, ε, π, θ, ρ1, ρ2, ρ3, GABAB receptor 1A, GABAB receptor 1B, GABAB receptor 1C, GABAB receptor 1D, GABAB receptor 1E, and GABAB receptor 2;

K is selected from the group consisting of α1, α2, α3, α4, α5, α6, β1, β2S, β2L, β3.1, β3.2, γ1, γ2S, γ2L, γ3, δ, ε, π, θ, ρ1, ρ2, ρ3, GABAB receptor 1A, GABAB receptor 1B, GABAB receptor 1C, GABAB receptor 1D, GABAB receptor 1E, and GABAB receptor 2;

L is selected from the group consisting of α1, α2, α3, α4, α5, α6, β1, β2S, β2L, β3.1, β3.2, γ1, γ2S, γ2L, γ3, δ, ε, π, θ, ρ1, ρ2, ρ3, GABAB receptor 1A, GABAB receptor 1B, GABAB receptor 1C, GABAB receptor 1D, GABAB receptor 1E, and GABAB receptor 2;

M is selected from the group consisting of α1, α2, α3, α4, α5, α6, β1, β2S, β2L, β3.1, β3.2, γ1, γ2S, γ2L, γ3, δ, ε, π, θ, ρ1, ρ2, ρ3, GABAB receptor 1A, GABAB receptor 1B, GABAB receptor 1C, GABAB receptor 1D, GABAB receptor 1E, and GABAB receptor 2;

N is selected from the group consisting of α1, α2, α3, α4, α5, α6, β1, β2S, β2L, β3.1, β3.2, γ1, γ2S, γ2L, γ3, δ, ε, π, θ, ρ1, ρ2, ρ3, GABAB receptor 1A, GABAB receptor 1B, GABAB receptor 1C, GABAB receptor 1D, GABAB receptor 1E, and GABAB receptor 2;

O is selected from the group consisting of α1, α2, α3, α4, α5, α6, β1, β2S, β2L, β3.1, β3.2, γ1, γ2S, γ2L, γ3, δ, ε, π, θ, ρ1, ρ2, ρ3, GABAB receptor 1A, GABAB receptor 1B, GABAB receptor 1C, GABAB receptor 1D, GABAB receptor 1E, and GABAB receptor 2;

P is selected from the group consisting of α1, α2, α3, α4, α5, α6, β1, β2S, β2L, β3.1, β3.2, γ1, γ2S, γ2L, γ3, δ, ε, π, θ, ρ1, ρ2, ρ3, GABAB receptor 1A, GABAB receptor 1B, GABAB receptor 1C, GABAB receptor 1D, GABAB receptor 1E, and GABAB receptor 2;

Q is selected from the group consisting of α1, α2, α3, α4, α5, α6, β1, β2S, β2L, β3.1, β3.2, γ1, γ2S, γ2L, γ3, δ, ε, π, θ, ρ1, ρ2, ρ3, GABAB receptor 1A, GABAB receptor 1B, GABAB receptor 1C, GABAB receptor 1D, GABAB receptor 1E, and GABAB receptor 2;

R is selected from the group consisting of α1, α2, α3, α4, α5, α6, β1, β2S, β2L, β3.1, β3.2, γ1, γ2S, γ2L, γ3, δ, ε, π, θ, ρ1, ρ2, ρ3, GABAB receptor 1A, GABAB receptor 1B, GABAB receptor 1C, GABAB receptor 1D, GABAB receptor 1E, and GABAB receptor 2;

S is selected from the group consisting of α1, α2, α3, α4, α5, α6, β1, β2S, β2L, β3.1, β3.2, γ1, γ2S, γ2L, γ3, δ, ε, π, θ, ρ1, ρ2, ρ3, GABAB receptor 1A, GABAB receptor 1B, GABAB receptor 1C, GABAB receptor 1D, GABAB receptor 1E, and GABAB receptor 2;

T is selected from the group consisting of α1, α2, α3, α4, α5, α6, β1, β2S, β2L, β3.1, β3.2, γ1, γ2S, γ2L, γ3, δ, ε, π, θ, ρ1, ρ2, ρ3, GABAB receptor 1A, GABAB receptor 1B, GABAB receptor 1C, GABAB receptor 1D, GABAB receptor 1E, and GABAB receptor 2;

U is selected from the group consisting of α1, α2, α3, α4, α5, α6, β1, β2S, β2L, β3.1, β3.2, γ1, γ2S, γ2L, γ3, δ, ε, π, θ, ρ1, ρ2, ρ3, GABAB receptor 1A, GABAB receptor 1B, GABAB receptor 1C, GABAB receptor 1D, GABAB receptor 1E, and GABAB receptor 2;

V is selected from the group consisting of α1, α2, α3, α4, α5, α6, β1, β2S, β2L, β3.1, β3.2, γ1, γ2S, γ2L, γ3, δ, ε, π, θ, ρ1, ρ2, ρ3, GABAB receptor 1A, GABAB receptor 1B, GABAB receptor 1C, GABAB receptor 1D, GABAB receptor 1E, and GABAB receptor 2;

W is selected from the group consisting of α1, α2, α3, α4, α5, α6, β1, β2S, β2L, β3.1, β3.2, γ1, γ2S, γ2L, γ3, δ, ε, π, θ, ρ1, ρ2, ρ3, GABAB receptor 1A, GABAB receptor 1B, GABAB receptor 1C, GABAB receptor 1D, GABAB receptor 1E, and GABAB receptor 2;

X is selected from the group consisting of α1, α2, α3, α4, α5, α6, β1, β2S, β2L, β3.1, β3.2, γ1, γ2S, γ2L, γ3, δ, ε, π, θ, ρ1, ρ2, ρ3, GABAB receptor 1A, GABAB receptor 1B, GABAB receptor 1C, GABAB receptor 1D, GABAB receptor 1E, and GABAB receptor 2;

Y is selected from the group consisting of α1, α2, α3, α4, α5, α6, β1, β2S, β2L, β3.1, β3.2, γ1, γ2S, γ2L, γ3, δ, ε, π, θ, ρ1, ρ2, ρ3, GABAB receptor 1A, GABAB receptor 1B, GABAB receptor 1C, GABAB receptor 1D, GABAB receptor 1E, and GABAB receptor 2;

Z is selected from the group consisting of α1, α2, α3, α4, α5, α6, β1, β2S, β2L, β3.1, β3.2, γ1, γ2S, γ2L, γ3, δ, ε, π, θ, ρ1, ρ2, ρ3, GABAB receptor 1A, GABAB receptor 1B, GABAB receptor 1C, GABAB receptor 1D, GABAB receptor 1E, and GABAB receptor 2;

ε is selected from the group consisting of α1, α2, α3, α4, α5, α6, β1, β2S, β2L, β3.1, β3.2, γ1, γ2S, γ2L, γ3, δ, ε, π, θ, ρ1, ρ2, ρ3, GABAB receptor 1A, GABAB receptor 1B, GABAB receptor 1C, GABAB receptor 1D, GABAB receptor 1E, and GABAB receptor 2; and

£ is selected from the group consisting of α1, α2, α3, α4, α5, α6, β1, β2S, β2L, β3.1, β3.2, γ1, γ2S, γ2L, γ3, δ, ε, π, θ, ρ1, ρ2, ρ3, GABAB receptor 1A, GABAB receptor 1B, GABAB receptor 1C, GABAB receptor 1D, GABAB receptor 1E, and GABAB receptor 2.

In some embodiments, the invention encompasses GABA_{A} molecules or receptors comprising any combination of two alpha subunits and, optionally a gamma, delta, epsilon, pi or theta subunit as shown in Table 1. The gamma subunits envisioned are indicated inside of each cell of Table 1 (0 indicating that no gamma subunit is present). For example, the upper left cell of Table 1 represents the possible α1α1 (no gamma subunit), α1α1γ1, α1α1γ2S, α1α1γ2L, α1α1γ3, α1α1δ, α1α1ε, α1α1π, or α1α1θ combinations.

TABLE 1

| | α1 | α2 | α3 | α4 | α5 | α6 |
|---|---|---|---|---|---|---|
| α1 | 0/γ1/γ2S | 0γ1/γ2S | 0/γ1/γ2S | 0/γ1/γ2S | 0/γ1/γ2S | 0/γ1/γ2S |
| | γ2L/γ3/δ | γ2L/γ3/δ | γ2L/γ3/δ | γ2L/γ3/δ | γ2L/γ3/δ | γ2L/γ3/δ |
| | ε/π/θ | ε/π/θ | ε/π/θ | ε/π/θ | ε/π/θ | ε/π/θ |
| α2 | 0/γ1/γ2S | 0/γ1/γ2S | 0/γ1/γ2S | 0/γ1/γ2S | 0/γ1/γ2S | 0/γ1/γ2S |
| | γ2L/γ2/δ | γ2L/γ3/δ | γ2L/γ3/δ | γ2L/γ3/δ | γ2L/γ3/δ | γ2L/γ3/δ |
| | ε/π/θ | ε/π/θ | ε/π/θ | ε/π/θ | ε/π/θ | ε/π/θ |
| α3 | 0/γ1/γ2S | 0/γ1/γ2S | 0/γ1/γ2S | 0/γ1/γ2S | 0/γ1/γ2S | 0/γ1/γ2S |
| | γ2L/γ3/δ | γ2L/γ3/δ | γ2L/γ3/δ | γ2L/γ3/δ | γ2L/γ3/δ | γ2L/γ3/δ |
| | ε/π/θ | ε/π/θ | ε/π/θ | ε/π/θ | ε/π/θ | ε/π/θ |
| α4 | 0/γ1/γ2S | 0/γ1/γ2S | 0/γ1/γ2S | 0/γ1/γ2S | 0/γ1/γ2S | 0/γ1/γ2S |
| | γ2L/γ3/δ | γ2L/γ3/δ | γ2L/γ3/δ | γ2L/γ3/δ | γ2L/γ3/δ | γ2L/γ3/δ |
| | ε/π/θ | ε/π/θ | ε/π/θ | ε/π/θ | ε/π/θ | ε/π/θ |
| α5 | 0/γ1/γ2S | 0γ1/γ2S | 0/γ1/γ2S | 0/γ1/γ2S | 0/γ1/γ2S | 0/γ1/γ2S |
| | γ2L/γ3/δ | γ2L/γ3/δ | γ2L/γ3/δ | γ2L/γ3/δ | γ2L/γ3/δ | γ2L/γ3/δ |
| | ε/π/θ | ε/π/θ | ε/π/θ | ε/π/θ | ε/π/θ | ε/π/θ |
| α6 | 0/γ1/γ2S | 0γ1/γ2S | 0/γ1/γ2S | 0/γ1/γ2S | 0/γ1/γ2S | 0/γ1/γ2S |
| | γ2L/γ3/δ | γ2L/γ3/δ | γ2L/γ3/δ | γ2L/γ3/δ | γ2L/γ3/δ | γ2L/γ3/δ |
| | ε/π/θ | ε/π/θ | ε/π/θ | ε/π/θ | ε/π/θ | ε/π/θ |

In other embodiments, the invention also encompasses GABA_{A} molecules comprising any combination of two beta subunits and, optionally, a gamma, delta, epsilon, pi or theta subunit as shown in Table 2. The gamma subunits envisioned are indicated inside of each cell of Table 2 (0 indicates that no gamma subunit is present). For example, the upper left cell of Table 2 represents the possible β1β1 (no gamma subunit), β1β1γ1, β1β1γ2S, β1β1γ2L, β1β1γ3, β1β1δ, β1β1ε, β1β1π, or β1β1θ combinations.

TABLE 2

| | β1 | β2L | β2S | β3.1 | β3.2 |
|---|---|---|---|---|---|
| β1 | 0/γ1/γ2S | 0/γ1/γ2S | 0/γ1/γ2S | 0/γ1/γ2S | 0/γ1/γ2S |
| | γ2L/γ3/δ | γ2L/γ3/δ | γ2L/γ3/δ | γ2L/γ3/δ | γ2L/γ3/δ |
| | ε/π/θ | ε/π/θ | ε/π/θ | ε/π/θ | ε/π/θ |
| β2 | 0/γ1/γ2S | 0/γ1/γ2S | 0/γ1/γ2S | 0/γ1/γ2S | 0/γ1/γ2S |
| | γ2L/γ3/δ | γ2L/γ3/δ | γ2L/γ3/δ | γ2L/γ3/δ | γ2L/γ3/δ |
| | ε/π/θ | ε/π/θ | ε/π/θ | ε/π/θ | ε/π/θ |
| β2S | 0/γ1/γ2S | 0/γ1/γ2S | 0/γ1/γ2S | 0/γ1/γ2S | 0/γ1/γ2S |
| | γ2L/γ3/δ | γ2L/γ3/δ | γ2L/γ3/δ | γ2L/γ3/δ | γ2L/γ3/δ |
| | ε/π/θ | ε/π/θ | ε/π/θ | ε/π/θ | ε/π/θ |
| β3.1 | 0/γ1/γ2S | 0/γ1/γ2S | 0/γ1/γ2S | 0/γ1/γ2S | 0/γ1/γ2S |
| | γ2L/γ3/δ | γ2L/γ3/δ | γ2L/γ3/δ | γ2L/γ3/δ | γ2L/γ3/δ |
| | ε/π/θ | ε/π/θ | ε/π/θ | ε/π/θ | ε/π/θ |
| β3.2 | 0/γ1/γ2S | 0/γ1/γ2S | 0/γ1/γ2S | 0/γ1/γ2S | 0/γ1/γ2S |
| | γ2L/γ3/δ | γ2L/γ3/δ | γ2L/γ3/δ | γ2L/γ3/δ | γ2L/γ3/δ |
| | ε/π/θ | ε/π/θ | ε/π/θ | ε/π/θ | ε/π/θ |

In further embodiments, the invention also encompasses GABA_{A} molecules comprising any combination of two alpha subunits, two beta subunits, and optionally a gamma, delta, epsilon, pi or theta subunit as shown in Table 3a and Table 3b. The gamma subunits envisioned are indicated inside of each cell of Tables 3a and 3b (0 indicates that no gamma subunit is present). For example, the upper left cell of Table 3a represents the possible α1α1β1β1 (no gamma subunit), α1α1β1β1γ1, α1α1β1β1γ2S, α1α1β1β1γ2L, α1α1β1β1γ3, α1α1β1β1δ, α1α1β1β1ε, α1α1β1β1π, and α1α1β1β1θ combinations.

TABLE 3a

| | β1β1 | β1β2 | β1β2S | β1β3.1 | β1β3.2 | β2Lβ2L | β2Lβ2S | β2Lβ3.1 |
|---|---|---|---|---|---|---|---|---|
| α1 | 0/γ1/γ2S | 0/γ1/γ2S | 0/γ1/γ2S | 0/γ1/γ2S | 0/γ1/γ2S | 0/γ1/γ2S | 0/γ1/γ2S | 0/γ1/γ2S |
| α1 | γ2L/γ3/δ | γ2L/γ3/δ | γ2L/γ3/δ | γ2L/γ3/δ | γ2L/γ3/δ | γ2L/γ3/δ | γ2L/γ3/δ | γ2L/γ3/δ |
| | ε/π/θ | ε/π/θ | ε/π/θ | ε/π/θ | ε/π/θ | ε/π/θ | ε/π/θ | ε/π/θ |
| α1 | 0/γ1/γ2S | 0/γ1/γ2S | 0/γ1/γ2S | 0/γ1/γ2S | 0/γ1/γ2S | 0/γ1/γ2S | 0/γ1/γ2S | 0/γ1/γ2S |
| α2 | γ2L/γ3/δ | γ2L/γ3/δ | γ2L/γ3/δ | γ2L/γ3/δ | γ2L/γ3/δ | γ2L/γ3/δ | γ2L/γ3/δ | γ2L/γ3/δ |
| | ε/π/θ | ε/π/θ | ε/π/θ | ε/π/θ | ε/π/θ | ε/π/θ | ε/π/θ | ε/π/θ |
| α1 | 0/γ1/γ2S | 0/γ1/γ2S | 0/γ1/γ2S | 0/γ1/γ2S | 0/γ1/γ2S | 0/γ1/γ2S | 0/γ1/γ2S | 0/γ1/γ2S |
| α3 | γ2L/γ3/δ | γ2L/γ3/δ | γ2L/γ3/δ | γ2L/γ3/δ | γ2L/γ3/δ | γ2L/γ3/δ | γ2L/γ3/δ | γ2L/γ3/δ |
| | ε/π/θ | ε/π/θ | ε/π/θ | ε/π/θ | ε/π/θ | ε/π/θ | ε/π/θ | ε/π/θ |
| α1 | 0/γ1/γ2S | 0/γ1/γ2S | 0/γ1/γ2S | 0/γ1/γ2S | 0/γ1/γ2S | 0/γ1/γ2S | 0/γ1/γ2S | 0/γ1/γ2S |
| α4 | γ2L/γ3/δ | γ2L/γ3/δ | γ2L/γ3/δ | γ2L/γ3/δ | γ2L/γ3/δ | γ2L/γ3/δ | γ2L/γ3/δ | γ2L/γ3/δ |
| | ε/π/θ | ε/π/θ | ε/π/θ | ε/π/θ | ε/π/θ | ε/π/θ | ε/π/θ | ε/π/θ |
| α1 | 0/γ1/γ2S | 0/γ1/γ2S | 0/γ1/γ2S | 0/γ1/γ2S | 0/γ1/γ2S | 0/γ1/γ2S | 0/γ1/γ2S | 0/γ1/γ2S |
| α5 | γ2L/γ3/δ | γ2L/γ3/δ | γ2L/γ3/δ | γ2L/γ3/δ | γ2L/γ3/δ | γ2L/γ3/δ | γ2L/γ3/δ | γ2L/γ3/δ |
| | ε/π/θ | ε/π/θ | ε/π/θ | ε/π/θ | ε/π/θ | ε/π/θ | ε/π/θ | ε/π/θ |
| α1 | 0/γ1/γ2S | 0/γ1/γ2S | 0/γ1/γ2S | 0/γ1/γ2S | 0/γ1/γ2S | 0/γ1/γ2S | 0/γ1/γ2S | 0/γ1/γ2S |
| α6 | γ2L/γ3/δ | γ2L/γ3/δ | γ2L/γ3/δ | γ2L/γ3/δ | γ2L/γ3/δ | γ2L/γ3/δ | γ2L/γ3/δ | γ2L/γ3/δ |
| | ε/π/θ | ε/π/θ | ε/π/θ | ε/π/θ | ε/π/θ | ε/π/θ | ε/π/θ | ε/π/θ |
| α2 | 0/γ1/γ2S | 0/γ1/γ2S | 0/γ1/γ2S | 0/γ1/γ2S | 0/γ1/γ2S | 0/γ1/γ2S | 0/γ1/γ2S | 0/γ1/γ2S |
| α2 | γ2L/γ3/δ | γ2L/γ3/δ | γ2L/γ3/δ | γ2L/γ3/δ | γ2L/γ3/δ | γ2L/γ3/δ | γ2L/γ3/δ | γ2L/γ3/δ |
| | ε/π/θ | ε/π/θ | ε/π/θ | ε/π/θ | ε/π/θ | ε/π/θ | ε/π/θ | ε/π/θ |
| α2 | 0/γ1/γ2S | 0/γ1/γ2S | 0/γ1/γ2S | 0/γ1/γ2S | 0/γ1/γ2S | 0/γ1/γ2S | 0/γ1/γ2S | 0/γ1/γ2S |
| α3 | γ2L/γ3/δ | γ2L/γ3/δ | γ2L/γ3/δ | γ2L/γ3/δ | γ2L/γ3/δ | γ2L/γ3/δ | γ2L/γ3/δ | γ2L/γ3/δ |
| | ε/π/θ | ε/π/θ | ε/π/θ | ε/π/θ | ε/π/θ | ε/π/θ | ε/π/θ | ε/π/θ |
| α2 | 0/γ1/γ2S | 0/γ1/γ2S | 0/γ1/γ2S | 0/γ1/γ2S | 0/γ1/γ2S | 0/γ1/γ2S | 0/γ1/γ2S | 0/γ1/γ2S |
| α4 | γ2L/γ3/δ | γ2L/γ3/δ | γ2L/γ3/δ | γ2L/γ3/δ | γ2L/γ3/δ | γ2L/γ3/δ | γ2L/γ3/δ | γ2L/γ3/δ |
| | ε/π/θ | ε/π/θ | ε/π/θ | ε/π/θ | ε/π/θ | ε/π/θ | ε/π/θ | ε/π/θ |
| α2 | 0/γ1/γ2S | 0/γ1/γ2S | 0/γ1/γ2S | 0/γ1/γ2S | 0/γ1/γ2S | 0/γ1/γ2S | 0/γ1/γ2S | 0/γ1/γ2S |
| α5 | γ2L/γ3/δ | γ2L/γ3/δ | γ2L/γ3/δ | γ2L/γ3/δ | γ2L/γ3/δ | γ2L/γ3/δ | γ2L/γ3/δ | γ2L/γ3/δ |
| | ε/π/θ | ε/π/θ | ε/π/θ | ε/π/θ | ε/π/θ | ε/π/θ | ε/π/θ | ε/π/θ |
| α2 | 0/γ1/γ2S | 0/γ1/γ2S | 0/γ1/γ2S | 0/γ1/γ2S | 0/γ1/γ2S | 0/γ1/γ2S | 0/γ1/γ2S | 0/γ1/γ2S |
| α6 | γ2L/γ3/δ | γ2L/γ3/δ | γ2L/γ3/δ | γ2L/γ3/δ | γ2L/γ3/δ | γ2L/γ3/δ | γ2L/γ3/δ | γ2L/γ3/δ |
| | ε/π/θ | ε/π/θ | ε/π/θ | ε/π/θ | ε/π/θ | ε/π/θ | ε/π/θ | ε/π/θ |
| α3 | 0/γ1/γ2S | 0/γ1/γ2S | 0/γ1/γ2S | 0/γ1/γ2S | 0/γ1/γ2S | 0/γ1/γ2S | 0/γ1/γ2S | 0/γ1/γ2S |
| α3 | γ2L/γ3/δ | γ2L/γ3/δ | γ2L/γ3/δ | γ2L/γ3/δ | γ2L/γ3/δ | γ2L/γ3/δ | γ2L/γ3/δ | γ2L/γ3/δ |
| | ε/π/θ | ε/π/θ | ε/π/θ | ε/π/θ | ε/π/θ | ε/π/θ | ε/π/θ | ε/π/θ |
| α3 | 0/γ1/γ2S | 0/γ1/γ2S | 0/γ1/γ2S | 0/γ1/γ2S | 0/γ1/γ2S | 0/γ1/γ2S | 0/γ1/γ2S | 0/γ1/γ2S |
| α4 | γ2L/γ3/δ | γ2L/γ3/δ | γ2L/γ3/δ | γ2L/γ3/δ | γ2L/γ3/δ | γ2L/γ3/δ | γ2L/γ3/δ | γ2L/γ3/δ |
| | ε/π/θ | ε/π/θ | ε/π/θ | ε/π/θ | ε/π/θ | ε/π/θ | ε/π/θ | ε/π/θ |
| α3 | 0/γ1/γ2S | 0/γ1/γ2S | 0/γ1/γ2S | 0/γ1/γ2S | 0/γ1/γ2S | 0/γ1/γ2S | 0/γ1/γ2S | 0/γ1/γ2S |
| α5 | γ2L/γ3/δ | γ2L/γ3/δ | γ2L/γ3/δ | γ2L/γ3/δ | γ2L/γ3/δ | γ2L/γ3/δ | γ2L/γ3/δ | γ2L/γ3/δ |
| | ε/π/θ | ε/π/θ | ε/π/θ | ε/π/θ | ε/π/θ | ε/π/θ | ε/π/θ | ε/π/θ |
| α3 | 0/γ1/γ2S | 0/γ1/γ2S | 0/γ1/γ2S | 0/γ1/γ2S | 0/γ1/γ2S | 0/γ1/γ2S | 0/γ1/γ2S | 0/γ1/γ2S |
| α6 | γ2L/γ3/δ | γ2L/γ3/δ | γ2L/γ3/δ | γ2L/γ3/δ | γ2L/γ3/δ | γ2L/γ3/δ | γ2L/γ3/δ | γ2L/γ3/δ |
| | ε/π/θ | ε/π/θ | ε/π/θ | ε/π/θ | ε/π/θ | ε/π/θ | ε/π/θ | ε/π/θ |
| α4 | 0/γ1/γ2S | 0/γ1/γ2S | 0/γ1/γ2S | 0/γ1/γ2S | 0/γ1/γ2S | 0/γ1/γ2S | 0/γ1/γ2S | 0/γ1/γ2S |
| α4 | γ2L/γ3/δ | γ2L/γ3/δ | γ2L/γ3/δ | γ2L/γ3/δ | γ2L/γ3/δ | γ2L/γ3/δ | γ2L/γ3/δ | γ2L/γ3/δ |
| | ε/π/θ | ε/π/θ | ε/π/θ | ε/π/θ | ε/π/θ | ε/π/θ | ε/π/θ | ε/π/θ |
| α4 | 0/γ1/γ2S | 0/γ1/γ2S | 0/γ1/γ2S | 0/γ1/γ2S | 0/γ1/γ2S | 0/γ1/γ2S | 0/γ1/γ2S | 0/γ1/γ2S |
| α5 | γ2L/γ3/δ | γ2L/γ3/δ | γ2L/γ3/δ | γ2L/γ3/δ | γ2L/γ3/δ | γ2L/γ3/δ | γ2L/γ3/δ | γ2L/γ3/δ |
| | ε/π/θ | ε/π/θ | ε/π/θ | ε/π/θ | ε/π/θ | ε/π/θ | ε/π/θ | ε/π/θ |
| α4 | 0/γ1/γ2S | 0/γ1/γ2S | 0/γ1/γ2S | 0/γ1/γ2S | 0/γ1/γ2S | 0/γ1/γ2S | 0/γ1/γ2S | 0/γ1/γ2S |
| α6 | γ2L/γ3/δ | γ2L/γ3/δ | γ2L/γ3/δ | γ2L/γ3/δ | γ2L/γ3/δ | γ2L/γ3/δ | γ2L/γ3/δ | γ2L/γ3/δ |
| | ε/π/θ | ε/π/θ | ε/π/θ | ε/π/θ | ε/π/θ | ε/π/θ | ε/π/θ | ε/π/θ |
| α5 | 0/γ1/γ2S | 0/γ1/γ2S | 0/γ1/γ2S | 0/γ1/γ2S | 0/γ1/γ2S | 0/γ1/γ2S | 0/γ1/γ2S | 0/γ1/γ2S |
| α5 | γ2L/γ3/δ | γ2L/γ3/δ | γ2L/γ3/δ | γ2L/γ3/δ | γ2L/γ3/δ | γ2L/γ3/δ | γ2L/γ3/δ | γ2L/γ3/δ |
| | ε/π/θ | ε/π/θ | ε/π/θ | ε/π/θ | ε/π/θ | ε/π/θ | ε/π/θ | ε/π/θ |
| α5 | 0/γ1/γ2S | 0/γ1/γ2S | 0/γ1/γ2S | 0/γ1/γ2S | 0/γ1/γ2S | 0/γ1/γ2S | 0/γ1/γ2S | 0/γ1/γ2S |
| α6 | γ2L/γ3/δ | γ2L/γ3/δ | γ2L/γ3/δ | γ2L/γ3/δ | γ2L/γ3/δ | γ2L/γ3/δ | γ2L/γ3/δ | γ2L/γ3/δ |
| | ε/π/θ | ε/π/θ | ε/π/θ | ε/π/θ | ε/π/θ | ε/π/θ | ε/π/θ | ε/π/θ |
| α6 | 0/γ1/γ2S | 0/γ1/γ2S | 0/γ1/γ2S | 0/γ1/γ2S | 0/γ1/γ2S | 0/γ1/γ2S | 0/γ1/γ2S | 0/γ1/γ2S |
| α6 | γ2L/γ3/δ | γ2L/γ3/δ | γ2L/γ3/δ | γ2L/γ3/δ | γ2L/γ3/δ | γ2L/γ3/δ | γ2L/γ3/δ | γ2L/γ3/δ |
| | ε/π/θ | ε/π/θ | ε/π/θ | ε/π/θ | ε/π/θ | ε/π/θ | ε/π/θ | ε/π/θ |

TABLE 3b

| | β2Lβ3.2 | β2Sβ2S | β2Sβ3.1 | β2Sβ3.2 | β3.1β3.1 | β3.1β3.2 | β3.2β3.2 |
|---|---|---|---|---|---|---|---|
| α1 | 0/γ1/γ2S | 0/γ1/γ2S | 0/γ1/γ2S | 0/γ1/γ2S | 0/γ1/γ2S | 0/γ1/γ2S | 0/γ1/γ2S |
| α1 | γ2L/γ3/δ | γ2L/γ3/δ | γ2L/γ3/δ | γ2L/γ3/δ | γ2L/γ3/δ | γ2L/γ3/δ | γ2L/γ3/δ |
| | ε/π/θ | ε/π/θ | ε/π/θ | ε/π/θ | ε/π/θ | ε/π/θ | ε/π/θ |
| α1 | 0/γ1/γ2S | 0/γ1/γ2S | 0/γ1/γ2S | 0/γ1/γ2S | 0/γ1/γ2S | 0/γ1/γ2S | 0/γ1/γ2S |
| α2 | γ2L/γ3/δ | γ2L/γ3/δ | γ2L/γ3/δ | γ2L/γ3/δ | γ2L/γ3/δ | γ2L/γ3/δ | γ2L/γ3/δ |
| | ε/π/θ | ε/π/θ | ε/π/θ | ε/π/θ | ε/π/θ | ε/π/θ | ε/π/θ |
| α1 | 0/γ1/γ2S | 0/γ1/γ2S | 0/γ1/γ2S | 0/γ1/γ2S | 0/γ1/γ2S | 0/γ1/γ2S | 0/γ1/γ2S |
| α3 | γ2L/γ3/δ | γ2L/γ3/δ | γ2L/γ3/δ | γ2L/γ3/δ | γ2L/γ3/δ | γ2L/γ3/δ | γ2L/γ3/δ |
| | ε/π/θ | ε/π/θ | ε/π/θ | ε/π/θ | ε/π/θ | ε/π/θ | ε/π/θ |
| α1 | 0/γ1/γ2S | 0/γ1/γ2S | 0/γ1/γ2S | 0/γ1/γ2S | 0/γ1/γ2S | 0/γ1/γ2S | 0/γ1/γ2S |
| α4 | γ2L/γ3/δ | γ2L/γ3/δ | γ2L/γ3/δ | γ2L/γ3/δ | γ2L/γ3/δ | γ2L/γ3/δ | γ2L/γ3/δ |
| | ε/π/θ | ε/π/θ | ε/π/θ | ε/π/θ | ε/π/θ | ε/π/θ | ε/π/θ |
| α1 | 0/γ1/γ2S | 0/γ1/γ2S | 0/γ1/γ2S | 0/γ1/γ2S | 0/γ1/γ2S | 0/γ1/γ2S | 0/γ1/γ2S |
| α5 | γ2L/γ3/δ | γ2L/γ3/δ | γ2L/γ3/δ | γ2L/γ3/δ | γ2L/γ3/δ | γ2L/γ3/δ | γ2L/γ3/δ |
| | ε/π/θ | ε/π/θ | ε/π/θ | ε/π/θ | ε/π/θ | ε/π/θ | ε/π/θ |
| α1 | 0/γ1/γ2S | 0/γ1/γ2S | 0/γ1/γ2S | 0/γ1/γ2S | 0/γ1/γ2S | 0/γ1/γ2S | 0/γ1/γ2S |
| α6 | γ2L/γ3/δ | γ2L/γ3/δ | γ2L/γ3/δ | γ2L/γ3/δ | γ2L/γ3/δ | γ2L/γ3/δ | γ2L/γ3/δ |
| | ε/π/θ | ε/π/θ | ε/π/θ | ε/π/θ | ε/π/θ | ε/π/θ | ε/π/θ |
| α2 | 0/γ1/γ2S | 0/γ1/γ2S | 0/γ1/γ2S | 0/γ1/γ2S | 0/γ1/γ2S | 0/γ1/γ2S | 0/γ1/γ2S |
| α2 | γ2L/γ3/δ | γ2L/γ3/δ | γ2L/γ3/δ | γ2L/γ3/δ | γ2L/γ3/δ | γ2L/γ3/δ | γ2L/γ3/δ |
| | ε/π/θ | ε/π/θ | ε/π/θ | ε/π/θ | ε/π/θ | ε/π/θ | ε/π/θ |
| α12 | 0/γ1/γ2S | 0/γ1/γ2S | 0/γ1/γ2S | 0/γ1/γ2S | 0/γ1/γ2S | 0/γ1/γ2S | 0/γ1/γ2S |
| α3 | γ2L/γ3/δ | γ2L/γ3/δ | γ2L/γ3/δ | γ2L/γ3/δ | γ2L/γ3/δ | γ2L/γ3/δ | γ2L/γ3/δ |
| | ε/π/θ | ε/π/θ | ε/π/θ | ε/π/θ | ε/π/θ | ε/π/θ | ε/π/θ |
| α2 | 0/γ1/γ2S | 0/γ1/γ2S | 0/γ1/γ2S | 0/γ1/γ2S | 0/γ1/γ2S | 0/γ1/γ2S | 0/γ1/γ2S |
| α4 | γ2L/γ3/δ | γ2L/γ3/δ | γ2L/γ3/δ | γ2L/γ3/δ | γ2L/γ3/δ | γ2L/γ3/δ | γ2L/γ3/δ |
| | ε/π/θ | ε/π/θ | ε/π/θ | ε/π/θ | ε/π/θ | ε/π/θ | ε/π/θ |
| α2 | 0/γ1/γ2S | 0/γ1/γ2S | 0/γ1/γ2S | 0/γ1/γ2S | 0/γ1/γ2S | 0/γ1/γ2S | 0/γ1/γ2S |
| α5 | γ2L/γ3/δ | γ2L/γ3/δ | γ2L/γ3/δ | γ2L/γ3/δ | γ2L/γ3/δ | γ2L/γ3/δ | γ2L/γ3/δ |
| | ε/π/θ | ε/π/θ | ε/π/θ | ε/π/θ | ε/π/θ | ε/π/θ | ε/π/θ |
| α2 | 0/γ1/γ2S | 0/γ1/γ2S | 0/γ1/γ2S | 0/γ1/γ2S | 0/γ1/γ2S | 0/γ1/γ2S | 0/γ1/γ2S |
| α6 | γ2L/γ3/δ | γ2L/γ3/δ | γ2L/γ3/δ | γ2L/γ3/δ | γ2L/γ3/δ | γ2L/γ3/δ | γ2L/γ3/δ |
| | ε/π/θ | ε/π/θ | ε/π/θ | ε/π/θ | ε/π/θ | ε/π/θ | ε/π/θ |
| α3 | 0/γ1/γ2S | 0/γ1/γ2S | 0/γ1/γ2S | 0/γ1/γ2S | 0/γ1/γ2S | 0/γ1/γ2S | 0/γ1/γ2S |
| α3 | γ2L/γ3/δ | γ2L/γ3/δ | γ2L/γ3/δ | γ2L/γ3/δ | γ2L/γ3/δ | γ2L/γ3/δ | γ2L/γ3/δ |
| | ε/π/θ | ε/π/θ | ε/π/θ | ε/π/θ | ε/π/θ | ε/π/θ | ε/π/θ |
| α3 | 0/γ1/γ2S | 0/γ1/γ2S | 0/γ1/γ2S | 0/γ1/γ2S | 0/γ1/γ2S | 0/γ1/γ2S | 0/γ1/γ2S |
| α4 | γ2L/γ3/δ | γ2L/γ3/δ | γ2L/γ3/δ | γ2L/γ3/δ | γ2L/γ3/δ | γ2L/γ3/δ | γ2L/γ3/δ |
| | ε/π/θ | ε/π/θ | ε/π/θ | ε/π/θ | ε/π/θ | ε/π/θ | ε/π/θ |
| α3 | 0/γ1/γ2S | 0/γ1/γ2S | 0/γ1/γ2S | 0/γ1/γ2S | 0/γ1/γ2S | 0/γ1/γ2S | 0/γ1/γ2S |
| α5 | γ2L/γ3/δ | γ2L/γ3/δ | γ2L/γ3/δ | γ2L/γ3/δ | γ2L/γ3/δ | γ2L/γ3/δ | γ2L/γ3/δ |
| | ε/π/θ | ε/π/θ | ε/π/θ | ε/π/θ | ε/π/θ | ε/π/θ | ε/π/θ |
| α3 | 0/γ1/γ2S | 0/γ1/γ2S | 0/γ1/γ2S | 0/γ1/γ2S | 0/γ1/γ2S | 0/γ1/γ2S | 0/γ1/γ2S |
| α6 | γ2L/γ3/δ | γ2L/γ3/δ | γ2L/γ3/δ | γ2L/γ3/δ | γ2L/γ3/δ | γ2L/γ3/δ | γ2L/γ3/δ |
| | ε/π/θ | ε/π/θ | ε/π/θ | ε/π/θ | ε/π/θ | ε/π/θ | ε/π/θ |
| α4 | 0/γ1/γ2S | 0/γ1/γ2S | 0/γ1/γ2S | 0/γ1/γ2S | 0/γ1/γ2S | 0/γ1/γ2S | 0/γ1/γ2S |
| α4 | γ2L/γ3/δ | γ2L/γ3/δ | γ2L/γ3/δ | γ2L/γ3/δ | γ2L/γ3/δ | γ2L/γ3/δ | γ2L/γ3/δ |
| | ε/π/θ | ε/π/θ | ε/π/θ | ε/π/θ | ε/π/θ | ε/π/θ | ε/π/θ |
| α4 | 0/γ1/γ2S | 0/γ1/γ2S | 0/γ1/γ2S | 0/γ1/γ2S | 0/γ1/γ2S | 0/γ1/γ2S | 0/γ1/γ2S |
| α5 | γ2L/γ3/δ | γ2L/γ3/δ | γ2L/γ3/δ | γ2L/γ3/δ | γ2L/γ3/δ | γ2L/γ3/δ | γ2L/γ3/δ |
| | ε/π/θ | ε/π/θ | ε/π/θ | ε/π/θ | ε/π/θ | ε/π/θ | ε/π/θ |
| α4 | 0/γ1/γ2S | 0/γ1/γ2S | 0/γ1/γ2S | 0/γ1/γ2S | 0/γ1/γ2S | 0/γ1/γ2S | 0/γ1/γ2S |
| α6 | γ2L/γ3/δ | γ2L/γ3/δ | γ2L/γ3/δ | γ2L/γ3/δ | γ2L/γ3/δ | γ2L/γ3/δ | γ2L/γ3/δ |
| | ε/π/θ | ε/π/θ | ε/π/θ | ε/π/θ | ε/π/θ | ε/π/θ | ε/π/θ |
| α5 | 0/γ1/γ2S | 0/γ1/γ2S | 0/γ1/γ2S | 0/γ1/γ2S | 0/γ1/γ2S | 0/γ1/γ2S | 0/γ1/γ2S |
| α5 | γ2L/γ3/δ | γ2L/γ3/δ | γ2L/γ3/δ | γ2L/γ3/δ | γ2L/γ3/δ | γ2L/γ3/δ | γ2L/γ3/δ |
| | ε/π/θ | ε/π/θ | ε/π/θ | ε/π/θ | ε/π/θ | ε/π/θ | ε/π/θ |
| α5 | 0/γ1/γ2S | 0/γ1/γ2S | 0/γ1/γ2S | 0/γ1/γ2S | 0/γ1/γ2S | 0/γ1/γ2S | 0/γ1/γ2S |
| α6 | γ2L/γ3/δ | γ2L/γ3/δ | γ2L/γ3/δ | γ2L/γ3/δ | γ2L/γ3/δ | γ2L/γ3/δ | γ2L/γ3/δ |
| | ε/π/θ | ε/π/θ | ε/π/θ | ε/π/θ | ε/π/θ | ε/π/θ | ε/π/θ |
| α6 | 0/γ1/γ2S | 0/γ1/γ2S | 0/γ1/γ2S | 0/γ1/γ2S | 0/γ1/γ2S | 0/γ1/γ2S | 0/γ1/γ2S |
| α6 | γ2L/γ3/δ | γ2L/γ3/δ | γ2L/γ3/δ | γ2L/γ3/δ | γ2L/γ3/δ | γ2L/γ3/δ | γ2L/γ3/δ |
| | ε/π/θ | ε/π/θ | ε/π/θ | ε/π/θ | ε/π/θ | ε/π/θ | ε/π/θ |

This invention also solves a difficulty in generating stable GABA_{A} receptor expressing cells and cell lines. In some embodiments, the cell lines of the invention express GABA_{A} subunits in isolation from other modulating factors found in endogenous cells.

According to the invention, the GABA receptor expressed by a cell or cell line can be from any mammal, such as, but not limited to, human, non-human primate, bovine, porcine, feline, rat, marsupial, murine, canine, ovine, caprine, rabbit, guinea pig and hamster. Table 4 (below) comprises a non-limiting list of GABA receptor subunits in various species. The GABA subunits can be from the same or different species. In some embodiments, the GABA subunits form a functional GABA receptor. In preferred embodiments the GABA receptor is a GABA_{A} receptor. In other embodiments, the GABA_{A} receptor is a human GABA_{A} receptor, comprising human alpha; human beta; and human gamma, delta, epsilon, pi, theta, or rho subunits.

TABLE 4

| Receptor subunit | Gene name | Species |
|---|---|---|
| GABA_{A}: | | |
| gamma-aminobutyric acid (GABA) A receptor, alpha 1 | GABRA1 | Homo sapiens |
| gamma-aminobutyric acid (GABA) A receptor, alpha 1 | Gabra1 | Mus musculus |
| gamma-aminobutyric acid (GABA) A receptor, alpha 1 | gabra1 | Danio rerio |
| gamma-aminobutyric acid (GABA) A receptor, alpha 1 | GABRA1 | Pan troglodytes |
| gamma-aminobutyric acid (GABA) A receptor, alpha 1 | GABRA1 | Bos taurus |
| | (variant 1) | |
| gamma-aminobutyric acid (GABA) A receptor, alpha 1 | GABRA1 | Bos taurus |
| | (variant 2) | |
| gamma-aminobutyric acid (GABA) A receptor, alpha 1 | GABRA1 | Gallus gallus |
| gamma-aminobutyric acid (GABA) A receptor, alpha 1 | GABRA1 | Canis familiaris |
| gamma-aminobutyric acid (GABA) A receptor, alpha 1 | Gabra1 | Rattus |
| | | norvegicus |
| gamma-aminobutyric acid (GABA) A receptor, alpha 2 | GABRA2 | Homo sapiens |
| gamma-aminobutyric acid (GABA) A receptor, alpha 2 | Gabra2 | Mus musculus |
| gamma-aminobutyric acid (GABA) A receptor, alpha 2 | LOC100150704 | Danio rerio |
| gamma-aminobutyric acid (GABA) A receptor, alpha 2 | GABRA2 | Pan troglodytes |
| gamma-aminobutyric acid (GABA) A receptor, alpha 2 | GABRA2 | Bos taurus |
| gamma-aminobutyric acid (GABA) A receptor, alpha 2 | GABRA2 | Gallus gallus |
| gamma-aminobutyric acid (GABA) A receptor, alpha 2 | GABRA2 | Canis familiaris |
| gamma-aminobutyric acid (GABA) A receptor, alpha 2 | LOC289606 | Rattus |
| | | norvegicus |
| gamma-aminobutyric acid (GABA) A receptor, alpha 3 | GABRA3 | Homo sapiens |
| gamma-aminobutyric acid (GABA) A receptor, alpha 3 | Gabra3 | Mus musculus |
| gamma-aminobutyric acid (GABA) A receptor, alpha 3 | Grd | Drosophila |
| | | melanogaster |
| gamma-aminobutyric acid (GABA) A receptor, alpha 3 | GABRA3 | Bos taurus |
| gamma-aminobutyric acid (GABA) A receptor, alpha 3 | GABRA3 | Gallus gallus |
| gamma-aminobutyric acid (GABA) A receptor, alpha 3 | GABRA3 | Canis familiaris |
| gamma-aminobutyric acid (GABA) A receptor, alpha 3 | Gabra3 | Rattus |
| | | norvegicus |
| gamma-aminobutyric acid (GABA) A receptor, alpha 4 | GABRA4 | Homo sapiens |
| gamma-aminobutyric acid (GABA) A receptor, alpha 4 | Gabra4 | Mus musculus |
| gamma-aminobutyric acid (GABA) A receptor, alpha 4 | zgc:110204 | Danio rerio |
| gamma-aminobutyric acid (GABA) A receptor, alpha 4 | GABRA4 | Pan troglodytes |
| gamma-aminobutyric acid (GABA) A receptor, alpha 4 | GABRA4 | Bos taurus |
| gamma-aminobutyric acid (GABA) A receptor, alpha 4 | GABRA4 | Gallus gallus |
| gamma-aminobutyric acid (GABA) A receptor, alpha 4 | GABRA4 | Canis familiaris |
| gamma-aminobutyric acid (GABA) A receptor, alpha 4 | Gabra4 | Rattus |
| | | norvegicus |
| gamma-aminobutyric acid (GABA) A receptor, alpha 5 | GABRA5 | Homo sapiens |
| gamma-aminobutyric acid (GABA) A receptor, alpha 5 | Gabra5 | Mus musculus |
| gamma-aminobutyric acid (GABA) A receptor, alpha 5 | CG8916 | Drosophila |
| | | melanogaster |
| gamma-aminobutyric acid (GABA) A receptor, alpha 5 | Igc-37 | Caenorhabditis |
| | | elegans |
| gamma-aminobutyric acid (GABA) A receptor, alpha 5 | LOC799124 | Danio rerio |
| gamma-aminobutyric acid (GABA) A receptor, alpha 5 | GABRA5 | Bos taurus |
| gamma-aminobutyric acid (GABA) A receptor, alpha 5 | GABRA5 | Gallus gallus |
| gamma-aminobutyric acid (GABA) A receptor, alpha 5 | GABRA5 | Canis familiaris |
| gamma-aminobutyric acid (GABA) A receptor, alpha 5 | Gabra5 | Rattus |
| | | norvegicus |
| gamma-aminobutyric acid (GABA) A receptor, alpha 6 | GABRA6 | Homo sapiens |
| gamma-aminobutyric acid (GABA) A receptor, alpha 6 | Gabra6 | Mus musculus |
| gamma-aminobutyric acid (GABA) A receptor, alpha 6 | Rdl | Drosophila |
| | | melanogaster |
| gamma-aminobutyric acid (GABA) A receptor, alpha 6 | Igc-38 | Caenorhabditis |
| | | elegans |
| gamma-aminobutyric acid (GABA) A receptor, alpha 6 | gabra6a | Danio rerio |
| gamma-aminobutyric acid (GABA) A receptor, alpha 6 | gabra6b | Danio rerio |
| gamma-aminobutyric acid (GABA) A receptor, alpha 6 | GABRA6 | Pan troglodytes |
| gamma-aminobutyric acid (GABA) A receptor, alpha 6 | GABRA6 | Bos taurus |
| gamma-aminobutyric acid (GABA) A receptor, alpha 6 | GABRA6 | Gallus gallus |
| gamma-aminobutyric acid (GABA) A receptor, alpha 6 | GABRA6 | Canis familiaris |
| gamma-aminobutyric acid (GABA) A receptor, alpha 6 | Gabra6 | Rattus |
| | | norvegicus |
| gamma-aminobutyric acid (GABA) A receptor, beta 1 | GABRB1 | Homo sapiens |
| gamma-aminobutyric acid (GABA) A receptor, beta 1 | Gabrb1 | Mus musculus |
| gamma-aminobutyric acid (GABA) A receptor, beta 1 | GABRB1 | Pan troglodytes |
| gamma-aminobutyric acid (GABA) A receptor, beta 1 | GABRB1 | Bos taurus |
| gamma-aminobutyric acid (GABA) A receptor, beta 1 | GABRB1 | Gallus gallus |
| gamma-aminobutyric acid (GABA) A receptor, beta 1 | Gabrb1 | Rattus |
| | | norvegicus |
| gamma-aminobutyric acid (GABA) A receptor, beta 2 | GABRB2 | Homo sapiens |
| gamma-aminobutyric acid (GABA) A receptor, beta 2 | Gabrb2 | Mus musculus |
| gamma-aminobutyric acid (GABA) A receptor, beta 2 | gabrb2 | Danio rerio |
| gamma-aminobutyric acid (GABA) A receptor, beta 2 | GABRB2 | Pan troglodytes |
| gamma-aminobutyric acid (GABA) A receptor, beta 2 | GABRB2 | Bos taurus |
| gamma-aminobutyric acid (GABA) A receptor, beta 2 | GABRB2 | Gallus gallus |
| gamma-aminobutyric acid (GABA) A receptor, beta 2 | GABRB2 | Canis familiaris |
| gamma-aminobutyric acid (GABA) A receptor, beta 2 | Gabrb2 | Rattus |
| | | norvegicus |
| gamma-aminobutyric acid (GABA) A receptor, beta 3 | GABRB3 | Homo sapiens |
| gamma-aminobutyric acid (GABA) A receptor, beta 3 | Gabrb3 | Mus musculus |
| gamma-aminobutyric acid (GABA) A receptor, beta 3 | Lcch3 | Drosophila |
| | | melanogaster |
| gamma-aminobutyric acid (GABA) A receptor, beta 3 | gab-1 | Caenorhabditis |
| | | elegans |
| gamma-aminobutyric acid (GABA) A receptor, beta 3 | LOC566922 | Danio rerio |
| gamma-aminobutyric acid (GABA) A receptor, beta 3 | GABRB3 | Pan troglodytes |
| gamma-aminobutyric acid (GABA) A receptor, beta 3 | GABRB3 | Gallus gallus |
| gamma-aminobutyric acid (GABA) A receptor, beta 3 | GABRB3 | Canis familiaris |
| gamma-aminobutyric acid (GABA) A receptor, beta 3 | Gabrb3 | Rattus |
| | | norvegicus |
| gamma-aminobutyric acid (GABA) A receptor, gamma 1 | GABRG1 | Homo sapiens |
| gamma-aminobutyric acid (GABA) A receptor, gamma 1 | Gabrg1 | Mus musculus |
| gamma-aminobutyric acid (GABA) A receptor, gamma 1 | LOC556202 | Danio rerio |
| gamma-aminobutyric acid (GABA) A receptor, gamma 1 | GABRG1 | Pan troglodytes |
| gamma-aminobutyric acid (GABA) A receptor, gamma 1 | GABRG1 | Bos taurus |
| gamma-aminobutyric acid (GABA) A receptor, gamma 1 | GABRG1 | Gallus gallus |
| gamma-aminobutyric acid (GABA) A receptor, gamma 1 | GABRG1 | Canis familiaris |
| gamma-aminobutyric acid (GABA) A receptor, gamma 1 | Gabrg1 | Rattus |
| | | norvegicus |
| gamma-aminobutyric acid (GABA) A receptor, gamma 2 | GABRG2 | Homo sapiens |
| gamma-aminobutyric acid (GABA) A receptor, gamma 2 | Gabrg2 | Mus musculus |
| gamma-aminobutyric acid (GABA) A receptor, gamma 2 | LOC553402 | Danio rerio |
| gamma-aminobutyric acid (GABA) A receptor, gamma 2 | GABRG2 | Bos taurus |
| gamma-aminobutyric acid (GABA) A receptor, gamma 2 | GABRG2 | Gallus gallus |
| gamma-aminobutyric acid (GABA) A receptor, gamma 2 | GABRG2 | Canis familiaris |
| gamma-aminobutyric acid (GABA) A receptor, gamma 2 | Gabrg2 | Rattus |
| | | norvegicus |
| gamma-aminobutyric acid (GABA) A receptor, gamma 3 | GABRG3 | Homo sapiens |
| gamma-aminobutyric acid (GABA) A receptor, gamma 3 | Gabrg3 | Mus musculus |
| gamma-aminobutyric acid (GABA) A receptor, gamma 3 | LOC567057 | Danio rerio |
| gamma-aminobutyric acid (GABA) A receptor, gamma 3 | GABRG3 | Gallus gallus |
| gamma-aminobutyric acid (GABA) A receptor, gamma 3 | Gabrg3 | Rattus |
| | | norvegicus |
| gamma-aminobutyric acid (GABA) A receptor, delta | GABRD | Homo sapiens |
| gamma-aminobutyric acid (GABA) A receptor, delta | Gabrd | Mus musculus |
| gamma-aminobutyric acid (GABA) A receptor, delta | DKEYP- | Danio rerio |
| | 87A12.2 | |
| gamma-aminobutyric acid (GABA) A receptor, delta | GABRD | Pan troglodytes |
| gamma-aminobutyric acid (GABA) A receptor, delta | GABRD | Bos taurus |
| gamma-aminobutyric acid (GABA) A receptor, delta | GABRD | Gallus gallus |
| gamma-aminobutyric acid (GABA) A receptor, delta | GABRD | Canis familiaris |
| gamma-aminobutyric acid (GABA) A receptor, delta | Gabrd | Rattus |
| | | norvegicus |
| gamma-aminobutyric acid (GABA) A receptor, epsilon | GABRE | Homo sapiens |
| gamma-aminobutyric acid (GABA) A receptor, epsilon | Gabre | Mus musculus |
| gamma-aminobutyric acid (GABA) A receptor, epsilon | GABRE | Pan troglodytes |
| gamma-aminobutyric acid (GABA) A receptor, epsilon | GABRE | Bos taurus |
| gamma-aminobutyric acid (GABA) A receptor, epsilon | GABRE | Canis familiaris |
| gamma-aminobutyric acid (GABA) A receptor, epsilon | Gabre | Rattus |
| | | norvegicus |
| gamma-aminobutyric acid (GABA) A receptor, pi | GABRP | Homo sapiens |
| gamma-aminobutyric acid (GABA) A receptor, pi | Gabrp | Mus musculus |
| gamma-aminobutyric acid (GABA) A receptor, pi | GABRP | Pan troglodytes |
| gamma-aminobutyric acid (GABA) A receptor, pi | GABRP | Bos taurus |
| gamma-aminobutyric acid (GABA) A receptor, pi | GABRP | Gallus gallus |
| gamma-aminobutyric acid (GABA) A receptor, pi | GABRP | Canis familiaris |
| gamma-aminobutyric acid (GABA) A receptor, pi | Gabrp | Rattus |
| | | norvegicus |
| gamma-aminobutyric acid (GABA) A receptor, theta | GABRQ | Homo sapiens |
| gamma-aminobutyric acid (GABA) A receptor, theta | Gabrq | Mus musculus |
| gamma-aminobutyric acid (GABA) A receptor, theta | GABRQ | Pan troglodytes |
| gamma-aminobutyric acid (GABA) A receptor, theta | GABRQ | Bos taurus |
| gamma-aminobutyric acid (GABA) A receptor, theta | GABRQ | Canis familiaris |
| gamma-aminobutyric acid (GABA) A receptor, theta | Gabrq | Rattus |
| | | norvegicus |
| GABA_{B}: | | |
| gamma-aminobutyric acid (GABA) B receptor, 1 | GABBR1 | Homo sapiens |
| gamma-aminobutyric acid (GABA) B receptor, 1 | Gabbr1 | Mus musculus |
| gamma-aminobutyric acid (GABA) B receptor, 1 | GABA-B-R1 | Drosophila |
| | | melanogaster |
| gamma-aminobutyric acid (GABA) B receptor, 1 | Y41G9A.4 | Caenorhabditis |
| | | elegans |
| gamma-aminobutyric acid (GABA) B receptor, 1 | gabbr1 | Danio rerio |
| gamma-aminobutyric acid (GABA) B receptor, 1 | GABBR1 | Pan troglodytes |
| gamma-aminobutyric acid (GABA) B receptor, 1 | GABBR1 | Bos taurus |
| gamma-aminobutyric acid (GABA) B receptor, 1 | GABBR1 | Canis familiaris |
| gamma-aminobutyric acid (GABA) B receptor, 1 | Gabbr1 | Rattus |
| | | norvegicus |
| gamma-aminobutyric acid (GABA) B receptor, 2 | GABBR2 | Homo sapiens |
| gamma-aminobutyric acid (GABA) B receptor, 2 | Gabbr2 | Mus musculus |
| gamma-aminobutyric acid (GABA) B receptor, 2 | GABA-B-R2 | Drosophila |
| | | melanogaster |
| gamma-aminobutyric acid (GABA) B receptor, 2 | si:dkey-190I1.2 | Danio rerio |
| gamma-aminobutyric acid (GABA) B receptor, 2 | GABBR2 | Pan troglodytes |
| gamma-aminobutyric acid (GABA) B receptor, 2 | GABBR2 | Bos taurus |
| gamma-aminobutyric acid (GABA) B receptor, 2 | GABBR2 | Gallus gallus |
| gamma-aminobutyric acid (GABA) B receptor, 2 | GABBR2 | Canis familiaris |
| gamma-aminobutyric acid (GABA) B receptor, 2 | Gabbr2 | Rattus |
| | | norvegicus |
| GABA_{C}: | | |
| gamma-aminobutyric acid (GABA) A receptor, rho1 | GABRR1 | Homo sapiens |
| gamma-aminobutyric acid (GABA) A receptor, rho1 | Gabrr1 | Mus musculus |
| gamma-aminobutyric acid (GABA) A receptor, rho1 | gabrr1 | Danio rerio |
| gamma-aminobutyric acid (GABA) A receptor, rho1 | GABRR1 | Pan troglodytes |
| gamma-aminobutyric acid (GABA) A receptor, rho1 | GABRR1 | Bos taurus |
| gamma-aminobutyric acid (GABA) A receptor, rho1 | GABRR1 | Gallus gallus |
| gamma-aminobutyric acid (GABA) A receptor, rho1 | GABRR1 | Canis familiaris |
| gamma-aminobutyric acid (GABA) A receptor, rho1 | Gabrr1 | Rattus |
| | | norvegicus |
| gamma-aminobutyric acid (GABA) A receptor, rho2 | GABRR2 | Homo sapiens |
| gamma-aminobutyric acid (GABA) A receptor, rho2 | Gabrr2 | Mus musculus |
| gamma-aminobutyric acid (GABA) A receptor, rho2 | si:dkey-181i3.1 | Danio rerio |
| gamma-aminobutyric acid (GABA) A receptor, rho2 | GABRR2 | Pan troglodytes |
| gamma-aminobutyric acid (GABA) A receptor, rho2 | GABRR2 | Gallus gallus |
| gamma-aminobutyric acid (GABA) A receptor, rho2 | GABRR2 | Canis familiaris |
| gamma-aminobutyric acid (GABA) A receptor, rho2 | Gabrr2 | Rattus |
| | | norvegicus |
| gamma-aminobutyric acid (GABA) A receptor, rho3 | GABRR3 | Homo sapiens |
| gamma-aminobutyric acid (GABA) A receptor, rho3 | Gabrr3 | Mus musculus |
| gamma-aminobutyric acid (GABA) A receptor, rho3 | zgc:194845 | Danio rerio |
| gamma-aminobutyric acid (GABA) A receptor, rho3 | GABRR3 | Pan troglodytes |
| gamma-aminobutyric acid (GABA) A receptor, rho3 | GABRR3 | VBos taurus |
| gamma-aminobutyric acid (GABA) A receptor, rho3 | GABRR3 | Gallus gallus |
| gamma-aminobutyric acid (GABA) A receptor, rho3 | Gabrr3 | Rattus |
| | | norvegicus |

In particular embodiments, a GABA_{A} alpha subunit from any species may be co-expressed with any GABA_{A} beta subunit from any species, and any GABA_{A} gamma subunit from any species in a cell or cell line of the invention. Similarly, any GABA_{A} alpha subunit from any species may be co-expressed with any GABA_{A} beta subunit from any species, and a GABA_{A} delta, epsilon, pi, theta, or rho subunit from any species in a cell line of the invention. In some embodiments, a GABA_{A} subunit may be a chimeric subunit comprising sequences form two or more species. In some embodiments, the novel cell and cell line stably expresses human GABA_{A} subunits, for example a cell or cell line that expresses at least one human GABA_{A} alpha subunit (SEQ ID NO: 1-6); at least one human GABA_{A} beta subunit (SEQ ID NO: 7-11); and at least one human GABA_{A} gamma, delta, epsilon, pi, theta, or rho subunit (SEQ ID NO: 12-22). In some embodiments, the novel cell line is triply transfected to expresses a human GABA_{A} alpha subunit, a human GABA_{A} beta subunit and a human GABA_{A} gamma, delta, epsilon, pi, theta, or rho subunit.

In some embodiments, a cell or cell line of the invention may comprise a nucleic acid sequence that encodes any human GABA_{A} alpha subunit; any human GABA_{A} beta subunit; and any human GABA_{A} gamma, delta, epsilon, pi, theta, or rho subunit. In some embodiments, the human GABA_{A} alpha subunit is encoded by a nucleic acid selected from the group consisting of SEQ ID NOS: 1-6, the human GABA_{A} beta subunit is encoded by a nucleic acid selected from the group consisting of SEQ ID NOS: 7-11, the human GABA_{A} gamma subunit is encoded by a nucleic acid selected from the group consisting of SEQ ID NOS: 12-15, the human GABA_{A} delta subunit is encoded by the nucleic acid set forth in SEQ ID NO: 16, the human GABA_{A} epsilon subunit is encoded by the nucleic acid set forth in SEQ ID NO: 17, the human GABA_{A} pi subunit is encoded by the nucleic acid set forth in SEQ ID NO: 18, the human GABA_{A} theta subunit is encoded by the nucleic acid set forth in SEQ ID NO: 19, and the human GABA_{A} rho subunits are encoded by the nucleic acids set forth in SEQ ID NO: 20-22.

The nucleic acid encoding the GABA_{A} alpha, beta, gamma, delta, epsilon, pi, theta, or rho subunit can be genomic DNA or cDNA. In some embodiments, the nucleic acid encoding the GABA_{A} subunit comprises one or more substitutions, mutations or deletions, as compared to a wild-type GABA_{A} subunit, that may or may not result in an amino acid substitution. In some embodiments, the nucleic acid is a fragment of a nucleic acid sequence encoding a GABA_{A} subunit. Preferably, the GABA_{A} fragments or GABA_{A} mutants retain at least one biological property of a GABA_{A}, e.g., its ability to conduct chloride ions, or to be modulated by GABA.

The invention also encompasses cells and cell lines stably expressing a subunit-encoding nucleotide sequence that is at least about 85% identical to a sequence disclosed herein. In some embodiments, the subunit-encoding sequence identity is at least 85%, 86%, 87%, 88%, 89%, 90%, 91%, 92%, 93%, 94%, 95%, 96%, 97%, 98%, 99% or higher compared to a subunit sequence provided herein. The invention also encompasses cells and cell lines wherein a nucleic acid encoding a GABA_{A} subunit hybridizes under stringent conditions to a nucleic acid provided herein encoding the subunit.

In some embodiments, the cell or cell line comprises a GABA_{A} subunit-encoding nucleic acid sequence comprising a substitution compared to a sequence provided herein by at least one but less than 10, 20, 30, or 40 nucleotides, up to or equal to 1%, 5%, 10% or 20% of the nucleotide sequence or from a sequence substantially identical thereto (e.g., a sequence at least 85%, 86%, 87%, 88%, 89%, 90%, 91%, 92%, 93%, 94%, 95%, 96%, 97%, 98%, 99% or higher identical thereto, or that is capable of hybridizing under stringent conditions to the sequences disclosed). In some embodiments, the cell or cell line comprises a GABA_{A} subunit-encoding nucleic acid sequence comprising an insertion into or deletion from the sequences provided herein by less than 10, 20, 30, or 40 nucleotides up to or equal to 1%, 5%, 10% or 20% of the nucleotide sequence or from a sequence substantially identical thereto (e.g., a sequence at least 85%, 86%, 87%, 88%, 89%, 90%, 91%, 92%, 93%, 94%, 95%, 96%, 97%, 98%, 99% or higher identical thereto, or that is capable of hybridizing under stringent conditions to the sequences disclosed). The substitutions, insertions and deletions described herein may occur in any of the polynucleotides encoding GABA_{A} subunits in the cells or cell lines of the invention.

In some embodiments, where the nucleic acid substitution or modification results in an amino acid change, such as an amino acid substitution, the native amino acid may be replaced by a conservative or non-conservative substitution. In some embodiments, the sequence identity between the original and modified polypeptide sequence can differ by about 1%, 5%, 10% or 20% of the polypeptide sequence or from a sequence substantially identical thereto (e.g., a sequence at least 85%, 86%, 87%, 88%, 89%, 90%, 91%, 92%, 93%, 94%, 95%, 96%, 97%, 98%, 99% or higher identical thereto). Those of skill in the art will understand that a conservative amino acid substitution is one in which the amino acid side chains are similar in structure and/or chemical properties and the substitution should not substantially change the structural characteristics of the parent sequence. In embodiments comprising a nucleic acid comprising a mutation, the mutation may be a random mutation or a site-specific mutation.

Conservative modifications will produce GABA_{A} receptor having functional and chemical characteristics similar to those of the unmodified GABA_{A} receptor. A “conservative amino acid substitution” is one in which an amino acid residue is substituted by another amino acid residue having a side chain R group with similar chemical properties to the parent amino acid residue (e.g., charge or hydrophobicity). In general, a conservative amino acid substitution will not substantially change the functional properties of a protein. In cases where two or more amino acid sequences differ from each other by conservative substitutions, the percent sequence identity or degree of similarity may be adjusted upwards to correct for the conservative nature of the substitution. Means for making this adjustment are well-known to those of skill in the art. See, e.g., Pearson, Methods Mol. Biol. 243:307-31 (1994).

Examples of groups of amino acids that have side chains with similar chemical properties include 1) aliphatic side chains: glycine, alanine, valine, leucine, and isoleucine; 2) aliphatic-hydroxyl side chains: serine and threonine; 3) amide-containing side chains: asparagine and glutamine; 4) aromatic side chains: phenylalanine, tyrosine, and tryptophan; 5) basic side chains: lysine, arginine, and histidine; 6) acidic side chains: aspartic acid and glutamic acid; and 7) sulfur-containing side chains: cysteine and methionine. Preferred conservative amino acids substitution groups are: valine-leucine-isoleucine, phenylalanine-tyrosine, lysine-arginine, alanine-valine, glutamate-aspartate, and asparagine-glutamine. Alternatively, a conservative amino acid substitution is any change having a positive value in the PAM250 log-likelihood matrix disclosed in Gonnet et al., Science 256:1443-45 (1992). A “moderately conservative” replacement is any change having a nonnegative value in the PAM250 log-likelihood matrix.

In some embodiments, the GABA_{A} subunit-encoding nucleic acid sequence further comprises a tag. Such tags may encode, for example, a HIS tag, a myc tag, a hemagglutinin (HA) tag, protein C, VSV-G, FLU, yellow fluorescent protein (YFP), mutant YFP (meYFP), green fluorescent protein, FLAG, BCCP, maltose binding protein tag, Nus-tag, Softag-1, Softag-2, Strep-tag, S-tag, thioredoxin, GST, V5, TAP or CBP. A tag may be used as a marker to determine GABA_{A} expression levels, intracellular localization, protein-protein interactions, GABA_{A} regulation, or GABA_{A} function. Tags may also be used to purify or fractionate GABA_{A}.

GABA_{B} receptors have been reported to signal through G-proteins to regulate potassium channels. There are two families of G-protein: trimeric and monomeric. Only trimeric G-proteins interact with G-protein coupled receptors. There are three classes of trimeric G-proteins: Gα, G β and Gγ. In the inactive state Gα, G β and Gγ form a tight trimer. Upon ligand binding to a G-protein coupled receptor (GPCR), Gα separates from Gβγ. There are four main families of G alpha: Gs (stimulatory) which activates adenylate cyclase to increase cAMP synthesis. Gi (inhibitory) which inhibits adenylate cyclase, the G12/13 family which is important for regulating the cytoskeleton, cell junctions, and other processes related to movements and Gq which stimulates phospholipase C and calcium signaling. Overexpression of a particular family type will force the majority of signaling through that pathway (e.g., overexpression of Galpha15 will couple activation of most GPCRs to a calcium flux). The β and γ subunits are closely bound to one another and are referred to as the beta-gamma complex. The Gβγ complex is released from the Gα subunit after its GDP-GTP exchange. The free Gββ complex can act as a signaling molecule itself, by activating other second messengers or by gating ion channels directly.

Further, GABA_{A} receptor ion-channels are regulated by a host of cellular accessory proteins. Examples of such accessory proteins are listed in Table 5. Thus, studying these ion channels in cell lines that endogenously or heterologously express these G-proteins or GABA_{A} receptor accessory proteins may result in a more complete functional characterization of the channel. The current invention allows for the generation of multi-gene stable cell-lines that reliably express proteins of interest. This lends a strong advantage in undertaking a thorough functional characterization of this critical ion-channel when co-expressed with accessory proteins.

TABLE 5

| | | |
|---|---|---|
| | | GABA_{A} |
| | | interacting |
| Protein | Effect(s) | subunits |

GABA_{A} receptor interacting proteins

| | | |
|---|---|---|
| AP2 | Regulates surface expression | β1, β3 |
| BIG2 | Regulates surface expression | β2 |
| GABARAP | Receptor trafficking, clustering, | γ2 |
| | conductance | |
| GODZ | Plamitoyltransferase, receptor trafficking | γ1, γ2 |
| GRIF-1 | O-glcNAc transferase, receptor trafficking | β2 |
| NSF | Receptor trafficking, membrane fusion | — |
| | events | |
| Trak-1 | Receptor trafficking | ? |

Modulators of vesicular trafficking of GABA_{A} receptors

| | | |
|---|---|---|
| Hap-1 | Inhibits degradation of internalized | β |
| | GABA_{A} receptors | |
| Plic-1 | Inhibits degradation of internalized | α2, α3, α6, β |
| | GABA_{A} receptors | |

GABA_{A} receptor associated adaptors for Kinases and Phosphatases

| | | |
|---|---|---|
| AKAP | Regulates target specific phosphorylation, | β1, β3 |
| | PKA | |
| PRIP-1 | Regulates target specific phosphorylation, | β |
| and -2 | PKA | |
| RACK 1 | Regulates target specific phosphorylation, | β |
| | PKC | |

Kinases/Phosphatases modulating Phosphorylation-state
of GABA_{A} receptors

| | | |
|---|---|---|
| Akt | Phosphorylates b2 | β, γ2 |
| Calcineurin | Phosphorylates g2 | γ2 |
| PKC | Phosphorylates b2, b3, g2 | β2, β3, γ2 |
| PKA/PP1C | Phosphorylates/dephosphorylates b1, b3 | β1, β3 |
| PKC/PP2A | Phosphorylates/dephosphorylates b3 | β3 |
| Src | Phosphorylates g2 | β, γ2 |

GABA_{A} receptor-cytoskeleton crosslinker proteins

| | | |
|---|---|---|
| Gephyrin | Synaptic clustering of GABA_{A} receptors | γ2 |
| Radixin | Extrasynaptic clustering of GABA_{A} | α5 |
| | recpetors | |

Inhibitory post-synaptic protein

| | | |
|---|---|---|
| Neuroligin-2 | Organizing molecule, adhesion molecule | ? |
| Dystrophin- | ? | — |
| associated | | |
| protein | | |
| complex | | |

Host cells used to produce a cell or cell line of the invention may express in their native state one or more endogenous GABA_{A} subunits or lack expression of any GABA_{A} subunit. The host cell may be a primary, germ, or stem cell, including an embryonic stem cell. The host cell may also be an immortalized cell. Primary or immortalized host cells may be derived from mesoderm, ectoderm or endoderm layers of eukaryotic organisms. The host cell may be endothelial; epidermal; mesenchymal; neural; renal; hepatic; hematopoietic; immune cells such as B cell, T cell (Cytotoxic T cell, Natural Killer T cell, Regulatory T cell, T helper cell, gd T cell, Natural killer cell, granulocytes (Basophil granulocyte, Eosinophil granulocyte, Neutrophil granulocyte/Hypersegmented neutrophil), Monocyte/Macrophage; Red blood cell (Reticulocyte); Mast cell; Thrombocyte/Megakaryocyte; Dendritic cell; endocrine cells such as: thyroid (Thyroid epithelial cell, Parafollicular cell), parathyroid (Parathyroid chief cell, Oxyphil cell), adrenal (Chromaffin cell); nervous system cells such as: glial cells (Astrocyte, Microglia), Magnocellular neurosecretory cell, Stellate cell, Nuclear chain cell, Boettcher cell; pituitary, (Gonadotrope, Corticotrope, Thyrotrope, Somatotrope, Lactotroph); respiratory system cells such as Pneumocyte (Type I pneumocyte, Type II pneumocyte), Clara cell, Goblet cell; circulatory system cells such as Myocardiocyte,• Pericyte; digestive system cells such as stomach (Gastric chief cell, Parietal cell), Goblet cell, Paneth cell, G cells, D cells, ECL cells, I cells, K cells, Enteroendocrine cells, Enterochromaffin cell; APUD cell; liver (Hepatocyte, Kupffer cell); pancreas (beta cells, alpha cells); gallbladder; cartilage/bone/muscle/integumentary system cells such as Osteoblast• Osteocyte Osteoclast, tooth cells (Cementoblast, Ameloblast), cartilage cells: Chondroblast• Chondrocyte, skin/hair cells: Trichocyte, •Keratinocyte, Melanocyte muscle cells: Myocyte; Adipocyte; Fibroblast; urinary system cells such as Podocyte, Juxtaglomerular cell, Intraglomerular mesangial cell/Extraglomerular mesangial cell, Kidney proximal tubule brush border cell, Macula densa cell; reproductive system cells such as Spermatozoon, Sertoli cell, Leydig cell, Ovum, Ovarian follicle cell; sensory cells such as organ of Corti cells, olfactory epithelium, temperature sensitive sensory neurons, Merckel cells, olfactory receptor neuron, pain sensitive neurons, photoreceptor cells, taste bud cells, hair cells of the vestibular apparatus, and carotid body cells. The host cells may be eukaryotic, prokaryotic, mammalian, human, non-human primate, bovine, porcine, feline, rat, marsupial, murine, canine, ovine, caprine, rabbit, guinea pig and hamster. The host cells may also be nonmammalian, such as yeast, insect, fungus, plant, lower eukaryotes, prokaryotes, avian, chicken, reptile, amphibian, frog, lizard, snake, fish, worms, squid, lobster, Tasmanian devil, sea urchin, a sea slug, a sea squirt, fly, squid, hydra, arthropods, beetles, chicken, lamprey, ricefish, Rhesus macaque, zebra finch, pufferfish, and Zebrafish. Such host cells may provide backgrounds that are more divergent for testing GABA_{A} receptor modulators with a greater likelihood for the absence of expression products provided by the cell that may interact with the target. In preferred embodiments, the host cell is a mammalian cell. Examples of host cells that may be used to produce a cell or cell line of the invention include but are not limited to: Chinese hamster ovary (CHO) cells, established neuronal cell lines, pheochromocytomas, neuroblastomas fibroblasts, rhabdomyosarcomas, dorsal root ganglion cells, NS0 cells, CV-1 (ATCC CCL 70), COS-1 (ATCC CRL 1650), COS-7 (ATCC CRL 1651), CHO-K1 (ATCC CCL 61), 3T3 (ATCC CCL 92), NIH/3T3 (ATCC CRL 1658), HeLa (ATCC CCL 2), C1271 (ATCC CRL 1616), BS-C-1 (ATCC CCL 26), MRC-5 (ATCC CCL 171), L-cells, HEK-293 (ATCC CRL1573) and PC12 (ATCC CRL-1721), HEK293T (ATCC CRL-11268), RBL (ATCC CRL-1378), SH-SY5Y (ATCC CRL-2266), MDCK (ATCC CCL-34), SJ-RH30 (ATCC CRL-2061), HepG2 (ATCC HB-8065), ND7/23 (ECACC 92090903), CHO (ECACC 85050302), Vero (ATCC CCL 81), Caco-2 (ATCC HTB 37), K562 (ATCC CCL 243), Jurkat (ATCC TIB-152), Per.C6 (Crucell, Leiden, The Netherlands), Huvec (ATCC Human Primary PCS100-010, Mouse CRL 2514, CRL 2515, CRL 2516), HuH-7D12 (ECACC 01042712), HEK-293 (ATCC CRL 10852), A549 (ATCC CCL 185), IMR-90 (ATCC CCL 186), MCF-7 (ATC HTB-22), U-20S (ATCC HTB-96), T84 (ATCC CCL 248), or any established cell line (polarized or nonpolarized) or any cell line available from repositories such as American Type Culture Collection (ATCC, 10801 University Blvd. Manassas, Va. 20110-2209 USA) or European Collection of Cell Cultures (ECACC, Salisbury Wiltshire SP4 OJG England). In some embodiments, the host cell is a CHO cell or a HEK-293 cell. In a preferred embodiment, the host cell is a CHO cell.

In one embodiment, the host cell is an embryonic stem cell that is then used as the basis for the generation of transgenic animals. In some embodiments one or more subunits may be expressed with desired temporal and/or tissue specific expression. Embryonic stem cells may be implanted into organisms directly, or their nuclei may be transferred into other recipient cells and these may then be implanted, or they may be used to create transgenic animals.

As will be appreciated by those of skill in the art, any vector that is suitable for use with the host cell may be used to introduce a nucleic acid encoding a GABA_{A} subunit into the host cell. The vectors comprising the various GABA_{A} subunits may be the same type or may be of different types. Examples of vectors that may be used to introduce the GABA_{A} subunit encoding nucleic acids into host cells include but are not limited to plasmids, viruses, including retroviruses and lentiviruses, cosmids, artificial chromosomes and may include for example, Pcmv-Script, pcDNA3.1 Hygro, pcDNA3.1neo, pcDNA3.1puro, pSV2neo, pIRES puro, pSV2 zeo, pFN11A (BIND) Flexi®, pGL4.31, pFC14A (HaloTag® 7) CMV Flexi®,pFC14K (HaloTag® 7) CMV Flexi®,pFN24A (HaloTag® 7) CMVd3 Flexi®, pFN24K (HaloTag® 7) CMVd3 Flexi®, HaloTag™ pHT2, pACT, pAdVAntage™, pALTER®-MAX, pBIND, pCAT®3-Basic, pCAT®3-Control, pCAT®3-Enhancer, pCAT®3-Promoter, pCI, pCMVTNT™, pGSluc, pSI, pTARGET™, pTNT™, pF12A RM Flexi®, pF12K RM Flexi®, pReg neo, pYES2/GS, pAd/CMV/V5-DEST Gateway® Vector, pAd/PL-DEST™ Gateway®, Vector, Gateway®, pDEST™, 27 Vector, Gateway®, pEF-DEST51 Vector, Gateway®, pcDNA™-DEST47 vector, pCMV/Bsd Vector, pEF6/His A, B, & c, pcDNA™ 6.2-DEST, pLenti6/TR, pLP-AcGFP1-C, pLPS-AcGFP1-N, pLP-IRESneo, pLP-TRE2, pLP-RevTRE, pLP-LNCX, pLP-CMV-HA, pLP-CMV-Myc, pLP-RetroQ, pLP-CMVneo. In some embodiments, the vectors comprise expression control sequences such as constitutive or conditional promoters. One of ordinary skill in the art will be able to select the appropriate sequences. For example, suitable promoters include but are not limited to CMV, TK, SV40 and EF-1α. In some embodiments, the promoters are inducible, temperature regulated, tissue specific, repressible, heat-shock, developmental, cell lineage specific, eukaryotic, prokaryotic or temporal promoters or a combination or recombination of unmodified or mutagenized, randomized, shuffled sequences of any one or more of the above. In other embodiments, GABA_{A} receptor subunits are expressed by gene activation, wherein an exogenous promoter is inserted in a host cell's genome by homologous recombination to drive expression of a GABA_{A} subunit gene that is not normally expressed in that host cell. In some embodiments the gene encoding a GABA_{A} subunit is episomal. Nucleic acids encoding GABA_{A} subunits are preferably constitutively expressed.

In some embodiments, the vector lacks a selectable marker or drug resistance gene. In other embodiments, the vector optionally comprises a nucleic acid encoding a selectable marker such as a protein that confers drug or antibiotic resistance. Each vector for a sequence encoding a different GABA_{A} subunit may have the same or a different drug resistance or other selectable marker. If more than one of the drug resistance markers are the same, simultaneous selection may be achieved by increasing the level of the drug. Suitable markers well-known to those of skill in the art include, but are not limited to, genes conferring resistance to any one of the following: Neomycin/G418, Puromycin, hygromycin, Zeocin, methotrexate and blasticidin. Although drug selection, or selection using any other suitable selection marker, (i.e. selective pressure) is not a required step, it may be used to enrich the transfected cell population for stably transfected cells, provided that the transfected constructs are designed to confer drug resistance. When signaling probes are used for the selection of cells expressing GABA_{A}, GABA_{B}, or GABA_{C} receptors or GABA_{A}, GABA_{B}, or GABA_{C} subunits, false positives (i.e., cells which are transiently transfected test positive as if they were stably transfected) may occur if selection occurs too soon following transfection. This can be minimized, however, by allowing sufficient cell passage allowing for dilution of transient expression in transfected cells.

In some embodiments, the vector comprises a nucleic acid sequence encoding an RNA tag sequence. “Tag sequence” refers to a nucleic acid sequence that is an expressed RNA or portion of an RNA that is to be detected by a signaling probe. Signaling probes may detect a variety of RNA sequences. Any of these RNAs may be used as tags. Signaling probes may be directed against the RNA tag by designing the probes to include a portion that is complementary to the sequence of the tag. The tag sequence may be a 3′ untranslated region of the plasmid that is cotranscribed and comprises a target sequence for signaling probe binding. The RNA encoding the gene of interest may include the tag sequence or the tag sequence may be located within a 5′-untranslated region or 3′-untranslated region. In some embodiments, the tag is not with the RNA encoding the gene of interest. The tag sequence can be in frame with the protein-coding portion of the message of the gene or out of frame with it, depending on whether one wishes to tag the protein produced. Thus, the tag sequence does not have to be translated for detection by the signaling probe. The tag sequences may comprise multiple target sequences that are the same or different, wherein one signaling probe hybridizes to each target sequence. The tag sequences may encode an RNA having secondary structure. The structure may be a three-arm junction structure. Examples of tag sequences that may be used in the invention, and to which signaling probes may be prepared, include but are not limited to the RNA transcript of epitope tags such as, for example, a HIS tag, a myc tag, a hemagglutinin (HA) tag, protein C, VSV-G, FLU, yellow fluorescent protein (YFP), green fluorescent protein, FLAG, BCCP, maltose binding protein tag, Nus-tag, Softag-1, Softag-2, Strep-tag, S-tag, thioredoxin, GST, V5, TAP or CBP. As described herein, one of ordinary skill in the art could create his or her own RNA tag sequences.

To make cells and cell lines of the invention, one can use, for example, the technology described in U.S. Pat. No. 6,692,965 and International Patent Publication WO/2005/079462. Both of these documents are incorporated herein by reference in their entirety for all purposes. This technology provides real-time assessment of millions of cells such that any desired number of clones (from hundreds to thousands of clones) may be selected. Using cell sorting techniques, such as flow cytometric cell sorting (e.g., with a FACS machine) or magnetic cell sorting (e.g., with a MACS machine), one cell per well may be automatically deposited with high statistical confidence in a culture vessel (such as a 96 well culture plate). The speed and automation of the technology allows multigene cell lines to be readily isolated.

Using the technology, the RNA sequence for each GABA_{A} subunit may be detected using a signaling probe, also referred to as a molecular beacon or fluorogenic probe. In some embodiments, the molecular beacon recognizes a target tag sequence as described above. In another embodiment, the molecular beacon recognizes a sequence within the GABA_{A} subunit itself. Signaling probes may be directed against the RNA tag or GABA_{A} subunit sequence by designing the probes to include a portion that is complementary to the RNA sequence of the tag or the GABA_{A} subunit, respectively.

Nucleic acids comprising a sequence encoding a GABA_{A} subunit, or the sequence of a GABA_{A} subunit and a tag sequence, and optionally a nucleic acid encoding a selectable marker may be introduced into selected host cells by well known methods. The methods include but not limited to transfection, viral delivery, protein or peptide mediated insertion, coprecipitation methods, lipid based delivery reagents (lipofection), cytofection, lipopolyamine delivery, dendrimer delivery reagents, electroporation or mechanical delivery. Examples of transfection reagents are GENEPORTER, GENEPORTER2, LIPOFECTAMINE, LIPOFECTAMINE 2000, FUGENE 6, FUGENE HD, TFX-10, TFX-20, TFX-50, OLIGOFECTAMINE, TRANSFAST, TRANSFECTAM, GENESHUTTLE, TROJENE, GENESILENCER, X-TREMEGENE, PERFECTIN, CYTOFECTIN, SIPORT, UNIFECTOR, SIFECTOR, TRANSIT-LT1, TRANSIT-LT2, TRANSIT-EXPRESS, IFECT, RNAI SHUTTLE, METAFECTENE, LYOVEC, LIPOTAXI, GENEERASER, GENEJUICE, CYTOPURE, JETSI, JETPEI, MEGAFECTIN, POLYFECT, TRANSMESSANGER, RNAiFECT, SUPERFECT, EFFECTENE, TF-PEI-KIT, CLONFECTIN, AND METAFECTINE.

Following the introduction of the GABA_{A} subunit coding sequences into host cells and optional subsequent drug selection, molecular beacons (e.g., fluorogenic probes) are introduced into the cells. Subsequently, cell sorting is used to isolate cells positive for the molecular beacon signals. Multiple rounds of sorting may be carried out, if desired. In one embodiment, the flow cytometric cell sorter is a FACS machine. MACS (magnetic cell sorting) or laser ablation of negative cells using laser-enabled analysis and processing can also be used. According to this method, cells expressing at least one alpha; one beta; and one gamma, delta, epsilon, pi, theta, or rho subunit are detected and recovered. The GABA_{A} subunit sequences may be integrated at different locations of the genome in the cell. The expression level of the introduced genes encoding the GABA_{A} subunits may vary based upon integration site. The skilled worker will recognize that sorting can be gated for any desired expression level. Further, stable cell lines may be obtained wherein one or more of the introduced genes encoding a GABA_{A} subunit is episomal or results from gene activation.

Signaling probes (such as molecular beacons) useful in this invention are known in the art and generally are oligonucleotides comprising a sequence complementary to a target sequence and a signal emitting system so arranged that no signal is emitted when the probe is not bound to the target sequence and a signal is emitted when the probe binds to the target sequence. By way of non-limiting illustration, the signaling probe may comprise a fluorophore and a quencher positioned in the probe so that the quencher and fluorophore are brought together in the unbound probe. Upon binding between the probe and the target sequence, the quencher and fluorophore separate, resulting in emission of a signal. International publication WO/2005/079462, for example, describes a number of signaling probes that may be used in the production of the cells and cell lines of this invention. Where tag sequences (to which signaling probes bind) are used, the vector for each of the GABA_{A} subunit can comprise the same or a different tag sequence. Whether the tag sequences are the same or different, the signaling probes may comprise different signal emitters, such as different colored fluorophores, so that (RNA) expression of each subunit may be separately detected. By way of illustration, the signaling probe that specifically detects GABA_{A} alpha subunit mRNA can comprise an orange fluorophore, the probe that detects the first GABA_{A} beta subunit (RNA) can comprise a red fluorophore and the probe that detects the GABA_{A} gamma subunit (RNA) can comprise a green fluorophore. Those of skill in the art will be aware of other means for differentially detecting the expression of the three subunits with a signaling probe in a triply transfected cell.

Nucleic acids encoding signaling probes may be introduced into the selected host cell by any of numerous means that will be well-known to those of skill in the art, including but not limited to transfection, coprecipitation methods, lipid based delivery reagents (lipofection), cytofection, lipopolyamine delivery, dendrimer delivery reagents, electroporation or mechanical delivery. Examples of transfection reagents are GENEPORTER, GENEPORTER2, LIPOFECTAMINE, LIPOFECTAMINE 2000, FUGENE 6, FUGENE HD, TFX-10, TFX-20, TFX-50, OLIGOFECTAMINE, TRANSFAST, TRANSFECTAM, GENESHUTTLE, TROJENE, GENESILENCER, X-TREMEGENE, PERFECTIN, CYTOFECTIN, SIPORT, UNIFECTOR, SIFECTOR, TRANSIT-LT1, TRANSIT-LT2, TRANSIT-EXPRESS, IFECT, RNAI SHUTTLE, METAFECTENE, LYOVEC, LIPOTAXI, GENEERASER, GENEJUICE, CYTOPURE, JETSI, JETPEI, MEGAFECTIN, POLYFECT, TRANSMESSANGER, RNAiFECT, SUPERFECT, EFFECTENE, TF-PEI-KIT, CLONFECTIN, AND METAFECTINE.

In one embodiment, the signaling probes are designed to be complementary to either a portion of the RNA encoding a GABA_{A} subunit or to portions of their 5′ or 3′ untranslated regions. Even if the signaling probe designed to recognize a messenger RNA of interest is able to spuriously detect endogenously existing target sequences, the proportion of these in comparison to the proportion of the sequence of interest produced by transfected cells is such that the sorter is able to discriminate the two cell types.

In another embodiment of the invention, adherent cells can be adapted to suspension before or after cell sorting and isolating single cells. In other embodiments, isolated cells may be grown individually or pooled to give rise to populations of cells. Individual or multiple cell lines may also be grown separately or pooled. If a pool of cell lines is producing a desired activity or has a desired property, it can be further fractionated until the cell line or set of cell lines having this effect is identified. Pooling cells or cell lines may make it easier to maintain large numbers of cell lines without the requirements for maintaining each separately. Thus, a pool of cells or cell lines may be enriched for positive cells. An enriched pool may have at least 50%, at least 60%, at least 70%, at least 80% or at least 90%, or 100% are positive for the desired property or activity.

The expression level of a GABA_{A} subunit may vary from cell or cell line to cell or cell line. The expression level in a cell or cell line also may decrease over time due to epigenetic events such as DNA methylation and gene silencing and loss of transgene copies. These variations can be attributed to a variety of factors, for example, the copy number of the transgene taken up by the cell, the site of genomic integration of the transgene, and the integrity of the transgene following genomic integration. One may use FACS or other cell sorting methods (i.e., MACS) to evaluate expression levels. Additional rounds of introducing signaling probes may be used, for example, to determine if and to what extent the cells remain positive over time for any one or more of the RNAs for which they were originally isolated.

In one embodiment, isolated GABA_{A}-expressing cells may be grown individually or pooled to give rise to populations of cells. Individual or multiple cells or cell lines may also be grown separately or pooled. If a pool of cells or cell lines is producing a desired activity, it can be further fractionated until the cell or cell line or set of cells or cell lines having this effect is identified. This may make it easier to maintain large numbers of cells and cell lines without the requirements for maintaining each separately.

In some embodiments, clones of individual cells which have been identified as expressing the introduced GABA_{A} subunits of interest are further screened for functionality. In our studies, we found that after isolating hundreds of unique clones that expressed the GABA_{A} subunits of interest, very few (e.g., 2%) responded to physiological doses of GABA ligand. Without wishing to be limited by any theory, this low rate of functionality may be due to the importance of the stoichiometry of subunits expressed. Even though the transfected cells are isolated based on expression of the subunits of interest, it is difficult to isolate cells based on stoichiometry for several reasons (e.g., varying affinity of different probes for their target sequences, post-translational regulation of subunit expression can not be detect by the signaling probes, and the critical stoichiometries are unknown). Additionally, the expression of other (known or unknown) cellular factors may be required for physiological GABA_{A} functionality. For at least these reasons, a large number of cells that are positive for expression need to be screened for functionality. In some embodiments, robotic cell culture conditions are used to tightly regulate cell culture conditions (e.g., cell density, media conditions, treatment with a compound, and synchronization). Such robotic procedures make it possible to screen a sufficient number of clones to identify clones that express properly functioning GABA_{A}. In some embodiments, the methods of making GABA receptor expressing cell lines are used to make other heteromultimeric protein expressing cell lines.

In some embodiments, the invention provides cells and cell lines that stably express a GABA_{A} receptor. In some embodiments, the expressed GABA_{A} receptors conduct chloride ions and are modulated by GABA (its endogenous ligand), muscimol, isoguvacine hydrochloride or bicuculline. In further embodiments, the GABA_{A} receptor cells and cell lines of the invention have enhanced properties compared to cells and cell lines made by conventional methods. For example, the GABA_{A} receptor cells and cell lines have enhanced stability of expression as compared to cells and cell lines produced by conventional methods (even when maintained in culture without selective antibiotics). To identify stable expression, a cell or cell line's expression of each GABA_{A} subunit is measured over a timecourse and the expression levels are compared. Stable cell lines will continue expressing GABA_{A} alpha, beta and gamma or delta subunits throughout the timecourse. In some aspects of the invention, the timecourse may be for at least one week, two weeks, three weeks, etc., or at least one month, or at least two, three, four, five, six, seven, eight or nine months, or any length of time in between. Isolated cells and cell lines can be further characterized by methods such as qRT-PCR and single end-point RT-PCR to determine the absolute amounts and relative amounts of each GABA_{A} subunit being expressed. In some embodiments, stable expression is measured by comparing the results of functional assays over a timecourse. The measurement of stability based on functional assays provides the benefit of identifying clones that not only stably express the mRNA of the gene of interest, but also stably produce and properly process (e.g., post-translational modification, subunit assembly, and localization within the cell) the protein encoded by the gene of interest that functions appropriately.

In various embodiments, the cell or cell line of the invention expresses GABA_{A} alpha, beta and gamma subunits at a consistent level of expression for at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100, 110, 120, 130, 140, 150, 160, 170, 180, 190, 200 days or over 200 days, where consistent expression refers to a level of expression that does not vary by more than:

1%, 2%, 3%, 4%, 5%, 6%, 7%, 8% 9% or 10% over 2 to 4 days of continuous cell culture; 2%, 4%, 6%, 8%, 10% or 12% over 5 to 15 days of continuous cell culture; 2%, 4%, 6%, 8%, 10%, 12%, 14%, 16%, 18% or 20% over 16 to 20 days of continuous cell culture; 2%, 4%, 6%, 8%, 10%, 12%, 14%, 16%, 18%, 20%, 22%, 24% over 21 to 30 days of continuous cell culture; 2%, 4%, 6%, 8%, 10%, 12%, 14%, 16%, 18%, 20%, 22%, 24%, 26%, 28% or 30% over 30 to 40 days of continuous cell culture; 2%, 4%, 6%, 8%, 10%, 12%, 14%, 16%, 18%, 20%, 22%, 24%, 26%, 28% or 30% over 41 to 45 days of continuous cell culture; 2%, 4%, 6%, 8%, 10%, 12%, 14%, 16%, 18%, 20%, 22%, 24%, 26%, 28% or 30% over 45 to 50 days of continuous cell culture; 2%, 4%, 6%, 8%, 10%, 12%, 14%, 16%, 18%, 20%, 22%, 24%, 26%, 28%, 30% or 35% over 45 to 50 days of continuous cell culture, 2%, 4%, 6%, 8%, 10%, 12%, 14%, 16%, 18%, 20%, 22%, 24%, 26%, 28% or 30% over 50 to 55 days of continuous cell culture, 2%, 4%, 6%, 8%, 10%, 12%, 14%, 16%, 18%, 20%, 22%, 24%, 26%, 28%, 30% or 35% over 50 to 55 days of continuous cell culture; 1%, 2%, 3%, 4%, 5%, 10%, 15%, 20%, 25%, 30%, 35% or 40% over 55 to 75 days of continuous cell culture;1%, 2%, 3%, 4%, 5%, 6%, 10%, 15%, 20%, 25%, 30%, 35%, 40% or 45% over 75 to 100 days of continuous cell culture; 1%, 2%, 3%, 4%, 5%, 6%, 10%, 15%, 20%, 25%, 30%, 35%, 40% or 45% over 101 to 125 days of continuous cell culture; 1%, 2%, 3%, 4%, 5%, 6%, 10%, 15%, 20%, 25%, 30%, 35%, 40% or 45% over 126 to 150 days of continuous cell culture; 1%, 2%, 3%, 4%, 5%, 6%, 10%, 15%, 20%, 25%, 30%, 35%, 40% or 45% over 151 to 175 days of continuous cell culture; 1%, 2%, 3%, 4%, 5%, 6%, 10%, 15%, 20%, 25%, 30%, 35%, 40% or 45% over 176 to 200 days of continuous cell culture; 1%, 2%, 3%, 4%, 5%, 6%, 10%, 15%, 20%, 25%, 30%, 35%, 40% or 45% over more than 200 days of continuous cell culture.

Cells and cell lines of the invention have the further advantageous property of providing assays with high reproducibility as evidenced by their Z′ factor. See Zhang J H, Chung T D, Oldenburg K R, “A Simple Statistical Parameter for Use in Evaluation and Validation of High Throughput Screening Assays.” J. Biomol. Screen. 1999; 4(2):67-73. Z′ values pertain to the quality of a cell or cell line because it reflects the degree to which a cell or cell line will respond consistently to modulators. Z′ is a statistical calculation that takes into account the signal-to-noise range and signal variability (i.e., from well to well) of the functional response to a reference compound across a multiwell plate. Z′ is calculated using data obtained from multiple wells with a positive control and multiple wells with a negative control. The ratio of their summated standard deviations multiplied by a factor of three to the difference in their mean values is subtracted from one to give the Z′ factor, according the equation below:

Z′ factor=1−((3σ_{positive control}+3σ_{negative control})/(μ_{positive control}−μ_{negative control}))

The theoretical maximum Z′ factor is 1.0, which would indicate an ideal assay with no variability and limitless dynamic range. As used herein, a “high Z′” refers to a Z′ factor of Z′ of at least 0.6, at least 0.7, at least 0.75 or at least 0.8, or any decimal in between 0.6 and 1.0. In the case of a complex target such as GABA_{A} receptor, a high Z′ means a Z′ of at least 0.4 or greater. A low score (close to 0) is undesirable because it indicates that there is overlap between positive and negative controls. In the industry, for simple cell-based assays, Z′ scores up to 0.3 are considered marginal scores, Z′ scores between 0.3 and 0.5 are considered acceptable, and Z′ scores above 0.5 are considered excellent. Cell-free or biochemical assays may approach higher Z′ scores, but Z′ scores for cell-based systems tend to be lower because cell-based systems are complex.

As those of ordinary skill in the art will recognize, historically, cell-based assays using cells expressing even a single chain protein do not typically achieve a Z′ higher than 0.5 to 0.6. Further, assays utilizing cells with engineered GABA receptor expression (via, for example, introduced coding sequences or gene activation methods) of multi-subunit proteins tend to exhibit lower Z′ values due to their added complexity. Such cells would not be reliable to use in an assay because the results are not reproducible. Cells and cell lines of the invention, on the other hand, have high Z′ values and advantageously produce consistent results in assays. GABA_{A} expressing cells and cell lines of the invention are useful for high throughput screening (HTS) compatible assays because they generally have Z′ factors of at least 0.4. In some aspects of the invention, the cells and cell lines exhibit Z′ values of at least 0.3, at least 0.4, at least 0.5, at least 0.6, at least 0.7, or at least 0.8. It is noted that because heteromeric proteins, such as GABA_{A}, have been traditionally difficult to express, even cells and cell lines exhibiting Z′ values of 0.3-0.4 are advantageous in these systems. In other aspects of the invention, the cells and cell lines of the invention exhibit a Z′ of at least 0.4, at least 0.5 or at least 0.55 maintained for multiple passages (e.g., between 5-20 passages, including any integer in between 5 and 20). In some aspects of the invention, the cells and cell lines exhibit a Z′ of at least 0.4, at least 0.5 or at least 0.55 maintained for 1, 2, 3, 4 or 5 weeks or 2, 3, 4, 5, 6, 7, 8 or 9 months, including any period of time in between.

In a further aspect, the invention provides a method for producing the cells and cell lines of the invention. In one embodiment, the method comprises the steps of:
a) providing a plurality of cells that express mRNA encoding a GABA receptor subunit or combination of GABA receptor subunits;
b) dispersing cells individually into individual culture vessels, thereby providing a plurality of separate cell cultures
c) culturing the cells under a set of desired culture conditions using automated cell culture methods characterized in that the conditions are substantially identical for each of the separate cell cultures, during which culturing the number of cells in each separate cell culture is normalized, and wherein the separate cultures are passaged on the same schedule;
d) assaying the separate cell cultures for at least one desired characteristic of the GABA receptor at least twice; and
e) identifying a separate cell culture that has the desired characteristic in both assays.

According to the method, the cells are cultured under a desired set of culture conditions. The conditions can be any desired conditions. Those of skill in the art will understand what parameters are comprised within a set of culture conditions. For example, culture conditions include but are not limited to: the media (Base media (DMEM, MEM, RPMI, serum-free, with serum, fully chemically defined, without animal-derived components), mono and divalent ion (sodium, potassium, calcium, magnesium) concentration, additional components added (amino acids, antibiotics, glutamine, glucose or other carbon source, HEPES, channel blockers, modulators of other targets, vitamins, trace elements, heavy metals, co-factors, growth factors, anti-apoptosis reagents), fresh or conditioned media, with HEPES, pH, depleted of certain nutrients or limiting (amino acid, carbon source)), level of confluency at which cells are allowed to attain before split/passage, feeder layers of cells, or gamma-irradiated cells, CO2, a three gas system (oxygen, nitrogen, carbon dioxide), humidity, temperature, still or on a shaker, and the like, which will be well known to those of skill in the art.

The cell culture conditions may be chosen for convenience or for a particular desired use of the cells. Advantageously, the invention provides cells and cell lines that are optimally suited for a particular desired use. That is, in embodiments of the invention in which cells are cultured under conditions for a particular desired use, cells are selected that have desired characteristics under the condition for the desired use.

By way of illustration, if cells will be used in assays in plates where it is desired that the cells are adherent, cells that display adherence under the conditions of the assay may be selected. Similarly, if the cells will be used for protein production, cells may be cultured under conditions appropriate for protein production and selected for advantageous properties for this use.

In some embodiments, the method comprises the additional step of measuring the growth rates of the separate cell cultures. Growth rates may be determined using any of a variety of techniques means that will be well known to the skilled worker. Such techniques include but are not limited to measuring ATP, cell confluency, light scattering, optical density (e.g., OD 260 for DNA). Preferably growth rates are determined using means that minimize the amount of time that the cultures spend outside the selected culture conditions.

In some embodiments, cell confluency is measured and growth rates are calculated from the confluency values. In some embodiments, cells are dispersed and clumps removed prior to measuring cell confluency for improved accuracy. Means for monodispersing cells are well-known and can be achieved, for example, by addition of a dispersing reagent to a culture to be measured. Dispersing agents are well-known and readily available, and include but are not limited to enzymatic dispering agents, such as trypsin, and EDTA-based dispersing agents. Growth rates can be calculated from confluency date using commercially available software for that purpose such as HAMILTON VECTOR. Automated confluency measurement, such as using an automated microscopic plate reader is particularly useful. Plate readers that measure confluency are commercially available and include but are not limited to the CLONE SELECT IMAGER (Genetix). Typically, at least 2 measurements of cell confluency are made before calculating a growth rate. The number of confluency values used to determine growth rate can be any number that is convenient or suitable for the culture. For example, confluency can be measured multiple times over e.g., a week, 2 weeks, 3 weeks or any length of time and at any frequency desired.

When the growth rates are known, according to the method, the plurality of separate cell cultures are divided into groups by similarity of growth rates. By grouping cultures into growth rate bins, one can manipulate the cultures in the group together, thereby providing another level of standardization that reduces variation between cultures. For example, the cultures in a bin can be passaged at the same time, treated with a desired reagent at the same time, etc. Further, functional assay results are typically dependent on cell density in an assay well. A true comparison of individual clones is only accomplished by having them plated and assayed at the same density. Grouping into specific growth rate cohorts enables the plating of clones at a specific density that allows them to be functionally characterized in a high throughput format

The range of growth rates in each group can be any convenient range. It is particularly advantageous to select a range of growth rates that permits the cells to be passaged at the same time and avoid frequent renormalization of cell numbers. Growth rate groups can include a very narrow range for a tight grouping, for example, average doubling times within an hour of each other. But according to the method, the range can be up to 2 hours, up to 3 hours, up to 4 hours, up to 5 hours or up to 10 hours of each other or even broader ranges. The need for renormalization arises when the growth rates in a bin are not the same so that the number of cells in some cultures increases faster than others. To maintain substantially identical conditions for all cultures in a bin, it is necessary to periodically remove cells to renormalize the numbers across the bin. The more disparate the growth rates, the more frequently renormalization is needed.

In step d) the cells and cell lines may be tested for and selected for any physiological property including but not limited to: a change in a cellular process encoded by the genome; a change in a cellular process regulated by the genome; a change in a pattern of chromosomal activity; a change in a pattern of chromosomal silencing; a change in a pattern of gene silencing; a change in a pattern or in the efficiency of gene activation; a change in a pattern or in the efficiency of gene expression; a change in a pattern or in the efficiency of RNA expression; a change in a pattern or in the efficiency of RNAi expression; a change in a pattern or in the efficiency of RNA processing; a change in a pattern or in the efficiency of RNA transport; a change in a pattern or in the efficiency of protein translation; a change in a pattern or in the efficiency of protein folding; a change in a pattern or in the efficiency of protein assembly; a change in a pattern or in the efficiency of protein modification; a change in a pattern or in the efficiency of protein transport; a change in a pattern or in the efficiency of transporting a membrane protein to a cell surface change in growth rate; a change in cell size; a change in cell shape; a change in cell morphology; a change in % RNA content; a change in % protein content; a change in % water content; a change in % lipid content; a change in ribosome content; a change in mitochondrial content; a change in ER mass; a change in plasma membrane surface area; a change in cell volume; a change in lipid composition of plasma membrane; a change in lipid composition of nuclear envelope; a change in protein composition of plasma membrane; a change in protein; composition of nuclear envelope; a change in number of secretory vesicles; a change in number of lysosomes; a change in number of vacuoles; a change in the capacity or potential of a cell for: protein production, protein secretion, protein folding, protein assembly, protein modification, enzymatic modification of protein, protein glycosylation, protein phosphorylation, protein dephosphorylation, metabolite biosynthesis, lipid biosynthesis, DNA synthesis, RNA synthesis, protein synthesis, nutrient absorption, cell growth, mitosis, meiosis, cell division, to dedifferentiate, to transform into a stem cell, to transform into a pluripotent cell, to transform into a omnipotent cell, to transform into a stem cell type of any organ (i.e. liver, lung, skin, muscle, pancreas, brain, testis, ovary, blood, immune system, nervous system, bone, cardiovascular system, central nervous system, gastro-intestinal tract, stomach, thyroid, tongue, gall bladder, kidney, nose, eye, nail, hair, taste bud), to transform into a differentiated any cell type (i.e. muscle, heart muscle, neuron, skin, pancreatic, blood, immune, red blood cell, white blood cell, killer T-cell, enteroendocrine cell, taste, secretory cell, kidney, epithelial cell, endothelial cell, also including any of the animal or human cell types already listed that can be used for introduction of nucleic acid sequences), to uptake DNA, to uptake small molecules, to uptake fluorogenic probes, to uptake RNA, to adhere to solid surface, to adapt to serum-free conditions, to adapt to serum-free suspension conditions, to adapt to scaled-up cell culture, for use for large scale cell culture, for use in drug discovery, for use in high throughput screening, for use in a functional cell based assay, for use in membrane potential assays, for use in calcium flux assays, for use in G-protein reporter assays, for use in reporter cell based assays, for use in ELISA studies, for use in in vitro assays, for use in vivo applications, for use in secondary testing, for use in compound testing, for use in a binding assay, for use in panning assay, for use in an antibody panning assay, for use in imaging assays, for use in microscopic imaging assays, for use in multiwell plates, for adaptation to automated cell culture, for adaptation to miniaturized automated cell culture, for adaptation to large-scale automated cell culture, for adaptation to cell culture in multiwell plates (6, 12, 24, 48, 96, 384, 1536 or higher density), for use in cell chips, for use on slides, for use on glass slides, for microarray on slides or glass slides, for immunofluorescence studies, for use in protein purification, for use in biologics production, for use in the production of industrial enzymes, for use in the production of reagents for research, for use in vaccine development, for use in cell therapy, for use in implantation into animals or humans, for use in isolation of factors secreted by the cell, for preparation of cDNA libraries, for purification of RNA, for purification of DNA, for infection by pathogens, viruses or other agent, for resistance to infection by pathogens, viruses or other agents, for resistance to drugs, for suitability to be maintained under automated miniaturized cell culture conditions, for use in the production of protein for characterization, including: protein crystallography, vaccine development, stimulation of the immune system, antibody production or generation or testing of antibodies. Those of skill in the art will readily recognize suitable tests for any of the above-listed properties.

Tests that may be used to characterize cells and cell lines of the invention and/or matched panels of the invention include but are not limited to: Amino acid analysis, DNA sequencing, Protein sequencing, NMR, A test for protein transport, A test for nucelocytoplasmic transport, A test for subcellular localization of proteins, A test for subcellular localization of nucleic acids, Microscopic analysis, Submicroscopic analysis, Fluorescence microscopy, Electron microscopy, Confocal microscopy, Laser ablation technology, Cell counting and Dialysis. The skilled worker would understand how to use any of the above-listed tests. According to the method, cells may be cultured in any cell culture format so long as the cells or cell lines are dispersed in individual cultures prior to the step of measuring growth rates. For example, for convenience, cells may be initially pooled for culture under the desired conditions and then individual cells separated one cell per well or vessel.

Cells may be cultured in multi-well tissue culture plates with any convenient number of wells. Such plates are readily commercially available and will be well knows to a person of skill in the art. In some cases, cells may preferably be cultured in vials or in any other convenient format, the various formats will be known to the skilled worker and are readily commercially available.

In embodiments comprising the step of measuring growth rate, prior to measuring growth rates, the cells are cultured for a sufficient length of time for them to acclimate to the culture conditions. As will be appreciated by the skilled worker, the length of time will vary depending on a number of factors such as the cell type, the chosen conditions, the culture format and may be any amount of time from one day to a few days, a week or more.

Preferably, each individual culture in the plurality of separate cell cultures is maintained under substantially identical conditions a discussed below, including a standardized maintenance schedule. Another advantageous feature of the method is that large numbers of individual cultures can be maintained simultaneously, so that a cell with a desired set of traits may be identified even if extremely rare. For those and other reasons, according to the invention, the plurality of separate cell cultures are cultured using automated cell culture methods so that the conditions are substantially identical for each well. Automated cell culture prevents the unavoidable variability inherent to manual cell culture.

Any automated cell culture system may be used in the method of the invention. A number of automated cell culture systems are commercially available and will be well-known to the skilled worker. In some embodiments, the automated system is a robotic system. Preferably, the system includes independently moving channels, a multichannel head (for instance a 96-tip head) and a gripper or cherry-picking arm and a HEPA filtration device to maintain sterility during the procedure. The number of channels in the pipettor should be suitable for the format of the culture. Convenient pipettors have, e.g., 96 or 384 channels. Such systems are known and are commercially available. For example, a MICROLAB STAR™ instrument (Hamilton) may be used in the method of the invention. The automated system should be able to perform a variety of desired cell culture tasks. Such tasks will be known by a person of skill in the art. They include but are not limited to: removing media, replacing media, adding reagents, cell washing, removing wash solution, adding a dispersing agent, removing cells from a culture vessel, adding cells to a culture vessel an the like.

The production of a cell or cell line of the invention may include any number of separate cell cultures. However, the advantages provided by the method increase as the number of cells increases. There is no theoretical upper limit to the number of cells or separate cell cultures that can be utilized in the method. According to the invention, the number of separate cell cultures can be two or more but more advantageously is at least 3, 4, 5, 6, 7, 8, 9, 10 or more separate cell cultures, for example, at least 12, at least 15, at least 20, at least 24, at least 25, at least 30, at least 35, at least 40, at least 45, at least 48, at least 50, at least 75, at least 96, at least 100, at least 200, at least 300, at least 384, at least 400, at least 500, at least 1000, at least 10,000, at least 100,000, at least 500,000 or more.

In another aspect of the invention, cells and cell lines that express GABA_{A} receptors can be characterized for chloride ion conductance. In some embodiments, the cells and cell lines of the invention express GABA_{A} receptors with “physiologically relevant” activity. As used herein, physiological relevance refers to a property of a cell or cell line expressing a GABA_{A} receptor whereby the GABA_{A} receptor conducts chloride ions as naturally occurring GABA_{A} receptors of the same type (e.g., an expressed α1β3γ2S-GABA_{A} receptor behaves as an endogenous α1β3γ2S-GABA_{A} receptor) and responds to modulators as naturally occurring GABA_{A} receptors of the same type. Preferably, GABA_{A} cells and cell lines of this invention function comparably to cells that endogenously express GABA_{A} receptors when used in a functional assay. Examples of such assays include a membrane potential assay in response to GABA activation, quenching halide-sensitive YFP response to GABA activation, or by electrophysiology following activation with GABA. Such comparisons are used to determine a cell or cell line's physiological relevance.

In some embodiments, the cells and cell lines of the invention have increased sensitivity to modulators of the GABA_{A} receptor when compared to previously reported sensitivities (e.g. from oocytes microinjected with GABA_{A} receptor subunits). In preferred embodiments, cells and cell lines of the invention respond to modulators and conduct chloride ions with physiological range EC₅₀ or IC₅₀ values for GABA_{A} receptor.

A further advantageous property of the cells and cell lines of the invention stems from their physiological relevance. As one of skill in the art would recognize, compounds identified in traditional screening assays typically need to be optimized (e.g., by combinatorial chemistry, medicinal chemistry, or synthetic chemistry) for use in subsequent secondary functional assays. Such an optimization process can be tedious and expensive. However, due to the physiological relevance of the cells and cell lines of the present invention, their use in initial screening assays yields physiologically relevant compounds and, thus, may eliminate the need for optimization and/or secondary functional assays of such hits.

One aspect of the invention provides a collection of clonal cells and cell lines, each expressing GABA_{A} receptor comprising same set of subunits. The collection may include, for example, cells or cell lines expressing combinations of different subunits, or full length or fragments of subunits.

A further advantageous property of the GABA_{A}-expressing cells and cell lines of the invention is that they stably express at least one alpha, at least one beta and at least one gamma or delta subunit in the absence of drug selection pressure. Thus, in preferred embodiments, cells and cell lines of the invention are maintained in culture in the absence of a selective drug. In further embodiments, cells and cell lines are maintained in the absence of antibiotics. As used herein, cell maintenance refers to culturing cells after they have been selected for their GABA_{A} receptor expression. Maintenance does not refer to the optional step of growing cells in a selective drug (e.g., an antibiotic) prior to cell sorting where drug resistance marker(s) introduced into the cells allow enrichment of stable transfectants in a mixed population.

Drug-free cell maintenance provides a number of advantages. For example, drug-resistant cells do not always express the co-transfected transgene of interest at adequate levels, because the selection relies on survival of the cells that have taken up the drug resistant gene, with or without the transgene. Further, selective drugs are often mutagenic or otherwise interfere with the physiology of the cells, leading to less relevant results in cell-based assays. For example, selective drugs may decrease susceptibility to apoptosis (Robinson et al., Biochemistry, 36(37):11169-11178 (1997)), increase DNA repair and drug metabolism (Deffie et al., Cancer Res. 48(13):3595-3602 (1988)), increase cellular pH (Thiebaut et al., J Histochem Cytochem. 38(5):685-690 (1990); Roepe et al., Biochemistry. 32(41):11042-11056 (1993); Simon et al., Proc Natl Acad Sci USA. 91(3):1128-1132 (1994)), decrease lysosomal and endosomal pH (Schindler et al., Biochemistry. 35(9):2811-2817 (1996); Altan et al., J Exp Med. 187(10):1583-1598 (1998)), decrease plasma membrane potential (Roepe et al., Biochemistry. 32(41):11042-11056 (1993)), increase plasma membrane conductance to chloride (Gill et al., Cell. 71(1):23-32 (1992)) and ATP (Abraham et al., Proc Natl Acad Sci USA. 90(1):312-316 (1993)), and increase rates of vesicle transport (Altan et al., Proc Natl Acad Sci USA. 96(8):4432-4437 (1999)). Thus, the cells and cell lines of this invention allow screening assays that are free from any artifact caused by selective drugs. In some preferred embodiments, the cells and cell lines of this invention are not cultured with selective drugs such as antibiotics before or after cell sorting, so that cells and cell lines with desired properties are isolated by sorting, even when not beginning with an enriched cell population.

In some embodiments, properties of the cells and cell lines of the invention, such as stability, physiological relevance, reproducibility in an assay (Z′), or physiological EC₅₀ or IC₅₀ values, are achievable under specific culture conditions. In some embodiments, the culture conditions are standardized and rigorously maintained without variation, for example, by automation. Culture conditions may include any suitable conditions under which the cells or cell lines are grown and may include those known in the art. A variety of culture conditions may result in advantageous biological properties for any of the GABA receptors, or their mutants or allelic variants.

In other embodiments, the cells and cell lines of the invention with desired properties, such as stability, physiological relevance, reproducibility in an assay (Z′), or physiological EC₅₀ or IC₅₀ values, can be obtained within one month or less. For example, the cells or cell lines may be obtained within 2, 3, 4, 5, or 6 days, or within 1, 2, 3 or 4 weeks, or any length of time in between.

When collections or panels of cells or cell lines are produced, e.g., for drug screening, the cells or cell lines in the collection or panel may be matched such that they are the same (including substantially the same) with regard to one or more selective physiological properties. The “same physiological property” in this context means that the selected physiological property is similar enough amongst the members in the collection or panel such that the cell collection or panel can produce reliable results in drug screening assays; for example, variations in readouts in a drug screening assay will be due to, e.g., the different biological activities of test compounds on cells expressing different forms of GABA receptor, rather than due to inherent variations in the cells. For example, the cells or cell lines may be matched to have the same growth rate, i.e., growth rates with no more than one, two, three, four, or five hour difference amongst the members of the cell collection or panel. This may be achieved by, for example, binning cells by their growth rate into five, six, seven, eight, nine, or ten groups, and creating a panel using cells from the same binned group. Methods of determining cell growth rate are well known in the art. The cells or cell lines in a panel also can be matched to have the same Z′ factor (e.g., Z′ factors that do not differ by more than 0.1), GABA receptor subunit expression level (e.g., GABA receptor subunit expression levels that do not differ by more than 5%, 10%, 15%, 20%, 25%, or 30%), adherence to tissue culture surfaces, and the like. Matched cells and cell lines can be grown under identical conditions, achieved by, e.g., automated parallel processing, to maintain the selected physiological property.

Matched cell panels of the invention can be used to, for example, identify modulators with defined activity (e.g., agonist or antagonist) on GABA receptor; to profile compound activity across different forms of GABA receptor; to identify modulators active on just one form of GABA receptor; and to identify modulators active on just a subset of GABA receptors. The matched cell panels of the invention allow high throughput screening. Screenings that used to take months to accomplish can now be accomplished within weeks.

In another aspect, the invention provides methods of using the cells and cell lines of the invention. The cells and cell lines of the invention may be used in any application for which functional GABA_{A} subunits or GABA_{A} ion channels are needed. For example, the cells and cell lines may be used, for example, but not limited to, in an in vitro cell-based assay or an in vivo assay (where the cells are implanted in an animal (e.g., a non-human mammal)) to, e.g., screen for GABA_{A} receptor modulators; to produce proteins for crystallography and binding studies; to investigate compound selectivity and dosing; to investigate receptor/compound binding kinetic and stability; and to study the effects of receptor expression on cellular physiology (e.g., electrophysiology, protein trafficking, protein folding, and protein regulation). The cells and cell lines of the invention may also be used in knock down studies to study the roles of specific GABA_{A} subunits.

The cells and cell lines of the invention comprising functional GABA_{A} receptors can be used to identify modulators of GABA_{A} receptor function. These modulators may be useful as therapeutics for treating GABA_{A} receptor disease states. For example, the modulators may increase or decrease the ion conductance mediated by GABA_{A} receptor.

While many combinations of GABA_{A} subunits are possible, only a handful of GABA_{A} receptors have been reported. Thus, additional, yet to be identified, combinations of GABA_{A} subunits (which may form GABA_{A} receptors) may exist in vivo. Accordingly, the cells and cell lines of the present invention comprising various combinations of GABA subunits may be used to identify physiologically relevant, yet to be described, combinations of subunits, as well as novel modulators of previously described and yet to be described combinations of subunits. Once previously unreported combinations of subunits (i.e. novel GABA receptors) have been identified, their in vivo expression pattern can be determined by methods known in the art (e.g. immunohistochemistry, in situ hybridization, radio-ligand binding assays). Further, specific modulators of the novel GABA receptors can be used to determine where in the body the novel GABA receptors are expressed using methods known in the art (e.g. tissue slices, MRI, functional MRI, PET, CT, SPECT). For example, if such specific modulators bind to novel GABA receptors in the brain, specific regions/nuclei of brain can be identified.

Once the in vivo expression pattern of a novel GABA receptor has been characterized, the physiological and patho-physiological relevance of the novel GABA receptor can be determined. Indeed, the expression profile itself may give some indication of the physiological or pathological role of the novel GABA receptor. Based on the expression profile of known GABA receptors, possible physiological and patho-physiological roles include, but are not limited to: anxiety, sedation, cognition/memory/learning, ethanol dependence, chronic pain, epilepsy, addiction, dependence, depression, well-being and mood disturbances, sleep, appetite, diabetes, endocrine/hormonal indications, vision regulation (i.e. retinal bipolar cells, eye blink conditioning paradigms, other vision indications), lung cancer, prostate cancer, breast cancer and other carcinomas, glucose metabolic response, anorexia, prostaglandin induced thermogenesis, cardiac baro-receptor reflex and other reflex abnormalities. Knowledge of the expression profile may also be useful in studies on the amelioration of side effects of other medications such as risk of dependence, sedation, anxiety, mood disturbances, sleep disruption, sleep disturbances (such as sleep walking and sleep eating) suicidal thoughts, aggression, and addiction. Further, the specific modulators of novel GABA receptors may be used in in vitro and in vivo studies to determine the physiological relevance of these previously undescribed GABA subunit combinations.

Cells and cell lines expressing various combinations of subunits can be used separately or together to identify GABA_{A} receptor modulators, including those specific for a particular set of GABA_{A} subunits or a particular subunit of GABA_{A} and to obtain information about the activities of individual subunits. The present cells and cell lines may be used to identify the roles of different forms of GABA_{A} receptors in different GABA_{A} receptor pathologies by correlating the identity of in vivo forms of GABA_{A} receptors with the identify of known forms of GABA_{A} receptors based on their response to various modulators. This allows for the selection of disease- or tissue-specific GABA_{A} receptor modulators for highly targeted treatment of such GABA_{A} receptor-related pathologies. Further, such a combinatorial panel may be used to identify modulators that act on specific GABA_{A} receptor targets localized in discrete regions or nuclei of the brain. In addition, known GABA_{A} modulators have often failed in clinical trials due to unexpected side-effects or toxicity. A combinatorial panel may identify interactions of such modulators with previously unidentified combinations of GABA_{A} subunits that may be responsible for side-effects. Such a combinatorial panel could be used to identify modulators lacking off-target activity (i.e. modulators that demonstrate high specificity for a particular GABA_{A} receptor combination).

Modulators include any substance or compound that alters an activity of GABA_{A} receptor or a GABA_{A} receptor subunit. The modulator can be a GABA_{A} receptor agonist (potentiator or activator) or antagonist (inhibitor or blocker), including partial agonists or antagonists, selective agonists or antagonists and inverse agonists, and can be an allosteric modulator. A substance or compound is a modulator even if its modulating activity changes under different conditions or concentrations or with respect to different forms of GABA_{A} receptor. In other aspects, a modulator may change the ability of another modulator to affect the function of a GABA_{A} receptor. For example, a modulator of a form of GABA_{A} receptor that is not induced by GABA may render that form of GABA_{A} receptor susceptible to induction by GABA.

To identify a GABA_{A} receptor modulator, one can expose a novel cell or cell line of the invention to a test compound under conditions in which the GABA_{A} receptor would be expected to be functional and then detect a statistically significant change (e.g., p<0.05) in GABA_{A} receptor activity compared to a suitable control, e.g., cells that are not exposed to the test compound. Positive and/or negative controls using known agonists or antagonists and/or cells expressing different combinations of GABA_{A} subunits may also be used. In some embodiments, the GABA_{A} receptor activity to be detected and/or measured is membrane depolarization, change in membrane potential, fluorescence resulting from such membrane changes, or quenching of a halide-sensitive YFP. One of ordinary skill in the art would understand that various assay parameters, e.g., signal to noise ratio, may be optimized.

In some embodiments, one or more cells or cell lines of the invention are exposed to a plurality of test compounds, for example, a library of test compounds. A library of test compounds can be screened using the cell lines of the invention to identify one or more modulators. The test compounds can be chemical moieties (such as small molecules), polypeptides, peptides, peptide mimetics, antibodies or antigen-binding portions thereof. In the case of antibodies, they may be non-human antibodies, chimeric antibodies, humanized antibodies, or fully human antibodies. The antibodies may be intact antibodies comprising a full complement of heavy and light chains, antigen-binding portions of any antibody (including antibody fragments (such as Fab, Fab′, F(ab′)₂, Fd, Fv, dAb and the like)), single chain antibodies (scFv), single domain antibodies, a heavy or light chain variable region, or an antigen-binding portion of a heavy chain or light chain variable region.

In some embodiments, prior to exposure to a test compound, the cells or cell lines of the invention may be modified by pretreatment with, for example, enzymes, including but not limited to mammalian or other animal enzymes, plant enzymes, bacterial enzymes, enzymes from lysed cells, protein modifying enzymes, lipid modifying enzymes, and enzymes in the oral cavity, gastrointestinal tract, stomach or saliva. Such enzymes can include, for example, kinases, proteases, phosphatases, glycosidases, oxidoreductases, transferases, hydrolases, lyases, isomerases, ligases and the like. Alternatively, the cells and cell lines may be exposed to the test compound first followed by treatment to identify compounds that alter the modification of the GABA_{A} by the treatment.

In some embodiments, large compound collections are tested for GABA_{A} receptor modulating activity in a cell-based, functional, high-throughput screen (HTS), e.g., using a 96 well, 384 well, 1536 well or higher format. In some embodiments, a test compound or multiple test compounds including a library of test compounds may be screened using more than one cell or cell line of the invention. In the case of a cell or cell line of the invention that expresses a human GABA_{A} receptor, one can expose the cells to a test compound to identify a compound that modulates GABA_{A} receptor activity (either increasing or decreasing) for use in the treatment of disease or condition characterized by undesired GABA_{A} receptor activity, or the decrease or absence of desired GABA_{A} receptor activity.

These and other embodiments of the invention may be further illustrated in the following non-limiting Examples.

### EXAMPLES

### Example 1

### Generating a Stable GABA_{A}-Expressing Cell Line

Generating Expression Vectors

Plasmid expression vectors that allowed streamlined cloning were generated based on pCMV-SCRIPT (Stratagene) and contained various necessary components for transcription and translation of a gene of interest, including: CMV and SV40 eukaryotic promoters; SV40 and HSV-TK polyadenylation sequences; multiple cloning sites; Kozak sequences; and neomycin/kanamycin resistance cassettes.

### Step 1—Transfection

We transfected both 293T and CHO cells. The example focuses on CHO cells, where the CHO cells were cotransfected with three separate plasmids, one encoding a human GABA alpha subunit (SEQ ID NO: 1-3 or 5), one encoding the human GABA beta 3 subunit (SEQ ID NO: 10) and the other encoding the human GABA gamma 2 subunit (SEQ ID NO: 13) in the following combinations: α1β3γ2s (α1), α2β3γ2s (α2), α3β3γ2s (α3) and α5β3γ2s (α5). As will be appreciated by those of skill in the art, any reagent that is suitable for use with a chosen host cell may be used to introduce a nucleic acid, e.g. plasmid, oligonucleotide, labeled oligonucleotide, into a host cell with proper optimization. Examples of reagents that may be used to introduce nucleic acids into host cells include but are not limited to Lipofectamine, Lipofectamine 2000, Oligofectamine, TFX reagents, Fugene 6, DOTAP/DOPE, Metafectine, or Fecturin.

Although drug selection is optional in the methods of this invention, we included one drug resistance marker per plasmid. The sequences were under the control of the CMV promoter. An untranslated sequence encoding a tag for detection by a signaling probe was also present along with a sequence encoding a drug resistance marker. The target sequences utilized were Target Sequence 1 (SEQ ID NO: 58), Target Sequence 2 (SEQ ID NO: 59) and Target Sequence 3 (SEQ ID NO: 60). In these examples, the GABA alpha subunit gene-containing vector contained Target Sequence 1, the GABA beta subunit gene-containing vector contained Target Sequence 2 and the GABA gamma subunit gene-containing vector contained the Target Sequence 3.

### Step 2—Selection Step

Transfected cells were grown for 2 days in HAMF12-FBS, followed by 14 days in antibiotic-containing HAMF12-FBS. The antibiotic containing period had antibiotics added to the media as follows: Puromycin (3.5 ug/ml), Hygromycin (150 ug/ml), and G418/Neomycin (300 ug/ml)

### Step 3—Cell Passaging

Following antibiotic selection, and prior to introduction of fluorogenic probes, cells were passaged 6 to 18 times in the absence of antibiotics to allow time for expression that is not stable over the selected period of time to subside.

Step 4—Exposure of cells to fluorogenic probes

Cells were harvested and transfected with signaling probes (SEQ ID NO: 61-63). As will be appreciated by those of skill in the art, any reagent that is suitable for use with a chosen host cell may be used to introduce a nucleic acid, e.g. plasmid, oligonucleotide, labeled oligonucleotide, into a host cell with proper optimization. Examples of reagents that may be used to introduce nucleic acids into host cells include but are not limited to Lipofectamine, Lipofectamine 2000, Oligofectamine, TFX reagents, Fugene 6, DOTAP/DOPE, Metafectine, or Fecturin. Signaling Probe 1 binds Target Sequence 1, Signaling Probe 2 binds Target Sequence 2 and Signaling Probe 3 binds Target Sequence 3. The cells were then collected for analysis and sorted using a fluorescence activated cell sorter (below).

Target Sequences Detected by Signaling Probes

Target 1

(SEQ ID NO: 58)

5′-GTTCTTAAGGCACAGGAACTGGGAC-3′
(alpha subunit)
Target 2

(SEQ ID NO: 59)

5′-GAAGTTAACCCTGTCGTTCTGCGAC-3′
(beta subunit)
Target 3

(SEQ ID NO: 60)

5′-GTTCTATAGGGTCTGCTTGTCGCTC-3′
(gamma subunit)

### Signaling Probes

Supplied as 100 μM stocks

A similar probe using a Quasar Dye (BioSearch) with spectral properties similar to Cy5 was used in certain experiments. Note also that 5-MedC and 2-aminodA mixmer probes rather than DNA probes were used in some instances.

Signaling probe 1 - binds (Target 1)

(SEQ ID NO: 61)

5′-Cy5 GCCAGTCCCAGTTCCTGTGCCTTAAGAACCTCGC
BHQ3 quench-3′
Signaling probe 2 - binds (Target 2)

(SEQ ID NO: 62)

5′-Cy5.5 GCGAGTCGCAGAACGACAGGGTTAACTTCCTCGC
BHQ3 quench-3′

Note that BHQ3 could be substituted with BHQ2 or a gold particle in Probe 1 or Probe 2.

Signaling probe 3 - binds (Target 3)

(SEQ ID NO: 63)

5′-Fam GCGAGAGCGACAAGCAGACCCTATAGAACCTCGC
BHQ1 quench-3′′

Note that BHQ1 could be substituted with BHQ2 or Dabcyl in Probe 3.

### Step 5—Isolation of Positive Cells

The cells were dissociated and collected for analysis and sorting using a fluorescence activated cell sorter. Standard analytical methods were used to gate cells fluorescing above background and to isolate individual cells falling within the gate into barcoded 96-well plates. The gating hierarchy was as follows: Gating hierarchy: coincidence gate>singlets gate>live gate>Sort gate. With this gating strategy, the top 0.04-0.4% of triple positive cells were marked for sorting into barcoded 96-well plates.

Step 6—Additional Cycles of Steps 1-5 and/or 3-5

Steps 1 to 5 and/or 3-5 were repeated to obtain a greater number of cells. Two independent rounds of steps 1-5 were completed, and for each of these cycles, at least three internal cycles of steps 3-5 were performed for the sum of independent rounds.

### Step 7—Estimation of Growth Rates for the Populations of Cells

The plates were transferred to a Hamilton Microlabstar automated liquid handler. Cells were incubated for 5-7 days in a 1:1 mix of 2-3 day conditioned growth medium:fresh growth medium (growth medium is Ham's F12/10% FBS) supplemented with 100 units penicillin/ml plus 0.1 mg/ml streptomycin and then dispersed by trypsinization with 0.25% trypsin to minimize clumps and transferred to new 96-well plates. After the clones were dispersed, plates were imaged to determine confluency of wells (Genetix). Each plate was focused for reliable image acquisition across the plate. Reported confluencies of greater than 70% were not relied upon. Confluency measurements were obtained at days every 3 times over 9 days (between days 1 and 10 post-dispersal) and used to calculate growth rates.

### Step 8—Binning Populations of Cells According to Growth Rate Estimates

Cells were binned (independently grouped and plated as a cohort) according to growth rate between 10-11 days following the dispersal step in step 7. Bins were independently collected and plated on individual 96 well plates for downstream handling, and there could be more than one target plate per specific bin. Bins were calculated by considering the spread of growth rates and bracketing a range covering a high percentage of the total number of populations of cells. Depending on the sort iteration (see Step 5), between 5 and 6 growth bins were used with a partition of 1-4 days. Therefore each bin corresponded to a growth rate or population doubling time between 12 and 14.4 hours depending on the iteration.

### Step 9—Replica Plating to Speed Parallel Processing and Provide Stringent QC

The plates were incubated under standard and fixed conditions (humidified 37° C., 5% CO₂/95% air) in Ham's F12 media/10% FBS without antibiotics. The plates of cells were split to produce 4 sets (the set consists of all plates with all growth bins—these steps ensure there are 4 replicates of the initial set) of target plates. Up to 2 target plate sets were committed for cryopreservation (see below), and the remaining set was scaled and further replica plated for passage and for functional assay experiments. Distinct and independent tissue culture reagents, incubators, personnel and carbon dioxide sources were used for each independently carried set of plates. Quality control steps were taken to ensure the proper production and quality of all tissue culture reagents: each component added to each bottle of media prepared for use was added by one designated person in one designated hood with only that reagent in the hood while a second designated person monitored to avoid mistakes. Conditions for liquid handling were set to eliminate cross contamination across wells. Fresh tips were used for all steps or stringent tip washing protocols were used. Liquid handling conditions were set for accurate volume transfer, efficient cell manipulation, washing cycles, pipetting speeds and locations, number of pipetting cycles for cell dispersal, and relative position of tip to plate.

### Step 10—Freezing Early Passage Stocks of Populations of Cells

At least two sets of plates were frozen at −70 to −80 C. Plates in each set were first allowed to attain confluencies of 70 to 100%. Media was aspirated and 90% FBS and 10% DMSO was added. The plates were sealed with Parafilm and then individually surrounded by 1 to 5 cm of foam and placed into a −80 C freezer.

### Step 11—Methods and Conditions for Initial Transformative Steps to Produce VSF

The remaining set of plates were maintained as described in step 9 (above).

All cell splitting was performed using automated liquid handling steps, including media removal, cell washing, trypsin addition and incubation, quenching and cell dispersal steps.

### Step 12—Normalization Methods to Correct any Remaining Variability of Growth Rates

The consistency and standardization of cell and culture conditions for all populations of cells was controlled. Any differences across plates due to slight differences in growth rates could be controlled by periodic normalization of cell numbers across plates.

### Step 13—Characterization of Population of Cells

The cells were maintained for 6 to 8 weeks of cell culture to allow for their in vitro evolution under these conditions. During this time, we observed size, morphology, fragility, response to trypsinization or dissociation, roundness/average circularity post-dissociation, percentage viability, tendency towards microconfluency, or other aspects of cell maintenance such as adherence to culture plate surfaces.

### Step 14—Assessment of Potential Functionality of Populations of Cells Under VSF Conditions

Populations of cells were tested using functional criteria. Membrane potential assay kits (Molecular Devices/MDS) were used according to manufacturer's instructions. Cells were tested at multiple different densities in 96 or 384-well plates and responses were analyzed. A variety of time points post plating were used, for instance 12-48 hours post plating. Different densities of plating were also tested for assay response differences.

### Step 15

The functional responses from experiments performed at low and higher passage numbers were compared to identify cells with the most consistent responses over defined periods of time, ranging from 3 to 9 weeks. Other characteristics of the cells that changed over time are also noted, including morphology, tendency toward microconfluency, and time to attach to culture matrices post-plating.

### Step 16

Populations of cells meeting functional and other criteria were further evaluated to determine those most amenable to production of viable, stable and functional cell lines. Selected populations of cells were expanded in larger tissue culture vessels and the characterization steps described above were continued or repeated under these conditions. At this point, additional standardization steps were introduced for consistent and reliable passages. These included different plating cell densities, time of passage, culture dish size/format and coating, fluidics optimization, cell dissociation optimization (type, volume used, and length of time), as well as washing steps. Assay Z′ scores were stable when tested every few days over the course of four weeks in culture.

Also, viability of cells at each passage were determined. Manual intervention was increased and cells were more closely observed and monitored. This information was used to help identify and select final cell lines that retained the desired properties Final cell lines and back-up cell lines were selected that showed consistent growth, appropriate adherence, as well as functional response.

### Step 17—Establishment of Cell Banks

The low passage frozen plates (see above) corresponding to the final cell line and back-up cell lines were thawed at 37° C., washed two times with Ham's F12/10% FBS and incubated in humidified 37° C./5% CO2 conditions. The cells were then expanded for a period of 2-3 weeks. Cell banks for each final and back-up cell line consisting of 25 vials each with 10 million cells were established.

### Step 18

At least one vial from the cell bank was thawed and expanded in culture. The resulting cells were tested to confirm that they met the same characteristics for which they were originally selected.

### Example 2

### Verification of GABA_{A} Cell Lines Response to GABA Ligand

The response of CHO cell lines expressing GABA_{A} (subunit combinations of α1β3γ2s (α1), α2β3γ2s (α2), α3β3γ2s (α3) and α5β3γ2s (α5)) GABA, the endogenous GABA_{A} ligand, was evaluated. Interaction of cell lines with GABA was evaluated by measuring the membrane potential of GABA_{A}, in response to GABA using the following protocol.

Cells were plated 24 hours prior to assay at 10-25,000 cells per well in 384 well plates in growth media (Ham's F-12 media plus FBS and glutamine). Media removal was followed by the addition of membrane potential dye diluted in load buffer (137 mM NaCl, 5 mMKCl, 1.25 mM CaCl, 25 mM HEPES, 10 mM Glucose). Incubation was for 1 hour, followed by plate loading onto the high throughput fluorescent plate reader (Hamamastu FDSS). GABA ligand was diluted in MP assay buffer (137 mM NaCl, 5 mM KGluconate,1.25 mM CaCl, 25 mM HEPES, 10 mM Glucose) to the desired concentration (when needed, serial dilutions of GABA were generated, concentrations used: 3 nM, 10 nM, 30 nM, 100 nM, 300 nM, 1 uM, 3 uM, 10 uM) and added to each well. The plates were read for 90 seconds.

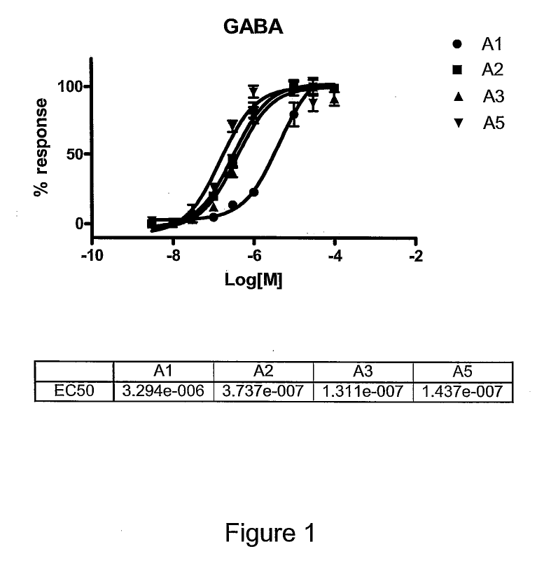

FIG. 1 and Table 6 (below) demonstrate that each of the cell lines generated responds to GABA ligand. These results indicate that the GABA_{A} cell lines produced, which respond as expected to the endogenous ligand, are physiologically relevant for use in high-throughput screening assays. Further, the replicate wells produced precise EC₅₀ values from well to well indicating high reproducibility of the GABA_{A} cell lines. Z′ values generated using the membrane potential assay were α1β3γ2s 0.58, α2β3γ2s 0.67, α3β3γ2s 0.69 and α5β3γ2s 0.62.

### Example 3

### Additional Verification of GABA_{A} Cell Lines Using A Known GABA_{A} Modulator

The GABA_{A} cell lines and membrane potential assay were verified by the methods described in Example 2 using serial dilutions in assay buffer of bicuculline (a known antagonist) at 30 uM, 10 uM, 3 uM, 1 uM, 300 nM, 100 nM and 30 nM

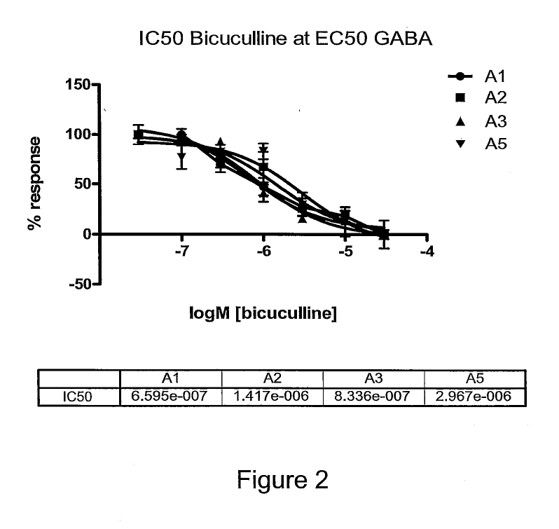

Bicuculline was found to interact with all four GABA_{A} cell lines in the presence of EC₅₀ concentrations of GABA (FIG. 2). These results indicate that the GABA_{A} cell lines produced, which respond as expected to this known modulator of GABA_{A}, are physiologically and pharmacologically relevant for use in high-throughput screening assays.

### Example 4

### Characterization of Cell Line Expressing GABA_{A} for Native GABA_{A} Function Using Membrane Potential Assay

The interaction of CHO cell lines expressing GABA_{A} (subunit combinations of α1β3γ2s (α1), α2β3γ2s (α2), α3β3γ2s (α3) and α5β3γ2s (α5)) with 1280 compounds from the LOPAC 1280 (Library of Pharmacologically Active Compounds) was evaluated (Sigma-RBI Prod. No. L01280). The LOPAC 1280 library contains high purity, small organic ligands with well documented pharmacological activities. Interaction of cell lines with test compounds was evaluated by measuring the membrane potential of GABA_{A}, in response to test compounds using the following protocol.

Cells were plated 24 hours prior to assay at 10-25,000 cells per well in 384 well plates in growth media (Ham's F-12 media plus FBS and glutamine). Media removal was followed by the addition of membrane potential dye diluted in load buffer (137 mM NaCl, 5 mMKC1, 1.25 mM CaCl, 25 mM HEPES, 10 mM Glucose). Incubation was for 1 hour, followed by plate loading onto the high throughput fluorescent plate reader (Hamamastu FDSS). Test compounds were diluted in MP assay buffer (137 mM NaCl, 5 mM KGluconate, 1.25 mM CaCl, 25 mM HEPES, 10 mM Glucose) to the desired concentration (when needed, serial dilutions of each test compound were generated, concentrations used: 3 nM, 10 nM, 30 nM, 100 nM, 300 nM, 1 uM, 3 uM, 10 uM) and added to each well. The plates were read for 90 seconds.

### Results

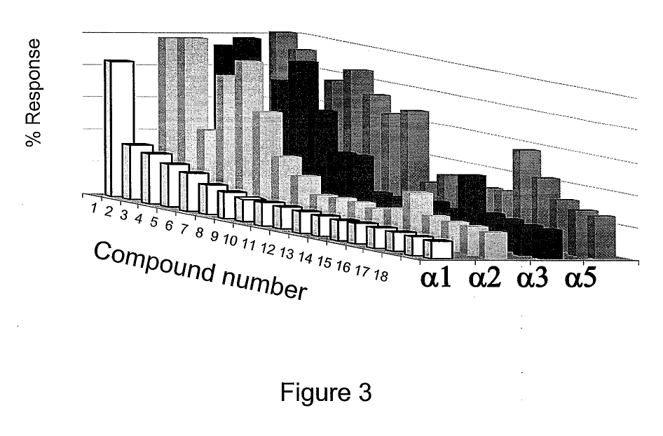

The activity of each compound towards the GABA_{A} cell lines produced was measured and compounds which exhibited similar or greater activity as GABA (the endogenous ligand) were scored as positive hits. Of the 1280 compounds screened, 34 activated at least one cell line (i.e., either α1, α2, α3 and α5) as well as, if not better, than GABA. Relative activities of the top 18 hits found are plotted in FIG. 3. The interaction of 17 of these compounds with the produced GABA_{A} cell lines was confirmed in the following dose response studies. Modulators which require GABA to be present, partial agonists and low potency compounds were not included in the list.

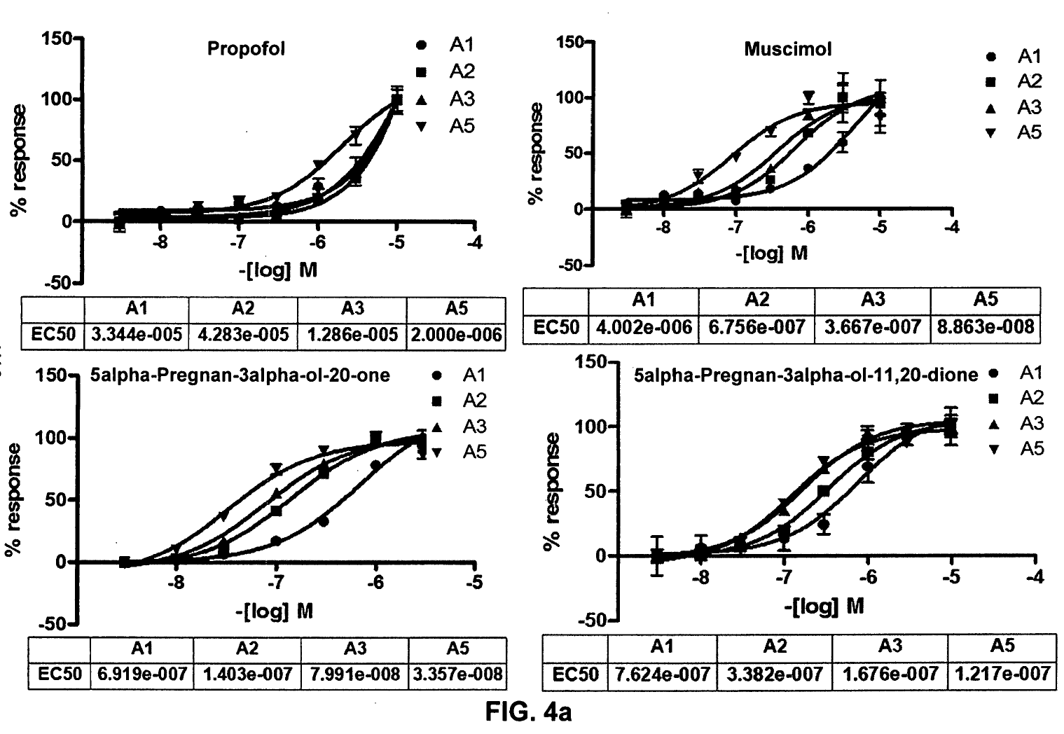

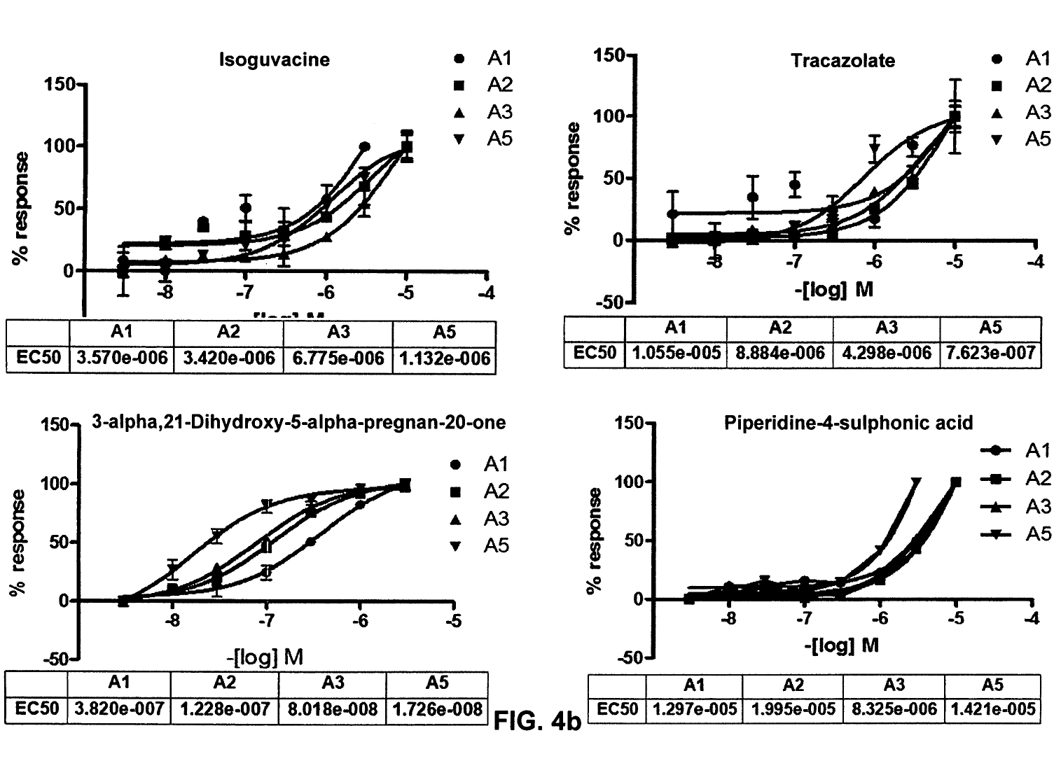

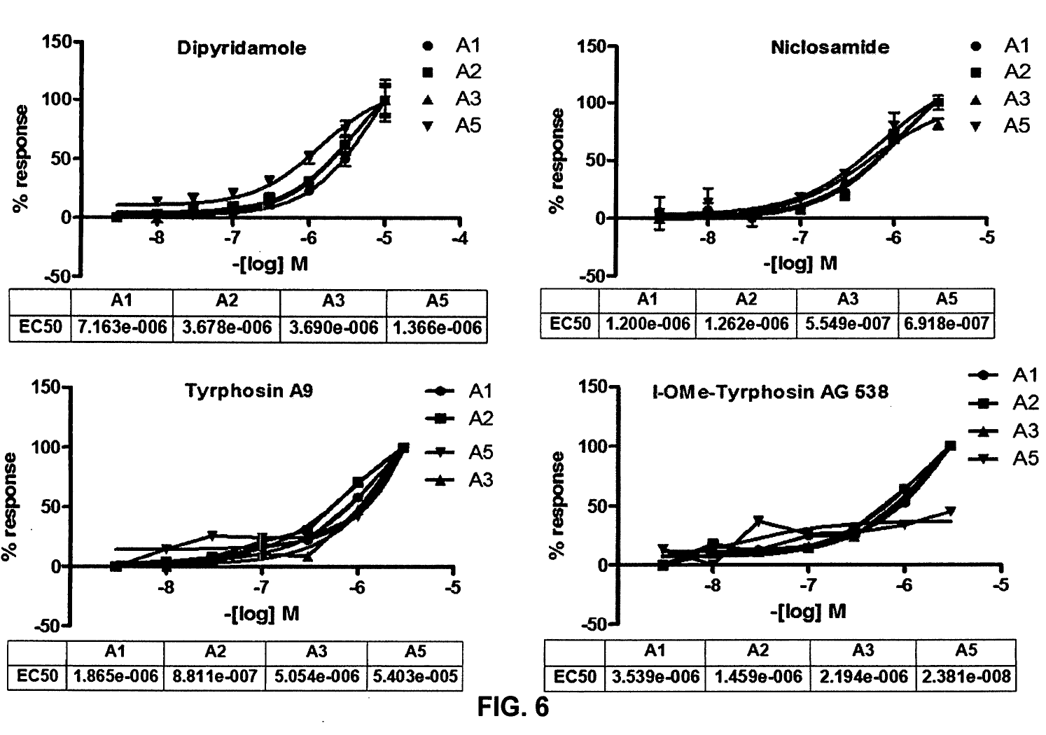

The screening assay identified each of the GABA_{A} agonists in the LOPAC library: GABA (endogenous ligand), propofol, isoguvacine hydrochloride, muscimol hydrobromide, piperidine-4-sulphonic acid, 3-alpha,21-dihydroxy-5-alpha-pregnan-20-one (a neurosteroid), 5-alpha-pregnan-3alpha-ol-11,20-dione (a neurosteroid), 5-alpha-pegnan-3alpha-ol-20-one (a neurosteroid), and tracazolate. The results (FIGS. 6a and 6b) indicate that the produced GABA_{A} cell lines respond in a physiologically relevant manner (e.g., they respond to agonists of the endogenous receptor). EC₅₀ values for these eight agonists were determined and are included in FIGS. 4a and 4b and in Table 6 (below).

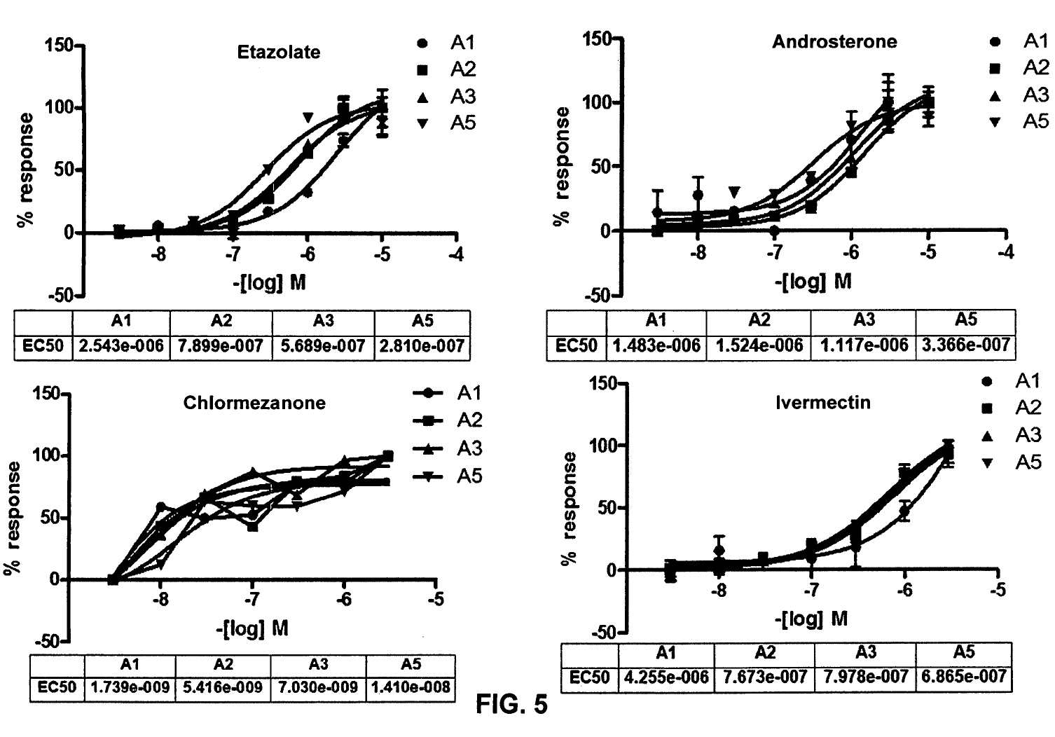

The screening assay also identified four compounds in the LOPAC library not described as GABA agonist but known to have other activities associated with GABA_{A} which we noted: etazolate (a phosphodiesterase inhibitor), androsterone (a steroid hormone), chlormezanone (a muscle relaxant), and ivermectin (an anti-parasitic known to effect chlorine channels). EC₅₀ values for these four compounds were determined and are summarized in FIG. 5 and in Table 6 (below).

The screening assay further identified four compounds in the LOPAC library which, until now, were not known to interact with GABA_{A}. These novel compounds include: dipyrimidole (an adenosine deaminase inhibitor), niclosamide (an anti-parasitic), tyrphosin A9 (a PDGFR inhibitor), and I-Ome-Tyrphosin AG 538 (an IGF RTK inhibitor). EC₅₀ values for these four compounds were determined and are summarized in FIG. 6 and in Table 6 (below).

The results of the screening assays summarized in Table 6:

| | | Chromocell | |
|---|---|---|---|
| Compound | Description | Target | EC₅₀ Values |
| GABA | endogenous ligand | α1, α2, α3, α5 | α1 3.29 μM |
| | | | α2 374 nM |
| | | | α3 131 nM |
| | | | α5 144 nM |
| Muscimol | agonist | α1, α2, α3, α5 | α1 4 μM |
| | | | α2 675 nM |
| | | | α3 367 nM |
| | | | α5 80 nM |
| Propofol | agonist | α1, α2, α3, α5 | α1 33.4 μM |
| | | | α2 42.8 μM |
| | | | α3 12.9 μM |
| | | | α5 2.0 μM |
| Isoguvacine | agonist | α1, α2, α3, α5 | α1 3.57 μM |
| hydrochloride | | | α2 3.42 μM |
| | | | α3 6.78 μM |
| | | | α5 1.13 μM |
| Piperidine-4- | agonist | α1, α2, α3, α5 | α1 13 μM |
| sulphonic acid | | | α2 20 μM |
| | | | α3 8.33 μM |
| | | | α5 14.2 μM |
| 3-alpha, 21- | neurosteroid | α1, α2, α3, α5 | α1 382 nM |
| dihydroxy-5- | (agonist) | | α2 123 nM |
| alpha-pregnan- | | | α3 80.2 nM |
| 20-one | | | α5 17.3 nM |
| 5-alpha-Pregnan- | neurosteroid | α1, α2, α3, α5 | α1 762 nM |
| 3alpha-ol-11,20- | (agonist) | | α2 338 nM |
| dione | | | α3 168 nM |
| | | | α5 122 nM |
| 5-alpha-Pregnan- | neurosteroid | α1, α2, α3, α5 | α1 692 nM |
| 3alpha-ol-20-one | (agonist) | | α2 140 nM |
| | | | α3 80.0 nM |
| | | | α5 33.6 nM |
| Tracazolate | agonist | α1, α2, α3, α5 | α1 10.6 μM |
| | | | α2 8.9 μM |
| | | | α3 4.3 μM |
| | | | α5 762 nM |
| Androsterone | Steroid with | α1, α2, α3, α5 | α1 1.48 μM |
| | GABA_{A} receptor | | α2 1.52 μM |
| | activity | | α3 1.12 μM |
| | | | α5 337 nM |
| Ivermectin | Phospho-diesterase | α1, α2, α3, α5 | α1 4.26 μM |
| | inhibitor: Known | | α2 767 nM |
| | GABAergic | | α3 798 nM |
| | | | α5 687 nM |
| Chlormezanone | Muscle relaxant: | α1, α2, α3, α5 | α1 1.74 nM |
| | known GABA | | α2 5.42 nM |
| | ligand | | α3 7.0 nM |
| | | | α5 14.1 nM |
| Etazolate | Anti-parasitic: | α1, α2, α3, α5 | α1 2.54 μM |
| | known effector of | | α2 790 nM |
| | chlorine channels | | α3 569 nM |
| | | | α5 281 nM |
| Dipyridamole | Adenosine | α1, α2, α3, α5 | α1 7.16 μM |
| | inhibitor known to | | α2 3.68 μM |
| | effect GABA | | α3 3.69 μM |
| | release in neurons | | α5 1.37 μM |
| | (not known to bind | | |
| | to GABA_{A}) | | |
| Niclosamide | Anti parasitic (side | α1, α2, α3, α5 | α1 1.2 μM |
| | effects include | | α2 1.26 μM |
| | drowsiness and | | α3 0.55 μM |
| | dizziness) | | α5 0.69 μM |
| Tyrphostin A9 | PDGFR inhibitor | α1, α2, α3, α5 | α1 1.8 μM |
| | | | α2 0.88 μM |
| | | | α3 5.0 μM |
| | | | α5 54.0 μM |
| I-OMe Tyrphostin | IGF RTK inhibitor | α1, α2, α3, α5 | α1 3.5 μM |
| 538 | | | α2 1.5 μM |
| | | | α3 2.2 μM |
| | | | α5 Not active |

### Example 5

### Characterization GABA_{A}-CHO Cells for Native GABA_{A} Function Using Electrophysiological Assay

The following voltage-clamp protocol was used: the membrane potential was clamped to a holding potential of −60 mV. Currents were evoked by 2-sec applications of increasing concentrations of GABA (0.10-100 μM) with intermediate wash with buffer.

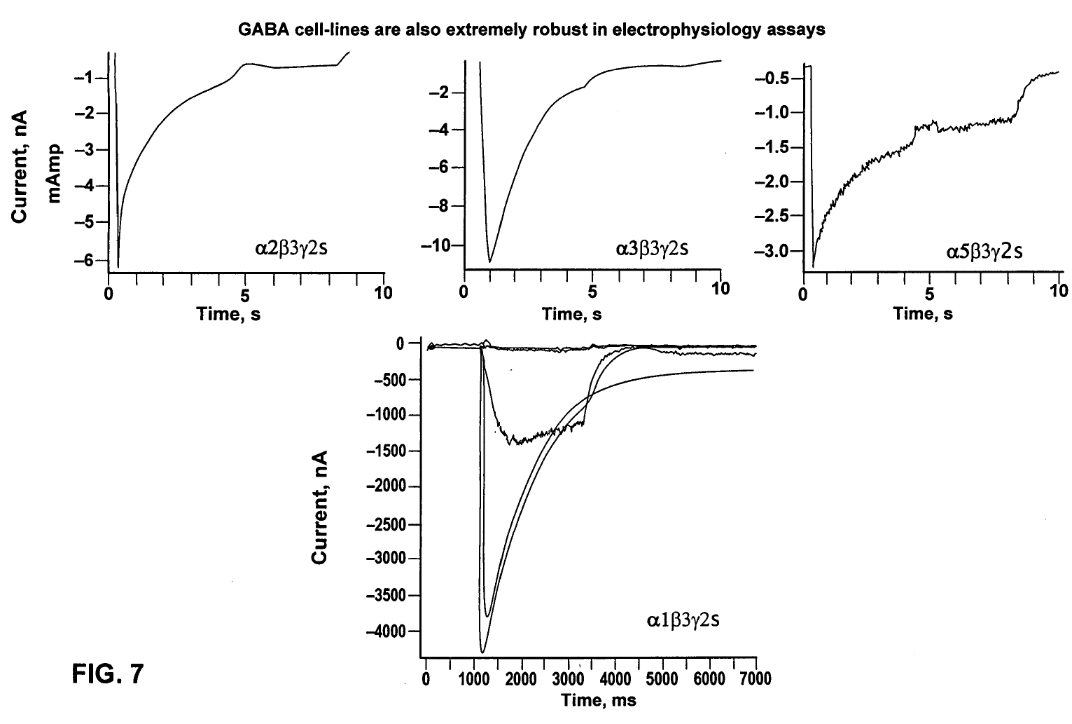

Whole cell receptor current traces for the α2, α3, and α5 GABA_{A} cell lines in response to 100 uM GABA, and the α1 GABA_{A} cell line in response to increasing concentrations of GABA (0.10-100 μM in log increments), confirm that the GABA_{A} cell lines can be used in traditional electrophysiology assays in addition to the High-Throughput Screening assays described above. These electrophysiology assay results, along with the membrane potential assay of Example 2, confirm the physiological and pharmacological relevance of the GABA_{A} cell lines produced herein. Electrophysiology is accepted as a reliable method of detecting modulators of GABA_{A} receptors. Our data indicate that the cell lines of the invention can produce similarly reliable results using a membrane potential assay. Cell lines of the prior art are not reliable or sensitive enough to effectively utilize this membrane potential assay, which is cheaper and faster than electrophysiology. Thus, the cell lines of the invention allow screening on a much larger scale than is available using electrophysiology (10,000's of assays per day using the membrane potential assay compared to less than 100 per day using electrophysiology). See FIG. 7.

### Example 6

### Characterization of an in-Cell Readout Assay for Native GABA_{A} Function Using Halide-Sensitive meYFP

The response of GABA_{A} (subunit combinations of α1β3γ2s (A1), α2β3γ2s (A2), α3β3γ2s (A3) and α5β3γ2s (A5)) expressing CHO cells of the invention to test compounds was evaluated using the following protocol for an in-cell readout assay.

Cells were plated 24 hours prior to assay at 10-25,000 cells per well in 384 well plates in growth media (Ham's F-12 media plus FBS and glutamine). Media removal was followed by the addition of loading buffer (135 mM NaCl, 5 mM KCl, 2 mM CaCl₂, 1 mM MgCl₂, 10 mM HEPES, 10 mM glucose) and incubation for 1 hour. The assay plates were then loaded on the FDSS (Hamamatsu Corporation). Test compounds (e.g. GABA ligand) were diluted in assay buffer (150 mM NaI, 5 mM KCl, 1.25 mM CaCl₂, 1 mM MgCl₂, 25 mM HEPES, 10 mM glucose) to the desired concentration (when needed, serial dilutions of each test compound were generated, effective concentrations used: 3 nM, 10 nM, 30 nM, 100 nM, 300 nM, 1 uM, 3 uM, 10 uM) and added to each well. The plates were read for 90 seconds.

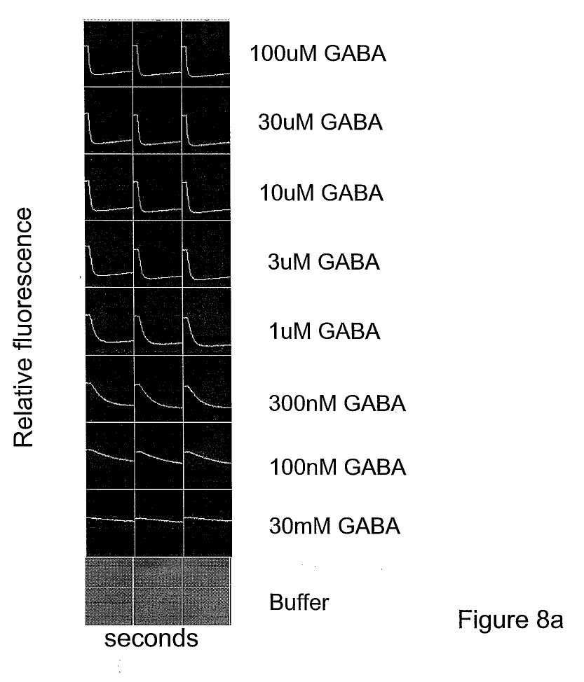

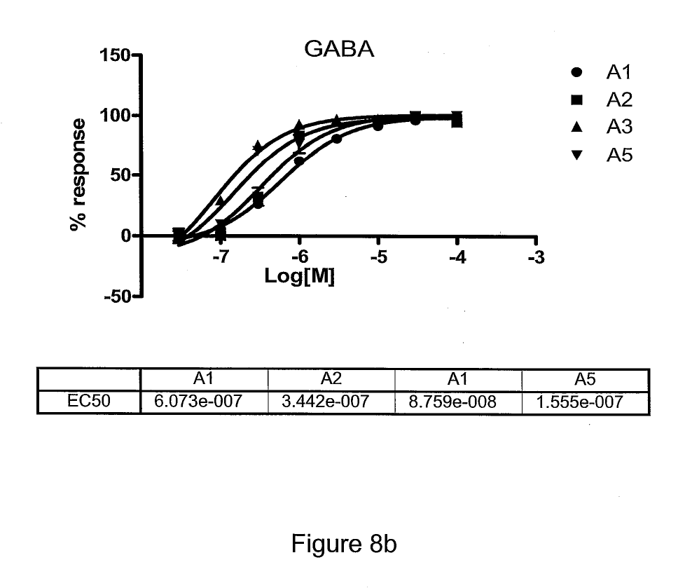

In response to increasing concentrations of GABA ligand, GABA_{A}-meYFP—CHO cells show increasing quench of meYFP signal (FIG. 8a). This quench can be used to calculate dose response curves for GABA activation (FIG. 8b). The GABA dose response curves generated by the in-cell readout assay are similar to the curves generated by the Membrane Potential Blue assay described in Example 3. These data demonstrate that the cells of the invention can be used in an in-cell readout assay to determine modulators of GABA_{A}.

SEQUENCE LISTING
Human GABA_{A} receptor alpha 1 subunit cDNA
(SEQ ID NO: 1)
ATGAGGAAAAGTCCAGGTCTGTCTGACTGTCTTTGGGCCTGGATCCTCCTT
CTGAGCACACTGACTGGAAGAAGCTATGGACAGCCGTCATTACAAGATGA
ACTTAAAGACAATACCACTGTCTTCACCAGGATTTTGGACAGACTCCTAG
ATGGTTATGACAATCGCCTGAGACCAGGATTGGGAGAGCGTGTAACCGAA
GTGAAGACTGATATCTTCGTCACCAGTTTCGGACCCGTTTCAGACCATGAT
ATGGAATATACAATAGATGTATTTTTCCGTCAAAGCTGGAAGGATGAAAG
GTTAAAATTTAAAGGACCTATGACAGTCCTCCGGTTAAATAACCTAATGG
CAAGTAAAATCTGGACTCCGGACACATTTTTCCACAATGGAAAGAAGTCA
GTGGCCCACAACATGACCATGCCCAACAAACTCCTGCGGATCACAGAGGA
TGGCACCTTGCTGTACACCATGAGGCTGACAGTGAGAGCTGAATGTCCGA
TGCATTTGGAGGACTTCCCTATGGATGCCCATGCTTGCCCACTAAAATTTG
GAAGTTATGCTTATACAAGAGCAGAAGTTGTTTATGAATGGACCAGAGAG
CCAGCACGCTCAGTGGTTGTAGCAGAAGATGGATCACGTCTAAACCAGTA
TGACCTTCTTGGACAAACAGTAGACTCTGGAATTGTCCAGTCAAGTACAG
GAGAATATGTTGTTATGACCACTCATTTCCACTTGAAGAGAAAGATTGGCT
ACTTTGTTATTCAAACATACCTGCCATGCATAATGACAGTGATTCTCTCAC
AAGTCTCCTTCTGGCTCAACAGAGAGTCTGTACCAGCAAGAACTGTCTTTG
GAGTAACAACTGTGCTCACCATGACAACATTGAGCATCAGTGCCAGAAAC
TCCCTCCCTAAGGTGGCTTATGCAACAGCTATGGATTGGTTTATTGCCGTG
TGCTATGCCTTTGTGTTCTCAGCTCTGATTGAGTTTGCCACAGTAAACTATT
TCACTAAGAGAGGTTATGCATGGGATGGCAAAAGTGTGGTTCCAGAAAAG
CCAAAGAAAGTAAAGGATCCTCTTATTAAGAAAAACAACACTTACGCTCC
AACAGCAACCAGCTACACCCCTAATTTGGCCAGGGGCGACCCGGGCTTAG
CCACCATTGCTAAAAGTGCAACCATAGAACCTAAAGAGGTCAAGCCCGAA
ACAAAACCACCAGAACCCAAGAAAACCTTTAACAGTGTCAGCAAAATTGA
CCGACTGTCAAGAATAGCCTTCCCGCTGCTATTTGGAATCTTTAACTTAGT
CTACTGGGCTACGTATTTAAACAGAGAGCCTCAGCTAAAAGCCCCCACAC
CACATCAATAG
Human GABA_{A} receptor alpha 2 subunit cDNA
(SEQ ID NO: 2)
ATGAAGACAAAATTGAACATCTACAACATGCAGTTCCTGCTTTTTGTTTTC
TTGGTGTGGGACCCTGCCAGGTTGGTGCTGGCTAACATCCAAGAAGATGA
GGCTAAAAATAACATTACCATCTTTACGAGAATTCTTGACAGACTTCTGGA
TGGTTACGATAATCGGCTTAGACCAGGACTGGGAGACAGTATTACTGAAG
TCTTCACTAACATCTACGTGACCAGTTTTGGCCCTGTCTCAGATACAGATA
TGGAATATACAATTGATGTTTTCTTTCGACAAAAATGGAAAGATGAACGT
TTAAAATTTAAAGGTCCTATGAATATCCTTCGACTAAACAATTTAATGGCT
AGCAAAATCTGGACTCCAGATACCTTTTTTCACAATGGGAAAAAATCAGT
AGCTCATAATATGACAATGCCAAATAAGTTGCTTCGAATTCAGGATGATG
GGACTCTGCTGTATACCATGAGGCTTACAGTTCAAGCTGAATGCCCAATG
CACTTGGAGGATTTCCCAATGGATGCTCATTCATGTCCTCTGAAATTTGGC
AGCTATGCATATACAACTTCAGAGGTCACTTATATTTGGACTTACAATGCA
TCTGATTCAGTACAGGTTGCTCCTGATGGCTCTAGGTTAAATCAATATGAC
CTGCTGGGCCAATCAATCGGAAAGGAGACAATTAAATCCAGTACAGGTGA
ATATACTGTAATGACAGCTCATTTCCACCTGAAAAGAAAAATTGGGTATTT
TGTGATTCAAACCTATCTGCCTTGCATCATGACTGTCATTCTCTCCCAAGTT
TCATTCTGGCTTAACAGAGAATCTGTGCCTGCAAGAACTGTGTTTGGAGTA
ACAACTGTCCTAACAATGACAACTCTAAGCATCAGTGCTCGGAATTCTCTC
CCCAAAGTGGCTTATGCAACTGCCATGGACTGGTTTATTGCTGTTTGTTAT
GCATTTGTGTTCTCTGCCCTAATTGAATTTGCAACTGTTAATTACTTCACCA
AAAGAGGATGGACTTGGGATGGGAAGAGTGTAGTAAATGACAAGAAAAA
AGAAAAGGCTTCCGTTATGATACAGAACAACGCTTATGCAGTGGCTGTTG
CCAATTATGCCCCGAATCTTTCAAAAGATCCAGTTCTCTCCACCATCTCCA
AGAGTGCAACCACGCCAGAACCCAACAAGAAGCCAGAAAACAAGCCAGC
TGAAGCAAAGAAAACTTTCAACAGTGTTAGCAAAATTGACAGAATGTCCA
GAATAGTTTTTCCAGTTTTGTTTGGTACCTTTAATTTAGTTTACTGGGCTAC
ATATTTAAACAGAGAACCTGTATTAGGGGTCAGTCCTTGA
Human GABA_{A} receptor alpha 3 subunit cDNA
(SEQ ID NO: 3)
ATGATAATCACACAAACAAGTCACTGTTACATGACCAGCCTTGGGATTCTT
TTCCTGATTAATATTCTCCCTGGAACCACTGGTCAAGGGGAATCAAGACG
ACAAGAACCCGGGGACTTTGTGAAGCAGGACATTGGCGGGCTGTCTCCTA
AGCATGCCCCAGATATTCCTGATGACAGCACTGACAACATCACTATCTTCA
CCAGAATCTTGGATCGTCTTCTGGACGGCTATGACAACCGGCTGCGACCT
GGGCTTGGAGATGCAGTGACTGAAGTGAAGACTGACATCTACGTGACCAG
TTTTGGCCCTGTGTCAGACACTGACATGGAGTACACTATTGATGTATTTTT
TCGGCAGACATGGCATGATGAAAGACTGAAATTTGATGGCCCCATGAAGA
TCCTTCCACTGAACAATCTCCTGGCTAGTAAGATCTGGACACCGGACACCT
TCTTCCACAATGGCAAGAAATCAGTGGCTCATAACATGACCACGCCCAAC
AAGCTGCTCAGATTGGTGGACAACGGAACCCTCCTCTATACAATGAGGTT
AACAATTCATGCTGAGTGTCCCATGCATTTGGAAGATTTTCCCATGGATGT
GCATGCCTGCCCACTGAAGTTTGGAAGCTATGCCTATACAACAGCTGAAG
TGGTTTATTCTTGGACTCTCGGAAAGAACAAATCCGTGGAAGTGGCACAG
GATGGTTCTCGCTTGAACCAGTATGACCTTTTGGGCCATGTTGTTGGGACA
GAGATAATCCGGTCTAGTACAGGAGAATATGTCGTCATGACAACCCACTT
CCATCTCAAGCGAAAAATTGGCTACTTTGTGATCCAGACCTACTTGCCATG
TATCATGACTGTCATTCTGTCACAAGTGTCGTTCTGGCTCAACAGAGAGTC
TGTTCCTGCCCGTACAGTCTTTGGTGTCACCACTGTGCTTACCATGACCAC
CTTGAGTATCAGTGCCAGAAATTCCTTACCTAAAGTGGCATATGCGACGG
CCATGGACTGGTTCATAGCCGTCTGTTATGCCTTTGTATTTTCTGCACTGAT
TGAATTTGCCACTGTCAACTATTTCACCAAGCGGAGTTGGGCTTGGGAAG
GCAAGAAGGTGCCAGAGGCCCTGGAGATGAAGAAGAAAACACCAGCAGC
CCCAGCAAAGAAAACCAGCACTACCTTCAACATCGTGGGGACCACCTATC
CCATCAACCTGGCCAAGGACACTGAATTTTCCACCATCTCCAAGGGCGCT
GCTCCCAGTGCCTCCTCAACCCCAACAATCATTGCTTCACCCAAGGCCACC
TACGTGCAGGACAGCCCGACTGAGACCAAGACCTACAACAGTGTCAGCAA
GGTTGACAAAATTTCCCGCATCATCTTTCCTGTGCTCTTTGCCATATTCAAT
CTGGTCTATTGGGCCACATATGTCAACCGGGAGTCAGCTATCAAGGGCAT
GATCCGCAAACAGTAG
Human GABA_{A} receptor alpha 4 subunit cDNA
(SEQ ID NO: 4)
ATGGTTTCTGCCAAGAAGGTACCCGCGATCGCTCTGTCCGCCGGGGTCAG
TTTCGCCCTCCTGCGCTTCCTGTGCCTGGCGGTTTGTTTAAACGAATCCCC
AGGACAGAACCAAAAGGAGGAGAAATTGTGCACAGAAAATTTCACCCGC
ATCCTGGACAGTTTGCTCGATGGTTATGACAACAGGCTGCGTCCTGGATTT
GGGGGTCCTGTTACAGAAGTGAAAACTGACATATATGTCACCAGCTTTGG
ACCTGTTTCTGATGTTGAAATGGAATACACAATGGATGTGTTCTTCAGGCA
GACATGGATTGACAAAAGATTAAAATATGACGGCCCCATTGAAATTTTGA
GATTGAACAATATGATGGTAACGAAAGTGTGGACCCCTGATACTTTCTTC
AGGAATGGAAAGAAATCTGTCTCACATAATATGACAGCTCCAAATAAGCT
TTTTAGAATTATGAGAAATGGTACTATTTTATACACAATGAGACTCACCAT
AAGTGCGGAGTGTCCCATGAGATTGGTGGATTTTCCCATGGATGGTCATG
CATGCCCTTTGAAATTCGGGAGTTATGCCTATCCAAAGAGTGAGATGATCT
ATACCTGGACAAAAGGTCCTGAGAAATCAGTTGAAGTTCCGAAGGAGTCT
TCCAGCTTAGTTCAATATGATTTGATTGGGCAAACCGTATCAAGTGAAACC
ATCAAATCAATTACGGGTGAATATATTGTTATGACGGTTTACTTCCACCTC
AGACGGAAGATGGGTTATTTTATGATTCAGACCTATATTCCGTGCATTATG
ACAGTGATTCTTTCTCAAGTTTCATTTTGGATAAATAAAGAATCAGTTCCC
GCTAGGACTGTATTTGGAATAACAACTGTCCTCACCATGACCACACTAAG
CATCAGTGCACGACATTCTTTGCCCAAAGTGTCCTATGCTACCGCCATGGA
CTGGTTCATAGCTGTCTGCTTTGCTTTTGTATTTTCGGCCCTTATCGAGTTT
GCTGCTGTCAACTATTTCACCAATATTCAAATGGAAAAAGCCAAAAGGAA
GACATCAAAGCCCCCTCAGGAAGTTCCCGCTGCTCCAGTGCAGAGAGAGA
AGCATCCTGAAGCCCCTCTGCAGAATACAAATGCCAATTTGAACATGAGA
AAAAGAACAAATGCTTTGGTTCACTCTGAATCTGATGTTGGCAACAGAAC
TGAGGTGGGAAACCATTCAAGCAAATCTTCCACAGTTGTTCAAGAATCTT
CTAAAGGCACACCTCGGTCTTACTTAGCTTCCAGTCCAAACCCATTCAGCC
GTGCAAATGCAGCTGAAACCATATCTGCAGCAAGAGCACTTCCATCTGCT
TCTCCTACTTCTATCCGAACTGGATATATGCCTCGAAAGGCTTCAGTTGGA
TCTGCTTCTACTCGTCACGTGTTTGGATCAAGACTGCAGAGGATAAAGACC
ACAGTTAATACCATAGGGGCTACTGGGAAGTTGTCAGCTACTCCTCCTCCA
TCGGCTCCACCACCTTCTGGATCTGGCACAAGTAAAATAGACAAATATGC
CCGTATTCTCTTTCCAGTCACATTTGGGGCATTTAACATGGTTTATTGGGTT
GTTTATTTATCTAAGGACACTATGGAGAAATCAGAAAGTCTAATGTAA
Human GABA_{A} receptor alpha 5 subunit cDNA
(SEQ ID NO: 5)
ATGGACAATGGAATGTTCTCTGGTTTTATCATGATCAAAAACCTCCTTCTC
TTTTGTATTTCCATGAACTTATCCAGTCACTTTGGCTTTTCACAGATGCCAA
CCAGTTCAGTGAAAGATGAGACCAATGACAACATCACGATATTTACCAGG
ATCTTGGATGGGCTCTTGGATGGCTACGACAACAGACTTCGGCCCGGGCT
GGGAGAGCGCATCACTCAGGTGAGGACCGACATCTACGTCACCAGCTTCG
GCCCGGTGTCCGACACGGAAATGGAGTACACCATAGACGTGTTTTTCCGA
CAAAGCTGGAAAGATGAAAGGCTTCGGTTTAAGGGGCCCATGCAGCGCCT
CCCTCTCAACAACCTCCTTGCCAGCAAGATCTGGACCCCAGACACGTTCTT
CCACAACGGGAAGAAGTCCATCGCTCACAACATGACCACGCCCAACAAGC
TGCTGCGGCTGGAGGACGACGGCACCCTGCTCTACACCATGCGCTTGACC
ATCTCTGCAGAGTGCCCCATGCAGCTTGAGGACTTCCCGATGGATGCGCA
CGCTTGCCCTCTGAAATTTGGCAGCTATGCGTACCCTAATTCTGAAGTCGT
CTACGTCTGGACCAACGGCTCCACCAAGTCGGTGGTGGTGGCGGAAGATG
GCTCCAGACTGAACCAGTACCACCTGATGGGGCAGACGGTGGGCACTGAG
AACATCAGCACCAGCACAGGCGAATACACAATCATGACAGCTCACTTCCA
CCTGAAAAGGAAGATTGGCTACTTTGTCATCCAGACCTACCTTCCCTGCAT
AATGACCGTGATCTTATCACAGGTGTCCTTTTGGCTGAACCGGGAATCAGT
CCCAGCCAGGACAGTTTTTGGGGTCACCACGGTGCTGACCATGACGACCC
TCAGCATCAGCGCCAGGAACTCTCTGCCCAAAGTGGCCTACGCCACCGCC
ATGGACTGGTTCATAGCCGTGTGCTATGCCTTCGTCTTCTCGGCGCTGATA
GAGTTTGCCACGGTCAATTACTTTACCAAGAGAGGCTGGGCCTGGGATGG
CAAAAAAGCCTTGGAAGCAGCCAAGATCAAGAAAAAGCGTGAAGTCATA
CTAAATAAGTCAACAAACGCTTTTACAACTGGGAAGATGTCTCACCCCCC
AAACATTCCGAAGGAACAGACCCCAGCAGGGACGTCGAATACAACCTCA
GTCTCAGTAAAACCCTCTGAAGAGAAGACTTCTGAAAGCAAAAAGACTTA
CAACAGTATCAGCAAAATTGACAAAATGTCCCGAATCGTATTCCCAGTCT
TGTTCGGCACTTTCAACTTAGTTTACTGGGCAACGTATTTGAATAGGGAGC
CGGTGATAAAAGGAGCCGCCTCTCCAAAATAA
Human GABA_{A} receptor alpha 6 subunit cDNA
(SEQ ID NO: 6)
ATGGCGTCATCTCTGCCCTGGCTGTGCATTATTCTGTGGCTAGAAAATGCC
CTAGGGAAACTCGAAGTTGAAGGCAACTTCTACTCAGAAAACGTCAGTCG
GATCCTGGACAACTTGCTTGAAGGCTATGACAATCGGCTGCGGCCGGGAT
TTGGAGGTGCTGTCACTGAAGTCAAAACAGACATTTATGTGACCAGTTTTG
GGCCCGTGTCAGATGTGGAGATGGAGTATACGATGGATGTTTTTTTCCGCC
AGACCTGGACTGATGAGAGGTTGAAGTTTGGGGGGCCAACTGAGATTCTG
AGTCTGAATAATTTGATGGTCAGTAAAATCTGGACGCCTGACACCTTTTTC
AGAAATGGTAAAAAGTCCATTGCTCACAACATGACAACTCCTAATAAACT
CTTCAGAATAATGCAGAATGGAACCATTTTATACACCATGAGGCTTACCA
TCAATGCTGACTGTCCCATGAGGCTGGTTAACTTTCCTATGGATGGGCATG
CTTGTCCACTCAAGTTTGGGAGCTATGCTTATCCCAAAAGTGAAATCATAT
ATACGTGGAAAAAAGGACCACTTTACTCAGTAGAAGTCCCAGAAGAATCT
TCAAGCCTTCTCCAGTATGATCTGATTGGACAAACAGTATCTAGTGAGAC
AATTAAATCTAACACAGGTGAATACGTTATAATGACAGTTTACTTCCACTT
GCAAAGGAAGATGGGCTACTTCATGATACAGATATACACTCCTTGCATTA
TGACAGTCATTCTTTCCCAGGTGTCTTTCTGGATTAATAAGGAGTCCGTCC
CAGCAAGAACTGTTTTTGGGATCACCACTGTTTTAACTATGACCACTTTGA
GCATCAGTGCCCGGCACTCTTTGCCAAAAGTGTCATATGCCACTGCCATGG
ATTGGTTCATAGCTGTTTGCTTTGCATTCGTCTTCTCTGCTCTTATCGAGTT
CGCAGCTGTCAACTACTTTACCAATCTTCAGACACAGAAGGCGAAAAGGA
AGGCACAGTTTGCAGCCCCACCCACAGTGACAATATCAAAAGCTACTGAA
CCTTTGGAAGCTGAGATTGTTTTGCATCCTGACTCCAAATATCATCTGAAG
AAAAGGATCACTTCTCTGTCTTTGCCAATAGTTTCATCTTCCGAGGCCAAT
AAAGTGCTCACGAGAGCGCCCATCTTACAATCAACACCTGTCACACCCCC
ACCACTCTCGCCAGCCTTTGGAGGCACCAGTAAAATAGACCAGTATTCTC
GAATTCTCTTCCCAGTTGCATTTGCAGGATTCAACCTTGTGTACTGGGTAG
TTTATCTTTCCAAAGATACAATGGAAGTGAGTAGCAGTGTTGAATAG
Human GABA_{A} receptor beta 1 subunit cDNA
(SEQ ID NO: 7)
ATGTGGACAGTACAAAATCGAGAGAGTCTGGGGCTTCTCTCTTTCCCTGTG
ATGATTACCATGGTCTGTTGTGCACACAGCACCAATGAACCCAGCAACAT
GYCATACGTGAAAGAGACAGTGGACAGATTGCTCAAAGGATATGACATTC
GCTTGCGGCCGGACTTCGGAGGGCCCCCCGTCGACGTTGGGATGCGGATC
GATGTCGCCAGCATAGACATGGTCTCCGAAGTGAATATGGATTATACACT
CACCATGTATTTCCAGCAGTCTTGGAAAGACAAAAGGCTTTCTTATTCTGG
AATCCCACTGAACCTCACCCTAGACAATAGGGTAGCTGACCAACTCTGGG
TACCAGACACCTACTTTCTGAATGACAAGAAATCATTTGTGCATGGGGTC
ACAGTGAAAAATCGAATGATTCGACTGCATCCTGATGGAACAGTTCTCTA
TGGACTCCGAATCACAACCACAGCTGCATGTATGATGGATCTTCGAAGAT
ATCCACTGGATGAGCAGAACTGCACCCTGGAGATCGAAAGTTATGGCTAT
ACCACTGATGACATTGAATTTTACTGGAATGGAGGAGAAGGGGCAGTCAC
TGGTGTTAATAAAATCGAACTTCCTCAATTTTCAATTGTTGACTACAAGAT
GGTGTCTAAGAAGGTGGAGTTCACAACAGGAGCGTATCCACGACTGTCAC
TAAGTTTTCGTCTAAAGAGAAACATTGGTTACTTCATTTTGCAAACCTACA
TGCCTTCTACACTGATTACAATTCTGTCCTGGGTGTCTTTTTGGATCAACTA
TGATGCATCTGCAGCCAGAGTCGCACTAGGAATCACGACGGTGCTTACAA
TGACAACCATCAGCACCCACCTCAGGGAGACCCTGCCAAAGATCCCTTAT
GTCAAAGCGATTGATATTTATCTGATGGGTTGCTTTGTGTTTGTGTTCCTG
GCTCTGCTGGAGTATGCCTTTGTAAATTACATCTTCTTTGGGAAAGGCCCT
CAGAAAAAGGGAGCTAGCAAACAAGACCAGAGTGCCAATGAGAAGAATA
AACTGGAGATGAATAAAGTCCAGGTCGACGCCCACGGTAACATTCTCCTC
AGCACCCTGGAAATCCGGAATGAGACGAGTGGCTCGGAAGTGCTCACGA
GCGTGAGCGACCCCAAGGCCACCATGTACTCCTATGACAGCGCCAGCATC
CAGTACCGCAAGCCCCTGAGCAGCCGCGAGGCCTACGGGCGCGCCCTGGA
CCGGCACGGGGTACCCAGCAAGGGGCGCATCCGCAGGCGTGCCTCCCAGC
TCAAAGTCAAGATCCCCGACTTGACTGATGTGAATTCCATAGACAAGTGG
TCCCGAATGTTTTTCCCCATCACCTTTTCTCTTTTTAATGTCGTCTATTGGC
TTTACTATGTACACTGA
Human GABA_{A} receptor beta 2 variant 1 (long) subunit cDNA
(SEQ ID NO: 8)
ATGTGGAGAGTGCGGAAAAGGGGCTACTTTGGGATTTGGTCCTTCCCCTT
AATAATCGCCGCTGTCTGTGCGCAGAGTGTCAATGACCCTAGTAATATGTC
GCTGGTTAAAGAGACGGTGGATAGACTCCTGAAAGGCTATGACATTCGTC
TGAGACCAGATTTTGGAGGTCCCCCCGTGGCTGTGGGGATGAACATTGAC
ATTGCCAGCATCGATATGGTTTCTGAAGTCAATATGGATTATACCTTGACA
ATGTACTTTCAACAAGCCTGGAGAGATAAGAGGCTGTCCTATAATGTAAT
ACCTTTAAACTTGACTCTGGACAACAGAGTGGCAGACCAGCTCTGGGTGC
CTGATACCTATTTCCTGAACGATAAGAAGTCATTTGTGCACGGAGTGACTG
TTAAGAACCGCATGATTCGCCTGCATCCTGATGGCACCGTCCTTTATGGAC
TCAGAATCACAACCACAGCTGCCTGCATGATGGACCTAAGGAGGTACCCA
CTGGATGAACAAAACTGCACCTTGGAAATTGAGAGCTATGGATACACAAC
TGATGACATTGAGTTTTACTGGCGTGGCGATGATAATGCAGTAACAGGAG
TAACGAAAATTGAACTTCCACAGTTCTCTATTGTAGATTACAAACTTATCA
CCAAGAAGGTTGTTTTTTCCACAGGTTCCTATCCCAGGTTATCCCTCAGCT
TTAAGCTTAAGAGAAACATTGGCTACTTTATCCTGCAAACATACATGCCTT
CCATCCTGATTACCATCCTCTCCTGGGTCTCCTTCTGGATTAATTACGATGC
TTCAGCTGCAAGGGTGGCATTAGGAATCACAACTGTCCTCACAATGACCA
CAATCAACACCCACCTCCGGGAAACTCTCCCTAAAATCCCCTATGTGAAG
GCCATTGACATGTACCTGATGGGGTGCTTTGTCTTCGTTTTCATGGCCCTTC
TGGAATATGCCCTAGTCAACTACATCTTCTTTGGGAGGGGGCCCCAACGC
CAAAAGAAAGCAGCTGAGAAGGCTGCCAGTGCCAACAATGAGAAGATGC
GCCTGGATGTCAACAAGATTTTTTATAAAGATATTAAACAAAATGGGACC
CAATATCGATCCTTGTGGGACCCTACTGGAAACCTCTCCCCAACTAGACG
GACTACCAATTACGATTTCTCTCTGTATACGATGGACCCCCATGAGAACAT
CTTACTGAGCACTCTCGAGATAAAAAATGAAATGGCCACATCTGAGGCTG
TGATGGGACTTGGAGACCCCAGAAGCACAATGCTAGCCTATGATGCCTCC
AGCATCCAGTATCGGAAAGCTGGGTTGCCCAGGCATAGTTTTGGCCGAAA
TGCTCTGGAACGACATGTGGCGCAAAAGAAAAGTCGCCTGAGGAGACGC
GCCTCCCAACTGAAAATCACCATCCCTGACTTGACTGATGTGAATGCCATA
GATCGGTGGTCCCGCATATTCTTCCCAGTGGTTTTTTCCTTCTTCAACATCG
TCTATTGGCTTTACTATGTGAACTAA
Human GABA_{A} receptor beta 2 variant 2 (short) subunit cDNA
(SEQ ID NO: 9)
ATGTGGAGAGTGCGGAAAAGGGGCTACTTTGGGATTTGGTCCTTCCCCTT
AATAATCGCCGCTGTCTGTGCGCAGAGTGTCAATGACCCTAGTAATATGTC
GCTGGTTAAAGAGACGGTGGATAGACTCCTGAAAGGCTATGACATTCGTC
TGAGACCAGATTTTGGAGGTCCCCCCGTGGCTGTGGGGATGAACATTGAC
ATTGCCAGCATCGATATGGTTTCTGAAGTCAATATGGATTATACCTTGACA
ATGTACTTTCAACAAGCCTGGAGAGATAAGAGGCTGTCCTATAATGTAAT
ACCTTTAAACTTGACTCTGGACAACAGAGTGGCAGACCAGCTCTGGGTGC
CTGATACCTATTTCCTGAACGATAAGAAGTCATTTGTGCACGGAGTGACTG
TTAAGAACCGCATGATTCGCCTGCATCCTGATGGCACCGTCCTTTATGGAC
TCAGAATCACAACCACAGCTGCCTGCATGATGGACCTAAGGAGGTACCCA
CTGGATGAACAAAACTGCACCTTGGAAATTGAGAGCTATGGATACACAAC
TGATGACATTGAGTTTTACTGGCGTGGCGATGATAATGCAGTAACAGGAG
TAACGAAAATTGAACTTCCACAGTTCTCTATTGTAGATTACAAACTTATCA
CCAAGAAGGTTGTTTTTTCCACAGGTTCCTATCCCAGGTTATCCCTCAGCT
TTAAGCTTAAGAGAAACATTGGCTACTTTATCCTGCAAACATACATGCCTT
CCATCCTGATTACCATCCTCTCCTGGGTCTCCTTCTGGATTAATTACGATGC
TTCAGCTGCAAGGGTGGCATTAGGAATCACAACTGTCCTCACAATGACCA
CAATCAACACCCACCTCCGGGAAACTCTCCCTAAAATCCCCTATGTGAAG
GCCATTGACATGTACCTGATGGGGTGCTTTGTCTTCGTTTTCATGGCCCTTC
TGGAATATGCCCTAGTCAACTACATCTTCTTTGGGAGGGGGCCCCAACGC
CAAAAGAAAGCAGCTGAGAAGGCTGCCAGTGCCAACAATGAGAAGATGC
GCCTGGATGTCAACAAGATGGACCCCCATGAGAACATCTTACTGAGCACT
CTCGAGATAAAAAATGAAATGGCCACATCTGAGGCTGTGATGGGACTTGG
AGACCCCAGAAGCACAATGCTAGCCTATGATGCCTCCAGCATCCAGTATC
GGAAAGCTGGGTTGCCCAGGCATAGTTTTGGCCGAAATGCTCTGGAACGA
CATGTGGCGCAAAAGAAAAGTCGCCTGAGGAGACGCGCCTCCCAACTGA
AAATCACCATCCCTGACTTGACTGATGTGAATGCCATAGATCGGTGGTCCC
GCATATTCTTCCCAGTGGTTTTTTCCTTCTTCAACATCGTCTATTGGCTTTA
TTATGTGAACTAA
Human GABA_{A} receptor beta 3 variant 1 subunit cDNA
(SEQ ID NO: 10)
ATGTGGGGCCTTGCGGGAGGAAGGCTTTTCGGCATCTTCTCGGCCCCGGT
GCTGGTGGCTGTGGTGTGCTGCGCCCAGAGTGTGAACGATCCCGGGAACA
TGTCCTTTGTGAAGGAGACGGTGGACAAGCTGTTGAAAGGCTACGACATT
CGCCTAAGACCCGACTTCGGGGGTCCCCCGGTCTGCGTGGGGATGAACAT
CGACATCGCCAGCATCGACATGGTTTCCGAAGTCAACATGGATTATACCTT
AACCATGTATTTTCAACAATATTGGAGAGATAAAAGGCTCGCCTATTCTG
GGATCCCTCTCAACCTCACGCTTGACAATCGAGTGGCTGACCAGCTATGG
GTGCCCGACACATATTTCTTAAATGACAAAAAGTCATTTGTGCATGGAGT
GACAGTGAAAAACCGCATGATCCGTCTTCACCCTGATGGGACAGTGCTGT
ATGGGCTCAGAATCACCACGACAGCAGCATGCATGATGGACCTCAGGAGA
TACCCCCTGGACGAGCAGAACTGCACTCTGGAAATTGAAAGCTATGGCTA
CACCACGGATGACATTGAGTTTTACTGGCGAGGCGGGGACAAGGCTGTTA
CCGGAGTGGAAAGGATTGAGCTCCCGCAGTTCTCCATCGTGGAGCACCGT
CTGGTCTCGAGGAATGTTGTCTTCGCCACAGGTGCCTATCCTCGACTGTCA
CTGAGCTTTCGGTTGAAGAGGAACATTGGATACTTCATTCTTCAGACTTAT
ATGCCCTCTATACTGATAACGATTCTGTCGTGGGTGTCCTTCTGGATCAAT
TATGATGCATCTGCTGCTAGAGTTGCCCTCGGGATCACAACTGTGCTGACA
ATGACAACCATCAACACCCACCTTCGGGAGACCTTGCCCAAAATCCCCTA
TGTCAAAGCCATTGACATGTACCTTATGGGCTGCTTCGTCTTTGTGTTCCT
GGCCCTTCTGGAGTATGCCTTTGTCAACTACATTTTCTTTGGAAGAGGCCC
TCAAAGGCAGAAGAAGCTTGCAGAAAAGACAGCCAAGGCAAAGAATGAC
CGTTCAAAGAGCGAAAGCAACCGGGTGGATGCTCATGGAAATATTCTGTT
GACATCGCTGGAAGTTCACAATGAAATGAATGAGGTCTCAGGCGGCATTG
GCGATACCAGGAATTCAGCAATATCCTTTGACAACTCAGGAATCCAGTAC
AGGAAACAGAGCATGCCTCGAGAAGGGCATGGGCGATTCCTGGGGGACA
GAAGCCTCCCGCACAAGAAGACCCATCTACGGAGGAGGTCTTCACAGCTC
AAAATTAAAATACCTGATCTAACCGATGTGAATGCCATAGACAGATGGTC
CAGGATCGTGTTTCCATTCACTTTTTCTCTTTTCAACTTAGTTTACTGGCTG
TACTATGTTAACTGA
Human GABA_{A} receptor beta 3 variant 2 subunit cDNA
(SEQ ID NO: 11)
ATGTGCTCCGGGCTCCTGGAGCTCCTGCTGCCCATCTGGCTCTCCTGGACC
CTGGGGACCCGAGGCTCTGAGCCCCGCAGTGTGAACGATCCCGGGAACAT
GTCCTTTGTGAAGGAGACGGTGGACAAGCTGTTGAAAGGCTACGACATTC
GCCTAAGACCCGACTTCGGGGGTCCCCCGGTCTGCGTGGGGATGAACATC
GACATCGCCAGCATCGACATGGTTTCCGAAGTCAACATGGATTATACCTT
AACCATGTATTTTCAACAATATTGGAGAGATAAAAGGCTCGCCTATTCTG
GGATCCCTCTCAACCTCACGCTTGACAATCGAGTGGCTGACCAGCTATGG
GTGCCCGACACATATTTCTTAAATGACAAAAAGTCATTTGTGCATGGAGT
GACAGTGAAAAACCGCATGATCCGTCTTCACCCTGATGGGACAGTGCTGT
ATGGGCTCAGAATCACCACGACAGCAGCATGCATGATGGACCTCAGGAGA
TACCCCCTGGACGAGCAGAACTGCACTCTGGAAATTGAAAGCTATGGCTA
CACCACGGATGACATTGAGTTTTACTGGCGAGGCGGGGACAAGGCTGTTA
CCGGAGTGGAAAGGATTGAGCTCCCGCAGTTCTCCATCGTGGAGCACCGT
CTGGTCTCGAGGAATGTTGTCTTCGCCACAGGTGCCTATCCTCGACTGTCA
CTGAGCTTTCGGTTGAAGAGGAACATTGGATACTTCATTCTTCAGACTTAT
ATGCCCTCTATACTGATAACGATTCTGTCGTGGGTGTCCTTCTGGATCAAT
TATGATGCATCTGCTGCTAGAGTTGCCCTCGGGATCACAACTGTGCTGACA
ATGACAACCATCAACACCCACCTTCGGGAGACCTTGCCCAAAATCCCCTA
TGTCAAAGCCATTGACATGTACCTTATGGGCTGCTTCGTCTTTGTGTTCCT
GGCCCTTCTGGAGTATGCCTTTGTCAACTACATTTTCTTTGGAAGAGGCCC
TCAAAGGCAGAAGAAGCTTGCAGAAAAGACAGCCAAGGCAAAGAATGAC
CGTTCAAAGAGCGAAAGCAACCGGGTGGATGCTCATGGAAATATTCTGTT
GACATCGCTGGAAGTTCACAATGAAATGAATGAGGTCTCAGGCGGCATTG
GCGATACCAGGAATTCAGCAATATCCTTTGACAACTCAGGAATCCAGTAC
AGGAAACAGAGCATGCCTCGAGAAGGGCATGGGCGATTCCTGGGGGACA
GAAGCCTCCCGCACAAGAAGACCCATCTACGGAGGAGGTCTTCACAGCTC
AAAATTAAAATACCTGATCTAACCGATGTGAATGCCATAGACAGATGGTC
CAGGATCGTGTTTCCATTCACTTTTTCTCTTTTCAACTTAGTTTACTGGCTG
TACTATGTTAACTGA
Human GABA_{A} receptor gamma 1 subunit cDNA
(SEQ ID NO: 12)
ATGGGTCCTTTGAAAGCTTTTCTCTTCTCCCCTTTTCTTCTGCGGAGTCAAA
GTAGAGGGGTGAGGTTGGTCTTCTTGTTACTGACCCTGCATTTGGGAAACT
GTGTTGATAAGGCAGATGATGAAGATGATGAGGATTTAACGGTGAACAAA
ACCTGGGTCTTGGCCCCAAAAATTCATGAAGGAGATATCACACAAATTCT
GAATTCATTGCTTCAAGGCTATGACAATAAACTTCGTCCAGATATAGGAG
TGAGGCCCACAGTAATTGAAACTGATGTTTATGTAAACAGCATTGGACCA
GTTGATCCAATTAATATGGAATATACAATAGATATAATTTTTGCCCAAACC
TGGTTTGACAGTCGTTTAAAATTCAATAGTACCATGAAAGTGCTTATGCTT
AACAGTAATATGGTTGGAAAAATTTGGATTCCTGACACTTTCTTCAGAAAC
TCAAGAAAATCTGATGCTCACTGGATAACAACTCCTAATCGTCTGCTTCGA
ATTTGGAATGATGGACGAGTTCTGTATACTCTAAGATTGACAATTAATGCA
GAATGTTATCTTCAGCTTCATAACTTTCCCATGGATGAACATTCCTGTCCA
CTGGAATTTTCAAGCTATGGATACCCTAAAAATGAAATTGAGTATAAGTG
GAAAAAGCCCTCCGTAGAAGTGGCTGATCCTAAATACTGGAGATTATATC
AGTTTGCATTTGTAGGGTTACGGAACTCAACTGAAATCACTCACACGATCT
CTGGGGATTATGTTATCATGACAATTTTTTTTGACCTGAGCAGAAGAATGG
GATATTTCACTATTCAGACCTACATTCCATGCATTCTGACAGTTGTTCTTTC
TTGGGTGTCTTTTTGGATCAATAAAGATGCAGTGCCTGCAAGAACATCGTT
GGGTATCACTACAGTTCTGACTATGACAACCCTGAGTACAATTGCCAGGA
AGTCTTTACCTAAGGTTTCTTATGTGACTGCGATGGATCTCTTTGTTTCTGT
TTGTTTCATTTTTGTTTTTGCAGCCTTGATGGAATATGGAACCTTGCATTAT
TTTACCAGCAACCAAAAAGGAAAGACTGCTACTAAAGACAGAAAGCTAA
AAAATAAAGCCTCGATGACTCCTGGTCTCCATCCTGGATCCACTCTGATTC
CAATGAATAATATTTCTGTGCCGCAAGAAGATGATTATGGGTATCAGTGTT
TGGAGGGCAAAGATTGTGCCAGCTTCTTCTGTTGCTTTGAAGACTGCAGA
ACAGGATCTTGGAGGGAAGGAAGGATACACATACGCATTGCCAAAATTG
ACTCTTATTCTAGAATATTTTTCCCAACCGCTTTTGCCCTGTTCAACTTGGT
TTATTGGGTTGGCTATCTTTACTTATAA
Human GABA_{A} receptor gamma 2 transcript variant 1 (short) subunit cDNA
(SEQ ID NO: 13)
ATGAGTTCGCCAAATATATGGAGCACAGGAAGCTCAGTCTACTCGACTCC
TGTATTTTCACAGAAAATGACGGTGTGGATTCTGCTCCTGCTGTCGCTCTA
CCCTGGCTTCACTAGCCAGAAATCTGATGATGACTATGAAGATTATGCTTC
TAACAAAACATGGGTCTTGACTCCAAAAGTTCCTGAGGGTGATGTCACTG
TCATCTTAAACAACCTGCTGGAAGGATATGACAATAAACTTCGGCCTGAT
ATAGGAGTGAAGCCAACGTTAATTCACACAGACATGTATGTGAATAGCAT
TGGTCCAGTGAACGCTATCAATATGGAATACACTATTGATATATTTTTTGC
GCAAACGTGGTATGACAGACGTTTGAAATTTAACAGCACCATTAAAGTCC
TCCGATTGAACAGCAACATGGTGGGGAAAATCTGGATTCCAGACACTTTC
TTCAGAAATTCCAAAAAAGCTGATGCACACTGGATCACCACCCCCAACAG
GATGCTGAGAATTTGGAATGATGGTCGAGTGCTCTACACCCTAAGGTTGA
CAATTGATGCTGAGTGCCAATTACAATTGCACAACTTTCCAATGGATGAA
CACTCCTGCCCCTTGGAGTTCTCAAGTTATGGCTATCCACGTGAAGAAATT
GTTTATCAATGGAAGCGAAGTTCTGTTGAAGTGGGCGACACAAGATCCTG
GAGGCTTTATCAATTCTCATTTGTTGGTCTAAGAAATACCACCGAAGTAGT
GAAGACAACTTCCGGAGATTATGTGGTCATGTCTGTCTACTTTGATCTGAG
CAGAAGAATGGGATACTTTACCATCCAGACCTATATCCCCTGCACACTCAT
TGTCGTCCTATCCTGGGTGTCTTTCTGGATCAATAAGGATGCTGTTCCAGC
CAGAACATCTTTAGGTATCACCACTGTCCTGACAATGACCACCCTCAGCAC
CATTGCCCGGAAATCGCTCCCCAAGGTCTCCTATGTCACAGCGATGGATCT
CTTTGTATCTGTTTGTTTCATCTTTGTCTTCTCTGCTCTGGTGGAGTATGGC
ACCTTGCATTATTTTGTCAGCAACCGGAAACCAAGCAAGGACAAAGATAA
AAAGAAGAAAAACCCTGCCCCTACCATTGATATCCGCCCAAGATCAGCAA
CCATTCAAATGAATAATGCTACACACCTTCAAGAGAGAGATGAAGAGTAC
GGCTATGAGTGTCTGGACGGCAAGGACTGTGCCAGTTTTTTCTGCTGTTTT
GAAGATTGTCGAACAGGAGCTTGGAGACATGGGAGGATACATATCCGCAT
TGCCAAAATGGACTCCTATGCTCGGATCTTCTTCCCCACTGCCTTCTGCCT
GTTTAATCTGGTCTATTGGGTCTCCTACCTCTACCTGTGA
Human GABA_{A} receptor gamma 2 transcript variant 3 (long) subunit cDNA
(SEQ ID NO: 14)
ATGAGTTCGCCAAATATATGGAGCACAGGAAGCTCAGTCTACTCGACTCC
TGTATTTTCACAGAAAATGACGGTGTGGATTCTGCTCCTGCTGTCGCTCTA
CCCTGGCTTCACTAGCCAGAAATCTGATGATGACTATGAAGATTATGCTTC
TAACAAAACATGGGTCTTGACTCCAAAAGTTCCTGAGGGTGATGTCACTG
TCATCTTAAACAACCTGCTGGAAGGATATGACAATAAACTTCGGCCTGAT
ATAGGAGTGAAGCCAACGTTAATTCACACAGACATGTATGTGAATAGCAT
TGGTCCAGTGAACGCTATCAATATGGAATACACTATTGATATATTTTTTGC
GCAAACGTGGTATGACAGACGTTTGAAATTTAACAGCACCATTAAAGTCC
TCCGATTGAACAGCAACATGGTGGGGAAAATCTGGATTCCAGACACTTTC
TTCAGAAATTCCAAAAAAGCTGATGCACACTGGATCACCACCCCCAACAG
GATGCTGAGAATTTGGAATGATGGTCGAGTGCTCTACACCCTAAGGTTGA
CAATTGATGCTGAGTGCCAATTACAATTGCACAACTTTCCAATGGATGAA
CACTCCTGCCCCTTGGAGTTCTCCAGTTGGTCTCGTTCTATTGCCCAGGCT
GGAATGTGCAGTGGTGTGATCTCGGCTCACTACAGCCTTCGCTTCTGGGGC
TCAACTGATCCTCCCACCTTGGCCTCCAGAGTAGCTGGGATTTCAGATGGC
TATCCACGTGAAGAAATTGTTTATCAATGGAAGCGAAGTTCTGTTGAAGT
GGGCGACACAAGATCCTGGAGGCTTTATCAATTCTCATTTGTTGGTCTAAG
AAATACCACCGAAGTAGTGAAGACAACTTCCGGAGATTATGTGGTCATGT
CTGTCTACTTTGATCTGAGCAGAAGAATGGGATACTTTACCATCCAGACCT
ATATCCCCTGCACACTCATTGTCGTCCTATCCTGGGTGTCTTTCTGGATCA
ATAAGGATGCTGTTCCAGCCAGAACATCTTTAGGTATCACCACTGTCCTGA
CAATGACCACCCTCAGCACCATTGCCCGGAAATCGCTCCCCAAGGTCTCCT
ATGTCACAGCGATGGATCTCTTTGTATCTGTTTGTTTCATCTTTGTCTTCTC
TGCTCTGGTGGAGTATGGCACCTTGCATTATTTTGTCAGCAACCGGAAACC
AAGCAAGGACAAAGATAAAAAGAAGAAAAACCCTCTTCTTCGGATGTTTT
CCTTCAAGGCCCCTACCATTGATATCCGCCCAAGATCAGCAACCATTCAA
ATGAATAATGCTACACACCTTCAAGAGAGAGATGAAGAGTACGGCTATGA
GTGTCTGGACGGCAAGGACTGTGCCAGTTTTTTCTGCTGTTTTGAAGATTG
TCGAACAGGAGCTTGGAGACATGGGAGGATACATATCCGCATTGCCAAAA
TGGACTCCTATGCTCGGATCTTCTTCCCCACTGCCTTCTGCCTGTTTAATCT
GGTCTATTGGGTCTCCTACCTCTACCTGTGA
Human GABA_{A} receptor gamma 3 subunit cDNA
(SEQ ID NO: 15)
ATGGCCCCGAAGCTGCTGCTCCTCCTCTGCCTGTCCTCGGGCTTGTACGCG
CGGTCCAGAAAGGTGGAAGAGGATGAATATGAAGATTCATCATCAAACC
AAAAGTGGGTCTTGGCTCCAAAATCCCAAGACACCGACGTGACTCTTATT
CTCAACAAGTTGCTAAGAGAATATGATAAAAAGCTGAGGCCAGATATTGG
AATAAAACCGACCGTAATTGACGTTGACATTTATGTTAACAGCATTGGTCC
TGTGTCATCAATAAACATGGAATACCAAATTGACATATTTTTTGCTCAGAC
CTGGACAGATAGTCGCCTTCGATTCAACAGCACAATGAAAATTCTTACTCT
GAACAGCAACATGGTGGGGTTAATCTGGATCCCAGACACCATCTTCCGCA
ATTCTAAAACCGCAGAGGCTCACTGGATCACCACACCCAATCAGCTCCTC
CGGATTTGGAATGACGGGAAAATCCTTTACACTTTGAGGCTCACCATCAA
TGCTGAGTGCCAGCTGCAGCTGCACAACTTCCCCATGGACGAACACTCCT
GCCCGCTGATTTTCTCCAGCTATGGCTATCCCAAAGAAGAAATGATTTATA
GATGGAGAAAAAATTCAGTGGAGGCAGCTGACCAGAAATCATGGCGGCT
TTATCAGTTTGACTTCATGGGCCTCAGAAACACCACAGAAATCGTGACAA
CGTCTGCAGGTGATTATGTTGTCATGACTATATATTTTGAATTGAGTAGAA
GAATGGGATACTTCACCATTCAGACATACATTCCCTGTATACTGACTGTGG
TTTTATCCTGGGTGTCATTTTGGATCAAAAAAGATGCTACGCCAGCAAGA
ACAGCATTAGGCATCACCACGGTGCTGACCATGACCACCCTGAGCACCAT
CGCCAGGAAGTCCTTGCCACGCGTGTCCTACGTGACCGCCATGGACCTTTT
TGTGACTGTGTGCTTCCTGTTTGTCTTCGCCGCGCTGATGGAGTATGCCAC
CCTCAACTACTATTCCAGCTGTAGAAAACCAACCACCACGAARAAGACAA
CATCGTTACTACATCCAGATTCCTCAAGATGGATTCCTGAGCGAATAAGCC
TACAAGCCCCTTCCAACTATTCCCTCCTGGACATGAGGCCACCACCACCTG
CGATGATCACTTTAAACAATTCCGTTTACTGGCAGGAATTTGAAGATACCT
GTGTCTATGAGTGTCTGGATGGCAAAGACTGTCAGAGCTTCTTCTGCTGCT
ATGAAGAATGTAAATCAGGATCCTGGAGGAAAGGGCGTATTCACATAGAC
ATCTTGGAGCTGGACTCGTACTCCCGGGTCTTTTTCCCCACGTCCTTCCTGC
TCTTTAAGCCTGGTCTACTGGGTTGGATACCTGTATCTCTAA
Human GABA_{A} receptor delta subunit cDNA
(SEQ ID NO: 16)
ATGGACGCGCCCGCCCGGCTGCTGGCCCCGCTCCTGCTCCTCTGCGCGCAG
CAGCTCCGCGGCACCAGAGCGATGAATGACATCGGCGACTACGTGGGCTC
CAACCTGGAGATCTCCTGGCTCCCCAACCTGGACGGGCTGATAGCCGGCT
ACGCCCGCAACTTCCGGCCTGGCATCGGAGGCCCCCCCGTGAATGTGGCC
CTTGCCCTGGAGGTGGCCAGCATCGACCACATCTCAGAGGCCAACATGGA
GTACACCATGACGGTGTTCCTGCACCAGAGCTGGCGGGACAGCAGGCTCT
CCTACAACCACACCAACGAGACCCTGGGTCTGGACAGCCGCTTCGTGGAC
AAGCTGTGGCTGCCCGACACCTTCATCGTGAACGCCAAGTCGGCCTGGTT
CCACGACGTGACGGTGGAGAACAAGCTCATCCGGCTGCAGCCCGACGGCG
TGATCCTGTACAGCATCCGAATCACCTCCACTGTGGCCTGCGACATGGACC
TGGCCAAATACCCCATGGACGAGCAGGAGTGCATGCTGGACCTGGAGAGC
TACGGTTACTCATCGGAGGACATCGTCTACTACTGGTCGGAGAGCCAGGA
GCACATCCACGGGCTGGACAAGCTGCAGCTGGCGCAGTTCACCATCACCA
GCTACCGCTTCACCACGGAGCTGATGAACTTCAAGTCCGCTGGCCAGTTCC
CACGGCTCAGCCTGCACTTCCACCTGCGGAGGAACCGCGGCGTGTACATC
ATCCAATCCTACATGCCCTCCGTCCTGCTGGTCGCCATGTCCTGGGTCTCC
TTCTGGATCAGCCAGGCGGCGGTGCCCGCCAGGGTGTCTCTAGGCATCAC
CACGGTGCTGACGATGACCACGCTCATGGTCAGTGCCCGCTCCTCCCTGCC
ACGGGCATCAGCCATCAAGGCACTGGACGTCTACTTCTGGATCTGCTATGT
CTTCGTGTTTGCCGCCCTGGTGGAGTACGCCTTTGCTCATTTCAACGCCGA
CTACAGGAAGAAGCAGAAGGCCAAGGTCAAGGTCTCCAGGCCGAGGGCA
GAGATGGACGTGAGGAACGCCATTGTCCTCTTCTCCCTCTCTGCTGCCGGC
GTCACGCAGGAGCTGGCCATCTCCCGCCGGCAGCGCCGCGTCCCGGGGAA
CCTGATGGGCTCCTACAGGTCGGTGGGGGTGGAGACAGGGGAGACGAAG
AAGGAGGGGGCAGCCCGCTCAGGAGGCCAGGGGGGCATCCGTGCCCGGC
TCAGGCCCATCGACGCAGACACCATTGACATTTACGCCCGCGCTGTGTTCC
CTGCGGCGTTTGCGGCCGTCAATGTCATCTACTGGGCGGCATACGCCATGT
GA
Human GABA_{A} receptor epsilon subunit cDNA
(SEQ ID NO: 17)
ATGGACGCGCCCGCCCGGCTGCTGGCCCCGCTCCTGCTCCTCTGCGCGCAG
CAGCTCCGCGGCACCAGAGCGATGAATGACATCGGCGACTACGTGGGCTC
CAACCTGGAGATCTCCTGGCTCCCCAACCTGGACGGGCTGATAGCCGGCT
ACGCCCGCAACTTCCGGCCTGGCATCGGAGGCCCCCCCGTGAATGTGGCC
CTTGCCCTGGAGGTGGCCAGCATCGACCACATCTCAGAGGCCAACATGGA
GTACACCATGACGGTGTTCCTGCACCAGAGCTGGCGGGACAGCAGGCTCT
CCTACAACCACACCAACGAGACCCTGGGTCTGGACAGCCGCTTCGTGGAC
AAGCTGTGGCTGCCCGACACCTTCATCGTGAACGCCAAGTCGGCCTGGTT
CCACGACGTGACGGTGGAGAACAAGCTCATCCGGCTGCAGCCCGACGGCG
TGATCCTGTACAGCATCCGAATCACCTCCACTGTGGCCTGCGACATGGACC
TGGCCAAATACCCCATGGACGAGCAGGAGTGCATGCTGGACCTGGAGAGC
TACGGTTACTCATCGGAGGACATCGTCTACTACTGGTCGGAGAGCCAGGA
GCACATCCACGGGCTGGACAAGCTGCAGCTGGCGCAGTTCACCATCACCA
GCTACCGCTTCACCACGGAGCTGATGAACTTCAAGTCCGCTGGCCAGTTCC
CACGGCTCAGCCTGCACTTCCACCTGCGGAGGAACCGCGGCGTGTACATC
ATCCAATCCTACATGCCCTCCGTCCTGCTGGTCGCCATGTCCTGGGTCTCC
TTCTGGATCAGCCAGGCGGCGGTGCCCGCCAGGGTGTCTCTAGGCATCAC
CACGGTGCTGACGATGACCACGCTCATGGTCAGTGCCCGCTCCTCCCTGCC
ACGGGCATCAGCCATCAAGGCACTGGACGTCTACTTCTGGATCTGCTATGT
CTTCGTGTTTGCCGCCCTGGTGGAGTACGCCTTTGCTCATTTCAACGCCGA
CTACAGGAAGAAGCAGAAGGCCAAGGTCAAGGTCTCCAGGCCGAGGGCA
GAGATGGACGTGAGGAACGCCATTGTCCTCTTCTCCCTCTCTGCTGCCGGC
GTCACGCAGGAGCTGGCCATCTCCCGCCGGCAGCGCCGCGTCCCGGGGAA
CCTGATGGGCTCCTACAGGTCGGTGGGGGTGGAGACAGGGGAGACGAAG
AAGGAGGGGGCAGCCCGCTCAGGAGGCCAGGGGGGCATCCGTGCCCGGC
TCAGGCCCATCGACGCAGACACCATTGACATTTACGCCCGCGCTGTGTTCC
CTGCGGCGTTTGCGGCCGTCAATGTCATCTACTGGGCGGCATACGCCATGT
GA
Human GABA_{A} receptor pi subunit cDNA
(SEQ ID NO: 18)
ATGAACTACAGCCTCCACTTGGCCTTCGTGTGTCTGAGTCTCTTCACTGAG
AGGATGTGCATCCAGGGGAGTCAGTTCAACGTCGAGGTCGGCAGAAGTGA
CAAGCTTTCCCTGCCTGGCTTTGAGAACCTCACAGCAGGATATAACAAATT
TCTCAGGCCCAATTTTGGTGGAGAACCCGTACAGATAGCGCTGACTCTGG
ACATTGCAAGTATCTCTAGCATTTCAGAGAGTAACATGGACTACACAGCC
ACCATATACCTCCGACAGCGCTGGATGGACCAGCGGCTGGTGTTTGAAGG
CAACAAGAGCTTCACTCTGGATGCCCGCCTCGTGGAGTTCCTCTGGGTGCC
AGATACTTACATTGTGGAGTCCAAGAAGTCCTTCCTCCATGAAGTCACTGT
GGGAAACAGGCTCATCCGCCTCTTCTCCAATGGCACGGTCCTGTATGCCCT
CAGAATCACGACAACTGTTGCATGTAACATGGATCTGTCTAAATACCCCA
TGGACACACAGACATGCAAGTTGCAGCTGGAAAGCTGGGGCTATGATGGA
AATGATGTGGAGTTCACCTGGCTGAGAGGGAACGACTCTGTGCGTGGACT
GGAACACCTGCGGCTTGCTCAGTACACCATAGAGCGGTATTTCACCTTAGT
CACCAGATCGCAGCAGGAGACAGGAAATTACACTAGATTGGTCTTACAGT
TTGAGCTTCGGAGGAATGTTCTGTATTTCATTTTGGAAACCTACGTTCCTT
CCACTTTCCTGGTGGTGTTGTCCTGGGTTTCATTTTGGATCTCTCTCGATTC
AGTCCCTGCAAGAACCTGCATTGGAGTGACGACCGTGTTATCAATGACCA
CACTGATGATCGGGTCCCGCACTTCTCTTCCCAACACCAACTGCTTCATCA
AGGCCATCGATGTGTACCTGGGGATCTGCTTTAGCTTTGTGTTTGGGGCCT
TGCTAGAATATGCAGTTGCTCACTACAGTTCCTTACAGCAGATGGCAGCC
AAAGATAGGGGGACAACAAAGGAAGTAGAAGAAGTCAGTATTACTAATA
TCATCAACAGCTCCATCTCCAGCTTTAAACGGAAGATCAGCTTTGCCAGCA
TTGAAATTTCCAGCGACAACGTTGACTACAGTGACTTGACAATGAAAACC
AGCGACAAGTTCAAGTTTGTCTTCCGAGAAAAGATGGGCAGGATTGTTGA
TTATTTCACAATTCAAAACCCCAGTAATGTTGATCACTATTCCAAACTACT
GTTTCCTTTGATTTTTATGCTAGCCAATGTATTTTACTGGGCATACTACATG
TATTTTTGA
Human GABA_{A} receptor theta subunit cDNA
(SEQ ID NO: 19)
ATGGGCATCCGAGGCATGCTGCGAGCCGCAGTGATCCTGCTGCTCATCAG
GACCTGGCTCGCGGAGGGCAACTACCCCAGTCCCATCCCGAAATTCCACT
TCGAGTTCTCCTCTGCTGTGCCCGAAGTCGTCCTGAACCTCTTCAACTGCA
AAAATTGTGCAAATGAAGCTGTGGTTCAAAAGATTTTGGACAGGGTGCTG
TCAAGATACGATGTCCGCCTGAGACCGAATTTTGGAGGTGCCCCTGTGCCT
GTGAGAATATCTATTTATGTCACGAGCATTGAACAGATCTCAGAAATGAA
TATGGACTACACGATCACGATGTTTTTTCATCAGACTTGGAAAGATTCACG
CTTAGCATACTATGAGACCACCCTGAACTTGACCCTGGACTATCGGATGC
ATGAGAAGTTGTGGGTCCCTGACTGCTACTTTCTGAACAGCAAGGATGCTT
TCGTGCATGATGTGACTGTGGAGAATCGCGTGTTTCAGCTTCACCCAGATG
GAACGGTGCGGTACGGCATCCGACTCACCACTACAGCAGCTTGTTCCCTG
GATCTGCATAAATTCCCTATGGACAAGCAGGCCTGCAACCTGGTGGTAGA
GAGCTATGGTTACACGGTTGAAGACATCATATTATTCTGGGATGACAATG
GGAACGCCATCCACATGACTGAGGAGCTGCATATCCCTCAGTTCACTTTCC
TGGGAAGGACGATTACTAGCAAGGAGGTGTATTTCTACACAGGTTCCTAC
ATACGCCTGATACTGAAGTTCCAGGTTCAGAGGGAAGTTAACAGCTACCT
TGTGCAAGTCTACTGGCCTACTGTCCTCACCACTATTACCTCTTGGATATC
GTTTTGGATGAACTATGATTCCTCTGCAGCCAGGGTGACAATTGGCTTAAC
TTCAATGCTCATCCTGACCACCATCGACTCACATCTGCGGGATAAGCTCCC
CAACATTTCCTGTATCAAGGCCATTGATATCTATATCCTCGTGTGCTTGTTC
TTTGTGTTCCTGTCCTTGCTGGAGTATGTCTACATCAACTATCTTTTCTACA
GTCGAGGACCTCGGCGCCAGCCTAGGCGACACAGGAGACCCCGAAGAGT
CATTGCCCGCTACCGCTACCAGCAAGTGGTGGTAGGAAACGTGCAGGATG
GCCTGATTAACGTGGAAGACGGAGTCAGCTCTCTCCCCATCACCCCAGCG
CAGGCCCCCCTGGCAAGCCCGGAAAGCCTCGGTTCTTTGACGTCCACCTCC
GAGCAGGCCCAGCTGGCCACCTCGGAAAGCCTCAGCCCACTCACTTCTCT
CTCAGGCCAGGCCCCCCTGGCCACTGGAGAAAGCCTGAGCGATCTCCCCT
CCACCTCAGAGCAGGCCCGGCACAGCTATGGTGTTCGCTTTAATGGTTTCC
AGGCTGATGACAGTATTATTCCTACCGAAATCCGCAACCGTGTCGAAGCC
CATGGCCATGGTGTTACCCATGACCATGAAGATTCCAATGAGAGCTTGAG
CTCGGATGAGCGCCATGGCCATGGCCCCAGTGGGAAGCCCATGCTTCACC
ATGGCGAGAAGGGTGTGCAAGAAGCAGGCTGGGACCTTGATGACAACAA
TGACAAGAGCGACTGCCTTGCCATTAAGGAGCAATTCAAGTGTGATACTA
ACAGTACCTGGGGCCTTAATGATGATGAGCTCATGGCCCATGGCCAAGAG
AAGGACAGTAGCTCAGAGTCTGAGGATAGTTGCCCCCCAAGCCCTGGGTG
CTCCTTCACTGAAGGGTTCTCCTTCGATCTCTTTAATCCTGACTACGTCCCA
AAGGTCGACAAGTGGTCCCGGTTCCTCTTCCCTCTGGCCTTTGGGTTGTTC
AACATTGTTTACTGGGTATACCATATGTATT-AG
Human GABA_{C} receptor rho 1 subunit cDNA
(SEQ ID NO: 20)
ATGTTGGCTGTCCCAAATATGAGATTTGGCATCTTTCTTTTGTGGTGGGGA
TGGGTTTTGGCCACTGAAAGCAGAATGCACTGGCCCGGAAGAGAAGTCCA
CGAGATGTCTAAGAAAGGCAGGCCCCAAAGACAAAGACGAGAAGTACAT
GAAGATGCCCACAAGCAAGTCAGCCCAATTCTGAGACGAAGTCCTGACAT
CACCAAATCGCCTCTGACAAAGTCAGAACAGCTTCTGAGGATAGATGACC
ATGATTTCAGCATGAGGCCTGGCTTTGGAGGCCCTGCCATTCCTGTTGGTG
TGGATGTGCAGGTGGAGAGTTTGGATAGCATCTCAGAGGTTGACATGGAC
TTTACGATGACCCTCTACCTGAGGCACTACTGGAAGGACGAGAGGCTGTC
TTTTCCAAGCACCAACAACCTCAGCATGACGTTTGATGGCCGGCTGGTCA
AGAAGATCTGGGTCCCTGACATGTTTTTCGTGCACTCCAAACGCTCCTTCA
TCCACGACACCACCACAGACAACGTCATGTTGCGGGTCCAGCCTGATGGG
AAAGTGCTCTATAGTCTCAGGGTTACAGTAACTGCAATGTGCAACATGGA
CTTCAGCCGATTTCCCTTGGACACACAAACGTGCTCTCTTGAAATTGAAAG
CTATGCCTATACAGAAGATGACCTCATGCTGTACTGGAAAAAGGGCAATG
ACTCCTTAAAGACAGATGAACGGATCTCACTCTCCCAGTTCCTCATTCAGG
AATTCCACACCACCACCAAACTGGCTTTCTACAGCAGCACAGGCTGGTAC
AACCGTCTCTACATTAATTTCACGTTGCGTCGCCACATCTTCTTCTTCTTGC
TCCAAACTTATTTCCCCGCTACCCTGATGGTCATGCTGTCCTGGGTGTCCTT
CTGGATCGACCGCAGAGCCGTGCCTGCCAGAGTCCCCTTAGGTATCACAA
CGGTGCTGACCATGTCCACCATCATCACGGGCGTGAATGCCTCCATGCCG
CGCGTCTCCTACATCAAGGCCGTGGACATCTACCTCTGGGTCAGCTTTGTG
TTCGTGTTCCTCTCGGTGCTGGAGTATGCGGCCGTCAACTACCTGACCACT
GTGCAGGAGAGGAAGGAACAGAAGCTGCGGGAGAAGCTTCCCTGCACCA
GCGGATTACCTCCGCCCCGCACTGCGATGCTGGACGGCAACTACAGTGAT
GGGGAGGTGAATGACCTGGACAACTACATGCCAGAGAATGGAGAGAAGC
CCGACAGGATGATGGTGCAGCTGACCCTGGCCTCAGAGAGGAGCTCCCCA
CAGAGGAAAAGTCAGAGAAGCAGCTATGTGAGCATGAGAATCGACACCC
ACGCCATTGATAAATACTCCAGGATCATCTTTCCAGCAGCATACATTTTAT
TCAATTTAATATACTGGTCTATTTTCTCCTAG
Human GABA_{C} receptor rho 2 subunit cDNA
(SEQ ID NO: 21)
ATGGTCAAGCCAGGGGGGATTTGCTCTGCCACAGGCTACTGGAAAGCAGC
TTTTTGCCTCACAGATGTCCACAAAATGCCTTATTTTACAAGACTCATTTT
GTTCTTGTTTTGCTTGATGGTTCTCGTGGAGAGCAGAAAACCCAAGAGGA
AGCGATGGACAGGGCAGGTGGAAATGCCCAAGCCAAGTCACTTATATAA
GAAGAACCTTGATGTGACCAAGATCCGGAAGGGAAAGCCTCAGCAGCTTC
TCAGAGTGGACGAGCACGACTTCAGCATGAGACCCGCCTTCGGAGGCCCT
GCCATCCCGGTGGGCGTGGACGTACAGGTGGAGAGCCTGGACAGCATCTC
CGAGGTGGACATGGACTTCACTATGACCCTGTACCTGCGGCATTACTGGA
AGGATGAGAGGCTAGCTTTCTCCAGCGCCAGCAACAAGAGCATGACCTTC
GATGGCCGGCTGGTGAAGAAGATCTGGGTCCCTGATGTCTTCTTTGTTCAC
TCCAAAAGATCGTTCACTCATGACACCACCACTGACAACATCATGCTGAG
GGTGTTCCCAGATGGACACGTGCTGTACAGCATGAGGATTACGGTCACTG
CCATGTGCAACATGGACTTCAGCCACTTTCCCCTGGACTCCCAGACCTGTT
CTTTGGAGCTGGAGAGCTATGCCTATACAGATGAAGATCTAATGCTGTAC
TGGAAGAATGGGGATGAATCCCTAAAAACAGATGAGAAGATCTCCTTGTC
TCAGTTTCTGATTCAGAAATTTCACACAACTTCCAGGCTGGCCTTCTACAG
CAGCACTGGCTGGTACAACCGTCTGTACATTAACTTCACGTTGCGTCGCCA
CATCTTCTTCTTCTTGCTCCAAACATATTTCCCTGCCACTCTGATGGTCATG
CTGTCCTGGGTGTCCTTCTGGATCGACCGCAGAGCTGTGCCTGCCAGAGTT
TCACTGGGTATCACGACGGTGCTGACCATGACCACCATCATCACGGGCGT
GAATGCCTCCATGCCGCGCGTCTCCTACGTCAAGGCCGTGGACATCTACCT
CTGGGTCAGCTTTGTGTTCGTGTTCCTCTCGGTGCTGGAGTATGCGGCTGT
CAACTACCTGACCACCGTGCAGGAGCGCAAGGAACGGAAGCTGCGGGAG
AAGTTCCCGTGCATGTGTGGAATGCTTCATTCAAAAACCATGATGCTGGAT
GGAAGCTACAGTGAGTCTGAGGCCAACAGCCTGGCTGGGTACCCCAGAAG
CCATATCCTGACAGAAGAAGAAAGGCAAGACAAAATAGTGGTCCACCTG
GGCCTGAGTGGTGAAGCCAACGCTGCCAGAAAGAAGGGGCTTCTGAAGG
GCCAGACGGGTTTTCGTATCTTCCAGAATACCCATGCCATTGACAAATACT
CTAGGTTGATATTCCCTGCCTCCTACATATTTTTCAACTTAATTTATTGGTC
AGTGTTTTCCTAG
Human GABA_{C} receptor rho 3 subunit cDNA
(SEQ ID NO: 22)
ATGGTCCTGGCTTTCCAGTTAGTCTCCTTCACCTACATCTGGATCATATTG
AAACCAAATGTTTGTGCTGCTTCTAACATCAAGATGACACACCAGCGGTG
CTCCTCTTCAATGAAACAAACCTGCAAACAAGAAACTAGAATGAAGAAAG
ATGACAGTACCAAAGCGCGGCCTCAGAAATATGAGCAACTTCTCCATATA
GAGGACAACGATTTCGCAATGAGACCTGGATTTGGAGGGTCTCCAGTGCC
AGTAGGTATAGATGTCCATGTTGAAAGCATTGACAGCATTTCAGAGACTA
ACATGGACTTTACAATGACTTTTTATCTCAGGCATTACTGGAAAGACGAG
AGGCTCTCCTTTCCTAGCACAGCAAACAAAAGCATGACATTTGATCATAG
ATTGACCAGAAAGATCTGGGTGCCTGATATCTTTTTTGTCCACTCTAAAAG
ATCCTTCATCCATGATACAACTATGGAGAATATCATGCTGCGCGTACACCC
TGATGGAAACGTCCTCCTAAGTCTCAGGATAACGGTTTCGGCCATGTGCTT
TATGGATTTCAGCAGGTTTCCTCTTGACACTCAAAATTGTTCTCTTGAACT
GGAAAGCTATGCCTACAATGAGGATGACCTAATGCTATACTGGAAACACG
GAAACAAGTCCTTAAATACTGAAGAACATATGTCCCTTTCTCAGTTCTTCA
TTGAAGACTTCAGTGCATCTAGTGGATTAGCTTTCTATAGCAGCACAGGTT
GGTACAATAGGCTTTTCATCAACTTTGTGCTAAGGAGGCATGTTTTCTTCT
TTGTGCTGCAAACCTATTTCCCAGCCATATTGATGGTGATGCTTTCATGGG
TTTCATTTTGGATTGACCGAAGAGCTGTTCCTGCAAGAGTTTCCCTGGGAA
TCACCACAGTGCTGACCATGTCCACAATCATCACTGCTGTGAGCGCCTCCA
TGCCCCAGGTGTCCTACCTCAAGGCTGTGGATGTGTACCTGTGGGTCAGCT
CCCTCTTTGTGTTCCTGTCAGTCATTGAGTATGCAGCTGTGAACTACCTCA
CCACAGTGGAAGAGCGGAAACAATTCAAGAAGACAGGAAAGATTTCTAG
GATGTACAATATTGATGCAGTTCAAGCTATGGCCTTTGATGGTTGTTACCA
TGACAGCGAGATTGACATGGACCAGACTTCCCTCTCTCTAAACTCAGAAG
ACTTCATGAGAAGAAAATCGATATGCAGCCCCAGCACCGATTCATCTCGG
ATAAAGAGAAGAAAATCCCTAGGAGGACATGTTGGTAGAATCATTCTGGA
AAACAACCATGTCATTGACACCTATTCTAGGATTTTATTCCCCATTGTGTA
TATTTTATTTAATTTGTTTTACTGGGGTGTATATGTATGA
Human GABAB receptor 1 isoform 1A subunit cDNA
(SEQ ID NO: 23)
ATGTTGCTGCTGCTGTTACTGGCGCCACTCTTCCTCCGCCCCCCGGGCGCG
GGCGGGGCGCAGACCCCCAACGCCACCTCAGAAGGTTGCCAGATCATACA
CCCGCCCTGGGAAGGGGGCATCAGGTACCGGGGCCTGACTCGGGACCAG
GTGAAGGCTATCAACTTCCTGCCAGTGGACTATGAGATTGAGTATGTGTG
CCGGGGGGAGCGCGAGGTGGTGGGGCCCAAGGTCCGCAAGTGCCTGGCC
AACGGCTCCTGGACAGATATGGACACACCCAGCCGCTGTGTCCGAATCTG
CTCCAAGTCTTATTTGACCCTGGAAAATGGGAAGGTTTTCCTGACGGGTGG
GGACCTCCCAGCTCTGGACGGAGCCCGGGTGGATTTCCGGTGTGACCCCG
ACTTCCATCTGGTGGGCAGCTCCCGGAGCATCTGTAGTCAGGGCCAGTGG
AGCACCCCCAAGCCCCACTGCCAGGTGAATCGAACGCCACACTCAGAACG
GCGCGCAGTGTACATCGGGGCACTGTTTCCCATGAGCGGGGGCTGGCCAG
GGGGCCAGGCCTGCCAGCCCGCGGTGGAGATGGCGCTGGAGGACGTGAA
TAGCCGCAGGGACATCCTGCCGGACTATGAGCTCAAGCTCATCCACCACG
ACAGCAAGTGTGATCCAGGCCAAGCCACCAAGTACCTATATGAGCTGCTC
TACAACGACCCTATCAAGATCATCCTTATGCCTGGCTGCAGCTCTGTCTCC
ACGCTGGTGGCTGAGGCTGCTAGGATGTGGAACCTCATTGTGCTTTCCTAT
GGCTCCAGCTCACCAGCCCTGTCAAACCGGCAGCGTTTCCCCACTTTCTTC
CGAACGCACCCATCAGCCACACTCCACAACCCTACCCGCGTGAAACTCTT
TGAAAAGTGGGGCTGGAAGAAGATTGCTACCATCCAGCAGACCACTGAG
GTCTTCACTTCGACTCTGGACGACCTGGAGGAACGAGTGAAGGAGGCTGG
AATTGAGATTACTTTCCGCCAGAGTTTCTTCTCAGATCCAGCTGTGCCCGT
CAAAAACCTGAAGCGCCAGGATGCCCGAATCATCGTGGGACTTTTCTATG
AGACTGAAGCCCGGAAAGTTTTTTGTGAGGTGTACAAGGAGCGTCTCTTT
GGGAAGAAGTACGTCTGGTTCCTCATTGGGTGGTATGCTGACAATTGGTTC
AAGATCTACGACCCTTCTATCAACTGCACAGTGGATGAGATGACTGAGGC
GGTGGAGGGCCACATCACAACTGAGATTGTCATGCTGAATCCTGCCAATA
CCCGCAGCATTTCCAACATGACATCCCAGGAATTTGTGGAGAAACTAACC
AAGCGACTGAAAAGACACCCTGAGGAGACAGGAGGCTTCCAGGAGGCAC
CGCTGGCCTATGATGCCATCTGGGCCTTGGCACTGGCCCTGAACAAGACA
TCTGGAGGAGGCGGCCGTTCTGGTGTGCGCCTGGAGGACTTCAACTACAA
CAACCAGACCATTACCGACCAAATCTACCGGGCAATGAACTCTTCGTCCTT
TGAGGGTGTCTCTGGCCATGTGGTGTTTGATGCCAGCGGCTCTCGGATGGC
ATGGACGCTTATCGAGCAGCTTCAGGGTGGCAGCTACAAGAAGATTGGCT
ACTATGACAGCACCAAGGATGATCTTTCCTGGTCCAAAACAGATAAATGG
ATTGGAGGGTCCCCCCCAGCTGACCAGACCCTGGTCATCAAGACATTCCG
CTTCCTGTCACAGAAACTCTTTATCTCCGTCTCAGTTCTCTCCAGCCTGGGC
ATTGTCCTAGCTGTTGTCTGTCTGTCCTTTAACATCTACAACTCACATGTCC
GTTATATCCAGAACTCACAGCCCAACCTGAACAACCTGACTGCTGTGGGC
TGCTCACTGGCTTTAGCTGCTGTCTTCCCCCTGGGGCTCGATGGTTACCAC
ATTGGGAGGAACCAGTTTCCTTTCGTCTGCCAGGCCCGCCTCTGGCTCCTG
GGCCTGGGCTTTAGTCTGGGCTACGGTTCCATGTTCACCAAGATTTGGTGG
GTCCACACGGTCTTCACAAAGAAGGAAGAAAAGAAGGAGTGGAGGAAGA
CTCTGGAACCCTGGAAGCTGTATGCCACAGTGGGCCTGCTGGTGGGCATG
GATGTCCTCACTCTCGCCATCTGGCAGATCGTGGACCCTCTGCACCGGACC
ATTGAGACATTTGCCAAGGAGGAACCTAAGGAAGATATTGACGTCTCTAT
TCTGCCCCAGCTGGAGCATTGCAGCTCCAGGAAGATGAATACATGGCTTG
GCATTTTCTATGGTTACAAGGGGCTGCTGCTGCTGCTGGGAATCTTCCTTG
CTTATGAGACCAAGAGTGTGTCCACTGAGAAGATCAATGATCACCGGGCT
GTGGGCATGGCTATCTACAATGTGGCAGTCCTGTGCCTCATCACTGCTCCT
GTCACCATGATTCTGTCCAGCCAGCAGGATGCAGCCTTTGCCTTTGCCTCT
CTTGCCATAGTTTTCTCCTCCTATATCACTCTTGTTGTGCTCTTTGTGCCCA
AGATGCGCAGGCTGATCACCCGAGGGGAATGGCAGTCGGAGGCGCAGGA
CACCATGAAGACAGGGTCATCGACCAACAACAACGAGGAGGAGAAGTCC
CGGCTGTTGGAGAAGGAGAACCGTGAACTGGAAAAGATCATTGCTGAGA
AAGAGGAGCGTGTCTCTGAACTGCGCCATCAACTCCAGTCTCGGCAGCAG
CTCCGCTCCCGGCGCCACCCACCGACACCCCCAGAACCCTCTGGGGGCCT
GCCCAGGGGACCCCCTGAGCCCCCCGACCGGCTTAGCTGTGATGGGAGTC
GAGTGCATTTGCTTTATAAGTGA
Human GABA_{B} receptor 1 isoform 1B subunit cDNA
(SEQ ID NO: 24)
ATGGGGCCCGGGGCCCCTTTTGCCCGGGTGGGGTGGCCACTGCCGCTTCT
GGTTGTGATGGCGGCAGGGGTGGCTCCGGTGTGGGCCTCCCACTCCCCCC
ATCTCCCGCGGCCTCACTCGCGGGTCCCCCCGCACCCCTCCTCAGAACGGC
GCGCAGTGTACATCGGGGCACTGTTTCCCATGAGCGGGGGCTGGCCAGGG
GGCCAGGCCTGCCAGCCCGCGGTGGAGATGGCGCTGGAGGACGTGAATA
GCCGCAGGGACATCCTGCCGGACTATGAGCTCAAGCTCATCCACCACGAC
AGCAAGTGTGATCCAGGCCAAGCCACCAAGTACCTATATGAGCTGCTCTA
CAACGACCCTATCAAGATCATCCTTATGCCTGGCTGCAGCTCTGTCTCCAC
GCTGGTGGCTGAGGCTGCTAGGATGTGGAACCTCATTGTGCTTTCCTATGG
CTCCAGCTCACCAGCCCTGTCAAACCGGCAGCGTTTCCCCACTTTCTTCCG
AACGCACCCATCAGCCACACTCCACAACCCTACCCGCGTGAAACTCTTTG
AAAAGTGGGGCTGGAAGAAGATTGCTACCATCCAGCAGACCACTGAGGTC
TTCACTTCGACTCTGGACGACCTGGAGGAACGAGTGAAGGAGGCTGGAAT
TGAGATTACTTTCCGCCAGAGTTTCTTCTCAGATCCAGCTGTGCCCGTCAA
AAACCTGAAGCGCCAGGATGCCCGAATCATCGTGGGACTTTTCTATGAGA
CTGAAGCCCGGAAAGTTTTTTGTGAGGTGTACAAGGAGCGTCTCTTTGGG
AAGAAGTACGTCTGGTTCCTCATTGGGTGGTATGCTGACAATTGGTTCAAG
ATCTACGACCCTTCTATCAACTGCACAGTGGATGAGATGACTGAGGCGGT
GGAGGGCCACATCACAACTGAGATTGTCATGCTGAATCCTGCCAATACCC
GCAGCATTTCCAACATGACATCCCAGGAATTTGTGGAGAAACTAACCAAG
CGACTGAAAAGACACCCTGAGGAGACAGGAGGCTTCCAGGAGGCACCGC
TGGCCTATGATGCCATCTGGGCCTTGGCACTGGCCCTGAACAAGACATCT
GGAGGAGGCGGCCGTTCTGGTGTGCGCCTGGAGGACTTCAACTACAACAA
CCAGACCATTACCGACCAAATCTACCGGGCAATGAACTCTTCGTCCTTTGA
GGGTGTCTCTGGCCATGTGGTGTTTGATGCCAGCGGCTCTCGGATGGCATG
GACGCTTATCGAGCAGCTTCAGGGTGGCAGCTACAAGAAGATTGGCTACT
ATGACAGCACCAAGGATGATCTTTCCTGGTCCAAAACAGATAAATGGATT
GGAGGGTCCCCCCCAGCTGACCAGACCCTGGTCATCAAGACATTCCGCTT
CCTGTCACAGAAACTCTTTATCTCCGTCTCAGTTCTCTCCAGCCTGGGCAT
TGTCCTAGCTGTTGTCTGTCTGTCCTTTAACATCTACAACTCACATGTCCGT
TATATCCAGAACTCACAGCCCAACCTGAACAACCTGACTGCTGTGGGCTG
CTCACTGGCTTTAGCTGCTGTCTTCCCCCTGGGGCTCGATGGTTACCACAT
TGGGAGGAACCAGTTTCCTTTCGTCTGCCAGGCCCGCCTCTGGCTCCTGGG
CCTGGGCTTTAGTCTGGGCTACGGTTCCATGTTCACCAAGATTTGGTGGGT
CCACACGGTCTTCACAAAGAAGGAAGAAAAGAAGGAGTGGAGGAAGACT
CTGGAACCCTGGAAGCTGTATGCCACAGTGGGCCTGCTGGTGGGCATGGA
TGTCCTCACTCTCGCCATCTGGCAGATCGTGGACCCTCTGCACCGGACCAT
TGAGACATTTGCCAAGGAGGAACCTAAGGAAGATATTGACGTCTCTATTC
TGCCCCAGCTGGAGCATTGCAGCTCCAGGAAGATGAATACATGGCTTGGC
ATTTTCTATGGTTACAAGGGGCTGCTGCTGCTGCTGGGAATCTTCCTTGCT
TATGAGACCAAGAGTGTGTCCACTGAGAAGATCAATGATCACCGGGCTGT
GGGCATGGCTATCTACAATGTGGCAGTCCTGTGCCTCATCACTGCTCCTGT
CACCATGATTCTGTCCAGCCAGCAGGATGCAGCCTTTGCCTTTGCCTCTCT
TGCCATAGTTTTCTCCTCCTATATCACTCTTGTTGTGCTCTTTGTGCCCAAG
ATGCGCAGGCTGATCACCCGAGGGGAATGGCAGTCGGAGGCGCAGGACA
CCATGAAGACAGGGTCATCGACCAACAACAACGAGGAGGAGAAGTCCCG
GCTGTTGGAGAAGGAGAACCGTGAACTGGAAAAGATCATTGCTGAGAAA
GAGGAGCGTGTCTCTGAACTGCGCCATCAACTCCAGTCTCGGCAGCAGCT
CCGCTCCCGGCGCCACCCACCGACACCCCCAGAACCCTCTGGGGGCCTGC
CCAGGGGACCCCCTGAGCCCCCCGACCGGCTTAGCTGTGATGGGAGTCGA
GTGCATTTGCTTTATAAGTGA
Human GABA_{B} receptor 1 isoform 1C subunit cDNA
(SEQ ID NO: 25)
ATGTTGCTGCTGCTGCTACTGGCGCCACTCTTCCTCCGCCCCCCGGGCGCG
GGCGGGGCGCAGACCCCCAACGCCACCTCAGAAGGTTGCCAGATCATACA
CCCGCCCTGGGAAGGGGGCATCAGGTACCGGGGCCTGACTCGGGACCAG
GTGAAGGCTATCAACTTCCTGCCAGTGGACTATGAGATTGAGTATGTGTG
CCGGGGGGAGCGCGAGGTGGTGGGGCCCAAGGTCCGCAAGTGCCTGGCC
AACGGCTCCTGGACAGATATGGACACACCCAGCCGCTGTGTGAATCGAAC
GCCACACTCAGAACGGCGCGCAGTGTACATCGGGGCACTGTTTCCCATGA
GCGGGGGCTGGCCAGGGGGCCAGGCCTGCCAGCCCGCGGTGGAGATGGC
GCTGGAGGACGTGAATAGCCGCAGGGACATCCTGCCGGACTATGAGCTCA
AGCTCATCCACCACGACAGCAAGTGTGATCCAGGCCAAGCCACCAAGTAC
CTATATGAGCTGCTCTACAACGACCCTATCAAGATCATCCTTATGCCTGGC
TGCAGCTCTGTCTCCACGCTGGTGGCTGAGGCTGCTAGGATGTGGAACCTC
ATTGTGCTTTCCTATGGCTCCAGCTCACCAGCCCTGTCAAACCGGCAGCGT
TTCCCCACTTTCTTCCGAACGCACCCATCAGCCACACTCCACAACCCTACC
CGCGTGAAACTCTTTGAAAAGTGGGGCTGGAAGAAGATTGCTACCATCCA
GCAGACCACTGAGGTCTTCACTTCGACTCTGGACGACCTGGAGGAACGAG
TGAAGGAGGCTGGAATTGAGATTACTTTCCGCCAGAGTTTCTTCTCAGATC
CAGCTGTGCCCGTCAAAAACCTGAAGCGCCAGGATGCCCGAATCATCGTG
GGACTTTTCTATGAGACTGAAGCCCGGAAAGTTTTTTGTGAGGTGTACAA
GGAGCGTCTCTTTGGGAAGAAGTACGTCTGGTTCCTCATTGGGTGGTATGC
TGACAATTGGTTCAAGATCTACGACCCTTCTATCAACTGCACAGTGGATGA
GATGACTGAGGCGGTGGAGGGCCACATCACAACTGAGATTGTCATGCTGA
ATCCTGCCAATACCCGCAGCATTTCCAACATGACATCCCAGGAATTTGTGG
AGAAACTAACCAAGCGACTGAAAAGACACCCTGAGGAGACAGGAGGCTT
CCAGGAGGCACCGCTGGCCTATGATGCCATCTGGGCCTTGGCACTGGCCC
TGAACAAGACATCTGGAGGAGGCGGCCGTTCTGGTGTGCGCCTGGAGGAC
TTCAACTACAACAACCAGACCATTACCGACCAAATCTACCGGGCAATGAA
CTCTTCGTCCTTTGAGGGTGTCTCTGGCCATGTGGTGTTTGATGCCAGCGG
CTCTCGGATGGCATGGACGCTTATCGAGCAGCTTCAGGGTGGCAGCTACA
AGAAGATTGGCTACTATGACAGCACCAAGGATGATCTTTCCTGGTCCAAA
ACAGATAAATGGATTGGAGGGTCCCCCCCAGCTGACCAGACCCTGGTCAT
CAAGACATTCCGCTTCCTGTCACAGAAACTCTTTATCTCCGTCTCAGTTCT
CTCCAGCCTGGGCATTGTCCTAGCTGTTGTCTGTCTGTCCTTTAACATCTAC
AACTCACATGTCCGTTATATCCAGAACTCACAGCCCAACCTGAACAACCT
GACTGCTGTGGGCTGCTCACTGGCTTTAGCTGCTGTCTTCCCCCTGGGGCT
CGATGGTTACCACATTGGGAGGAACCAGTTTCCTTTCGTCTGCCAGGCCCG
CCTCTGGCTCCTGGGCCTGGGCTTTAGTCTGGGCTACGGTTCCATGTTCAC
CAAGATTTGGTGGGTCCACACGGTCTTCACAAAGAAGGAAGAAAAGAAG
GAGTGGAGGAAGACTCTGGAACCCTGGAAGCTGTATGCCACAGTGGGCCT
GCTGGTGGGCATGGATGTCCTCACTCTCGCCATCTGGCAGATCGTGGACCC
TCTGCACCGGACCATTGAGACATTTGCCAAGGAGGAACCTAAGGAAGATA
TTGACGTCTCTATTCTGCCCCAGCTGGAGCATTGCAGCTCCAGGAAGATGA
ATACATGGCTTGGCATTTTCTATGGTTACAAGGGGCTGCTGCTGCTGCTGG
GAATCTTCCTTGCTTATGAGACCAAGAGTGTGTCCACTGAGAAGATCAAT
GATCACCGGGCTGTGGGCATGGCTATCTACAATGTGGCAGTCCTGTGCCTC
ATCACTGCTCCTGTCACCATGATTCTGTCCAGCCAGCAGGATGCAGCCTTT
GCCTTTGCCTCTCTTGCCATAGTTTTCTCCTCCTATATCACTCTTGTTGTGC
TCTTTGTGCCCAAGATGCGCAGGCTGATCACCCGAGGGGAATGGCAGTCG
GAGGCGCAGGACACCATGAAGACAGGGTCATCGACCAACAACAACGAGG
AGGAGAAGTCCCGGCTGTTGGAGAAGGAGAACCGTGAACTGGAAAAGAT
CATTGCTGAGAAAGAGGAGCGTGTCTCTGAACTGCGCCATCAACTCCAGT
CTCGGCAGCAGCTCCGCTCCCGGCGCCACCCACCGACACCCCCAGAACCC
TCTGGGGGCCTGCCCAGGGGACCCCCTGAGCCCCCCGACCGGCTTAGCTG
TGATGGGAGTCGAGTGCATTTGCTTTATAAGTGA
Human GABA_{B} receptor 1 isoform 1D subunit cDNA
(SEQ ID NO: 26)
ATGTTGCTGCTGCTGTTACTGGCGCCACTCTTCCTCCGCCCCCCGGGCGCG
GGCGGGGCGCAGACCCCCAACGCCACCTCAGAAGGTTGCCAGATCATACA
CCCGCCCTGGGAAGGGGGCATCAGGTACCGGGGCCTGACTCGGGACCAG
GTGAAGGCTATCAACTTCCTGCCAGTGGACTATGAGATTGAGTATGTGTG
CCGGGGGGAGCGCGAGGTGGTGGGGCCCAAGGTCCGCAAGTGCCTGGCC
AACGGCTCCTGGACAGATATGGACACACCCAGCCGCTGTGTCCGAATCTG
CTCCAAGTCTTATTTGACCCTGGAAAATGGGAAGGTTTTCCTGACGGGTGG
GGACCTCCCAGCTCTGGACGGAGCCCGGGTGGATTTCCGGTGTGACCCCG
ACTTCCATCTGGTGGGCAGCTCCCGGAGCATCTGTAGTCAGGGCCAGTGG
AGCACCCCCAAGCCCCACTGCCAGGTGAATCGAACGCCACACTCAGAACG
GCGCGCAGTGTACATCGGGGCACTGTTTCCCATGAGCGGGGGCTGGCCAG
GGGGCCAGGCCTGCCAGCCCGCGGTGGAGATGGCGCTGGAGGACGTGAA
TAGCCGCAGGGACATCCTGCCGGACTATGAGCTCAAGCTCATCCACCACG
ACAGCAAGTGTGATCCAGGCCAAGCCACCAAGTACCTATATGAGCTGCTC
TACAACGACCCTATCAAGATCATCCTTATGCCTGGCTGCAGCTCTGTCTCC
ACGCTGGTGGCTGAGGCTGCTAGGATGTGGAACCTCATTGTGCTTTCCTAT
GGCTCCAGCTCACCAGCCCTGTCAAACCGGCAGCGTTTCCCCACTTTCTTC
CGAACGCACCCATCAGCCACACTCCACAACCCTACCCGCGTGAAACTCTT
TGAAAAGTGGGGCTGGAAGAAGATTGCTACCATCCAGCAGACCACTGAG
GTCTTCACTTCGACTCTGGACGACCTGGAGGAACGAGTGAAGGAGGCTGG
AATTGAGATTACTTTCCGCCAGAGTTTCTTCTCAGATCCAGCTGTGCCCGT
CAAAAACCTGAAGCGCCAGGATGCCCGAATCATCGTGGGACTTTTCTATG
AGACTGAAGCCCGGAAAGTTTTTTGTGAGGTGTACAAGGAGCGTCTCTTT
GGGAAGAAGTACGTCTGGTTCCTCATTGGGTGGTATGCTGACAATTGGTTC
AAGATCTACGACCCTTCTATCAACTGCACAGTGGATGAGATGACTGAGGC
GGTGGAGGGCCACATCACAACTGAGATTGTCATGCTGAATCCTGCCAATA
CCCGCAGCATTTCCAACATGACATCCCAGGAATTTGTGGAGAAACTAACC
AAGCGACTGAAAAGACACCCTGAGGAGACAGGAGGCTTCCAGGAGGCAC
CGCTGGCCTATGATGCCATCTGGGCCTTGGCACTGGCCCTGAACAAGACA
TCTGGAGGAGGCGGCCGTTCTGGTGTGCGCCTGGAGGACTTCAACTACAA
CAACCAGACCATTACCGACCAAATCTACCGGGCAATGAACTCTTCGTCCTT
TGAGGGTGTCTCTGGCCATGTGGTGTTTGATGCCAGCGGCTCTCGGATGGC
ATGGACGCTTATCGAGCAGCTTCAGGGTGGCAGCTACAAGAAGATTGGCT
ACTATGACAGCACCAAGGATGATCTTTCCTGGTCCAAAACAGATAAATGG
ATTGGAGGGTCCCCCCCAGCTGACCAGACCCTGGTCATCAAGACATTCCG
CTTCCTGTCACAGAAACTCTTTATCTCCGTCTCAGTTCTCTCCAGCCTGGGC
ATTGTCCTAGCTGTTGTCTGTCTGTCCTTTAACATCTACAACTCACATGTCC
GTTATATCCAGAACTCACAGCCCAACCTGAACAACCTGACTGCTGTGGGC
TGCTCACTGGCTTTAGCTGCTGTCTTCCCCCTGGGGCTCGATGGTTACCAC
ATTGGGAGGAACCAGTTTCCTTTCGTCTGCCAGGCCCGCCTCTGGCTCCTG
GGCCTGGGCTTTAGTCTGGGCTACGGTTCCATGTTCACCAAGATTTGGTGG
GTCCACACGGTCTTCACAAAGAAGGAAGAAAAGAAGGAGTGGAGGAAGA
CTCTGGAACCCTGGAAGCTGTATGCCACAGTGGGCCTGCTGGTGGGCATG
GATGTCCTCACTCTCGCCATCTGGCAGATCGTGGACCCTCTGCACCGGACC
ATTGAGACATTTGCCAAGGAGGAACCTAAGGAAGATATTGACGTCTCTAT
TCTGCCCCAGCTGGAGCATTGCAGCTCCAGGAAGATGAATACATGGCTTG
GCATTTTCTATGGTTACAAGGGGCTGCTGCTGCTGCTGGGAATCTTCCTTG
CTTATGAGACCAAGAGTGTGTCCACTGAGAAGATCAATGATCACCGGGCT
GTGGGCATGGCTATCTACAATGTGGCAGTCCTGTGCCTCATCACTGCTCCT
GTCACCATGATTCTGTCCAGCCAGCAGGATGCAGCCTTTGCCTTTGCCTCT
CTTGCCATAGTTTTCTCCTCCTATATCACTCTTGTTGTGCTCTTTGTGCCCA
AGATGCGCAGGCTGATCACCCGAGGGGAATGGCAGTCGGAGGCGCAGGA
CACCATGAAGACAGGGTCATCGACCAACAACAACGAGGAGGAGAAGTCC
CGGCTGTTGGAGAAGGAGAACCGTGAACTGGAAAAGATCATTGCTGAGA
GCGGCGGCCTGCCCCGGGGCCCCCCTGAGCCCCCTGACCGGCTGAGCTGC
GACGGATCCAGAGTGCACCTGCTCTACAAG
Human GABA_{B} receptor 1 isoform 1E subunit cDNA
(SEQ ID NO: 27)
ATGTTGCTGCTGCTGCTACTGGCGCCACTCTTCCTCCGCCCCCCGGGCGCG
GGCGGGGCGCAGACCCCCAACGCCACCTCAGAAGGTTGCCAGATCATACA
CCCGCCCTGGGAAGGGGGCATCAGGTACCGGGGCCTGACTCGGGACCAG
GTGAAGGCTATCAACTTCCTGCCAGTGGACTATGAGATTGAGTATGTGTG
CCGGGGGGAGCGCGAGGTGGTGGGGCCCAAGGTCCGCAAGTGCCTGGCC
AACGGCTCCTGGACAGATATGGACACACCCAGCCGCTGTGTCCGAATCTG
CTCCAAGTCTTATTTGACCCTGGAAAATGGGAAGGTTTTCCTGACGGGTGG
GGACCTCCCAGCTCTGGACGGAGCCCGGGTGGATTTCCGGTGTGACCCCG
ACTTCCATCTGGTGGGCAGCTCCCGGAGCATCTGTAGTCAGGGCCAGTGG
AGCACCCCCAAGCCCCACTGCCAGGTGAATCGAACGCCACACTCAGAACG
GCGCGCAGTGTACATCGGGGCACTGTTTCCCATGAGCGGGGGCTGGCCAG
GGGGCCAGGCCTGCCAGCCCGCGGTGGAGATGGCGCTGGAGGACGTGAA
TAGCCGCAGGGACATCCTGCCGGACTATGAGCTCAAGCTCATCCACCACG
ACAGCAAGTGTGATCCAGGCCAAGCCACCAAGTACCTATATGAGCTGCTC
TACAACGACCCTATCAAGATCATCCTTATGCCTGGCTGCAGCTCTGTCTCC
ACGCTGGTGGCTGAGGCTGCTAGGATGTGGAACCTCATTGTGCTTTCCTAT
GGCTCCAGCTCACCAGCCCTGTCAAACCGGCAGCGTTTCCCCACTTTCTTC
CGAACGCACCCATCAGCCACACTCCACAACCCTACCCGCGTGAAACTCTT
TGAAAAGTGGGGCTGGAAGAAGATTGCTACCATCCAGCAGACCACTGAG
GTCTTCACTTCGACTCTGGACGACCTGGAGGAACGAGTGAAGGAGGCTGG
AATTGAGATTACTTTCCGCCAGAGTTTCTTCTCAGATCCAGCTGTGCCCGT
CAAAAACCTGAAGCGCCAGGATGCCCGAATCATCGTGGGACTTTTCTATG
AGACTGAAGCCCGGAAAGTTTTTTGTGAGGTGTACAAGGAGCGTCTCTTT
GGGAAGAAGTACGTCTGGTTCCTCATTGGGTGGTATGCTGACAATTGGTTC
AAGATCTACGACCCTTCTATCAACTGCACAGTGGATGAGATGACTGAGGC
GGTGGAGGGCCACATCACAACTGAGATTGTCATGCTGAATCCTGCCAATA
CCCGCAGCATTTCCAACATGACATCCCAGGAATTTGTGGAGAAACTAACC
AAGCGACTGAAAAGACACCCTGAGGAGACAGGAGGCTTCCAGGAGGCAC
CGCTGGCCTATGATGCCATCTGGGCCTTGGCACTGGCCCTGAACAAGACA
TCTGGAGGAGGCGGCCGTTCTGGTGTGCGCCTGGAGGACTTCAACTACAA
CAACCAGACCATTACCGACCAAATCTACCGGGCAATGAACTCTTCGTCCTT
TGAGGGTGTCTCTGGCCATGTGGTGTTTGATGCCAGCGGCTCTCGGATGGC
ATGGACGCTTATCGAGCAGCTTCAGGGTGGCAGCTACAAGAAGATTGGCT
ACTATGACAGCACCAAGGATGATCTTTCCTGGTCCAAAACAGATAAATGG
ATTGTTATATCCAGAACTCACAGCCCAACCTGA
Human GABA_{B} receptor 2 subunit cDNA
(SEQ ID NO: 28)
ATGGCTTCCCCGCGGAGCTCCGGGCAGCCCGGGCCGCCGCCGCCGCCGCC
ACCGCCGCCCGCGCGCCTGCTACTGCTACTGCTGCTGCCGCTGCTGCTGCC
TCTGGCGCCCGGGGCCTGGGGCTGGGCGCGGGGCGCCCCCCGGCCGCCGC
CCAGCAGCCCGCCGCTCTCCATCATGGGCCTCATGCCGCTCACCAAGGAG
GTGGCCAAGGGCAGCATCGGGCGCGGTGTGCTCCCCGCCGTGGAACTGGC
CATCGAGCAGATCCGCAACGAGTCACTCCTGCGCCCCTACTTCCTCGACCT
GCGGCTCTATGACACGGAGTGCGACAACGCAAAAGGGTTGAAAGCCTTCT
ACGATGCAATAAAATACGGGCCTAACCACTTGATGGTGTTTGGAGGCGTC
TGTCCATCCGTCACATCCATCATTGCAGAGTCCCTCCAAGGCTGGAATCTG
GTGCAGCTTTCTTTTGCTGCAACCACGCCTGTTCTAGCCGATAAGAAAAAA
TACCCTTATTTCTTTCGGACCGTCCCATCAGACAATGCGGTGAATCCAGCC
ATTCTGAAGTTGCTCAAGCACTACCAGTGGAAGCGCGTGGGCACGCTGAC
GCAAGACGTTCAGAGGTTCTCTGAGGTGCGGAATGACCTGACTGGAGTTC
TGTATGGCGAGGACATTGAGATTTCAGACACCGAGAGCTTCTCCAACGAT
CCCTGTACCAGTGTCAAAAAGCTGAAGGGGAATGATGTGCGGATCATCCT
TGGCCAGTTTGACCAGAATATGGCAGCAAAAGTGTTCTGTTGTGCATACG
AGGAGAACATGTATGGTAGTAAATATCAGTGGATCATTCCGGGCTGGTAC
GAGCCTTCTTGGTGGGAGCAGGTGCACACGGAAGCCAACTCATCCCGCTG
CCTCCGGAAGAATCTGCTTGCTGCCATGGAGGGCTACATTGGCGTGGATTT
CGAGCCCCTGAGCTCCAAGCAGATCAAGACCATCTCAGGAAAGACTCCAC
AGCAGTATGAGAGAGAGTACAACAACAAGCGGTCAGGCGTGGGGCCCAG
CAAGTTCCACGGGTACGCCTACGATGGCATCTGGGTCATCGCCAAGACAC
TGCAGAGGGCCATGGAGACACTGCATGCCAGCAGCCGGCACCAGCGGAT
CCAGGACTTCAACTACACGGACCACACGCTGGGCAGGATCATCCTCAATG
CCATGAACGAGACCAACTTCTTCGGGGTCACGGGTCAAGTTGTATTCCGG
AATGGGGAGAGAATGGGGACCATTAAATTTACTCAATTTCAAGACAGCAG
GGAGGTGAAGGTGGGAGAGTACAACGCTGTGGCCGACACACTGGAGATC
ATCAATGACACCATCAGGTTCCAAGGATCCGAACCACCAAAAGACAAGAC
CATCATCCTGGAGCAGCTGCGGAAGATCTCCCTACCTCTCTACAGCATCCT
CTCTGCCCTCACCATCCTCGGGATGATCATGGCCAGTGCTTTTCTCTTCTTC
AACATCAAGAACCGGAATCAGAAGCTCATAAAGATGTCGAGTCCATACAT
GAACAACCTTATCATCCTTGGAGGGATGCTCTCCTATGCTTCCATATTTCT
CTTTGGCCTTGATGGATCCTTTGTCTCTGAAAAGACCTTTGAAACACTTTG
CACCGTCAGGACCTGGATTCTCACCGTGGGCTACACGACCGCTTTTGGGG
CCATGTTTGCAAAGACCTGGAGAGTCCACGCCATCTTCAAAAATGTGAAA
ATGAAGAAGAAGATCATCAAGGACCAGAAACTGCTTGTGATCGTGGGGG
GCATGCTGCTGATCGACCTGTGTATCCTGATCTGCTGGCAGGCTGTGGACC
CCCTGCGAAGGACAGTGGAGAAGTACAGCATGGAGCCGGACCCAGCAGG
ACGGGATATCTCCATCCGCCCTCTCCTGGAGCACTGTGAGAACACCCATAT
GACCATCTGGCTTGGCATCGTCTATGCCTACAAGGGACTTCTCATGTTGTT
CGGTTGTTTCTTAGCTTGGGAGACCCGCAACGTCAGCATCCCCGCACTCAA
CGACAGCAAGTACATCGGGATGAGTGTCTACAACGTGGGGATCATGTGCA
TCATCGGGGCCGCTGTCTCCTTCCTGACCCGGGACCAGCCCAATGTGCAGT
TCTGCATCGTGGCTCTGGTCATCATCTTCTGCAGCACCATCACCCTCTGCC
TGGTATTCGTGCCGAAGCTCATCACCCTGAGAACAAACCCAGATGCAGCA
ACGCAGAACAGGCGATTCCAGTTCACTCAGAATCAGAAGAAAGAAGATTC
TAAAACGTCCACCTCGGTCACCAGTGTGAACCAAGCCAGCACATCCCGCC
TGGAGGGCCTACAGTCAGAAAACCATCGCCTGCGAATGAAGATCACAGA
GCTGGATAAAGACTTGGAAGAGGTCACCATGCAGCTGCAGGACACACCA
GAAAAGACCACCTACATTAAACAGAACCACTACCAAGAGCTCAATGACAT
CCTCAACCTGGGAAACTTCACTGAGAGCACAGATGGAGGAAAGGCCATTT
TAAAAAATCACCTCGATCAAAATCCCCAGCTACAGTGGAACACAACAGAG
CCCTCTCGAACATGCAAAGATCCTATAGAAGATATAAACTCTCCAGAACA
CATCCAGCGTCGGCTGTCCCTCCAGCTCCCCATCCTCCACCACGCCTACCT
CCCATCCATCGGAGGCGTGGACGCCAGCTGTGTCAGCCCCTGCGTCAGCC
CCACCGCCAGCCCCCGCCACAGACATGTGCCACCCTCCTTCCGAGTCATG
GTCTCGGGCCTGTAA
Human GABA_{A} receptor alpha 1 subunit amino acid
(SEQ ID NO: 29)
MRKSPGLSDCLWAWILLLSTLTGRSYGQPSLQDELKDNTTVFTRILDRLLDGY
DNRLRPGLGERVTEVKTDIFVTSFGPVSDHDMEYTIDVFFRQSWKDERLKFK
GPMTVLRLNNLMASKIWTPDTFFHNGKKSVAHNMTMPNKLLRITEDGTLLY
TMRLTVRAECPMHLEDFPMDAHACPLKFGSYAYTRAEVVYEWTREPARSVV
VAEDGSRLNQYDLLGQTVDSGIVQSSTGEYVVMTTHFHLKRKIGYFVIQTYL
PCIMTVILSQVSFWLNRESVPARTVFGVTTVLTMTTLSISARNSLPKVAYATA
MDWFIAVCYAFVFSALIEFATVNYFTKRGYAWDGKSVVPEKPKKVKDPLIKK
NNTYAPTATSYTPNLARGDPGLATIAKSATIEPKEVKPETKPPEPKKTFNSVSK
IDRLSRIAFPLLFGIFNLVYWATYLNREPQLKAPTPHQ
Human GABA_{A} receptor alpha 2 subunit amino acid
(SEQ ID NO: 30)
MKTKLNIYNMQFLLFVFLVWDPARLVLANIQEDEAKNNITIFTRILDRLLDGY
DNRLRPGLGDSITEVFTNIYVTSFGPVSDTDMEYTIDVFFRQKWKDERLKFKG
PMNILRLNNLMASKIWTPDTFFHNGKKSVAHNMTMPNKLLRIQDDGTLLYT
MRLTVQAECPMHLEDFPMDAHSCPLKFGSYAYTTSEVTYIWTYNASDSVQV
APDGSRLNQYDLLGQSIGKETIKSSTGEYTVMTAHFHLKRKIGYFVIQTYLPCI
MTVILSQVSFWLNRESVPARTVFGVTTVLTMTTLSISARNSLPKVAYATAMD
WFIAVCYAFVFSALIEFATVNYFTKRGWAWDGKSVVNDKKKEKASVMIQNN
AYAVAVANYAPNLSKDPVLSTISKSATTPEPNKKPENKPAEAKKTFNSVSKID
RMSRIVFPVLFGTFNLVYWATYLNREPVLGVSP
Human GABA_{A} receptor alpha 3 subunit amino acid
(SEQ ID NO: 31)
MIITQTSHCYMTSLGILFLINILPGTTGQGESRRQEPGDFVKQDIGGLSPKHAPD
IPDDSTDNITIFTRILDRLLDGYDNRLRPGLGDAVTEVKTDIYVTSFGPVSDTD
MEYTIDVFFRQTWHDERLKFDGPMKILPLNNLLASKIWTPDTFFHNGKKSVA
HNMTTPNKLLRLVDNGTLLYTMRLTIHAECPMHLEDFPMDVHACPLKFGSY
AYTTAEVVYSWTLGKNKSVEVAQDGSRLNQYDLLGHVVGTEIIRSSTGEYVV
MTTHFHLKRKIGYFVIQTYLPCIMTVILSQVSFWLNRESVPARTVFGVTTVLT
MTTLSISARNSLPKVAYATAMDWFIAVCYAFVFSALIEFATVNYFTKRSWAW
EGKKVPEALEMKKKTPAAPAKKTSTTFNIVGTTYPINLAKDTEFSTISKGAAPS
ASSTPTIIASPKATYVQDSPTETKTYNSVSKVDKISRIIFPVLFAIFNLVYWATY
VNRESAIKGMIRKQ
Human GABA_{A} receptor alpha 4 subunit amino acid
(SEQ ID NO: 32)
MVSAKKVPAIALSAGVSFALLRFLCLAVCLNESPGQNQKEEKLCTENFTRILD
SLLDGYDNRLRPGFGGPVTEVKTDIYVTSFGPVSDVEMEYTMDVFFRQTWID
KRLKYDGPIEILRLNNMMVTKVWTPDTFFRNGKKSVSHNMTAPNKLFRIMR
NGTILYTMRLTISAECPMRLVDFPMDGHACPLKFGSYAYPKSEMIYTWTKGP
EKSVEVPKESSSLVQYDLIGQTVSSETIKSITGEYIVMTVYFHLRRKMGYFMIQ
TYIPCIMTVILSQVSFWINKESVPARTVFGITTVLTMTTLSISARHSLPKVSYAT
AMDWFIAVCFAFVFSALIEFAAVNYFTNIQMEKAKRKTSKPPQEVPAAPVQR
EKHPEAPLQNTNANLNMRKRTNALVHSESDVGNRTEVGNHSSKSSTVVQESS
KGTPRSYLASSPNPFSRANAAETISAARALPSASPTSIRTGYMPRKASVGSAST
RHVFGSRLQRIKTTVNTIGATGKLSATPPPSAPPPSGSGTSKIDKYARILFPVTF
GAFNMVYWVVYLSKDTMEKSESLM
Human GABA_{A} receptor alpha 5 subunit amino acid
(SEQ ID NO: 33)
MDNGMFSGFIMIKNLLLFCISMNLSSHFGFSQMPTSSVKDETNDNITIFTRILD
GLLDGYDNRLRPGLGERITQVRTDIYVTSFGPVSDTEMEYTIDVFFRQSWKDE
RLRFKGPMQRLPLNNLLASKIWTPDTFFHNGKKSIAHNMTTPNKLLRLEDDG
TLLYTMRLTISAECPMQLEDFPMDAHACPLKFGSYAYPNSEVVYVWTNGSTK
SVVVAEDGSRLNQYHLMGQTVGTENISTSTGEYTIMTAHFHLKRKIGYFVIQT
YLPCIMTVILSQVSFWLNRESVPARTVFGVTTVLTMTTLSISARNSLPKVAYA
TAMDWFIAVCYAFVFSALIEFATVNYFTKRGWAWDGKKALEAAKIKKKREV
ILNKSTNAFTTGKMSHPPNIPKEQTPAGTSNTTSVSVKPSEEKTSESKKTYNSIS
KIDKMSRIVFPVLFGTFNLVYWATYLNREPVIKGAASPK
Human GABA_{A} receptor alpha 6 subunit amino acid
(SEQ ID NO: 34)
MASSLPWLCIILWLENALGKLEVEGNFYSENVSRILDNLLEGYDNRLRPGFGG
AVTEVKTDIYVTSFGPVSDVEMEYTMDVFFRQTWTDERLKFGGPTEILSLNN
LMVSKIWTPDTFFRNGKKSIAHNMTTPNKLFRIMQNGTILYTMRLTINADCPM
RLVNFPMDGHACPLKFGSYAYPKSEIIYTWKKGPLYSVEVPEESSSLLQYDLI
GQTVSSETIKSNTGEYVIMTVYFHLQRKMGYFMIQIYTPCIMTVILSQVSFWIN
KESVPARTVFGITTVLTMTTLSISARHSLPKVSYATAMDWFIAVCFAFVFSALI
EFAAVNYFTNLQTQKAKRKAQFAAPPTVTISKATEPLEAEIVLHPDSKYHLKK
RITSLSLPIVSSSEANKVLTRAPILQSTPVTPPPLSPAFGGTSKIDQYSRILFPVAF
AGFNLVYWVVYLSKDTMEVSSSVE
Human GABA_{A} receptor beta 1 subunit amino acid
(SEQ ID NO: 35)
MWTVQNRESLGLLSFPVMITMVCCAHSTNEPSNMSYVKETVDRLLKGYDIRL
RPDFGGPPVDVGMRIDVASIDMVSEVNMDYTLTMYFQQSWKDKRLSYSGIPL
NLTLDNRVADQLWVPDTYFLNDKKSFVHGVTVKNRMIRLHPDGTVLYGLRI
TTTAACMMDLRRYPLDEQNCTLEIESYGYTTDDIEFYWNGGEGAVTGVNKIE
LPQFSIVDYKMVSKKVEFTTGAYPRLSLSFRLKRNIGYFILQTYMPSTLITILSW
VSFWINYDASAARVALGITTVLTMTTISTHLRETLPKIPYVKAIDIYLMGCFVF
VFLALLEYAFVNYIFFGKGPQKKGASKQDQSANEKNKLEMNKVQVDAHGNI
LLSTLEIRNETSGSEVLTSVSDPKATMYSYDSASIQYRKPLSSREAYGRALDRH
GVPSKGRIRRRASQLKVKIPDLTDVNSIDKWSRMFFPITFSLFNVVYWLYYVH
Human GABA_{A} receptor beta 2 variant 1 (long) subunit amino acid
(SEQ ID NO: 36)
WRVRKRGYFGIWSFPLIIAAVCAQSVNDPSNMSLVKETVDRLLKGYDIRLRP
DFGGPPVAVGMNIDIASIDMVSEVNMDYTLTMYFQQAWRDKRLSYNVIPLN
LTLDNRVADQLWVPDTYFLNDKKSFVHGVTVKNRMIRLHPDGTVLYGLRIT
TTAACMMDLRRYPLDEQNCTLEIESYGYTTDDIEFYWRGDDNAVTGVTKIEL
PQFSIVDYKLITKKVVFSTGSYPRLSLSFKLKRNIGYFILQTYMPSILITILSWVS
FWINYDASAARVALGITTVLTMTTINTHLRETLPKIPYVKAIDMYLMGCFVFV
FMALLEYALVNYIFFGRGPQRQKKAAEKAASANNEKMRLDVNKIFYKDIKQ
NGTQYRSLWDPTGNLSPTRRTTNYDFSLYTMDPHENILLSTLEIKNEMATSEA
VMGLGDPRSTMLAYDASSIQYRKAGLPRHSFGRNALERHVAQKKSRLRRRA
SQLKITIPDLTDVNAIDRWSRIFFPVVFSFFNIVYWLYYVN
Human GABA_{A} receptor beta 2 variant 2 (short) subunit amino acid
(SEQ ID NO: 37)
MWRVRKRGYFGIWSFPLIIAAVCAQSVNDPSNMSLVKETVDRLLKGYDIRLR
PDFGGPPVAVGMNIDIASIDMVSEVNMDYTLTMYFQQAWRDKRLSYNVIPLN
LTLDNRVADQLWVPDTYFLNDKKSFVHGVTVKNRMIRLHPDGTVLYGLRIT
TTAACMMDLRRYPLDEQNCTLEIESYGYTTDDIEFYWRGDDNAVTGVTKIEL
PQFSIVDYKLITKKVVFSTGSYPRLSLSFKLKRNIGYFILQTYMPSILITILSWVS
FWINYDASAARVALGITTVLTMTTINTHLRETLPKIPYVKAIDMYLMGCFVFV
FMALLEYALVNYIFFGRGPQRQKKAAEKAASANNEKMRLDVNKMDPHENIL
LSTLEIKNEMATSEAVMGLGDPRSTMLAYDASSIQYRKAGLPRHSFGRNALE
RHVAQKKSRLRRRASQLKITIPDLTDVNAIDRWSRIFFPVVFSFFNIVYWLYYVN
Human GABA_{A} receptor beta 3 variant 1 subunit amino acid
(SEQ ID NO: 38)
MWGLAGGRLFGIFSAPVLVAVVCCAQSVNDPGNMSFVKETVDKLLKGYDIR
LRPDFGGPPVCVGMNIDIASIDMVSEVNMDYTLTMYFQQYWRDKRLAYSGIP
LNLTLDNRVADQLWVPDTYFLNDKKSFVHGVTVKNRMIRLHPDGTVLYGLR
ITTTAACMMDLRRYPLDEQNCTLEIESYGYTTDDIEFYWRGGDKAVTGVERI
ELPQFSIVEHRLVSRNVVFATGAYPRLSLSFRLKRNIGYFILQTYMPSILITILSW
VSFWINYDASAARVALGITTVLTMTTINTHLRETLPKIPYVKAIDMYLMGCFV
FVFLALLEYAFVNYIFFGRGPQRQKKLAEKTAKAKNDRSKSESNRVDAHGNI
LLTSLEVHNEMNEVSGGIGDTRNSAISFDNSGIQYRKQSMPREGHGRFLGDRS
LPHKKTHLRRRSSQLKIKIPDLTDVNAIDRWSRIVFPFTFSLFNLVYWLYYVN
Human GABA_{A} receptor beta 3 variant 2 subunit amino acid
(SEQ ID NO: 39)
MCSGLLELLLPIWLSWTLGTRGSEPRSVNDPGNMSFVKETVDKLLKGYDIRL
RPDFGGPPVCVGMNIDIASIDMVSEVNMDYTLTMYFQQYWRDKRLAYSGIPL
NLTLDNRVADQLWVPDTYFLNDKKSFVHGVTVKNRMIRLHPDGTVLYGLRI
TTTAACMMDLRRYPLDEQNCTLEIESYGYTTDDIEFYWRGGDKAVTGVERIE
LPQFSIVEHRLVSRNVVFATGAYPRLSLSFRLKRNIGYFILQTYMPSILITILSW
VSFWINYDASAARVALGITTVLTMTTINTHLRETLPKIPYVKAIDMYLMGCFV
FVFLALLEYAFVNYIFFGRGPQRQKKLAEKTAKAKNDRSKSESNRVDAHGNI
LLTSLEVHNEMNEVSGGIGDTRNSAISFDNSGIQYRKQSMPREGHGRFLGDRS
LPHKKTHLRRRSSQLKIKIPDLTDVNAIDRWSRIVFPFTFSLFNLVYWLYYVN
Human GABA_{A} receptor gamma 1 subunit amino acid
(SEQ ID NO: 40)
MGPLKAFLFSPFLLRSQSRGVRLVFLLLTLHLGNCVDKADDEDDEDLTVNKT
WVLAPKIHEGDITQILNSLLQGYDNKLRPDIGVRPTVIETDVYVNSIGPVDPIN
MEYTIDIIFAQTWFDSRLKFNSTMKVLMLNSNMVGKIWIPDTFFRNSRKSDAH
WITTPNRLLRIWNDGRVLYTLRLTINAECYLQLHNFPMDEHSCPLEFSSYGYP
KNEIEYKWKKPSVEVADPKYWRLYQFAFVGLRNSTEITHTISGDYVIMTIFFD
LSRRMGYFTIQTYIPCILTVVLSWVSFWINKDAVPARTSLGITTVLTMTTLSTI
ARKSLPKVSYVTAMDLFVSVCFIFVFAALMEYGTLHYFTSNQKGKTATKDRK
LKNKASMTPGLHPGSTLIPMNNISVPQEDDYGYQCLEGKDCASFFCCFEDCRT
GSWREGRIHIRIAKIDSYSRIFFPTAFALFNLVYWVGYLYL
Human GABA_{A} receptor gamma 2 variant 1 (short) subunit amino acid
(SEQ ID NO: 41)
MSSPNIWSTGSSVYSTPVFSQKMTVWILLLLSLYPGFTSQKSDDDYEDYASNK
TWVLTPKVPEGDVTVILNNLLEGYDNKLRPDIGVKPTLIHTDMYVNSIGPVNA
INMEYTIDIFFAQTWYDRRLKFNSTIKVLRLNSNMVGKIWIPDTFFRNSKKAD
AHWITTPNRMLRIWNDGRVLYTLRLTIDAECQLQLHNFPMDEHSCPLEFSSY
GYPREEIVYQWKRSSVEVGDTRSWRLYQFSFVGLRNTTEVVKTTSGDYVVM
SVYFDLSRRMGYFTIQTYIPCTLIVVLSWVSFWINKDAVPARTSLGITTVLTMT
TLSTIARKSLPKVSYVTAMDLFVSVCFIFVFSALVEYGTLHYFVSNRKPSKDK
DKKKKNPAPTIDIRPRSATIQMNNATHLQERDEEYGYECLDGKDCASFFCCFE
DCRTGAWRHGRIHIRIAKMDSYARIFFPTAFCLFNLVYWVSYLYL
Human GABA_{A} receptor gamma 2 variant 2 (long) subunit amino acid
(SEQ ID NO: 42)
MSSPNIWSTGSSVYSTPVFSQKMTVWILLLLSLYPGFTSQKSDDDYEDYASNK
TWVLTPKVPEGDVTVILNNLLEGYDNKLRPDIGVKPTLIHTDMYVNSIGPVNA
INMEYTIDIFFAQTWYDRRLKFNSTIKVLRLNSNMVGKIWIPDTFFRNSKKAD
AHWITTPNRMLRIWNDGRVLYTLRLTIDAECQLQLHNFPMDEHSCPLEFSSY
GYPREEIVYQWKRSSVEVGDTRSWRLYQFSFVGLRNTTEVVKTTSGDYVVM
SVYFDLSRRMGYFTIQTYIPCTLIVVLSWVSFWINKDAVPARTSLGITTVLTMT
TLSTIARKSLPKVSYVTAMDLFVSVCFIFVFSALVEYGTLHYFVSNRKPSKDK
DKKKKNPLLRMFSFKAPTIDIRPRSATIQMNNATHLQERDEEYGYECLDGKD
CASFFCCFEDCRTGAWRHGRIHIRIAKMDSYARIFFPTAFCLFNLVYWVSYLYL
Human GABA_{A} receptor gamma 3 subunit amino acid
(SEQ ID NO: 43)
MAPKLLLLLCLFSGLHARSRKVEEDEYEDSSSNQKWVLAPKSQDTDVTLILN
KLLREYDKKLRPDIGIKPTVIDVDIYVNSIGPVSSINMEYQIDIFFAQTWTDSRL
RFNSTMKILTLNSNMVGLIWIPDTIFRNSKTAEAHWITTPNQLLRIWNDGKILY
TLRLTINAECQLQLHNFPMDEHSCPLIFSSYGYPKEEMIYRWRKNSVEAADQK
SWRLYQFDFMGLRNTTEIVTTSAGDYVVMTIYFELSRRMGYFTIQTYIPCILTV
VLSWVSFWIKKDATPARTALGITTVLTMTTLSTIARKSLPRVSYVTAMDLFVT
VCFLFVFAALMEYATLNYYSSCRKPTTTKKTTSLLHPDSSRWIPERISLQAPSN
YSLLDMRPPPTAMITLNNSVYWQEFEDTCVYECLDGKDCQSFFCCYEECKSG
SWRKGRIHIDILELDSYSRVFFPTSFLLFNLVYWVGYLYL
Human GABA_{A} receptor delta subunit amino acid
(SEQ ID NO: 44)
MDAPARLLAPLLLLCAQQLRGTRAMNDIGDYVGSNLEISWLPNLDGLIAGYA
RNFRPGIGGPPVNVALALEVASIDHISEANMEYTMTVFLHQSWRDSRLSYNH
TNETLGLDSRFVDKLWLPDTFIVNAKSAWFHDVTVENKLIRLQPDGVILYSIRI
TSTVACDMDLAKYPMDEQECMLDLESYGYSSEDIVYYWSESQEHIHGLDKL
QLAQFTITSYRFTTELMNFKSAGQFPRLSLHFHLRRNRGVYIIQSYMPSVLLVA
MSWVSFWISQAAVPARVSLGITTVLTMTTLMVSARSSLPRASAIKALDVYFWI
CYVFVFAALVEYAFAHFNADYRKKQKAKVKVSRPRAEMDVRNAIVLFSLSA
AGVTQELAISRRQRRVPGNLMGSYRSVGVETGETKKEGAARSGGQGGIRARL
RPIDADTIDIYARAVFPAAFAAVNVIYWAAYAM
Human GABA_{A} receptor epsilon subunit amino acid
(SEQ ID NO: 45)
MLSKVLPVLLGILLILQSRVEGPQTESKNEASSRDVVYGPQPQPLENQLLSEET
KSTETETGSRVGKLPEASRILNTILSNYDHKLRPGIGEKPTVVTVEISVNSLGPL
SILDMEYTIDIIFSQTWYDERLCYNDTFESLVLNGNVVSQLWIPDTFFRNSKRT
HEHEITMPNQMVRIYKDGKVLYTIRMTIDAGCSLHMLRFPMDSHSCPLSFSSF
SYPENEMIYKWENFKLEINEKNSWKLFQFDFTGVSNKTEIITTPVGDFMVMTI
FFNVSRRFGYVAFQNYVPSSVTTMLSWVSFWIKTESAPARTSLGITSVLTMTT
LGTFSRKNFPRVSYITALDFYIAICFVFCFCALLEFAVLNFLIYNQTKAHASPKL
RHPRINSRAHARTRARSRACARQHQEAFVCQIVTTEGSDGEERPSCSAQQPPS
PGSPEGPRSLCSKLACCEWCKRFKKYFCMVPDCEGSTWQQGRLCIHVYRLDN
YSRVVFPVTFFFFNVLYWLVCLNL
Human GABA_{A} receptor pi subunit amino acid
(SEQ ID NO: 46)
MNYSLHLAFVCLSLFTERMCIQGSQFNVEVGRSDKLSLPGFENLTAGYNKFL
RPNFGGEPVQIALTLDIASISSISESNMDYTATIYLRQRWMDQRLVFEGNKSFT
LDARLVEFLWVPDTYIVESKKSFLHEVTVGNRLIRLFSNGTVLYALRITTTVA
CNMDLSKYPMDTQTCKLQLESWGYDGNDVEFTWLRGNDSVRGLEHLRLAQ
YTIERYFTLVTRSQQETGNYTRLVLQFELRRNVLYFILETYVPSTFLVVLSWVS
FWISLDSVPARTCIGVTTVLSMTTLMIGSRTSLPNTNCFIKAIDVYLGICFSFVF
GALLEYAVAHYSSLQQMAAKDRGTTKEVEEVSITNIINSSISSFKRKISFASIEIS
SDNVDYSDLTMKTSDKFKFVFREKMGRIVDYFTIQNPSNVDHYSKLLFPLIFM
LANVFYWAYYMYF
Human GABA_{A} receptor theta subunit amino acid
(SEQ ID NO: 47)
MGIRGMLRAAVILLLIRTWLAEGNYPSPIPKFHFEFSSAVPEVVLNLFNCKNC
ANEAVVQKILDRVLSRYDVRLRPNFGGAPVPVRISIYVTSIEQISEMNMDYTIT
MFFHQTWKDSRLAYYETTLNLTLDYRMHEKLWVPDCYFLNSKDAFVHDVT
VENRVFQLHPDGTVRYGIRLTTTAACSLDLHKFPMDKQACNLVVESYGYTVE
DIILFWDDNGNAIHMTEELHIPQFTFLGRTITSKEVYFYTGSYIRLILKFQVQRE
VNSYLVQVYWPTVLTTITSWISFWMNYDSSAARVTIGLTSMLILTTIDSHLRD
KLPNISCIKAIDIYILVCLFFVFLSLLEYVYINYLFYSRGPRRQPRRHRRPRRVIA
RYRYQQVVVGNVQDGLINVEDGVSSLPITPAQAPLASPESLGSLTSTSEQAQL
ATSESLSPLTSLSGQAPLATGESLSDLPSTSEQARHSYGVRFNGFQADDSIFPTE
IRNRVEAHGHGVTHDHEDSNESLSSDERHGHGPSGKPMLHHGEKGVQEAGW
DLDDNNDKSDCLAIKEQFKCDTNSTWGLNDDELMAHGQEKDSSSESEDSCPP
SPGCSFTEGFSFDLFNPDYVPKVDKWSRFLFPLAFGLFNIVYWVYHMY
Human GABA_{C} receptor rho 1 subunit amino acid
(SEQ ID NO: 48)
MRFGIFLLWWGWVLATESRMHWPGREVHEMSKKGRPQRQRREVHEDAHK
QVSPILRRSPDITKSPLTKSEQLLRIDDHDFSMRPGFGGPAIPVGVDVQVESLDS
ISEVDMDFTMTLYLRHYWKDERLSFPSTNNLSMTFDGRLVKKIWVPDMFFV
HSKRSFIHDTTTDNVMLRVQPDGKVLYSLRVTVTAMCNMDFSRFPLDTQTCS
LEIESYAYTEDDLMLYWKKGNDSLKTDERISLSQFLIQEFHTTTKLAFYSSTG
WYNRLYINFTLRRHIFFFLLQTYFPATLMVMLSWVSFWIDRRAVPARVPLGIT
TVLTMSTIITGVNASMPRVSYIKAVDIYLWVSFVFVFLSVLEYAAVNYLTTVQ
ERKEQKLREKLPCTSGLPPPRTAMLDGNYSDGEVNDLDNYMPENGEKPDRM
MVQLTLASERSSPQRKSQRSSYVSMRIDTHAIDKYSRIIFPAAYILFNLIYWSIFS
Human GABA_{C} receptor rho2 subunit amino acid
(SEQ ID NO: 49)
MPYFTRLILFLFCLMVLVESRKPKRKRWTGQVEMPKPSHLYKKNLDVTKIRK
GKPQQLLRVDEHDFSMRPAFGGPAIPVGVDVQVESLDSISEVDMDFTMTLYL
RHYWKDERLAFSSASNKSMTFDGRLVKKIWVPDVFFVHSKRSFTHDTTTDNI
MLRVFPDGHVLYSMRITVTAMCNMDFSHFPLDSQTCSLELESYAYTDEDLML
YWKNGDESLKTDEKISLSQFLIQKFHTTSRLAFYSSTGWYNRLYINFTLRRHIF
FFLLQTYFPATLMVMLSWVSFWIDRRAVPARVSLGITTVLTMTTIITGVNASM
PRVSYVKAVDIYLWVSFVFVFLSVLEYAAVNYLTTVQERKERKLREKFPCMC
GMLHSKTMMLDGSYSESEANSLAGYPRSHILTEEERQDKIVVHLGLSGEANA
ARKKGLLKGQTGFRIFQNTHAIDKYSRLIFPASYIFFNLIYWSVFS
Human GABA_{C} receptor rho3 subunit amino acid
(SEQ ID NO: 50)
MVLAFQLVSFTYIWIILKPNVCAASNIKMTHQRCSSSMKQTCKQETRMKKDD
STKARPQKYEQLLHIEDNDFAMRPGFGGSPVPVGIDVHVESIDSISETNMDFT
MTFYLRHYWKDERLSFPSTANKSMTFDHRLTRKIWVPDIFFVHSKRSFIHDTT
MENIMLRVHPDGNVLLSLRITVSAMCFMDFSRFPLDTQNCSLELESYAYNED
DLMLYWKHGNKSLNTEEHMSLSQFFIEDFSASSGLAFYSSTGWYNRLFINFVL
RRHVFFFVLQTYFPAILMVMLSWVSFWIDRRAVPARVSLGITTVLTMSTIITA
VSASMPQVSYLKAVDVYLWVSSLFVFLSVIEYAAVNYLTTVEERKQFKKTGK
ISRMYNIDAVQAMAFDGCYHDSEIDMDQTSLSLNSEDFMRRKSICSPSTDSSRI
KRRKSLGGHVGRIILENNHVIDTYSRILFPIVYILFNLFYWGVYV
Human GABA_{B} receptor 1 isoform 1A subunit amino acid
(SEQ ID NO: 51)
MLLLLLLAPLFLRPPGAGGAQTPNATSEGCQIIHPPWEGGIRYRGLTRDQVKA
INFLPVDYEIEYVCRGEREVVGPKVRKCLANGSWTDMDTPSRCVRICSKSYLT
LENGKVFLTGGDLPALDGARVDFRCDPDFHLVGSSRSICSQGQWSTPKPHCQ
VNRTPHSERRAVYIGALFPMSGGWPGGQACQPAVEMALEDVNSRRDILPDYE
LKLIHHDSKCDPGQATKYLYELLYNDPIKIILMPGCSSVSTLVAEAARMWNLI
VLSYGSSSPALSNRQRFPTFFRTHPSATLHNPTRVKLFEKWGWKKIATIQQTT
EVFTSTLDDLEERVKEAGIEITFRQSFFSDPAVPVKNLKRQDARIIVGLFYETEA
RKVFCEVYKERLFGKKYVWFLIGWYADNWFKIYDPSINCTVDEMTEAVEGHI
TTEIVMLNPANTRSISNMTSQEFVEKLTKRLKRHPEETGGFQEAPLAYDAIWA
LALALNKTSGGGGRSGVRLEDFNYNNQTITDQIYRAMNSSSFEGVSGHVVFD
ASGSRMAWTLIEQLQGGSYKKIGYYDSTKDDLSWSKTDKWIGGSPPADQTL
VIKTFRFLSQKLFISVSVLSSLGIVLAVVCLSFNIYNSHVRYIQNSQPNLNNLTA
VGCSLALAAVFPLGLDGYHIGRNQFPFVCQARLWLLGLGFSLGYGSMFTKIW
WVHTVFTKKEEKKEWRKTLEPWKLYATVGLLVGMDVLTLAIWQIVDPLHRT
IETFAKEEPKEDIDVSILPQLEHCSSRKMNTWLGIFYGYKGLLLLLGIFLAYET
KSVSTEKINDHRAVGMAIYNVAVLCLITAPVTMILSSQQDAAFAFASLAIVFSS
YITLVVLFVPKMRRLITRGEWQSEAQDTMKTGSSTNNNEEEKSRLLEKENRE
LEKIIAEKEERVSELRHQLQSRQQLRSRRHPPTPPEPSGGLPRGPPEPPDRLSCD
GSRVHLLYK
Human GABA_{B} receptor 1 isoform 1B subunit amino acid
(SEQ ID NO: 52)
MGPGAPFARVGWPLPLLVVMAAGVAPVWASHSPHLPRPHSRVPPHPSSERR
AVYIGALFPMSGGWPGGQACQPAVEMALEDVNSRRDILPDYELKLIHHDSKC
DPGQATKYLYELLYNDPIKIILMPGCSSVSTLVAEAARMWNLIVLSYGSSSPA
LSNRQRFPTFFRTHPSATLHNPTRVKLFEKWGWKKIATIQQTTEVFTSTLDDL
EERVKEAGIEITFRQSFFSDPAVPVKNLKRQDARIIVGLFYETEARKVFCEVYK
ERLFGKKYVWFLIGWYADNWFKIYDPSINCTVDEMTEAVEGHITTEIVMLNP
ANTRSISNMTSQEFVEKLTKRLKRHPEETGGFQEAPLAYDAIWALALALNKTS
GGGGRSGVRLEDFNYNNQTITDQIYRAMNSSSFEGVSGHVVFDASGSRMAW
TLIEQLQGGSYKKIGYYDSTKDDLSWSKTDKWIGGSPPADQTLVIKTFRFLSQ
KLFISVSVLSSLGIVLAVVCLSFNIYNSHVRYIQNSQPNLNNLTAVGCSLALAA
VFPLGLDGYHIGRNQFPFVCQARLWLLGLGFSLGYGSMFTKIWWVHTVFTK
KEEKKEWRKTLEPWKLYATVGLLVGMDVLTLAIWQIVDPLHRTIETFAKEEP
KEDIDVSILPQLEHCSSRKMNTWLGIFYGYKGLLLLLGIFLAYETKSVSTEKIN
DHRAVGMAIYNVAVLCLITAPVTMILSSQQDAAFAFASLAIVFSSYITLVVLFV
PKMRRLITRGEWQSEAQDTMKTGSSTNNNEEEKSRLLEKENRELEKIIAEKEE
RVSELRHQLQSRQQLRSRRHPPTPPEPSGGLPRGPPEPPDRLSCDGSRVHLLYK
Human GABA_{B} receptor 1 isoform 1C subunit amino acid
(SEQ ID NO: 53)
MLLLLLLAPLFLRPPGAGGAQTPNATSEGCQIIHPPWEGGIRYRGLTRDQVKA
INFLPVDYEIEYVCRGEREVVGPKVRKCLANGSWTDMDTPSRCVNRTPHSER
RAVYIGALFPMSGGWPGGQACQPAVEMALEDVNSRRDILPDYELKLIHHDSK
CDPGQATKYLYELLYNDPIKIILMPGCSSVSTLVAEAARMWNLIVLSYGSSSP
ALSNRQRFPTFFRTHPSATLHNPTRVKLFEKWGWKKIATIQQTTEVFTSTLDD
LEERVKEAGIEITFRQSFFSDPAVPVKNLKRQDARIIVGLFYETEARKVFCEVY
KERLFGKKYVWFLIGWYADNWFKIYDPSINCTVDEMTEAVEGHITTEIVMLN
PANTRSISNMTSQEFVEKLTKRLKRHPEETGGFQEAPLAYDAIWALALALNKT
SGGGGRSGVRLEDFNYNNQTITDQIYRAMNSSSFEGVSGHVVFDASGSRMA
WTLIEQLQGGSYKKIGYYDSTKDDLSWSKTDKWIGGSPPADQTLVIKTFRFLS
QKLFISVSVLSSLGIVLAVVCLSFNIYNSHVRYIQNSQPNLNNLTAVGCSLALA
AVFPLGLDGYHIGRNQFPFVCQARLWLLGLGFSLGYGSMFTKIWWVHTVFT
KKEEKKEWRKTLEPWKLYATVGLLVGMDVLTLAIWQIVDPLHRTIETFAKEE
PKEDIDVSILPQLEHCSSRKMNTWLGIFYGYKGLLLLLGIFLAYETKSVSTEKI
NDHRAVGMAIYNVAVLCLITAPVTMILSSQQDAAFAFASLAIVFSSYITLVVLF
VPKMRRLITRGEWQSEAQDTMKTGSSTNNNEEEKSRLLEKENRELEKIIAEKE
ERVSELRHQLQSRQQLRSRRHPPTPPEPSGGLPRGPPEPPDRLSCDGSRVHLLYK
Human GABA_{B} receptor 1 isoform 1D subunit amino acid
(SEQ ID NO: 54)
MLLLLLLAPLFLRPPGAGGAQTPNATSEGCQIIHPPWEGGIRYRGLTRDQVKA
INFLPVDYEIEYVCRGEREVVGPKVRKCLANGSWTDMDTPSRCVRICSKSYLT
LENGKVFLTGGDLPALDGARVDFRCDPDFHLVGSSRSICSQGQWSTPKPHCQ
VNRTPHSERRAVYIGALFPMSGGWPGGQACQPAVEMALEDVNSRRDILPDYE
LKLIHHDSKCDPGQATKYLYELLYNDPIKIILMPGCSSVSTLVAEAARMWNLI
VLSYGSSSPALSNRQRFPTFFRTHPSATLHNPTRVKLFEKWGWKKIATIQQTT
EVFTSTLDDLEERVKEAGIEITFRQSFFSDPAVPVKNLKRQDARIIVGLFYETEA
RKVFCEVYKERLFGKKYVWFLIGWYADNWFKIYDPSINCTVDEMTEAVEGHI
TTEIVMLNPANTRSISNMTSQEFVEKLTKRLKRHPEETGGFQEAPLAYDAIWA
LALALNKTSGGGGRSGVRLEDFNYNNQTITDQIYRAMNSSSFEGVSGHVVFD
ASGSRMAWTLIEQLQGGSYKKIGYYDSTKDDLSWSKTDKWIGGSPPADQTL
VIKTFRFLSQKLFISVSVLSSLGIVLAVVCLSFNIYNSHVRYIQNSQPNLNNLTA
VGCSLALAAVFPLGLDGYHIGRNQFPFVCQARLWLLGLGFSLGYGSMFTKIW
WVHTVFTKKEEKKEWRKTLEPWKLYATVGLLVGMDVLTLAIWQIVDPLHRT
IETFAKEEPKEDIDVSILPQLEHCSSRKMNTWLGIFYGYKGLLLLLGIFLAYET
KSVSTEKINDHRAVGMAIYNVAVLCLITAPVTMILSSQQDAAFAFASLAIVFSS
YITLVVLFVPKMRRLITRGEWQSEAQDTMKTGSSTNNNEEEKSRLLEKENRE
LEKIIAESGGLPRGPPEPPDRLSCDGSRVHLLYK
Human GABA_{B} receptor 1 isoform 1E subunit amino acid
(SEQ ID NO: 55)
MLLLLLLAPLFLRPPGAGGAQTPNATSEGCQIIHPPWEGGIRYRGLTRDQVKA
INFLPVDYEIEYVCRGEREVVGPKVRKCLANGSWTDMDTPSRCVRICSKSYLT
LENGKVFLTGGDLPALDGARVDFRCDPDFHLVGSSRSICSQGQWSTPKPHCQ
VNRTPHSERRAVYIGALFPMSGGWPGGQACQPAVEMALEDVNSRRDILPDYE
LKLIHHDSKCDPGQATKYLYELLYNDPIKIILMPGCSSVSTLVAEAARMWNLI
VLSYGSSSPALSNRQRFPTFFRTHPSATLHNPTRVKLFEKWGWKKIATIQQTT
EVFTSTLDDLEERVKEAGIEITFRQSFFSDPAVPVKNLKRQDARIIVGLFYETEA
RKVFCEVYKERLFGKKYVWFLIGWYADNWFKIYDPSINCTVDEMTEAVEGHI
TTEIVMLNPANTRSISNMTSQEFVEKLTKRLKRHPEETGGFQEAPLAYDAIWA
LALALNKTSGGGGRSGVRLEDFNYNNQTITDQIYRAMNSSSFEGVSGHVVFD
ASGSRMAWTLIEQLQGGSYKKIGYYDSTKDDLSWSKTDKWIVISRTHSPT
Human GABA_{B} receptor 2 subunit amino acid
(SEQ ID NO: 56)
MASPRSSGQPGPPPPPPPPPARLLLLLLLPLLLPLAPGAWGWARGAPRPPPSSP
PLSIMGLMPLTKEVAKGSIGRGVLPAVELAIEQIRNESLLRPYFLDLRLYDTEC
DNAKGLKAFYDAIKYGPNHLMVFGGVCPSVTSIIAESLQGWNLVQLSFAATT
PVLADKKKYPYFFRTVPSDNAVNPAILKLLKHYQWKRVGTLTQDVQRFSEV
RNDLTGVLYGEDIEISDTESFSNDPCTSVKKLKGNDVRIILGQFDQNMAAKVF
CCAYEENMYGSKYQWIIPGWYEPSWWEQVHTEANSSRCLRKNLLAAMEGYI
GVDFEPLSSKQIKTISGKTPQQYEREYNNKRSGVGPSKFHGYAYDGIWVIAKT
LQRAMETLHASSRHQRIQDFNYTDHTLGRIILNAMNETNFFGVTGQVVFRNG
ERMGTIKFTQFQDSREVKVGEYNAVADTLEIINDTIRFQGSEPPKDKTIILEQLR
KISLPLYSILSALTILGMIMASAFLFFNIKNRNQKLIKMSSPYMNNLIILGGMLS
YASIFLFGLDGSFVSEKTFETLCTVRTWILTVGYTTAFGAMFAKTWRVHAIFK
NVKMKKKIIKDQKLLVIVGGMLLIDLCILICWQAVDPLRRTVEKYSMEPDPA
GRDISIRPLLEHCENTHMTIWLGIVYAYKGLLMLFGCFLAWETRNVSIPALND
SKYIGMSVYNVGIMCIIGAAVSFLTRDQPNVQFCIVALVIIFCSTITLCLVFVPK
LITLRTNPDAATQNRRFQFTQNQKKEDSKTSTSVTSVNQASTSRLEGLQSENH
RLRMKITELDKDLEEVTMQLQDTPEKTTYIKQNHYQELNDILNLGNFTESTDG
GKAILKNHLDQNPQLQWNTTEPSRTCKDPIEDINSPEHIQRRLSLQLPILHHAY
LPSIGGVDASCVSPCVSPTASPRHRHVPPSFRVMVSGL
YFP mutant (meYFP-H148Q/I152L)
(SEQ ID NO: 57)
ATGGTGAGCAAGGGCGAGGAGCTGTTCACCGGGGTGGTGCCCATCCTGGT
CGAGCTGGACGGCGACGTAAACGGCCACAAGTTCAGCGTGTCCGGCGAG
GGCGAGGGCGATGCCACCTACGGCAAGCTGACCCTGAAGTTCATCTGCAC
CACCGGCAAGCTGCCCGTGCCCTGGCCCACCCTCGTGACCACCTTCGGCTA
CGGCCTGCAGTGCTTCGCCCGCTACCCCGACCACATGAAGCAGCACGACT
TCTTCAAGTCCGCCATGCCCGAAGGCTACGTCCAGGAGCGCACCATCTTCT
TCAAGGACGACGGCAACTACAAGACCCGCGCCGAGGTGAAGTTCGAGGG
CGACACCCTGGTGAACCGCATCGAGCTGAAGGGCATCGACTTCAAGGAGG
ACGGCAACATCCTGGGGCACAAGCTGGAGTACAACTACAACAGCCAAAA
CGTCTATCTCATGGCCGACAAGCAGAAGAACGGCATCAAGGTGAACTTCA
AGATCCGCCACAACATCGAGGACGGCAGCGTGCAGCTCGCCGACCACTAC
CAGCAGAACACCCCCATCGGCGACGGCCCCGTGCTGCTGCCCGACAACCA
CTACCTGAGCTACCAGTCCGCCCTGAGCAAAGACCCCAACGAGAAGCGCG
ATCACATGGTCCTGCTGGAGTTCGTGACCGCCGCCGGGATCACTCTCGGC
ATGGACGAGCTGTACAAGTAA
Target 1
(SEQ ID NO: 58)
5′-GTTCTTAAGGCACAGGAACTGGGAC-3′
Target 2
(SEQ ID NO: 59)
5′-GAAGTTAACCCTGTCGTTCTGCGAC-3′
Target 3
(SEQ ID NO: 60)
5′-GTTCTATAGGGTCTGCTTGTCGCTC-3′
Signal Probe 1
(SEQ ID NO: 61)
5′-Cy5 GCCAGTCCCAGTTCCTGTGCCTTAAGAACCTCGC BHQ3 quench-3′
Signal Probe 2
(SEQ ID NO: 62)
5′-Cy5.5 GCGAGTCGCAGAACGACAGGGTTAACTTCCTCGC BHQ3 quench-3′
Signal Probe 3
(SEQ ID NO: 63)
5′-Fam GCGAGAGCGACAAGCAGACCCTATAGAACCTCGC BHQ1 quench-3′′

1. A cell or cell line engineered to stably express a GABA receptor comprising one or more subunits at a consistent level over time, said subunits selected from the group consisting of: alpha 1, alpha 2, alpha 3, alpha 4, alpha 5, alpha 6, beta 1, beta 2 (short), beta 2 (long), beta 3 (isoform 1), beta 3 (isoform 2), gamma 1, gamma 2 (short), gamma 2 (long), gamma 3, delta, epsilon, pi, theta, rho 1, rho 2, rho 3, GABA_{B} receptor 1A, GABA_{B} receptor 1B, GABA_{B} receptor 1C, GABA_{B} receptor 1D, GABA_{B} receptor 1E, and GABA_{B} receptor 2.

2. A cell or cell line engineered to stably express a GABA_{A} receptor comprising one or more subunits at a consistent level over time, said subunits selected from the group consisting of: alpha 1, alpha 2, alpha 3, alpha 4, alpha 5, alpha 6, beta 1, beta 2 (short), beta 2 (long), beta 3 (isoform 1), beta 3 (isoform 2), gamma 1, gamma 2 (short), gamma 2 (long), gamma 3, delta, epsilon, pi, and theta.

3. A cell or cell line engineered to stably express a GABA_{B} receptor comprising one or more subunits at a consistent level over time, said subunits selected from the group consisting of: GABA_{B} receptor 1A, GABA_{B} receptor 1B, GABA_{B} receptor 1C, GABA_{B} receptor 1D, GABA_{B} receptor 1E, and GABA_{B} receptor 2.

4. A cell or cell line engineered to stably express a GABA_{c} receptor comprising one or more subunits at a consistent level over time, said subunits selected from the group consisting of rho 1, rho 2, and rho 3.

5. The cell or cell line according to claim 1 or 2, wherein said GABA, GABA_{A}, GABA_{B}, or GABA_{C} receptor comprises:
(a) two or more subunits;
(b) three or more subunits;
(c) four or more subunits; or
(d) five or more subunits.

6-8. (canceled)

9. The cell or cell line of claim 5, wherein said GABA receptor subunits are expressed from separate nucleic acids introduced into the cell line.

10. The cell or cell line of claim 5, wherein two or more of said GABA receptor subunits are expressed from the same nucleic acid introduced into the cell line.

11. The cell or cell line of claim 2, wherein said GABA_{A} receptor comprises:
i) a) at least one GABA_{A} alpha subunit;
b) at least one GABA_{A} beta subunit; and
c) at least one GABA_{A} subunit is selected from the group consisting of gamma 1, gamma 2 (short), gamma 2 (long), gamma 3, delta, epsilon, pi, or theta; or
ii) a) two alpha subunits
b) two beta subunits; and
c) one subunit selected from the group consisting of gamma 1, gamma 2 (short), gamma 2 (long), gamma 3, delta, epsilon, pi, or theta.

12. (canceled)

13. The cell or cell line of claim 1 or 2, wherein said cell or cell line:
a) is a eukaryotic cell or cell line;
b) is a mammalian cell or cell line;
c) does not endogenously express any GABA receptor subunit;
d) does not endogenously express said GABA receptor subunits; or
e) any combination of (a), (b) (c) and (d).

14-15. (canceled)

16. The cell or cell line of claim 1 or 2, wherein said GABA receptor subunits:
a) are mammalian;
b) lack a polypeptide tag at the amino terminus and the carboxyl terminus; or
c) are both a) and b).

17. The cell or cell line of claim 1 or 2, wherein all of said GABA receptor subunits are from the same species.

18. The cell or cell line of claim 17, wherein all of said GABA receptor subunits are human.

19. The cell or cell line of claim 1 or 2, wherein said GABA receptor subunits are from two or more different species.

20-22. (canceled)

23. The cell or cell line of claim 1 or 2, wherein said cell or cell line stably expresses the GABA receptor subunit in culture media without selection for at least 2 weeks, 4 weeks, 6 weeks, 3 months, 6 months or 9 months.

24. The cell or cell line of claim 1, wherein said GABA receptor subunits comprise at least one amino acid encoded by a nucleic acid selected from the group consisting of
a) any one of SEQ ID NOs: 1-28;
b) a nucleic acid that is at least 95% identical to any one of SEQ ID NOs: 1-28;
c) a nucleic acid that hybridizes to the reverse-complement of any one of SEQ ID NOs: 1-28 under stringent conditions; and
d) a nucleic acid that is an allelic variant of any one of SEQ ID NOS:1-28.

25. The cell or cell line of claim 24, wherein said GABA receptor subunits comprise:
a) at least one amino acid encoded by a nucleic acid selected from the group consisting of SEQ ID NO: 1-6;
b) at least one amino acid encoded by a nucleic acid selected from the group consisting of: SEQ ID NO: 7-11; and
c) at least one amino acid encoded by a nucleic acid selected from the group consisting of SEQ ID NO: 12-22.

26. The cell or cell line of claim 25, wherein said GABA receptor subunits comprise the amino acids encoded by the nucleic acids SEQ ID NO: 10 and SEQ ID NO: 13, and an amino acid encoded by a nucleic acid selected from the group consisting of:
a) SEQ ID NO: 1;
b) SEQ ID NO: 2;
c) SEQ ID NO: 3; and
d) SEQ ID NO: 5.

27. The cell or cell line of claim 1, wherein said GABA receptor subunits comprise at least one amino acid selected from the group consisting of
a) any one of SEQ ID NOs: 29-56;
b) an amino acid that is at least 95% identical to any one of SEQ ID NOS: 29-56;
c) an amino acid sequence encoded by a nucleic acid that hybridizes to the reverse-complement of any one of SEQ ID NOs: 1-28 under stringent conditions; and
d) an amino acid encoded by a nucleic acid that is an allelic variant of any one of SEQ ID NOs: 1-28.

28. The cell or cell line of claim 27, wherein said GABA receptor subunits comprise:
a) at least one amino acid selected from the group consisting of SEQ ID NO: 29-34;
b) at least one amino acid selected from the group consisting of SEQ ID NO: 35-39; and
c) at least one amino acid selected from the group consisting of: SEQ ID NO: 40-50.

29. The cell or cell line of claim 28, wherein said GABA receptor subunits comprise the amino acids of SEQ ID NO: 38 and SEQ ID NO: 42 and an amino acid selected from the group consisting of:
a) SEQ ID NO: 29;
b) SEQ ID NO: 30;
c) SEQ ID NO: 31; and
d) SEQ ID NO: 33.

30. The cell or cell line of claim 1, wherein said GABA receptor is a functional GABA receptor.

31. The cell or cell line of claim 30, wherein said functional GABA receptor is a functional GABA_{A} receptor.

32-34. (canceled)

35. The cell or cell line of claim 1, wherein said cell or cell line exhibits a change in intracellular chloride or potassium ions when contacted with GABA ligand.

36. The cell or cell line of claim 1, wherein said cell line produces a Z′ value of at least 0.4, 0.45, 0.5, 0.55, 0.6, 0.65, 0.7, 0.75, or 0.8 in a high-throughput screening assay.

37. The cell or cell line of claim 35 wherein the EC₅₀ value of the GABA ligand on intracellular chloride ion concentration change is
a) between 100 nM and 3.5 μM;
b) below 3.5 μM; or
c) below 400 nM.

38. (canceled)

39. The cell or cell line of claim 1, wherein the GABA receptor subunits are expressed from an endogenous nucleic acid by engineered gene activation.

40. The cell or cell line of claim 1, wherein said cell or cell line further expresses one or more GABA receptor accessory proteins.

41. The cell or cell line of claim 1, wherein said cell or cell line is grown in the absence of selective pressure.

42. A collection of cell lines comprising two or more cell lines, wherein each cell line has been engineered to stably express a GABA receptor subunit or combination of GABA receptor subunits at a consistent level over time.

43-46. (canceled)

47. A method of producing a cell or cells according to claim 1 or 2, comprising the steps of:
a) introducing into a plurality of cells a nucleic acid encoding one or more GABA receptor subunits;
b) introducing into the plurality of cells provided in step a) molecular beacons that detects expression of the GABA receptor subunits; and
c) isolating a cell or cells that express the one or more GABA receptor subunits.

48. The method of claim 47, said method further comprising the step of:
d) generating a cell line from the cell or cells isolated in step c).

49. The method of claim 47 or 48, wherein said step of isolating a cell that expresses said one or more GABA receptor subunits comprises a fluorescence activated cell sorter.

50. The method of claim 47 or 48, wherein said cells or cell lines stably express one or more endogenous GABA receptor accessory proteins, one or more exogenous GABA receptor accessory proteins, or both at a consistent level over time.

51-56. (canceled)

57. A method of identifying a modulator of a GABA receptor, comprising:
a) exposing a cell or cell line that stably expresses one or more GABA receptor subunits at a consistent level over time to a test compound; and
b) detecting a change in a function of the GABA receptor.

58-59. (canceled)

60. The method according to claim 57 wherein said cell or cell line in step a) is a cell or cell or cell line of claim 1 or 2.

61. The method of claim 57, wherein said detecting in step b) utilizes an assay that measures intracellular chloride or potassium ion concentrations.

62. The method of claim 61, wherein said intracellular chloride ion concentration is measured using one or more halide-sensitive fluorescent dyes and a fluorescence microscope.

63-66. (canceled)

67. The method of claim 57, wherein said method further comprises the step of exposing said cell or cell line to a known GABA receptor agonist or antagonist prior to, or simultaneously as, exposing said cell or cell line to said test compound.

68-78. (canceled)

79. The collection of cell lines of claim 42, wherein the cell lines are matched to share the same physiological property to allow parallel processing.

80. A GABA receptor modulator identified by any of the methods of any of claims 57, 60-62, and 67.

81-82. (canceled)

83. A cell engineered to stably express one or more GABA receptor subunits at a consistent level over time, the cell made by a method comprising the steps of
a) providing a plurality of cells that express mRNA(s) encoding said one or more GABA receptor subunits;
b) dispersing the cells individually into individual culture vessels, thereby providing a plurality of separate cell cultures;
c) culturing the cells under a set of desired culture conditions using automated cell culture methods characterized in that the conditions are substantially identical for each of the separate cell cultures, during which culturing the number of cells per separate cell culture is normalized, and wherein the separate cultures are passaged on the same schedule;
d) assaying the separate cell cultures to measure expression of said one or more GABA receptor subunits at least twice; and
e) identifying a separate cell culture that expresses said one or more GABA receptor subunits at a consistent level in both assays, thereby obtaining said cell.


SEQUENCE LISTING (63 sequences)

SEQ ID NO: 1 - DNA, 1371 bp, Homo sapiens
atgaggaaaagtccaggtctgtctgactgtctttgggcctggatcctccttctgagcacactgactggaagaagctatggacagccgtcattacaagatgaacttaaagacaataccactgtcttcaccaggattttggacagactcctagatggttatgacaatcgcctgagaccaggattgggagagcgtgtaaccgaagtgaagactgatatcttcgtcaccagtttcggacccgtttcagaccatgatatggaatatacaatagatgtatttttccgtcaaagctggaaggatgaaaggttaaaatttaaaggacctatgacagtcctccggttaaataacctaatggcaagtaaaatctggactccggacacatttttccacaatggaaagaagtcagtggcccacaacatgaccatgcccaacaaactcctgcggatcacagaggatggcaccttgctgtacaccatgaggctgacagtgagagctgaatgtccgatgcatttggaggacttccctatggatgcccatgcttgcccactaaaatttggaagttatgcttatacaagagcagaagttgtttatgaatggaccagagagccagcacgctcagtggttgtagcagaagatggatcacgtctaaaccagtatgaccttcttggacaaacagtagactctggaattgtccagtcaagtacaggagaatatgttgttatgaccactcatttccacttgaagagaaagattggctactttgttattcaaacatacctgccatgcataatgacagtgattctctcacaagtctccttctggctcaacagagagtctgtaccagcaagaactgtctttggagtaacaactgtgctcaccatgacaacattgagcatcagtgccagaaactccctccctaaggtggcttatgcaacagctatggattggtttattgccgtgtgctatgcctttgtgttctcagctctgattgagtttgccacagtaaactatttcactaagagaggttatgcatgggatggcaaaagtgtggttccagaaaagccaaagaaagtaaaggatcctcttattaagaaaaacaacacttacgctccaacagcaaccagctacacccctaatttggccaggggcgacccgggcttagccaccattgctaaaagtgcaaccatagaacctaaagaggtcaagcccgaaacaaaaccaccagaacccaagaaaacctttaacagtgtcagcaaaattgaccgactgtcaagaatagccttcccgctgctatttggaatctttaacttagtctactgggctacgtatttaaacagagagcctcagctaaaagcccccacaccacatcaatag

SEQ ID NO: 2 - DNA, 1356 bp, Homo sapiens
atgaagacaaaattgaacatctacaacatgcagttcctgctttttgttttcttggtgtgggaccctgccaggttggtgctggctaacatccaagaagatgaggctaaaaataacattaccatctttacgagaattcttgacagacttctggatggttacgataatcggcttagaccaggactgggagacagtattactgaagtcttcactaacatctacgtgaccagttttggccctgtctcagatacagatatggaatatacaattgatgttttctttcgacaaaaatggaaagatgaacgtttaaaatttaaaggtcctatgaatatccttcgactaaacaatttaatggctagcaaaatctggactccagataccttttttcacaatgggaaaaaatcagtagctcataatatgacaatgccaaataagttgcttcgaattcaggatgatgggactctgctgtataccatgaggcttacagttcaagctgaatgcccaatgcacttggaggatttcccaatggatgctcattcatgtcctctgaaatttggcagctatgcatatacaacttcagaggtcacttatatttggacttacaatgcatctgattcagtacaggttgctcctgatggctctaggttaaatcaatatgacctgctgggccaatcaatcggaaaggagacaattaaatccagtacaggtgaatatactgtaatgacagctcatttccacctgaaaagaaaaattgggtattttgtgattcaaacctatctgccttgcatcatgactgtcattctctcccaagtttcattctggcttaacagagaatctgtgcctgcaagaactgtgtttggagtaacaactgtcctaacaatgacaactctaagcatcagtgctcggaattctctccccaaagtggcttatgcaactgccatggactggtttattgctgtttgttatgcatttgtgttctctgccctaattgaatttgcaactgttaattacttcaccaaaagaggatggacttgggatgggaagagtgtagtaaatgacaagaaaaaagaaaaggcttccgttatgatacagaacaacgcttatgcagtggctgttgccaattatgccccgaatctttcaaaagatccagttctctccaccatctccaagagtgcaaccacgccagaacccaacaagaagccagaaaacaagccagctgaagcaaagaaaactttcaacagtgttagcaaaattgacagaatgtccagaatagtttttccagttttgtttggtacctttaatttagtttactgggctacatatttaaacagagaacctgtattaggggtcagtccttga

SEQ ID NO: 3 - DNA, 1479 bp, Homo sapiens
atgataatcacacaaacaagtcactgttacatgaccagccttgggattcttttcctgattaatattctccctggaaccactggtcaaggggaatcaagacgacaagaacccggggactttgtgaagcaggacattggcgggctgtctcctaagcatgccccagatattcctgatgacagcactgacaacatcactatcttcaccagaatcttggatcgtcttctggacggctatgacaaccggctgcgacctgggcttggagatgcagtgactgaagtgaagactgacatctacgtgaccagttttggccctgtgtcagacactgacatggagtacactattgatgtattttttcggcagacatggcatgatgaaagactgaaatttgatggccccatgaagatccttccactgaacaatctcctggctagtaagatctggacaccggacaccttcttccacaatggcaagaaatcagtggctcataacatgaccacgcccaacaagctgctcagattggtggacaacggaaccctcctctatacaatgaggttaacaattcatgctgagtgtcccatgcatttggaagattttcccatggatgtgcatgcctgcccactgaagtttggaagctatgcctatacaacagctgaagtggtttattcttggactctcggaaagaacaaatccgtggaagtggcacaggatggttctcgcttgaaccagtatgaccttttgggccatgttgttgggacagagataatccggtctagtacaggagaatatgtcgtcatgacaacccacttccatctcaagcgaaaaattggctactttgtgatccagacctacttgccatgtatcatgactgtcattctgtcacaagtgtcgttctggctcaacagagagtctgttcctgcccgtacagtctttggtgtcaccactgtgcttaccatgaccaccttgagtatcagtgccagaaattccttacctaaagtggcatatgcgacggccatggactggttcatagccgtctgttatgcctttgtattttctgcactgattgaatttgccactgtcaactatttcaccaagcggagttgggcttgggaaggcaagaaggtgccagaggccctggagatgaagaagaaaacaccagcagccccagcaaagaaaaccagcactaccttcaacatcgtggggaccacctatcccatcaacctggccaaggacactgaattttccaccatctccaagggcgctgctcccagtgcctcctcaaccccaacaatcattgcttcacccaaggccacctacgtgcaggacagcccgactgagaccaagacctacaacagtgtcagcaaggttgacaaaatttcccgcatcatctttcctgtgctctttgccatattcaatctggtctattgggccacatatgtcaaccgggagtcagctatcaagggcatgatccgcaaacagtag

SEQ ID NO: 4 - DNA, 1665 bp, Homo sapiens
atggtttctgccaagaaggtacccgcgatcgctctgtccgccggggtcagtttcgccctcctgcgcttcctgtgcctggcggtttgtttaaacgaatccccaggacagaaccaaaaggaggagaaattgtgcacagaaaatttcacccgcatcctggacagtttgctcgatggttatgacaacaggctgcgtcctggatttgggggtcctgttacagaagtgaaaactgacatatatgtcaccagctttggacctgtttctgatgttgaaatggaatacacaatggatgtgttcttcaggcagacatggattgacaaaagattaaaatatgacggccccattgaaattttgagattgaacaatatgatggtaacgaaagtgtggacccctgatactttcttcaggaatggaaagaaatctgtctcacataatatgacagctccaaataagctttttagaattatgagaaatggtactattttatacacaatgagactcaccataagtgcggagtgtcccatgagattggtggattttcccatggatggtcatgcatgccctttgaaattcgggagttatgcctatccaaagagtgagatgatctatacctggacaaaaggtcctgagaaatcagttgaagttccgaaggagtcttccagcttagttcaatatgatttgattgggcaaaccgtatcaagtgaaaccatcaaatcaattacgggtgaatatattgttatgacggtttacttccacctcagacggaagatgggttattttatgattcagacctatattccgtgcattatgacagtgattctttctcaagtttcattttggataaataaagaatcagttcccgctaggactgtatttggaataacaactgtcctcaccatgaccacactaagcatcagtgcacgacattctttgcccaaagtgtcctatgctaccgccatggactggttcatagctgtctgctttgcttttgtattttcggcccttatcgagtttgctgctgtcaactatttcaccaatattcaaatggaaaaagccaaaaggaagacatcaaagccccctcaggaagttcccgctgctccagtgcagagagagaagcatcctgaagcccctctgcagaatacaaatgccaatttgaacatgagaaaaagaacaaatgctttggttcactctgaatctgatgttggcaacagaactgaggtgggaaaccattcaagcaaatcttccacagttgttcaagaatcttctaaaggcacacctcggtcttacttagcttccagtccaaacccattcagccgtgcaaatgcagctgaaaccatatctgcagcaagagcacttccatctgcttctcctacttctatccgaactggatatatgcctcgaaaggcttcagttggatctgcttctactcgtcacgtgtttggatcaagactgcagaggataaagaccacagttaataccataggggctactgggaagttgtcagctactcctcctccatcggctccaccaccttctggatctggcacaagtaaaatagacaaatatgcccgtattctctttccagtcacatttggggcatttaacatggtttattgggttgtttatttatctaaggacactatggagaaatcagaaagtctaatgtaa

SEQ ID NO: 5 - DNA, 1389 bp, Homo sapiens
atggacaatggaatgttctctggttttatcatgatcaaaaacctccttctcttttgtatttccatgaacttatccagtcactttggcttttcacagatgccaaccagttcagtgaaagatgagaccaatgacaacatcacgatatttaccaggatcttggatgggctcttggatggctacgacaacagacttcggcccgggctgggagagcgcatcactcaggtgaggaccgacatctacgtcaccagcttcggcccggtgtccgacacggaaatggagtacaccatagacgtgtttttccgacaaagctggaaagatgaaaggcttcggtttaaggggcccatgcagcgcctccctctcaacaacctccttgccagcaagatctggaccccagacacgttcttccacaacgggaagaagtccatcgctcacaacatgaccacgcccaacaagctgctgcggctggaggacgacggcaccctgctctacaccatgcgcttgaccatctctgcagagtgccccatgcagcttgaggacttcccgatggatgcgcacgcttgccctctgaaatttggcagctatgcgtaccctaattctgaagtcgtctacgtctggaccaacggctccaccaagtcggtggtggtggcggaagatggctccagactgaaccagtaccacctgatggggcagacggtgggcactgagaacatcagcaccagcacaggcgaatacacaatcatgacagctcacttccacctgaaaaggaagattggctactttgtcatccagacctaccttccctgcataatgaccgtgatcttatcacaggtgtccttttggctgaaccgggaatcagtcccagccaggacagtttttggggtcaccacggtgctgaccatgacgaccctcagcatcagcgccaggaactctctgcccaaagtggcctacgccaccgccatggactggttcatagccgtgtgctatgccttcgtcttctcggcgctgatagagtttgccacggtcaattactttaccaagagaggctgggcctgggatggcaaaaaagccttggaagcagccaagatcaagaaaaagcgtgaagtcatactaaataagtcaacaaacgcttttacaactgggaagatgtctcaccccccaaacattccgaaggaacagaccccagcagggacgtcgaatacaacctcagtctcagtaaaaccctctgaagagaagacttctgaaagcaaaaagacttacaacagtatcagcaaaattgacaaaatgtcccgaatcgtattcccagtcttgttcggcactttcaacttagtttactgggcaacgtatttgaatagggagccggtgataaaaggagccgcctctccaaaataa

SEQ ID NO: 6 - DNA, 1362 bp, Homo sapiens
atggcgtcatctctgccctggctgtgcattattctgtggctagaaaatgccctagggaaactcgaagttgaaggcaacttctactcagaaaacgtcagtcggatcctggacaacttgcttgaaggctatgacaatcggctgcggccgggatttggaggtgctgtcactgaagtcaaaacagacatttatgtgaccagttttgggcccgtgtcagatgtggagatggagtatacgatggatgtttttttccgccagacctggactgatgagaggttgaagtttggggggccaactgagattctgagtctgaataatttgatggtcagtaaaatctggacgcctgacacctttttcagaaatggtaaaaagtccattgctcacaacatgacaactcctaataaactcttcagaataatgcagaatggaaccattttatacaccatgaggcttaccatcaatgctgactgtcccatgaggctggttaactttcctatggatgggcatgcttgtccactcaagtttgggagctatgcttatcccaaaagtgaaatcatatatacgtggaaaaaaggaccactttactcagtagaagtcccagaagaatcttcaagccttctccagtatgatctgattggacaaacagtatctagtgagacaattaaatctaacacaggtgaatacgttataatgacagtttacttccacttgcaaaggaagatgggctacttcatgatacagatatacactccttgcattatgacagtcattctttcccaggtgtctttctggattaataaggagtccgtcccagcaagaactgtttttgggatcaccactgttttaactatgaccactttgagcatcagtgcccggcactctttgccaaaagtgtcatatgccactgccatggattggttcatagctgtttgctttgcattcgtcttctctgctcttatcgagttcgcagctgtcaactactttaccaatcttcagacacagaaggcgaaaaggaaggcacagtttgcagccccacccacagtgacaatatcaaaagctactgaacctttggaagctgagattgttttgcatcctgactccaaatatcatctgaagaaaaggatcacttctctgtctttgccaatagtttcatcttccgaggccaataaagtgctcacgagagcgcccatcttacaatcaacacctgtcacacccccaccactctcgccagcctttggaggcaccagtaaaatagaccagtattctcgaattctcttcccagttgcatttgcaggattcaaccttgtgtactgggtagtttatctttccaaagatacaatggaagtgagtagcagtgttgaatag

SEQ ID NO: 7 - DNA, 1425 bp, Homo sapiens
atgtggacagtacaaaatcgagagagtctggggcttctctctttccctgtgatgattaccatggtctgttgtgcacacagcaccaatgaacccagcaacatgycatacgtgaaagagacagtggacagattgctcaaaggatatgacattcgcttgcggccggacttcggagggccccccgtcgacgttgggatgcggatcgatgtcgccagcatagacatggtctccgaagtgaatatggattatacactcaccatgtatttccagcagtcttggaaagacaaaaggctttcttattctggaatcccactgaacctcaccctagacaatagggtagctgaccaactctgggtaccagacacctactttctgaatgacaagaaatcatttgtgcatggggtcacagtgaaaaatcgaatgattcgactgcatcctgatggaacagttctctatggactccgaatcacaaccacagctgcatgtatgatggatcttcgaagatatccactggatgagcagaactgcaccctggagatcgaaagttatggctataccactgatgacattgaattttactggaatggaggagaaggggcagtcactggtgttaataaaatcgaacttcctcaattttcaattgttgactacaagatggtgtctaagaaggtggagttcacaacaggagcgtatccacgactgtcactaagttttcgtctaaagagaaacattggttacttcattttgcaaacctacatgccttctacactgattacaattctgtcctgggtgtctttttggatcaactatgatgcatctgcagccagagtcgcactaggaatcacgacggtgcttacaatgacaaccatcagcacccacctcagggagaccctgccaaagatcccttatgtcaaagcgattgatatttatctgatgggttgctttgtgtttgtgttcctggctctgctggagtatgcctttgtaaattacatcttctttgggaaaggccctcagaaaaagggagctagcaaacaagaccagagtgccaatgagaagaataaactggagatgaataaagtccaggtcgacgcccacggtaacattctcctcagcaccctggaaatccggaatgagacgagtggctcggaagtgctcacgagcgtgagcgaccccaaggccaccatgtactcctatgacagcgccagcatccagtaccgcaagcccctgagcagccgcgaggcctacgggcgcgccctggaccggcacggggtacccagcaaggggcgcatccgcaggcgtgcctcccagctcaaagtcaagatccccgacttgactgatgtgaattccatagacaagtggtcccgaatgtttttccccatcaccttttctctttttaatgtcgtctattggctttactatgtacactga

SEQ ID NO: 8 - DNA, 1539 bp, Homo sapiens
atgtggagagtgcggaaaaggggctactttgggatttggtccttccccttaataatcgccgctgtctgtgcgcagagtgtcaatgaccctagtaatatgtcgctggttaaagagacggtggatagactcctgaaaggctatgacattcgtctgagaccagattttggaggtccccccgtggctgtggggatgaacattgacattgccagcatcgatatggtttctgaagtcaatatggattataccttgacaatgtactttcaacaagcctggagagataagaggctgtcctataatgtaatacctttaaacttgactctggacaacagagtggcagaccagctctgggtgcctgatacctatttcctgaacgataagaagtcatttgtgcacggagtgactgttaagaaccgcatgattcgcctgcatcctgatggcaccgtcctttatggactcagaatcacaaccacagctgcctgcatgatggacctaaggaggtacccactggatgaacaaaactgcaccttggaaattgagagctatggatacacaactgatgacattgagttttactggcgtggcgatgataatgcagtaacaggagtaacgaaaattgaacttccacagttctctattgtagattacaaacttatcaccaagaaggttgttttttccacaggttcctatcccaggttatccctcagctttaagcttaagagaaacattggctactttatcctgcaaacatacatgccttccatcctgattaccatcctctcctgggtctccttctggattaattacgatgcttcagctgcaagggtggcattaggaatcacaactgtcctcacaatgaccacaatcaacacccacctccgggaaactctccctaaaatcccctatgtgaaggccattgacatgtacctgatggggtgctttgtcttcgttttcatggcccttctggaatatgccctagtcaactacatcttctttgggagggggccccaacgccaaaagaaagcagctgagaaggctgccagtgccaacaatgagaagatgcgcctggatgtcaacaagattttttataaagatattaaacaaaatgggacccaatatcgatccttgtgggaccctactggaaacctctccccaactagacggactaccaattacgatttctctctgtatacgatggacccccatgagaacatcttactgagcactctcgagataaaaaatgaaatggccacatctgaggctgtgatgggacttggagaccccagaagcacaatgctagcctatgatgcctccagcatccagtatcggaaagctgggttgcccaggcatagttttggccgaaatgctctggaacgacatgtggcgcaaaagaaaagtcgcctgaggagacgcgcctcccaactgaaaatcaccatccctgacttgactgatgtgaatgccatagatcggtggtcccgcatattcttcccagtggttttttccttcttcaacatcgtctattggctttactatgtgaactaa

SEQ ID NO: 9 - DNA, 1425 bp, Homo sapiens
atgtggagagtgcggaaaaggggctactttgggatttggtccttccccttaataatcgccgctgtctgtgcgcagagtgtcaatgaccctagtaatatgtcgctggttaaagagacggtggatagactcctgaaaggctatgacattcgtctgagaccagattttggaggtccccccgtggctgtggggatgaacattgacattgccagcatcgatatggtttctgaagtcaatatggattataccttgacaatgtactttcaacaagcctggagagataagaggctgtcctataatgtaatacctttaaacttgactctggacaacagagtggcagaccagctctgggtgcctgatacctatttcctgaacgataagaagtcatttgtgcacggagtgactgttaagaaccgcatgattcgcctgcatcctgatggcaccgtcctttatggactcagaatcacaaccacagctgcctgcatgatggacctaaggaggtacccactggatgaacaaaactgcaccttggaaattgagagctatggatacacaactgatgacattgagttttactggcgtggcgatgataatgcagtaacaggagtaacgaaaattgaacttccacagttctctattgtagattacaaacttatcaccaagaaggttgttttttccacaggttcctatcccaggttatccctcagctttaagcttaagagaaacattggctactttatcctgcaaacatacatgccttccatcctgattaccatcctctcctgggtctccttctggattaattacgatgcttcagctgcaagggtggcattaggaatcacaactgtcctcacaatgaccacaatcaacacccacctccgggaaactctccctaaaatcccctatgtgaaggccattgacatgtacctgatggggtgctttgtcttcgttttcatggcccttctggaatatgccctagtcaactacatcttctttgggagggggccccaacgccaaaagaaagcagctgagaaggctgccagtgccaacaatgagaagatgcgcctggatgtcaacaagatggacccccatgagaacatcttactgagcactctcgagataaaaaatgaaatggccacatctgaggctgtgatgggacttggagaccccagaagcacaatgctagcctatgatgcctccagcatccagtatcggaaagctgggttgcccaggcatagttttggccgaaatgctctggaacgacatgtggcgcaaaagaaaagtcgcctgaggagacgcgcctcccaactgaaaatcaccatccctgacttgactgatgtgaatgccatagatcggtggtcccgcatattcttcccagtggttttttccttcttcaacatcgtctattggctttattatgtgaactaa

SEQ ID NO: 10 - DNA, 1422 bp, Homo sapiens
atgtggggccttgcgggaggaaggcttttcggcatcttctcggccccggtgctggtggctgtggtgtgctgcgcccagagtgtgaacgatcccgggaacatgtcctttgtgaaggagacggtggacaagctgttgaaaggctacgacattcgcctaagacccgacttcgggggtcccccggtctgcgtggggatgaacatcgacatcgccagcatcgacatggtttccgaagtcaacatggattataccttaaccatgtattttcaacaatattggagagataaaaggctcgcctattctgggatccctctcaacctcacgcttgacaatcgagtggctgaccagctatgggtgcccgacacatatttcttaaatgacaaaaagtcatttgtgcatggagtgacagtgaaaaaccgcatgatccgtcttcaccctgatgggacagtgctgtatgggctcagaatcaccacgacagcagcatgcatgatggacctcaggagataccccctggacgagcagaactgcactctggaaattgaaagctatggctacaccacggatgacattgagttttactggcgaggcggggacaaggctgttaccggagtggaaaggattgagctcccgcagttctccatcgtggagcaccgtctggtctcgaggaatgttgtcttcgccacaggtgcctatcctcgactgtcactgagctttcggttgaagaggaacattggatacttcattcttcagacttatatgccctctatactgataacgattctgtcgtgggtgtccttctggatcaattatgatgcatctgctgctagagttgccctcgggatcacaactgtgctgacaatgacaaccatcaacacccaccttcgggagaccttgcccaaaatcccctatgtcaaagccattgacatgtaccttatgggctgcttcgtctttgtgttcctggcccttctggagtatgcctttgtcaactacattttctttggaagaggccctcaaaggcagaagaagcttgcagaaaagacagccaaggcaaagaatgaccgttcaaagagcgaaagcaaccgggtggatgctcatggaaatattctgttgacatcgctggaagttcacaatgaaatgaatgaggtctcaggcggcattggcgataccaggaattcagcaatatcctttgacaactcaggaatccagtacaggaaacagagcatgcctcgagaagggcatgggcgattcctgggggacagaagcctcccgcacaagaagacccatctacggaggaggtcttcacagctcaaaattaaaatacctgatctaaccgatgtgaatgccatagacagatggtccaggatcgtgtttccattcactttttctcttttcaacttagtttactggctgtactatgttaactga

SEQ ID NO: 11 - DNA, 1422 bp, Homo sapiens
atgtgctccgggctcctggagctcctgctgcccatctggctctcctggaccctggggacccgaggctctgagccccgcagtgtgaacgatcccgggaacatgtcctttgtgaaggagacggtggacaagctgttgaaaggctacgacattcgcctaagacccgacttcgggggtcccccggtctgcgtggggatgaacatcgacatcgccagcatcgacatggtttccgaagtcaacatggattataccttaaccatgtattttcaacaatattggagagataaaaggctcgcctattctgggatccctctcaacctcacgcttgacaatcgagtggctgaccagctatgggtgcccgacacatatttcttaaatgacaaaaagtcatttgtgcatggagtgacagtgaaaaaccgcatgatccgtcttcaccctgatgggacagtgctgtatgggctcagaatcaccacgacagcagcatgcatgatggacctcaggagataccccctggacgagcagaactgcactctggaaattgaaagctatggctacaccacggatgacattgagttttactggcgaggcggggacaaggctgttaccggagtggaaaggattgagctcccgcagttctccatcgtggagcaccgtctggtctcgaggaatgttgtcttcgccacaggtgcctatcctcgactgtcactgagctttcggttgaagaggaacattggatacttcattcttcagacttatatgccctctatactgataacgattctgtcgtgggtgtccttctggatcaattatgatgcatctgctgctagagttgccctcgggatcacaactgtgctgacaatgacaaccatcaacacccaccttcgggagaccttgcccaaaatcccctatgtcaaagccattgacatgtaccttatgggctgcttcgtctttgtgttcctggcccttctggagtatgcctttgtcaactacattttctttggaagaggccctcaaaggcagaagaagcttgcagaaaagacagccaaggcaaagaatgaccgttcaaagagcgaaagcaaccgggtggatgctcatggaaatattctgttgacatcgctggaagttcacaatgaaatgaatgaggtctcaggcggcattggcgataccaggaattcagcaatatcctttgacaactcaggaatccagtacaggaaacagagcatgcctcgagaagggcatgggcgattcctgggggacagaagcctcccgcacaagaagacccatctacggaggaggtcttcacagctcaaaattaaaatacctgatctaaccgatgtgaatgccatagacagatggtccaggatcgtgtttccattcactttttctcttttcaacttagtttactggctgtactatgttaactga

SEQ ID NO: 12 - DNA, 1398 bp, Homo sapiens
atgggtcctttgaaagcttttctcttctccccttttcttctgcggagtcaaagtagaggggtgaggttggtcttcttgttactgaccctgcatttgggaaactgtgttgataaggcagatgatgaagatgatgaggatttaacggtgaacaaaacctgggtcttggccccaaaaattcatgaaggagatatcacacaaattctgaattcattgcttcaaggctatgacaataaacttcgtccagatataggagtgaggcccacagtaattgaaactgatgtttatgtaaacagcattggaccagttgatccaattaatatggaatatacaatagatataatttttgcccaaacctggtttgacagtcgtttaaaattcaatagtaccatgaaagtgcttatgcttaacagtaatatggttggaaaaatttggattcctgacactttcttcagaaactcaagaaaatctgatgctcactggataacaactcctaatcgtctgcttcgaatttggaatgatggacgagttctgtatactctaagattgacaattaatgcagaatgttatcttcagcttcataactttcccatggatgaacattcctgtccactggaattttcaagctatggataccctaaaaatgaaattgagtataagtggaaaaagccctccgtagaagtggctgatcctaaatactggagattatatcagtttgcatttgtagggttacggaactcaactgaaatcactcacacgatctctggggattatgttatcatgacaattttttttgacctgagcagaagaatgggatatttcactattcagacctacattccatgcattctgacagttgttctttcttgggtgtctttttggatcaataaagatgcagtgcctgcaagaacatcgttgggtatcactacagttctgactatgacaaccctgagtacaattgccaggaagtctttacctaaggtttcttatgtgactgcgatggatctctttgtttctgtttgtttcatttttgtttttgcagccttgatggaatatggaaccttgcattattttaccagcaaccaaaaaggaaagactgctactaaagacagaaagctaaaaaataaagcctcgatgactcctggtctccatcctggatccactctgattccaatgaataatatttctgtgccgcaagaagatgattatgggtatcagtgtttggagggcaaagattgtgccagcttcttctgttgctttgaagactgcagaacaggatcttggagggaaggaaggatacacatacgcattgccaaaattgactcttattctagaatatttttcccaaccgcttttgccctgttcaacttggtttattgggttggctatctttacttataa

SEQ ID NO: 13 - DNA, 1404 bp, Homo sapiens
atgagttcgccaaatatatggagcacaggaagctcagtctactcgactcctgtattttcacagaaaatgacggtgtggattctgctcctgctgtcgctctaccctggcttcactagccagaaatctgatgatgactatgaagattatgcttctaacaaaacatgggtcttgactccaaaagttcctgagggtgatgtcactgtcatcttaaacaacctgctggaaggatatgacaataaacttcggcctgatataggagtgaagccaacgttaattcacacagacatgtatgtgaatagcattggtccagtgaacgctatcaatatggaatacactattgatatattttttgcgcaaacgtggtatgacagacgtttgaaatttaacagcaccattaaagtcctccgattgaacagcaacatggtggggaaaatctggattccagacactttcttcagaaattccaaaaaagctgatgcacactggatcaccacccccaacaggatgctgagaatttggaatgatggtcgagtgctctacaccctaaggttgacaattgatgctgagtgccaattacaattgcacaactttccaatggatgaacactcctgccccttggagttctcaagttatggctatccacgtgaagaaattgtttatcaatggaagcgaagttctgttgaagtgggcgacacaagatcctggaggctttatcaattctcatttgttggtctaagaaataccaccgaagtagtgaagacaacttccggagattatgtggtcatgtctgtctactttgatctgagcagaagaatgggatactttaccatccagacctatatcccctgcacactcattgtcgtcctatcctgggtgtctttctggatcaataaggatgctgttccagccagaacatctttaggtatcaccactgtcctgacaatgaccaccctcagcaccattgcccggaaatcgctccccaaggtctcctatgtcacagcgatggatctctttgtatctgtttgtttcatctttgtcttctctgctctggtggagtatggcaccttgcattattttgtcagcaaccggaaaccaagcaaggacaaagataaaaagaagaaaaaccctgcccctaccattgatatccgcccaagatcagcaaccattcaaatgaataatgctacacaccttcaagagagagatgaagagtacggctatgagtgtctggacggcaaggactgtgccagttttttctgctgttttgaagattgtcgaacaggagcttggagacatgggaggatacatatccgcattgccaaaatggactcctatgctcggatcttcttccccactgccttctgcctgtttaatctggtctattgggtctcctacctctacctgtga

SEQ ID NO: 14 - DNA, 1548 bp, Homo sapiens
atgagttcgccaaatatatggagcacaggaagctcagtctactcgactcctgtattttcacagaaaatgacggtgtggattctgctcctgctgtcgctctaccctggcttcactagccagaaatctgatgatgactatgaagattatgcttctaacaaaacatgggtcttgactccaaaagttcctgagggtgatgtcactgtcatcttaaacaacctgctggaaggatatgacaataaacttcggcctgatataggagtgaagccaacgttaattcacacagacatgtatgtgaatagcattggtccagtgaacgctatcaatatggaatacactattgatatattttttgcgcaaacgtggtatgacagacgtttgaaatttaacagcaccattaaagtcctccgattgaacagcaacatggtggggaaaatctggattccagacactttcttcagaaattccaaaaaagctgatgcacactggatcaccacccccaacaggatgctgagaatttggaatgatggtcgagtgctctacaccctaaggttgacaattgatgctgagtgccaattacaattgcacaactttccaatggatgaacactcctgccccttggagttctccagttggtctcgttctattgcccaggctggaatgtgcagtggtgtgatctcggctcactacagccttcgcttctggggctcaactgatcctcccaccttggcctccagagtagctgggatttcagatggctatccacgtgaagaaattgtttatcaatggaagcgaagttctgttgaagtgggcgacacaagatcctggaggctttatcaattctcatttgttggtctaagaaataccaccgaagtagtgaagacaacttccggagattatgtggtcatgtctgtctactttgatctgagcagaagaatgggatactttaccatccagacctatatcccctgcacactcattgtcgtcctatcctgggtgtctttctggatcaataaggatgctgttccagccagaacatctttaggtatcaccactgtcctgacaatgaccaccctcagcaccattgcccggaaatcgctccccaaggtctcctatgtcacagcgatggatctctttgtatctgtttgtttcatctttgtcttctctgctctggtggagtatggcaccttgcattattttgtcagcaaccggaaaccaagcaaggacaaagataaaaagaagaaaaaccctcttcttcggatgttttccttcaaggcccctaccattgatatccgcccaagatcagcaaccattcaaatgaataatgctacacaccttcaagagagagatgaagagtacggctatgagtgtctggacggcaaggactgtgccagttttttctgctgttttgaagattgtcgaacaggagcttggagacatgggaggatacatatccgcattgccaaaatggactcctatgctcggatcttcttccccactgccttctgcctgtttaatctggtctattgggtctcctacctctacctgtga

SEQ ID NO: 15 - DNA, 1405 bp, Homo sapiens
atggccccgaagctgctgctcctcctctgcctgtcctcgggcttgtacgcgcggtccagaaaggtggaagaggatgaatatgaagattcatcatcaaaccaaaagtgggtcttggctccaaaatcccaagacaccgacgtgactcttattctcaacaagttgctaagagaatatgataaaaagctgaggccagatattggaataaaaccgaccgtaattgacgttgacatttatgttaacagcattggtcctgtgtcatcaataaacatggaataccaaattgacatattttttgctcagacctggacagatagtcgccttcgattcaacagcacaatgaaaattcttactctgaacagcaacatggtggggttaatctggatcccagacaccatcttccgcaattctaaaaccgcagaggctcactggatcaccacacccaatcagctcctccggatttggaatgacgggaaaatcctttacactttgaggctcaccatcaatgctgagtgccagctgcagctgcacaacttccccatggacgaacactcctgcccgctgattttctccagctatggctatcccaaagaagaaatgatttatagatggagaaaaaattcagtggaggcagctgaccagaaatcatggcggctttatcagtttgacttcatgggcctcagaaacaccacagaaatcgtgacaacgtctgcaggtgattatgttgtcatgactatatattttgaattgagtagaagaatgggatacttcaccattcagacatacattccctgtatactgactgtggttttatcctgggtgtcattttggatcaaaaaagatgctacgccagcaagaacagcattaggcatcaccacggtgctgaccatgaccaccctgagcaccatcgccaggaagtccttgccacgcgtgtcctacgtgaccgccatggacctttttgtgactgtgtgcttcctgtttgtcttcgccgcgctgatggagtatgccaccctcaactactattccagctgtagaaaaccaaccaccacgaaraagacaacatcgttactacatccagattcctcaagatggattcctgagcgaataagcctacaagccccttccaactattccctcctggacatgaggccaccaccacctgcgatgatcactttaaacaattccgtttactggcaggaatttgaagatacctgtgtctatgagtgtctggatggcaaagactgtcagagcttcttctgctgctatgaagaatgtaaatcaggatcctggaggaaagggcgtattcacatagacatcttggagctggactcgtactcccgggtctttttccccacgtccttcctgctctttaagcctggtctactgggttggatacctgtatctctaa

SEQ ID NO: 16 - DNA, 1359 bp, Homo sapiens
atggacgcgcccgcccggctgctggccccgctcctgctcctctgcgcgcagcagctccgcggcaccagagcgatgaatgacatcggcgactacgtgggctccaacctggagatctcctggctccccaacctggacgggctgatagccggctacgcccgcaacttccggcctggcatcggaggcccccccgtgaatgtggcccttgccctggaggtggccagcatcgaccacatctcagaggccaacatggagtacaccatgacggtgttcctgcaccagagctggcgggacagcaggctctcctacaaccacaccaacgagaccctgggtctggacagccgcttcgtggacaagctgtggctgcccgacaccttcatcgtgaacgccaagtcggcctggttccacgacgtgacggtggagaacaagctcatccggctgcagcccgacggcgtgatcctgtacagcatccgaatcacctccactgtggcctgcgacatggacctggccaaataccccatggacgagcaggagtgcatgctggacctggagagctacggttactcatcggaggacatcgtctactactggtcggagagccaggagcacatccacgggctggacaagctgcagctggcgcagttcaccatcaccagctaccgcttcaccacggagctgatgaacttcaagtccgctggccagttcccacggctcagcctgcacttccacctgcggaggaaccgcggcgtgtacatcatccaatcctacatgccctccgtcctgctggtcgccatgtcctgggtctccttctggatcagccaggcggcggtgcccgccagggtgtctctaggcatcaccacggtgctgacgatgaccacgctcatggtcagtgcccgctcctccctgccacgggcatcagccatcaaggcactggacgtctacttctggatctgctatgtcttcgtgtttgccgccctggtggagtacgcctttgctcatttcaacgccgactacaggaagaagcagaaggccaaggtcaaggtctccaggccgagggcagagatggacgtgaggaacgccattgtcctcttctccctctctgctgccggcgtcacgcaggagctggccatctcccgccggcagcgccgcgtcccggggaacctgatgggctcctacaggtcggtgggggtggagacaggggagacgaagaaggagggggcagcccgctcaggaggccaggggggcatccgtgcccggctcaggcccatcgacgcagacaccattgacatttacgcccgcgctgtgttccctgcggcgtttgcggccgtcaatgtcatctactgggcggcatacgccatgtga

SEQ ID NO: 17 - DNA, 1359 bp, Homo sapiens; identical to SEQ ID NO: 16

SEQ ID NO: 18 - DNA, 1323 bp, Homo sapiens
atgaactacagcctccacttggccttcgtgtgtctgagtctcttcactgagaggatgtgcatccaggggagtcagttcaacgtcgaggtcggcagaagtgacaagctttccctgcctggctttgagaacctcacagcaggatataacaaatttctcaggcccaattttggtggagaacccgtacagatagcgctgactctggacattgcaagtatctctagcatttcagagagtaacatggactacacagccaccatatacctccgacagcgctggatggaccagcggctggtgtttgaaggcaacaagagcttcactctggatgcccgcctcgtggagttcctctgggtgccagatacttacattgtggagtccaagaagtccttcctccatgaagtcactgtgggaaacaggctcatccgcctcttctccaatggcacggtcctgtatgccctcagaatcacgacaactgttgcatgtaacatggatctgtctaaataccccatggacacacagacatgcaagttgcagctggaaagctggggctatgatggaaatgatgtggagttcacctggctgagagggaacgactctgtgcgtggactggaacacctgcggcttgctcagtacaccatagagcggtatttcaccttagtcaccagatcgcagcaggagacaggaaattacactagattggtcttacagtttgagcttcggaggaatgttctgtatttcattttggaaacctacgttccttccactttcctggtggtgttgtcctgggtttcattttggatctctctcgattcagtccctgcaagaacctgcattggagtgacgaccgtgttatcaatgaccacactgatgatcgggtcccgcacttctcttcccaacaccaactgcttcatcaaggccatcgatgtgtacctggggatctgctttagctttgtgtttggggccttgctagaatatgcagttgctcactacagttccttacagcagatggcagccaaagatagggggacaacaaaggaagtagaagaagtcagtattactaatatcatcaacagctccatctccagctttaaacggaagatcagctttgccagcattgaaatttccagcgacaacgttgactacagtgacttgacaatgaaaaccagcgacaagttcaagtttgtcttccgagaaaagatgggcaggattgttgattatttcacaattcaaaaccccagtaatgttgatcactattccaaactactgtttcctttgatttttatgctagccaatgtattttactgggcatactacatgtatttttga

SEQ ID NO: 19 - DNA, 1899 bp, Homo sapiens
atgggcatccgaggcatgctgcgagccgcagtgatcctgctgctcatcaggacctggctcgcggagggcaactaccccagtcccatcccgaaattccacttcgagttctcctctgctgtgcccgaagtcgtcctgaacctcttcaactgcaaaaattgtgcaaatgaagctgtggttcaaaagattttggacagggtgctgtcaagatacgatgtccgcctgagaccgaattttggaggtgcccctgtgcctgtgagaatatctatttatgtcacgagcattgaacagatctcagaaatgaatatggactacacgatcacgatgttttttcatcagacttggaaagattcacgcttagcatactatgagaccaccctgaacttgaccctggactatcggatgcatgagaagttgtgggtccctgactgctactttctgaacagcaaggatgctttcgtgcatgatgtgactgtggagaatcgcgtgtttcagcttcacccagatggaacggtgcggtacggcatccgactcaccactacagcagcttgttccctggatctgcataaattccctatggacaagcaggcctgcaacctggtggtagagagctatggttacacggttgaagacatcatattattctgggatgacaatgggaacgccatccacatgactgaggagctgcatatccctcagttcactttcctgggaaggacgattactagcaaggaggtgtatttctacacaggttcctacatacgcctgatactgaagttccaggttcagagggaagttaacagctaccttgtgcaagtctactggcctactgtcctcaccactattacctcttggatatcgttttggatgaactatgattcctctgcagccagggtgacaattggcttaacttcaatgctcatcctgaccaccatcgactcacatctgcgggataagctccccaacatttcctgtatcaaggccattgatatctatatcctcgtgtgcttgttctttgtgttcctgtccttgctggagtatgtctacatcaactatcttttctacagtcgaggacctcggcgccagcctaggcgacacaggagaccccgaagagtcattgcccgctaccgctaccagcaagtggtggtaggaaacgtgcaggatggcctgattaacgtggaagacggagtcagctctctccccatcaccccagcgcaggcccccctggcaagcccggaaagcctcggttctttgacgtccacctccgagcaggcccagctggccacctcggaaagcctcagcccactcacttctctctcaggccaggcccccctggccactggagaaagcctgagcgatctcccctccacctcagagcaggcccggcacagctatggtgttcgctttaatggtttccaggctgatgacagtattattcctaccgaaatccgcaaccgtgtcgaagcccatggccatggtgttacccatgaccatgaagattccaatgagagcttgagctcggatgagcgccatggccatggccccagtgggaagcccatgcttcaccatggcgagaagggtgtgcaagaagcaggctgggaccttgatgacaacaatgacaagagcgactgccttgccattaaggagcaattcaagtgtgatactaacagtacctggggccttaatgatgatgagctcatggcccatggccaagagaaggacagtagctcagagtctgaggatagttgccccccaagccctgggtgctccttcactgaagggttctccttcgatctctttaatcctgactacgtcccaaaggtcgacaagtggtcccggttcctcttccctctggcctttgggttgttcaacattgtttactgggtataccatatgtattag

SEQ ID NO: 20 - DNA, 1440 bp, Homo sapiens
atgttggctgtcccaaatatgagatttggcatctttcttttgtggtggggatgggttttggccactgaaagcagaatgcactggcccggaagagaagtccacgagatgtctaagaaaggcaggccccaaagacaaagacgagaagtacatgaagatgcccacaagcaagtcagcccaattctgagacgaagtcctgacatcaccaaatcgcctctgacaaagtcagaacagcttctgaggatagatgaccatgatttcagcatgaggcctggctttggaggccctgccattcctgttggtgtggatgtgcaggtggagagtttggatagcatctcagaggttgacatggactttacgatgaccctctacctgaggcactactggaaggacgagaggctgtcttttccaagcaccaacaacctcagcatgacgtttgatggccggctggtcaagaagatctgggtccctgacatgtttttcgtgcactccaaacgctccttcatccacgacaccaccacagacaacgtcatgttgcgggtccagcctgatgggaaagtgctctatagtctcagggttacagtaactgcaatgtgcaacatggacttcagccgatttcccttggacacacaaacgtgctctcttgaaattgaaagctatgcctatacagaagatgacctcatgctgtactggaaaaagggcaatgactccttaaagacagatgaacggatctcactctcccagttcctcattcaggaattccacaccaccaccaaactggctttctacagcagcacaggctggtacaaccgtctctacattaatttcacgttgcgtcgccacatcttcttcttcttgctccaaacttatttccccgctaccctgatggtcatgctgtcctgggtgtccttctggatcgaccgcagagccgtgcctgccagagtccccttaggtatcacaacggtgctgaccatgtccaccatcatcacgggcgtgaatgcctccatgccgcgcgtctcctacatcaaggccgtggacatctacctctgggtcagctttgtgttcgtgttcctctcggtgctggagtatgcggccgtcaactacctgaccactgtgcaggagaggaaggaacagaagctgcgggagaagcttccctgcaccagcggattacctccgccccgcactgcgatgctggacggcaactacagtgatggggaggtgaatgacctggacaactacatgccagagaatggagagaagcccgacaggatgatggtgcagctgaccctggcctcagagaggagctccccacagaggaaaagtcagagaagcagctatgtgagcatgagaatcgacacccacgccattgataaatactccaggatcatctttccagcagcatacattttattcaatttaatatactggtctattttctcctag

SEQ ID NO: 21 - DNA, 1473 bp, Homo sapiens
atggtcaagccaggggggatttgctctgccacaggctactggaaagcagctttttgcctcacagatgtccacaaaatgccttattttacaagactcattttgttcttgttttgcttgatggttctcgtggagagcagaaaacccaagaggaagcgatggacagggcaggtggaaatgcccaagccaagtcacttatataagaagaaccttgatgtgaccaagatccggaagggaaagcctcagcagcttctcagagtggacgagcacgacttcagcatgagacccgccttcggaggccctgccatcccggtgggcgtggacgtacaggtggagagcctggacagcatctccgaggtggacatggacttcactatgaccctgtacctgcggcattactggaaggatgagaggctagctttctccagcgccagcaacaagagcatgaccttcgatggccggctggtgaagaagatctgggtccctgatgtcttctttgttcactccaaaagatcgttcactcatgacaccaccactgacaacatcatgctgagggtgttcccagatggacacgtgctgtacagcatgaggattacggtcactgccatgtgcaacatggacttcagccactttcccctggactcccagacctgttctttggagctggagagctatgcctatacagatgaagatctaatgctgtactggaagaatggggatgaatccctaaaaacagatgagaagatctccttgtctcagtttctgattcagaaatttcacacaacttccaggctggccttctacagcagcactggctggtacaaccgtctgtacattaacttcacgttgcgtcgccacatcttcttcttcttgctccaaacatatttccctgccactctgatggtcatgctgtcctgggtgtccttctggatcgaccgcagagctgtgcctgccagagtttcactgggtatcacgacggtgctgaccatgaccaccatcatcacgggcgtgaatgcctccatgccgcgcgtctcctacgtcaaggccgtggacatctacctctgggtcagctttgtgttcgtgttcctctcggtgctggagtatgcggctgtcaactacctgaccaccgtgcaggagcgcaaggaacggaagctgcgggagaagttcccgtgcatgtgtggaatgcttcattcaaaaaccatgatgctggatggaagctacagtgagtctgaggccaacagcctggctgggtaccccagaagccatatcctgacagaagaagaaaggcaagacaaaatagtggtccacctgggcctgagtggtgaagccaacgctgccagaaagaaggggcttctgaagggccagacgggttttcgtatcttccagaatacccatgccattgacaaatactctaggttgatattccctgcctcctacatatttttcaacttaatttattggtcagtgttttcctag

SEQ ID NO: 22 - DNA, 1404 bp, Homo sapiens
atggtcctggctttccagttagtctccttcacctacatctggatcatattgaaaccaaatgtttgtgctgcttctaacatcaagatgacacaccagcggtgctcctcttcaatgaaacaaacctgcaaacaagaaactagaatgaagaaagatgacagtaccaaagcgcggcctcagaaatatgagcaacttctccatatagaggacaacgatttcgcaatgagacctggatttggagggtctccagtgccagtaggtatagatgtccatgttgaaagcattgacagcatttcagagactaacatggactttacaatgactttttatctcaggcattactggaaagacgagaggctctcctttcctagcacagcaaacaaaagcatgacatttgatcatagattgaccagaaagatctgggtgcctgatatcttttttgtccactctaaaagatccttcatccatgatacaactatggagaatatcatgctgcgcgtacaccctgatggaaacgtcctcctaagtctcaggataacggtttcggccatgtgctttatggatttcagcaggtttcctcttgacactcaaaattgttctcttgaactggaaagctatgcctacaatgaggatgacctaatgctatactggaaacacggaaacaagtccttaaatactgaagaacatatgtccctttctcagttcttcattgaagacttcagtgcatctagtggattagctttctatagcagcacaggttggtacaataggcttttcatcaactttgtgctaaggaggcatgttttcttctttgtgctgcaaacctatttcccagccatattgatggtgatgctttcatgggtttcattttggattgaccgaagagctgttcctgcaagagtttccctgggaatcaccacagtgctgaccatgtccacaatcatcactgctgtgagcgcctccatgccccaggtgtcctacctcaaggctgtggatgtgtacctgtgggtcagctccctctttgtgttcctgtcagtcattgagtatgcagctgtgaactacctcaccacagtggaagagcggaaacaattcaagaagacaggaaagatttctaggatgtacaatattgatgcagttcaagctatggcctttgatggttgttaccatgacagcgagattgacatggaccagacttccctctctctaaactcagaagacttcatgagaagaaaatcgatatgcagccccagcaccgattcatctcggataaagagaagaaaatccctaggaggacatgttggtagaatcattctggaaaacaaccatgtcattgacacctattctaggattttattccccattgtgtatattttatttaatttgttttactggggtgtatatgtatga

SEQ ID NO: 23 - DNA, 2886 bp, Homo sapiens
atgttgctgctgctgttactggcgccactcttcctccgccccccgggcgcgggcggggcgcagacccccaacgccacctcagaaggttgccagatcatacacccgccctgggaagggggcatcaggtaccggggcctgactcgggaccaggtgaaggctatcaacttcctgccagtggactatgagattgagtatgtgtgccggggggagcgcgaggtggtggggcccaaggtccgcaagtgcctggccaacggctcctggacagatatggacacacccagccgctgtgtccgaatctgctccaagtcttatttgaccctggaaaatgggaaggttttcctgacgggtggggacctcccagctctggacggagcccgggtggatttccggtgtgaccccgacttccatctggtgggcagctcccggagcatctgtagtcagggccagtggagcacccccaagccccactgccaggtgaatcgaacgccacactcagaacggcgcgcagtgtacatcggggcactgtttcccatgagcgggggctggccagggggccaggcctgccagcccgcggtggagatggcgctggaggacgtgaatagccgcagggacatcctgccggactatgagctcaagctcatccaccacgacagcaagtgtgatccaggccaagccaccaagtacctatatgagctgctctacaacgaccctatcaagatcatccttatgcctggctgcagctctgtctccacgctggtggctgaggctgctaggatgtggaacctcattgtgctttcctatggctccagctcaccagccctgtcaaaccggcagcgtttccccactttcttccgaacgcacccatcagccacactccacaaccctacccgcgtgaaactctttgaaaagtggggctggaagaagattgctaccatccagcagaccactgaggtcttcacttcgactctggacgacctggaggaacgagtgaaggaggctggaattgagattactttccgccagagtttcttctcagatccagctgtgcccgtcaaaaacctgaagcgccaggatgcccgaatcatcgtgggacttttctatgagactgaagcccggaaagttttttgtgaggtgtacaaggagcgtctctttgggaagaagtacgtctggttcctcattgggtggtatgctgacaattggttcaagatctacgacccttctatcaactgcacagtggatgagatgactgaggcggtggagggccacatcacaactgagattgtcatgctgaatcctgccaatacccgcagcatttccaacatgacatcccaggaatttgtggagaaactaaccaagcgactgaaaagacaccctgaggagacaggaggcttccaggaggcaccgctggcctatgatgccatctgggccttggcactggccctgaacaagacatctggaggaggcggccgttctggtgtgcgcctggaggacttcaactacaacaaccagaccattaccgaccaaatctaccgggcaatgaactcttcgtcctttgagggtgtctctggccatgtggtgtttgatgccagcggctctcggatggcatggacgcttatcgagcagcttcagggtggcagctacaagaagattggctactatgacagcaccaaggatgatctttcctggtccaaaacagataaatggattggagggtcccccccagctgaccagaccctggtcatcaagacattccgcttcctgtcacagaaactctttatctccgtctcagttctctccagcctgggcattgtcctagctgttgtctgtctgtcctttaacatctacaactcacatgtccgttatatccagaactcacagcccaacctgaacaacctgactgctgtgggctgctcactggctttagctgctgtcttccccctggggctcgatggttaccacattgggaggaaccagtttcctttcgtctgccaggcccgcctctggctcctgggcctgggctttagtctgggctacggttccatgttcaccaagatttggtgggtccacacggtcttcacaaagaaggaagaaaagaaggagtggaggaagactctggaaccctggaagctgtatgccacagtgggcctgctggtgggcatggatgtcctcactctcgccatctggcagatcgtggaccctctgcaccggaccattgagacatttgccaaggaggaacctaaggaagatattgacgtctctattctgccccagctggagcattgcagctccaggaagatgaatacatggcttggcattttctatggttacaaggggctgctgctgctgctgggaatcttccttgcttatgagaccaagagtgtgtccactgagaagatcaatgatcaccgggctgtgggcatggctatctacaatgtggcagtcctgtgcctcatcactgctcctgtcaccatgattctgtccagccagcaggatgcagcctttgcctttgcctctcttgccatagttttctcctcctatatcactcttgttgtgctctttgtgcccaagatgcgcaggctgatcacccgaggggaatggcagtcggaggcgcaggacaccatgaagacagggtcatcgaccaacaacaacgaggaggagaagtcccggctgttggagaaggagaaccgtgaactggaaaagatcattgctgagaaagaggagcgtgtctctgaactgcgccatcaactccagtctcggcagcagctccgctcccggcgccacccaccgacacccccagaaccctctgggggcctgcccaggggaccccctgagccccccgaccggcttagctgtgatgggagtcgagtgcatttgctttataagtga

SEQ ID NO: 24 - DNA, 2535 bp, Homo sapiens
atggggcccggggccccttttgcccgggtggggtggccactgccgcttctggttgtgatggcggcaggggtggctccggtgtgggcctcccactccccccatctcccgcggcctcactcgcgggtccccccgcacccctcctcagaacggcgcgcagtgtacatcggggcactgtttcccatgagcgggggctggccagggggccaggcctgccagcccgcggtggagatggcgctggaggacgtgaatagccgcagggacatcctgccggactatgagctcaagctcatccaccacgacagcaagtgtgatccaggccaagccaccaagtacctatatgagctgctctacaacgaccctatcaagatcatccttatgcctggctgcagctctgtctccacgctggtggctgaggctgctaggatgtggaacctcattgtgctttcctatggctccagctcaccagccctgtcaaaccggcagcgtttccccactttcttccgaacgcacccatcagccacactccacaaccctacccgcgtgaaactctttgaaaagtggggctggaagaagattgctaccatccagcagaccactgaggtcttcacttcgactctggacgacctggaggaacgagtgaaggaggctggaattgagattactttccgccagagtttcttctcagatccagctgtgcccgtcaaaaacctgaagcgccaggatgcccgaatcatcgtgggacttttctatgagactgaagcccggaaagttttttgtgaggtgtacaaggagcgtctctttgggaagaagtacgtctggttcctcattgggtggtatgctgacaattggttcaagatctacgacccttctatcaactgcacagtggatgagatgactgaggcggtggagggccacatcacaactgagattgtcatgctgaatcctgccaatacccgcagcatttccaacatgacatcccaggaatttgtggagaaactaaccaagcgactgaaaagacaccctgaggagacaggaggcttccaggaggcaccgctggcctatgatgccatctgggccttggcactggccctgaacaagacatctggaggaggcggccgttctggtgtgcgcctggaggacttcaactacaacaaccagaccattaccgaccaaatctaccgggcaatgaactcttcgtcctttgagggtgtctctggccatgtggtgtttgatgccagcggctctcggatggcatggacgcttatcgagcagcttcagggtggcagctacaagaagattggctactatgacagcaccaaggatgatctttcctggtccaaaacagataaatggattggagggtcccccccagctgaccagaccctggtcatcaagacattccgcttcctgtcacagaaactctttatctccgtctcagttctctccagcctgggcattgtcctagctgttgtctgtctgtcctttaacatctacaactcacatgtccgttatatccagaactcacagcccaacctgaacaacctgactgctgtgggctgctcactggctttagctgctgtcttccccctggggctcgatggttaccacattgggaggaaccagtttcctttcgtctgccaggcccgcctctggctcctgggcctgggctttagtctgggctacggttccatgttcaccaagatttggtgggtccacacggtcttcacaaagaaggaagaaaagaaggagtggaggaagactctggaaccctggaagctgtatgccacagtgggcctgctggtgggcatggatgtcctcactctcgccatctggcagatcgtggaccctctgcaccggaccattgagacatttgccaaggaggaacctaaggaagatattgacgtctctattctgccccagctggagcattgcagctccaggaagatgaatacatggcttggcattttctatggttacaaggggctgctgctgctgctgggaatcttccttgcttatgagaccaagagtgtgtccactgagaagatcaatgatcaccgggctgtgggcatggctatctacaatgtggcagtcctgtgcctcatcactgctcctgtcaccatgattctgtccagccagcaggatgcagcctttgcctttgcctctcttgccatagttttctcctcctatatcactcttgttgtgctctttgtgcccaagatgcgcaggctgatcacccgaggggaatggcagtcggaggcgcaggacaccatgaagacagggtcatcgaccaacaacaacgaggaggagaagtcccggctgttggagaaggagaaccgtgaactggaaaagatcattgctgagaaagaggagcgtgtctctgaactgcgccatcaactccagtctcggcagcagctccgctcccggcgccacccaccgacacccccagaaccctctgggggcctgcccaggggaccccctgagccccccgaccggcttagctgtgatgggagtcgagtgcatttgctttataagtga

SEQ ID NO: 25 - DNA, 2700 bp, Homo sapiens
atgttgctgctgctgctactggcgccactcttcctccgccccccgggcgcgggcggggcgcagacccccaacgccacctcagaaggttgccagatcatacacccgccctgggaagggggcatcaggtaccggggcctgactcgggaccaggtgaaggctatcaacttcctgccagtggactatgagattgagtatgtgtgccggggggagcgcgaggtggtggggcccaaggtccgcaagtgcctggccaacggctcctggacagatatggacacacccagccgctgtgtgaatcgaacgccacactcagaacggcgcgcagtgtacatcggggcactgtttcccatgagcgggggctggccagggggccaggcctgccagcccgcggtggagatggcgctggaggacgtgaatagccgcagggacatcctgccggactatgagctcaagctcatccaccacgacagcaagtgtgatccaggccaagccaccaagtacctatatgagctgctctacaacgaccctatcaagatcatccttatgcctggctgcagctctgtctccacgctggtggctgaggctgctaggatgtggaacctcattgtgctttcctatggctccagctcaccagccctgtcaaaccggcagcgtttccccactttcttccgaacgcacccatcagccacactccacaaccctacccgcgtgaaactctttgaaaagtggggctggaagaagattgctaccatccagcagaccactgaggtcttcacttcgactctggacgacctggaggaacgagtgaaggaggctggaattgagattactttccgccagagtttcttctcagatccagctgtgcccgtcaaaaacctgaagcgccaggatgcccgaatcatcgtgggacttttctatgagactgaagcccggaaagttttttgtgaggtgtacaaggagcgtctctttgggaagaagtacgtctggttcctcattgggtggtatgctgacaattggttcaagatctacgacccttctatcaactgcacagtggatgagatgactgaggcggtggagggccacatcacaactgagattgtcatgctgaatcctgccaatacccgcagcatttccaacatgacatcccaggaatttgtggagaaactaaccaagcgactgaaaagacaccctgaggagacaggaggcttccaggaggcaccgctggcctatgatgccatctgggccttggcactggccctgaacaagacatctggaggaggcggccgttctggtgtgcgcctggaggacttcaactacaacaaccagaccattaccgaccaaatctaccgggcaatgaactcttcgtcctttgagggtgtctctggccatgtggtgtttgatgccagcggctctcggatggcatggacgcttatcgagcagcttcagggtggcagctacaagaagattggctactatgacagcaccaaggatgatctttcctggtccaaaacagataaatggattggagggtcccccccagctgaccagaccctggtcatcaagacattccgcttcctgtcacagaaactctttatctccgtctcagttctctccagcctgggcattgtcctagctgttgtctgtctgtcctttaacatctacaactcacatgtccgttatatccagaactcacagcccaacctgaacaacctgactgctgtgggctgctcactggctttagctgctgtcttccccctggggctcgatggttaccacattgggaggaaccagtttcctttcgtctgccaggcccgcctctggctcctgggcctgggctttagtctgggctacggttccatgttcaccaagatttggtgggtccacacggtcttcacaaagaaggaagaaaagaaggagtggaggaagactctggaaccctggaagctgtatgccacagtgggcctgctggtgggcatggatgtcctcactctcgccatctggcagatcgtggaccctctgcaccggaccattgagacatttgccaaggaggaacctaaggaagatattgacgtctctattctgccccagctggagcattgcagctccaggaagatgaatacatggcttggcattttctatggttacaaggggctgctgctgctgctgggaatcttccttgcttatgagaccaagagtgtgtccactgagaagatcaatgatcaccgggctgtgggcatggctatctacaatgtggcagtcctgtgcctcatcactgctcctgtcaccatgattctgtccagccagcaggatgcagcctttgcctttgcctctcttgccatagttttctcctcctatatcactcttgttgtgctctttgtgcccaagatgcgcaggctgatcacccgaggggaatggcagtcggaggcgcaggacaccatgaagacagggtcatcgaccaacaacaacgaggaggagaagtcccggctgttggagaaggagaaccgtgaactggaaaagatcattgctgagaaagaggagcgtgtctctgaactgcgccatcaactccagtctcggcagcagctccgctcccggcgccacccaccgacacccccagaaccctctgggggcctgcccaggggaccccctgagccccccgaccggcttagctgtgatgggagtcgagtgcatttgctttataagtga

SEQ ID NO: 26 - DNA, 2793 bp, Homo sapiens
atgttgctgctgctgttactggcgccactcttcctccgccccccgggcgcgggcggggcgcagacccccaacgccacctcagaaggttgccagatcatacacccgccctgggaagggggcatcaggtaccggggcctgactcgggaccaggtgaaggctatcaacttcctgccagtggactatgagattgagtatgtgtgccggggggagcgcgaggtggtggggcccaaggtccgcaagtgcctggccaacggctcctggacagatatggacacacccagccgctgtgtccgaatctgctccaagtcttatttgaccctggaaaatgggaaggttttcctgacgggtggggacctcccagctctggacggagcccgggtggatttccggtgtgaccccgacttccatctggtgggcagctcccggagcatctgtagtcagggccagtggagcacccccaagccccactgccaggtgaatcgaacgccacactcagaacggcgcgcagtgtacatcggggcactgtttcccatgagcgggggctggccagggggccaggcctgccagcccgcggtggagatggcgctggaggacgtgaatagccgcagggacatcctgccggactatgagctcaagctcatccaccacgacagcaagtgtgatccaggccaagccaccaagtacctatatgagctgctctacaacgaccctatcaagatcatccttatgcctggctgcagctctgtctccacgctggtggctgaggctgctaggatgtggaacctcattgtgctttcctatggctccagctcaccagccctgtcaaaccggcagcgtttccccactttcttccgaacgcacccatcagccacactccacaaccctacccgcgtgaaactctttgaaaagtggggctggaagaagattgctaccatccagcagaccactgaggtcttcacttcgactctggacgacctggaggaacgagtgaaggaggctggaattgagattactttccgccagagtttcttctcagatccagctgtgcccgtcaaaaacctgaagcgccaggatgcccgaatcatcgtgggacttttctatgagactgaagcccggaaagttttttgtgaggtgtacaaggagcgtctctttgggaagaagtacgtctggttcctcattgggtggtatgctgacaattggttcaagatctacgacccttctatcaactgcacagtggatgagatgactgaggcggtggagggccacatcacaactgagattgtcatgctgaatcctgccaatacccgcagcatttccaacatgacatcccaggaatttgtggagaaactaaccaagcgactgaaaagacaccctgaggagacaggaggcttccaggaggcaccgctggcctatgatgccatctgggccttggcactggccctgaacaagacatctggaggaggcggccgttctggtgtgcgcctggaggacttcaactacaacaaccagaccattaccgaccaaatctaccgggcaatgaactcttcgtcctttgagggtgtctctggccatgtggtgtttgatgccagcggctctcggatggcatggacgcttatcgagcagcttcagggtggcagctacaagaagattggctactatgacagcaccaaggatgatctttcctggtccaaaacagataaatggattggagggtcccccccagctgaccagaccctggtcatcaagacattccgcttcctgtcacagaaactctttatctccgtctcagttctctccagcctgggcattgtcctagctgttgtctgtctgtcctttaacatctacaactcacatgtccgttatatccagaactcacagcccaacctgaacaacctgactgctgtgggctgctcactggctttagctgctgtcttccccctggggctcgatggttaccacattgggaggaaccagtttcctttcgtctgccaggcccgcctctggctcctgggcctgggctttagtctgggctacggttccatgttcaccaagatttggtgggtccacacggtcttcacaaagaaggaagaaaagaaggagtggaggaagactctggaaccctggaagctgtatgccacagtgggcctgctggtgggcatggatgtcctcactctcgccatctggcagatcgtggaccctctgcaccggaccattgagacatttgccaaggaggaacctaaggaagatattgacgtctctattctgccccagctggagcattgcagctccaggaagatgaatacatggcttggcattttctatggttacaaggggctgctgctgctgctgggaatcttccttgcttatgagaccaagagtgtgtccactgagaagatcaatgatcaccgggctgtgggcatggctatctacaatgtggcagtcctgtgcctcatcactgctcctgtcaccatgattctgtccagccagcaggatgcagcctttgcctttgcctctcttgccatagttttctcctcctatatcactcttgttgtgctctttgtgcccaagatgcgcaggctgatcacccgaggggaatggcagtcggaggcgcaggacaccatgaagacagggtcatcgaccaacaacaacgaggaggagaagtcccggctgttggagaaggagaaccgtgaactggaaaagatcattgctgagagcggcggcctgccccggggcccccctgagccccctgaccggctgagctgcgacggatccagagtgcacctgctctacaag

SEQ ID NO: 27 - DNA, 1737 bp, Homo sapiens
atgttgctgctgctgctactggcgccactcttcctccgccccccgggcgcgggcggggcgcagacccccaacgccacctcagaaggttgccagatcatacacccgccctgggaagggggcatcaggtaccggggcctgactcgggaccaggtgaaggctatcaacttcctgccagtggactatgagattgagtatgtgtgccggggggagcgcgaggtggtggggcccaaggtccgcaagtgcctggccaacggctcctggacagatatggacacacccagccgctgtgtccgaatctgctccaagtcttatttgaccctggaaaatgggaaggttttcctgacgggtggggacctcccagctctggacggagcccgggtggatttccggtgtgaccccgacttccatctggtgggcagctcccggagcatctgtagtcagggccagtggagcacccccaagccccactgccaggtgaatcgaacgccacactcagaacggcgcgcagtgtacatcggggcactgtttcccatgagcgggggctggccagggggccaggcctgccagcccgcggtggagatggcgctggaggacgtgaatagccgcagggacatcctgccggactatgagctcaagctcatccaccacgacagcaagtgtgatccaggccaagccaccaagtacctatatgagctgctctacaacgaccctatcaagatcatccttatgcctggctgcagctctgtctccacgctggtggctgaggctgctaggatgtggaacctcattgtgctttcctatggctccagctcaccagccctgtcaaaccggcagcgtttccccactttcttccgaacgcacccatcagccacactccacaaccctacccgcgtgaaactctttgaaaagtggggctggaagaagattgctaccatccagcagaccactgaggtcttcacttcgactctggacgacctggaggaacgagtgaaggaggctggaattgagattactttccgccagagtttcttctcagatccagctgtgcccgtcaaaaacctgaagcgccaggatgcccgaatcatcgtgggacttttctatgagactgaagcccggaaagttttttgtgaggtgtacaaggagcgtctctttgggaagaagtacgtctggttcctcattgggtggtatgctgacaattggttcaagatctacgacccttctatcaactgcacagtggatgagatgactgaggcggtggagggccacatcacaactgagattgtcatgctgaatcctgccaatacccgcagcatttccaacatgacatcccaggaatttgtggagaaactaaccaagcgactgaaaagacaccctgaggagacaggaggcttccaggaggcaccgctggcctatgatgccatctgggccttggcactggccctgaacaagacatctggaggaggcggccgttctggtgtgcgcctggaggacttcaactacaacaaccagaccattaccgaccaaatctaccgggcaatgaactcttcgtcctttgagggtgtctctggccatgtggtgtttgatgccagcggctctcggatggcatggacgcttatcgagcagcttcagggtggcagctacaagaagattggctactatgacagcaccaaggatgatctttcctggtccaaaacagataaatggattgttatatccagaactcacagcccaacctga

SEQ ID NO: 28 - DNA, 2826 bp, Homo sapiens
atggcttccccgcggagctccgggcagcccgggccgccgccgccgccgccaccgccgcccgcgcgcctgctactgctactgctgctgccgctgctgctgcctctggcgcccggggcctggggctgggcgcggggcgccccccggccgccgcccagcagcccgccgctctccatcatgggcctcatgccgctcaccaaggaggtggccaagggcagcatcgggcgcggtgtgctccccgccgtggaactggccatcgagcagatccgcaacgagtcactcctgcgcccctacttcctcgacctgcggctctatgacacggagtgcgacaacgcaaaagggttgaaagccttctacgatgcaataaaatacgggcctaaccacttgatggtgtttggaggcgtctgtccatccgtcacatccatcattgcagagtccctccaaggctggaatctggtgcagctttcttttgctgcaaccacgcctgttctagccgataagaaaaaatacccttatttctttcggaccgtcccatcagacaatgcggtgaatccagccattctgaagttgctcaagcactaccagtggaagcgcgtgggcacgctgacgcaagacgttcagaggttctctgaggtgcggaatgacctgactggagttctgtatggcgaggacattgagatttcagacaccgagagcttctccaacgatccctgtaccagtgtcaaaaagctgaaggggaatgatgtgcggatcatccttggccagtttgaccagaatatggcagcaaaagtgttctgttgtgcatacgaggagaacatgtatggtagtaaatatcagtggatcattccgggctggtacgagccttcttggtgggagcaggtgcacacggaagccaactcatcccgctgcctccggaagaatctgcttgctgccatggagggctacattggcgtggatttcgagcccctgagctccaagcagatcaagaccatctcaggaaagactccacagcagtatgagagagagtacaacaacaagcggtcaggcgtggggcccagcaagttccacgggtacgcctacgatggcatctgggtcatcgccaagacactgcagagggccatggagacactgcatgccagcagccggcaccagcggatccaggacttcaactacacggaccacacgctgggcaggatcatcctcaatgccatgaacgagaccaacttcttcggggtcacgggtcaagttgtattccggaatggggagagaatggggaccattaaatttactcaatttcaagacagcagggaggtgaaggtgggagagtacaacgctgtggccgacacactggagatcatcaatgacaccatcaggttccaaggatccgaaccaccaaaagacaagaccatcatcctggagcagctgcggaagatctccctacctctctacagcatcctctctgccctcaccatcctcgggatgatcatggccagtgcttttctcttcttcaacatcaagaaccggaatcagaagctcataaagatgtcgagtccatacatgaacaaccttatcatccttggagggatgctctcctatgcttccatatttctctttggccttgatggatcctttgtctctgaaaagacctttgaaacactttgcaccgtcaggacctggattctcaccgtgggctacacgaccgcttttggggccatgtttgcaaagacctggagagtccacgccatcttcaaaaatgtgaaaatgaagaagaagatcatcaaggaccagaaactgcttgtgatcgtggggggcatgctgctgatcgacctgtgtatcctgatctgctggcaggctgtggaccccctgcgaaggacagtggagaagtacagcatggagccggacccagcaggacgggatatctccatccgccctctcctggagcactgtgagaacacccatatgaccatctggcttggcatcgtctatgcctacaagggacttctcatgttgttcggttgtttcttagcttgggagacccgcaacgtcagcatccccgcactcaacgacagcaagtacatcgggatgagtgtctacaacgtggggatcatgtgcatcatcggggccgctgtctccttcctgacccgggaccagcccaatgtgcagttctgcatcgtggctctggtcatcatcttctgcagcaccatcaccctctgcctggtattcgtgccgaagctcatcaccctgagaacaaacccagatgcagcaacgcagaacaggcgattccagttcactcagaatcagaagaaagaagattctaaaacgtccacctcggtcaccagtgtgaaccaagccagcacatcccgcctggagggcctacagtcagaaaaccatcgcctgcgaatgaagatcacagagctggataaagacttggaagaggtcaccatgcagctgcaggacacaccagaaaagaccacctacattaaacagaaccactaccaagagctcaatgacatcctcaacctgggaaacttcactgagagcacagatggaggaaaggccattttaaaaaatcacctcgatcaaaatccccagctacagtggaacacaacagagccctctcgaacatgcaaagatcctatagaagatataaactctccagaacacatccagcgtcggctgtccctccagctccccatcctccaccacgcctacctcccatccatcggaggcgtggacgccagctgtgtcagcccctgcgtcagccccaccgccagcccccgccacagacatgtgccaccctccttccgagtcatggtctcgggcctgtaa

SEQ ID NO: 29 - protein, 456 aa, Homo sapiens
MRKSPGLSDCLWAWILLLSTLTGRSYGQPSLQDELKDNTTVFTRILDRLLDGYDNRLRPGLGERVTEVKTDIFVTSFGPVSDHDMEYTIDVFFRQSWKDERLKFKGPMTVLRLNNLMASKIWTPDTFFHNGKKSVAHNMTMPNKLLRITEDGTLLYTMRLTVRAECPMHLEDFPMDAHACPLKFGSYAYTRAEVVYEWTREPARSVVVAEDGSRLNQYDLLGQTVDSGIVQSSTGEYVVMTTHFHLKRKIGYFVIQTYLPCIMTVILSQVSFWLNRESVPARTVFGVTTVLTMTTLSISARNSLPKVAYATAMDWFIAVCYAFVFSALIEFATVNYFTKRGYAWDGKSVVPEKPKKVKDPLIKKNNTYAPTATSYTPNLARGDPGLATIAKSATIEPKEVKPETKPPEPKKTFNSVSKIDRLSRIAFPLLFGIFNLVYWATYLNREPQLKAPTPHQ

SEQ ID NO: 30 - protein, 451 aa, Homo sapiens
MKTKLNIYNMQFLLFVFLVWDPARLVLANIQEDEAKNNITIFTRILDRLLDGYDNRLRPGLGDSITEVFTNIYVTSFGPVSDTDMEYTIDVFFRQKWKDERLKFKGPMNILRLNNLMASKIWTPDTFFHNGKKSVAHNMTMPNKLLRIQDDGTLLYTMRLTVQAECPMHLEDFPMDAHSCPLKFGSYAYTTSEVTYIWTYNASDSVQVAPDGSRLNQYDLLGQSIGKETIKSSTGEYTVMTAHFHLKRKIGYFVIQTYLPCIMTVILSQVSFWLNRESVPARTVFGVTTVLTMTTLSISARNSLPKVAYATAMDWFIAVCYAFVFSALIEFATVNYFTKRGWAWDGKSVVNDKKKEKASVMIQNNAYAVAVANYAPNLSKDPVLSTISKSATTPEPNKKPENKPAEAKKTFNSVSKIDRMSRIVFPVLFGTFNLVYWATYLNREPVLGVSP

SEQ ID NO: 31 - protein, 492 aa, Homo sapiens
MIITQTSHCYMTSLGILFLINILPGTTGQGESRRQEPGDFVKQDIGGLSPKHAPDIPDDSTDNITIFTRILDRLLDGYDNRLRPGLGDAVTEVKTDIYVTSFGPVSDTDMEYTIDVFFRQTWHDERLKFDGPMKILPLNNLLASKIWTPDTFFHNGKKSVAHNMTTPNKLLRLVDNGTLLYTMRLTIHAECPMHLEDFPMDVHACPLKFGSYAYTTAEVVYSWTLGKNKSVEVAQDGSRLNQYDLLGHVVGTEIIRSSTGEYVVMTTHFHLKRKIGYFVIQTYLPCIMTVILSQVSFWLNRESVPARTVFGVTTVLTMTTLSISARNSLPKVAYATAMDWFIAVCYAFVFSALIEFATVNYFTKRSWAWEGKKVPEALEMKKKTPAAPAKKTSTTFNIVGTTYPINLAKDTEFSTISKGAAPSASSTPTIIASPKATYVQDSPTETKTYNSVSKVDKISRIIFPVLFAIFNLVYWATYVNRESAIKGMIRKQ

SEQ ID NO: 32 - protein, 554 aa, Homo sapiens [sequence omitted]

SEQ ID NO: 33 - protein, 462 aa, Homo sapiens
MDNGMFSGFIMIKNLLLFCISMNLSSHFGFSQMPTSSVKDETNDNITIFTRILDGLLDGYDNRLRPGLGERITQVRTDIYVTSFGPVSDTEMEYTIDVFFRQSWKDERLRFKGPMQRLPLNNLLASKIWTPDTFFHNGKKSIAHNMTTPNKLLRLEDDGTLLYTMRLTISAECPMQLEDFPMDAHACPLKFGSYAYPNSEVVYVWTNGSTKSVVVAEDGSRLNQYHLMGQTVGTENISTSTGEYTIMTAHFHLKRKIGYFVIQTYLPCIMTVILSQVSFWLNRESVPARTVFGVTTVLTMTTLSISARNSLPKVAYATAMDWFIAVCYAFVFSALIEFATVNYFTKRGWAWDGKKALEAAKIKKKREVILNKSTNAFTTGKMSHPPNIPKEQTPAGTSNTTSVSVKPSEEKTSESKKTYNSISKIDKMSRIVFPVLFGTFNLVYWATYLNREPVIKGAASPK

SEQ ID NO: 34 - protein, 453 aa, Homo sapiens
MASSLPWLCIILWLENALGKLEVEGNFYSENVSRILDNLLEGYDNRLRPGFGGAVTEVKTDIYVTSFGPVSDVEMEYTMDVFFRQTWTDERLKFGGPTEILSLNNLMVSKIWTPDTFFRNGKKSIAHNMTTPNKLFRIMQNGTILYTMRLTINADCPMRLVNFPMDGHACPLKFGSYAYPKSEIIYTWKKGPLYSVEVPEESSSLLQYDLIGQTVSSETIKSNTGEYVIMTVYFHLQRKMGYFMIQIYTPCIMTVILSQVSFWINKESVPARTVFGITTVLTMTTLSISARHSLPKVSYATAMDWFIAVCFAFVFSALIEFAAVNYFTNLQTQKAKRKAQFAAPPTVTISKATEPLEAEIVLHPDSKYHLKKRITSLSLPIVSSSEANKVLTRAPILQSTPVTPPPLSPAFGGTSKIDQYSRILFPVAFAGFNLVYWVVYLSKDTMEVSSSVE

SEQ ID NO: 35 - protein, 474 aa, Homo sapiens
MWTVQNRESLGLLSFPVMITMVCCAHSTNEPSNMSYVKETVDRLLKGYDIRLRPDFGGPPVDVGMRIDVASIDMVSEVNMDYTLTMYFQQSWKDKRLSYSGIPLNLTLDNRVADQLWVPDTYFLNDKKSFVHGVTVKNRMIRLHPDGTVLYGLRITTTAACMMDLRRYPLDEQNCTLEIESYGYTTDDIEFYWNGGEGAVTGVNKIELPQFSIVDYKMVSKKVEFTTGAYPRLSLSFRLKRNIGYFILQTYMPSTLITILSWVSFWINYDASAARVALGITTVLTMTTISTHLRETLPKIPYVKAIDIYLMGCFVFVFLALLEYAFVNYIFFGKGPQKKGASKQDQSANEKNKLEMNKVQVDAHGNILLSTLEIRNETSGSEVLTSVSDPKATMYSYDSASIQYRKPLSSREAYGRALDRHGVPSKGRIRRRASQLKVKIPDLTDVNSIDKWSRMFFPITFSLFNVVYWLYYVH

SEQ ID NO: 36 - protein, 511 aa, Homo sapiens
WRVRKRGYFGIWSFPLIIAAVCAQSVNDPSNMSLVKETVDRLLKGYDIRLRPDFGGPPVAVGMNIDIASIDMVSEVNMDYTLTMYFQQAWRDKRLSYNVIPLNLTLDNRVADQLWVPDTYFLNDKKSFVHGVTVKNRMIRLHPDGTVLYGLRITTTAACMMDLRRYPLDEQNCTLEIESYGYTTDDIEFYWRGDDNAVTGVTKIELPQFSIVDYKLITKKVVFSTGSYPRLSLSFKLKRNIGYFILQTYMPSILITILSWVSFWINYDASAARVALGITTVLTMTTINTHLRETLPKIPYVKAIDMYLMGCFVFVFMALLEYALVNYIFFGRGPQRQKKAAEKAASANNEKMRLDVNKIFYKDIKQNGTQYRSLWDPTGNLSPTRRTTNYDFSLYTMDPHENILLSTLEIKNEMATSEAVMGLGDPRSTMLAYDASSIQYRKAGLPRHSFGRNALERHVAQKKSRLRRRASQLKITIPDLTDVNAIDRWSRIFFPVVFSFFNIVYWLYYVN

SEQ ID NO: 37 - protein, 474 aa, Homo sapiens
MWRVRKRGYFGIWSFPLIIAAVCAQSVNDPSNMSLVKETVDRLLKGYDIRLRPDFGGPPVAVGMNIDIASIDMVSEVNMDYTLTMYFQQAWRDKRLSYNVIPLNLTLDNRVADQLWVPDTYFLNDKKSFVHGVTVKNRMIRLHPDGTVLYGLRITTTAACMMDLRRYPLDEQNCTLEIESYGYTTDDIEFYWRGDDNAVTGVTKIELPQFSIVDYKLITKKVVFSTGSYPRLSLSFKLKRNIGYFILQTYMPSILITILSWVSFWINYDASAARVALGITTVLTMTTINTHLRETLPKIPYVKAIDMYLMGCFVFVFMALLEYALVNYIFFGRGPQRQKKAAEKAASANNEKMRLDVNKMDPHENILLSTLEIKNEMATSEAVMGLGDPRSTMLAYDASSIQYRKAGLPRHSFGRNALERHVAQKKSRLRRRASQLKITIPDLTDVNAIDRWSRIFFPVVFSFFNIVYWLYYVN

SEQ ID NO: 38 - protein, 473 aa, Homo sapiens [sequence omitted]

SEQ ID NO: 39 - protein, 473 aa, Homo sapiens
MCSGLLELLLPIWLSWTLGTRGSEPRSVNDPGNMSFVKETVDKLLKGYDIRLRPDFGGPPVCVGMNIDIASIDMVSEVNMDYTLTMYFQQYWRDKRLAYSGIPLNLTLDNRVADQLWVPDTYFLNDKKSFVHGVTVKNRMIRLHPDGTVLYGLRITTTAACMMDLRRYPLDEQNCTLEIESYGYTTDDIEFYWRGGDKAVTGVERIELPQFSIVEHRLVSRNVVFATGAYPRLSLSFRLKRNIGYFILQTYMPSILITILSWVSFWINYDASAARVALGITTVLTMTTINTHLRETLPKIPYVKAIDMYLMGCFVFVFLALLEYAFVNYIFFGRGPQRQKKLAEKTAKAKNDRSKSESNRVDAHGNILLTSLEVHNEMNEVSGGIGDTRNSAISFDNSGIQYRKQSMPREGHGRFLGDRSLPHKKTHLRRRSSQLKIKIPDLTDVNAIDRWSRIVFPFTFSLFNLVYWLYYVN

SEQ ID NO: 40 - protein, 465 aa, Homo sapiens
MGPLKAFLFSPFLLRSQSRGVRLVFLLLTLHLGNCVDKADDEDDEDLTVNKTWVLAPKIHEGDITQILNSLLQGYDNKLRPDIGVRPTVIETDVYVNSIGPVDPINMEYTIDIIFAQTWFDSRLKFNSTMKVLMLNSNMVGKIWIPDTFFRNSRKSDAHWITTPNRLLRIWNDGRVLYTLRLTINAECYLQLHNFPMDEHSCPLEFSSYGYPKNEIEYKWKKPSVEVADPKYWRLYQFAFVGLRNSTEITHTISGDYVIMTIFFDLSRRMGYFTIQTYIPCILTVVLSWVSFWINKDAVPARTSLGITTVLTMTTLSTIARKSLPKVSYVTAMDLFVSVCFIFVFAALMEYGTLHYFTSNQKGKTATKDRKLKNKASMTPGLHPGSTLIPMNNISVPQEDDYGYQCLEGKDCASFFCCFEDCRTGSWREGRIHIRIAKIDSYSRIFFPTAFALFNLVYWVGYLYL

SEQ ID NO: 41 - protein, 467 aa, Homo sapiens
MSSPNIWSTGSSVYSTPVFSQKMTVWILLLLSLYPGFTSQKSDDDYEDYASNKTWVLTPKVPEGDVTVILNNLLEGYDNKLRPDIGVKPTLIHTDMYVNSIGPVNAINMEYTIDIFFAQTWYDRRLKFNSTIKVLRLNSNMVGKIWIPDTFFRNSKKADAHWITTPNRMLRIWNDGRVLYTLRLTIDAECQLQLHNFPMDEHSCPLEFSSYGYPREEIVYQWKRSSVEVGDTRSWRLYQFSFVGLRNTTEVVKTTSGDYVVMSVYFDLSRRMGYFTIQTYIPCTLIVVLSWVSFWINKDAVPARTSLGITTVLTMTTLSTIARKSLPKVSYVTAMDLFVSVCFIFVFSALVEYGTLHYFVSNRKPSKDKDKKKKNPAPTIDIRPRSATIQMNNATHLQERDEEYGYECLDGKDCASFFCCFEDCRTGAWRHGRIHIRIAKMDSYARIFFPTAFCLFNLVYWVSYLYL

SEQ ID NO: 42 - protein, 475 aa, Homo sapiens
MSSPNIWSTGSSVYSTPVFSQKMTVWILLLLSLYPGFTSQKSDDDYEDYASNKTWVLTPKVPEGDVTVILNNLLEGYDNKLRPDIGVKPTLIHTDMYVNSIGPVNAINMEYTIDIFFAQTWYDRRLKFNSTIKVLRLNSNMVGKIWIPDTFFRNSKKADAHWITTPNRMLRIWNDGRVLYTLRLTIDAECQLQLHNFPMDEHSCPLEFSSYGYPREEIVYQWKRSSVEVGDTRSWRLYQFSFVGLRNTTEVVKTTSGDYVVMSVYFDLSRRMGYFTIQTYIPCTLIVVLSWVSFWINKDAVPARTSLGITTVLTMTTLSTIARKSLPKVSYVTAMDLFVSVCFIFVFSALVEYGTLHYFVSNRKPSKDKDKKKKNPLLRMFSFKAPTIDIRPRSATIQMNNATHLQERDEEYGYECLDGKDCASFFCCFEDCRTGAWRHGRIHIRIAKMDSYARIFFPTAFCLFNLVYWVSYLYL

SEQ ID NO: 43 - protein, 467 aa, Homo sapiens
MAPKLLLLLCLFSGLHARSRKVEEDEYEDSSSNQKWVLAPKSQDTDVTLILNKLLREYDKKLRPDIGIKPTVIDVDIYVNSIGPVSSINMEYQIDIFFAQTWTDSRLRFNSTMKILTLNSNMVGLIWIPDTIFRNSKTAEAHWITTPNQLLRIWNDGKILYTLRLTINAECQLQLHNFPMDEHSCPLIFSSYGYPKEEMIYRWRKNSVEAADQKSWRLYQFDFMGLRNTTEIVTTSAGDYVVMTIYFELSRRMGYFTIQTYIPCILTVVLSWVSFWIKKDATPARTALGITTVLTMTTLSTIARKSLPRVSYVTAMDLFVTVCFLFVFAALMEYATLNYYSSCRKPTTTKKTTSLLHPDSSRWIPERISLQAPSNYSLLDMRPPPTAMITLNNSVYWQEFEDTCVYECLDGKDCQSFFCCYEECKSGSWRKGRIHIDILELDSYSRVFFPTSFLLFNLVYWVGYLYL

SEQ ID NO: 44 - protein, 452 aa, Homo sapiens [sequence omitted]

SEQ ID NO: 45 - protein, 506 aa, Homo sapiens
MLSKVLPVLLGILLILQSRVEGPQTESKNEASSRDVVYGPQPQPLENQLLSEETKSTETETGSRVGKLPEASRILNTILSNYDHKLRPGIGEKPTVVTVEISVNSLGPLSILDMEYTIDIIFSQTWYDERLCYNDTFESLVLNGNVVSQLWIPDTFFRNSKRTHEHEITMPNQMVRIYKDGKVLYTIRMTIDAGCSLHMLRFPMDSHSCPLSFSSFSYPENEMIYKWENFKLEINEKNSWKLFQFDFTGVSNKTEIITTPVGDFMVMTIFFNVSRRFGYVAFQNYVPSSVTTMLSWVSFWIKTESAPARTSLGITSVLTMTTLGTFSRKNFPRVSYITALDFYIAICFVFCFCALLEFAVLNFLIYNQTKAHASPKLRHPRINSRAHARTRARSRACARQHQEAFVCQIVTTEGSDGEERPSCSAQQPPSPGSPEGPRSLCSKLACCEWCKRFKKYFCMVPDCEGSTWQQGRLCIHVYRLDNYSRVVFPVTFFFFNVLYWLVCLNL

SEQ ID NO: 46 - protein, 440 aa, Homo sapiens
MNYSLHLAFVCLSLFTERMCIQGSQFNVEVGRSDKLSLPGFENLTAGYNKFLRPNFGGEPVQIALTLDIASISSISESNMDYTATIYLRQRWMDQRLVFEGNKSFTLDARLVEFLWVPDTYIVESKKSFLHEVTVGNRLIRLFSNGTVLYALRITTTVACNMDLSKYPMDTQTCKLQLESWGYDGNDVEFTWLRGNDSVRGLEHLRLAQYTIERYFTLVTRSQQETGNYTRLVLQFELRRNVLYFILETYVPSTFLVVLSWVSFWISLDSVPARTCIGVTTVLSMTTLMIGSRTSLPNTNCFIKAIDVYLGICFSFVFGALLEYAVAHYSSLQQMAAKDRGTTKEVEEVSITNIINSSISSFKRKISFASIEISSDNVDYSDLTMKTSDKFKFVFREKMGRIVDYFTIQNPSNVDHYSKLLFPLIFMLANVFYWAYYMYF

SEQ ID NO: 47 - protein, 632 aa, Homo sapiens
MGIRGMLRAAVILLLIRTWLAEGNYPSPIPKFHFEFSSAVPEVVLNLFNCKNCANEAVVQKILDRVLSRYDVRLRPNFGGAPVPVRISIYVTSIEQISEMNMDYTITMFFHQTWKDSRLAYYETTLNLTLDYRMHEKLWVPDCYFLNSKDAFVHDVTVENRVFQLHPDGTVRYGIRLTTTAACSLDLHKFPMDKQACNLVVESYGYTVEDIILFWDDNGNAIHMTEELHIPQFTFLGRTITSKEVYFYTGSYIRLILKFQVQREVNSYLVQVYWPTVLTTITSWISFWMNYDSSAARVTIGLTSMLILTTIDSHLRDKLPNISCIKAIDIYILVCLFFVFLSLLEYVYINYLFYSRGPRRQPRRHRRPRRVIARYRYQQVVVGNVQDGLINVEDGVSSLPITPAQAPLASPESLGSLTSTSEQAQLATSESLSPLTSLSGQAPLATGESLSDLPSTSEQARHSYGVRFNGFQADDSIFPTEIRNRVEAHGHGVTHDHEDSNESLSSDERHGHGPSGKPMLHHGEKGVQEAGWDLDDNNDKSDCLAIKEQFKCDTNSTWGLNDDELMAHGQEKDSSSESEDSCPPSPGCSFTEGFSFDLFNPDYVPKVDKWSRFLFPLAFGLFNIVYWVYHMY

SEQ ID NO: 48 - protein, 473 aa, Homo sapiens
MRFGIFLLWWGWVLATESRMHWPGREVHEMSKKGRPQRQRREVHEDAHKQVSPILRRSPDITKSPLTKSEQLLRIDDHDFSMRPGFGGPAIPVGVDVQVESLDSISEVDMDFTMTLYLRHYWKDERLSFPSTNNLSMTFDGRLVKKIWVPDMFFVHSKRSFIHDTTTDNVMLRVQPDGKVLYSLRVTVTAMCNMDFSRFPLDTQTCSLEIESYAYTEDDLMLYWKKGNDSLKTDERISLSQFLIQEFHTTTKLAFYSSTGWYNRLYINFTLRRHIFFFLLQTYFPATLMVMLSWVSFWIDRRAVPARVPLGITTVLTMSTIITGVNASMPRVSYIKAVDIYLWVSFVFVFLSVLEYAAVNYLTTVQERKEQKLREKLPCTSGLPPPRTAMLDGNYSDGEVNDLDNYMPENGEKPDRMMVQLTLASERSSPQRKSQRSSYVSMRIDTHAIDKYSRIIFPAAYILFNLIYWSIFS

SEQ ID NO: 49 - protein, 465 aa, Homo sapiens
MPYFTRLILFLFCLMVLVESRKPKRKRWTGQVEMPKPSHLYKKNLDVTKIRKGKPQQLLRVDEHDFSMRPAFGGPAIPVGVDVQVESLDSISEVDMDFTMTLYLRHYWKDERLAFSSASNKSMTFDGRLVKKIWVPDVFFVHSKRSFTHDTTTDNIMLRVFPDGHVLYSMRITVTAMCNMDFSHFPLDSQTCSLELESYAYTDEDLMLYWKNGDESLKTDEKISLSQFLIQKFHTTSRLAFYSSTGWYNRLYINFTLRRHIFFFLLQTYFPATLMVMLSWVSFWIDRRAVPARVSLGITTVLTMTTIITGVNASMPRVSYVKAVDIYLWVSFVFVFLSVLEYAAVNYLTTVQERKERKLREKFPCMCGMLHSKTMMLDGSYSESEANSLAGYPRSHILTEEERQDKIVVHLGLSGEANAARKKGLLKGQTGFRIFQNTHAIDKYSRLIFPASYIFFNLIYWSVFS

SEQ ID NO: 50 - protein, 467 aa, Homo sapiens
MVLAFQLVSFTYIWIILKPNVCAASNIKMTHQRCSSSMKQTCKQETRMKKDDSTKARPQKYEQLLHIEDNDFAMRPGFGGSPVPVGIDVHVESIDSISETNMDFTMTFYLRHYWKDERLSFPSTANKSMTFDHRLTRKIWVPDIFFVHSKRSFIHDTTMENIMLRVHPDGNVLLSLRITVSAMCFMDFSRFPLDTQNCSLELESYAYNEDDLMLYWKHGNKSLNTEEHMSLSQFFIEDFSASSGLAFYSSTGWYNRLFINFVLRRHVFFFVLQTYFPAILMVMLSWVSFWIDRRAVPARVSLGITTVLTMSTIITAVSASMPQVSYLKAVDVYLWVSSLFVFLSVIEYAAVNYLTTVEERKQFKKTGKISRMYNIDAVQAMAFDGCYHDSEIDMDQTSLSLNSEDFMRRKSICSPSTDSSRIKRRKSLGGHVGRIILENNHVIDTYSRILFPIVYILFNLFYWGVYV

SEQ ID NO: 51 - protein, 961 aa, Homo sapiens
MLLLLLLAPLFLRPPGAGGAQTPNATSEGCQIIHPPWEGGIRYRGLTRDQVKAINFLPVDYEIEYVCRGEREVVGPKVRKCLANGSWTDMDTPSRCVRICSKSYLTLENGKVFLTGGDLPALDGARVDFRCDPDFHLVGSSRSICSQGQWSTPKPHCQVNRTPHSERRAVYIGALFPMSGGWPGGQACQPAVEMALEDVNSRRDILPDYELKLIHHDSKCDPGQATKYLYELLYNDPIKIILMPGCSSVSTLVAEAARMWNLIVLSYGSSSPALSNRQRFPTFFRTHPSATLHNPTRVKLFEKWGWKKIATIQQTTEVFTSTLDDLEERVKEAGIEITFRQSFFSDPAVPVKNLKRQDARIIVGLFYETEARKVFCEVYKERLFGKKYVWFLIGWYADNWFKIYDPSINCTVDEMTEAVEGHITTEIVMLNPANTRSISNMTSQEFVEKLTKRLKRHPEETGGFQEAPLAYDAIWALALALNKTSGGGGRSGVRLEDFNYNNQTITDQIYRAMNSSSFEGVSGHVVFDASGSRMAWTLIEQLQGGSYKKIGYYDSTKDDLSWSKTDKWIGGSPPADQTLVIKTFRFLSQKLFISVSVLSSLGIVLAVVCLSFNIYNSHVRYIQNSQPNLNNLTAVGCSLALAAVFPLGLDGYHIGRNQFPFVCQARLWLLGLGFSLGYGSMFTKIWWVHTVFTKKEEKKEWRKTLEPWKLYATVGLLVGMDVLTLAIWQIVDPLHRTIETFAKEEPKEDIDVSILPQLEHCSSRKMNTWLGIFYGYKGLLLLLGIFLAYETKSVSTEKINDHRAVGMAIYNVAVLCLITAPVTMILSSQQDAAFAFASLAIVFSSYITLVVLFVPKMRRLITRGEWQSEAQDTMKTGSSTNNNEEEKSRLLEKENRELEKIIAEKEERVSELRHQLQSRQQLRSRRHPPTPPEPSGGLPRGPPEPPDRLSCDGSRVHLLYK

SEQ ID NO: 52 - protein, 844 aa, Homo sapiens
MGPGAPFARVGWPLPLLVVMAAGVAPVWASHSPHLPRPHSRVPPHPSSERRAVYIGALFPMSGGWPGGQACQPAVEMALEDVNSRRDILPDYELKLIHHDSKCDPGQATKYLYELLYNDPIKIILMPGCSSVSTLVAEAARMWNLIVLSYGSSSPALSNRQRFPTFFRTHPSATLHNPTRVKLFEKWGWKKIATIQQTTEVFTSTLDDLEERVKEAGIEITFRQSFFSDPAVPVKNLKRQDARIIVGLFYETEARKVFCEVYKERLFGKKYVWFLIGWYADNWFKIYDPSINCTVDEMTEAVEGHITTEIVMLNPANTRSISNMTSQEFVEKLTKRLKRHPEETGGFQEAPLAYDAIWALALALNKTSGGGGRSGVRLEDFNYNNQTITDQIYRAMNSSSFEGVSGHVVFDASGSRMAWTLIEQLQGGSYKKIGYYDSTKDDLSWSKTDKWIGGSPPADQTLVIKTFRFLSQKLFISVSVLSSLGIVLAVVCLSFNIYNSHVRYIQNSQPNLNNLTAVGCSLALAAVFPLGLDGYHIGRNQFPFVCQARLWLLGLGFSLGYGSMFTKIWWVHTVFTKKEEKKEWRKTLEPWKLYATVGLLVGMDVLTLAIWQIVDPLHRTIETFAKEEPKEDIDVSILPQLEHCSSRKMNTWLGIFYGYKGLLLLLGIFLAYETKSVSTEKINDHRAVGMAIYNVAVLCLITAPVTMILSSQQDAAFAFASLAIVFSSYITLVVLFVPKMRRLITRGEWQSEAQDTMKTGSSTNNNEEEKSRLLEKENRELEKIIAEKEERVSELRHQLQSRQQLRSRRHPPTPPEPSGGLPRGPPEPPDRLSCDGSRVHLLYK

SEQ ID NO: 53 - protein, 899 aa, Homo sapiens
MLLLLLLAPLFLRPPGAGGAQTPNATSEGCQIIHPPWEGGIRYRGLTRDQVKAINFLPVDYEIEYVCRGEREVVGPKVRKCLANGSWTDMDTPSRCVNRTPHSERRAVYIGALFPMSGGWPGGQACQPAVEMALEDVNSRRDILPDYELKLIHHDSKCDPGQATKYLYELLYNDPIKIILMPGCSSVSTLVAEAARMWNLIVLSYGSSSPALSNRQRFPTFFRTHPSATLHNPTRVKLFEKWGWKKIATIQQTTEVFTSTLDDLEERVKEAGIEITFRQSFFSDPAVPVKNLKRQDARIIVGLFYETEARKVFCEVYKERLFGKKYVWFLIGWYADNWFKIYDPSINCTVDEMTEAVEGHITTEIVMLNPANTRSISNMTSQEFVEKLTKRLKRHPEETGGFQEAPLAYDAIWALALALNKTSGGGGRSGVRLEDFNYNNQTITDQIYRAMNSSSFEGVSGHVVFDASGSRMAWTLIEQLQGGSYKKIGYYDSTKDDLSWSKTDKWIGGSPPADQTLVIKTFRFLSQKLFISVSVLSSLGIVLAVVCLSFNIYNSHVRYIQNSQPNLNNLTAVGCSLALAAVFPLGLDGYHIGRNQFPFVCQARLWLLGLGFSLGYGSMFTKIWWVHTVFTKKEEKKEWRKTLEPWKLYATVGLLVGMDVLTLAIWQIVDPLHRTIETFAKEEPKEDIDVSILPQLEHCSSRKMNTWLGIFYGYKGLLLLLGIFLAYETKSVSTEKINDHRAVGMAIYNVAVLCLITAPVTMILSSQQDAAFAFASLAIVFSSYITLVVLFVPKMRRLITRGEWQSEAQDTMKTGSSTNNNEEEKSRLLEKENRELEKIIAEKEERVSELRHQLQSRQQLRSRRHPPTPPEPSGGLPRGPPEPPDRLSCDGSRVHLLYK

SEQ ID NO: 54 - protein, 931 aa, Homo sapiens
MLLLLLLAPLFLRPPGAGGAQTPNATSEGCQIIHPPWEGGIRYRGLTRDQVKAINFLPVDYEIEYVCRGEREVVGPKVRKCLANGSWTDMDTPSRCVRICSKSYLTLENGKVFLTGGDLPALDGARVDFRCDPDFHLVGSSRSICSQGQWSTPKPHCQVNRTPHSERRAVYIGALFPMSGGWPGGQACQPAVEMALEDVNSRRDILPDYELKLIHHDSKCDPGQATKYLYELLYNDPIKIILMPGCSSVSTLVAEAARMWNLIVLSYGSSSPALSNRQRFPTFFRTHPSATLHNPTRVKLFEKWGWKKIATIQQTTEVFTSTLDDLEERVKEAGIEITFRQSFFSDPAVPVKNLKRQDARIIVGLFYETEARKVFCEVYKERLFGKKYVWFLIGWYADNWFKIYDPSINCTVDEMTEAVEGHITTEIVMLNPANTRSISNMTSQEFVEKLTKRLKRHPEETGGFQEAPLAYDAIWALALALNKTSGGGGRSGVRLEDFNYNNQTITDQIYRAMNSSSFEGVSGHVVFDASGSRMAWTLIEQLQGGSYKKIGYYDSTKDDLSWSKTDKWIGGSPPADQTLVIKTFRFLSQKLFISVSVLSSLGIVLAVVCLSFNIYNSHVRYIQNSQPNLNNLTAVGCSLALAAVFPLGLDGYHIGRNQFPFVCQARLWLLGLGFSLGYGSMFTKIWWVHTVFTKKEEKKEWRKTLEPWKLYATVGLLVGMDVLTLAIWQIVDPLHRTIETFAKEEPKEDIDVSILPQLEHCSSRKMNTWLGIFYGYKGLLLLLGIFLAYETKSVSTEKINDHRAVGMAIYNVAVLCLITAPVTMILSSQQDAAFAFASLAIVFSSYITLVVLFVPKMRRLITRGEWQSEAQDTMKTGSSTNNNEEEKSRLLEKENRELEKIIAESGGLPRGPPEPPDRLSCDGSRVHLLYK

SEQ ID NO: 55 - protein, 578 aa, Homo sapiens
MLLLLLLAPLFLRPPGAGGAQTPNATSEGCQIIHPPWEGGIRYRGLTRDQVKAINFLPVDYEIEYVCRGEREVVGPKVRKCLANGSWTDMDTPSRCVRICSKSYLTLENGKVFLTGGDLPALDGARVDFRCDPDFHLVGSSRSICSQGQWSTPKPHCQVNRTPHSERRAVYIGALFPMSGGWPGGQACQPAVEMALEDVNSRRDILPDYELKLIHHDSKCDPGQATKYLYELLYNDPIKIILMPGCSSVSTLVAEAARMWNLIVLSYGSSSPALSNRQRFPTFFRTHPSATLHNPTRVKLFEKWGWKKIATIQQTTEVFTSTLDDLEERVKEAGIEITFRQSFFSDPAVPVKNLKRQDARIIVGLFYETEARKVFCEVYKERLFGKKYVWFLIGWYADNWFKIYDPSINCTVDEMTEAVEGHITTEIVMLNPANTRSISNMTSQEFVEKLTKRLKRHPEETGGFQEAPLAYDAIWALALALNKTSGGGGRSGVRLEDFNYNNQTITDQIYRAMNSSSFEGVSGHVVFDASGSRMAWTLIEQLQGGSYKKIGYYDSTKDDLSWSKTDKWIVISRTHSPT

SEQ ID NO: 56 - protein, 941 aa, Homo sapiens
MASPRSSGQPGPPPPPPPPPARLLLLLLLPLLLPLAPGAWGWARGAPRPPPSSPPLSIMGLMPLTKEVAKGSIGRGVLPAVELAIEQIRNESLLRPYFLDLRLYDTECDNAKGLKAFYDAIKYGPNHLMVFGGVCPSVTSIIAESLQGWNLVQLSFAATTPVLADKKKYPYFFRTVPSDNAVNPAILKLLKHYQWKRVGTLTQDVQRFSEVRNDLTGVLYGEDIEISDTESFSNDPCTSVKKLKGNDVRIILGQFDQNMAAKVFCCAYEENMYGSKYQWIIPGWYEPSWWEQVHTEANSSRCLRKNLLAAMEGYIGVDFEPLSSKQIKTISGKTPQQYEREYNNKRSGVGPSKFHGYAYDGIWVIAKTLQRAMETLHASSRHQRIQDFNYTDHTLGRIILNAMNETNFFGVTGQVVFRNGERMGTIKFTQFQDSREVKVGEYNAVADTLEIINDTIRFQGSEPPKDKTIILEQLRKISLPLYSILSALTILGMIMASAFLFFNIKNRNQKLIKMSSPYMNNLIILGGMLSYASIFLFGLDGSFVSEKTFETLCTVRTWILTVGYTTAFGAMFAKTWRVHAIFKNVKMKKKIIKDQKLLVIVGGMLLIDLCILICWQAVDPLRRTVEKYSMEPDPAGRDISIRPLLEHCENTHMTIWLGIVYAYKGLLMLFGCFLAWETRNVSIPALNDSKYIGMSVYNVGIMCIIGAAVSFLTRDQPNVQFCIVALVIIFCSTITLCLVFVPKLITLRTNPDAATQNRRFQFTQNQKKEDSKTSTSVTSVNQASTSRLEGLQSENHRLRMKITELDKDLEEVTMQLQDTPEKTTYIKQNHYQELNDILNLGNFTESTDGGKAILKNHLDQNPQLQWNTTEPSRTCKDPIEDINSPEHIQRRLSLQLPILHHAYLPSIGGVDASCVSPCVSPTASPRHRHVPPSFRVMVSGL

SEQ ID NO: 57 - DNA, 720 bp, Artificial Sequence
  source: /note=“Description of Artificial Sequence Synthetic polynucleotide”
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcggctacggcctgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccaaaacgtctatctcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagctaccagtccgccctgagcaaagaccccaacgagaagcgcgatcacatggtcctgctggagttcgtgaccgccgccgggatcactctcggcatggacgagctgtacaagtaa

SEQ ID NO: 58 - DNA, 25 bp, Artificial Sequence
  source: /note=“Description of Artificial Sequence Synthetic oligonucleotide”
gttcttaaggcacaggaactgggac

SEQ ID NO: 59 - DNA, 25 bp, Artificial Sequence
  source: /note=“Description of Artificial Sequence Synthetic oligonucleotide”
gaagttaaccctgtcgttctgcgac

SEQ ID NO: 60 - DNA, 25 bp, Artificial Sequence
  source: /note=“Description of Artificial Sequence Synthetic oligonucleotide”
gttctatagggtctgcttgtcgctc

SEQ ID NO: 61 - DNA, 34 bp, Artificial Sequence
  source: /note=“Description of Artificial Sequence Synthetic probe”
gccagtcccagttcctgtgccttaagaacctcgc

SEQ ID NO: 62 - DNA, 34 bp, Artificial Sequence
  source: /note=“Description of Artificial Sequence Synthetic probe”
gcgagtcgcagaacgacagggttaacttcctcgc

SEQ ID NO: 63 - DNA, 34 bp, Artificial Sequence
  source: /note=“Description of Artificial Sequence Synthetic probe”
gcgagagcgacaagcagaccctatagaacctcgc